(19) United States
(12) Patent Application Publication
(10) Pub. No.: US 2017/0027168 A1
(43) Pub. Date: Feb. 2, 2017

(54) METHODS, PRODUCTS, AND SYSTEMS RELATING TO MAKING, PROVIDING, AND USING NANOCRYSTALLINE (NC) PRODUCTS COMPRISING NANOCRYSTALLINE CELLULOSE (NCC), NANOCRYSTALLINE (NC) POLYMERS AND/OR NANOCRYSTALLINE (NC) PLASTICS OR OTHER NANOCRYSTALS OF CELLULOSE COMPOSITES OR STRUCTURES, IN COMBINATION WITH OTHER MATERIALS

(71) Applicant: Stephan Heath, Littleton, CO (US)

(72) Inventor: Stephan Heath, Littleton, CO (US)

(21) Appl. No.: 15/220,046

(22) Filed: Jul. 26, 2016

(30) Foreign Application Priority Data
Jul. 27, 2015 (CA) 2898513

(51) Int. Cl. A01N 25/30; A61L 27/50; A61L 27/34

(52) U.S. Cl. CPC A01N 25/30; A61L 27/34; A61L 27/50; A61L 2400/12

(57) ABSTRACT

Methods, apparatus, products, and/or systems relating to making or using nanocrystalline (NC) products comprising a combination of one or more of nanocrystalline cellulose (NCC), nanocrystalline (NC) polymers, nanocrystalline (NC) plastics or other nanocrystals of cellulose composites or structures that have been processed into one or more of solid, flake, particles or other forms with vapor processing, solid state processing, liquid processing or other processing methods that can optionally be combined with other materials for different nanocrystalline (NC) applications, products or uses, and the like.

### FIELD OF THE INVENTION

The invention relates to methods, apparatus, products, and/or systems relating to making or using nanocrystalline (NC) products comprising a combination of one or more of nanocrystalline cellulose (NCC), nanocrystalline (NC) polymers, nanocrystalline (NC) plastics or other nanocrystals of cellulose composites or structures that have been processed into one or more of solid, flake, particles or other forms with vapor processing, solid state processing, liquid processing or other processing methods that can optionally be combined with other materials for different nanocrystalline (NC) applications, products or uses.

### BACKGROUND

Cellulose is probably the most abundant organic compound in the world, which mostly produced by plants. It is the most structural component in herbal cells and tissues. Cellulose is a natural long chain polymer that plays an important role in human food cycle indirectly. This polymer has versatile uses in many industries such as veterinary cellulose in foods, wood and paper, fibers and clothes, cosmetic and pharmaceutical industries as excipient. Cellulose has very semi-synthetic derivatives and many different nanocrystalline (NC) applications for the pharmaceutical, medical and cosmetic industries. Cellulose ethers and cellulose esters are two main groups of cellulose derivatives with different physicochemical and mechanical properties. These polymers are broadly used in the formulation of dosage forms and healthcare products.

Nanocrystals of cellulose (nanocrystalline cellulose, (NCC)) can be obtained by processing and/or purifying components of natural materials such as trees and/or willow shrubs to orange pulp and/or the pomace left behind after apple cider production. By mixing nanocrystalline cellulose (NCC) with other materials, such as plastics, structural bulk materials or metals, the strength of a product can be substantially increased. Production of nanocrystalline cellulose (NCC) starts with processed wood, which has had compounds such as lignin and hemicellulose removed. It is then milled into a pulp and hydrolyzed in acid to remove impurities before being separated and concentrated as crystals into a thick paste that can be applied to surfaces as a laminate or processed into strands, forming nanofibrils. These are hard, dense and tough, and can be forced into different shapes and sizes. When freeze-dried, nanocrystalline cellulose (NCC) is lightweight, absorbent and good for insulating.

In addition to being used as strengtheners, nanocrystalline cellulose (NCC) can optionally be used for many innovative biomedical applications uses as, a viral inhibitor, antiviral ointments, artificial joints, antibacterial medical coating applications, disposable medical equipment and clinical applications. Nanocrystalline cellulose (NCC) can optionally be used for many other medical or dietary uses, including cellulose vegetable or gelatin capsules for dietary supplements, medications, vitamins, marijuana oils, cannabis oils and other types of oils for cancer treatments, and other medical uses. There are many other uses for nanocrystalline cellulose (NCC) including smart packaging and intelligent labeling technology for food, beverages, pharmaceutical and household products. Thus, there is a need to provide a new class of super materials with superior properties using nanocrystalline cellulose (NCC) that can be combined with other materials for different nanocrystalline (NC) applications, products or uses.

### SUMMARY OF THE INVENTION

Alternative embodiments of the invention optionally relate to methods, apparatus, products, and/or systems relating to making or using nanocrystalline (NC) products comprising a combination of:
(i) one or more of nanocrystalline cellulose (NCC), nanocrystalline (NC) plastics or nanocrystalline (NC) polymers or other nanocrystals of cellulose composites or structures that have been processed into one or more of solid, flake, particles, liquid, non-liquid, spray dried, non-spray dried, bulk, cellulose, coating applications, composite material, components, powder, paste, pulp, fibers, foam, gel, resin, wax, wood chips, wood pulp, bamboo pulp, bleached pulp, wood-based fibers, plant fibers, pulp fibers, extract, seeds, encapsulated, grains, tablets, or other forms, optionally with vapor processing, solid state processing, liquid processing or other processing methods in combination with:
(ii) other materials:
   that are optionally combined with other materials to provide nanocrystalline (NC) products for different product applications, products and/or uses.
(iii) different types of metals, non-metals, amorphous metals or alloys, copper alloy, cobalt alloy, silver alloy, aluminum, steel, kevlar, cast iron, tungsten, chromium, titanium, mechanical alloying or other types of alloys, composite nanocrystalline (NC) coating agents, structural bulk materials, metals or elements, ultra hard nanocrystalline (NC) coating applications, construction applications, ceramics, plastics and/or other nanocrystals of cellulose materials that are optionally combined with other materials to provide for reinforcing materials for increased strength and/or hardness, nanocrystalline (NC) coating applications, compressibility and strength, corrosion resistance, nanocrystalline (NC) coating applications and thin films, conducts electricity, higher electrical resistance, increased specific heat capacity, thermal expansion, optical properties, mechanical properties, elastic properties, strength & hardness, ductility & toughness, electrical properties, magnetic properties, chemical properties, catalytic properties, barrier properties, nanocrystalline cores for large power transformers, lower thermal conductivity, insulation and/or improved thermal properties, optical properties, mechanical properties, elastic properties, strength & hardness, ductility & toughness, electrical properties, chemical properties, magnetic properties for different product applications, products and/or uses.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of bottles, water bottles, caps, engineered wood, furniture, hardwood floors, replacement of plastic or glass consumer products, packaged goods and other end use products with nanocrystalline (NC) products, replacement of petroleum-based or glass consumer products, packaged goods and other end use products with nanocrystalline (NC) products, containers, food and/or beverage containers, lids, plastics, personal care products, chemicals, cellulose in foods, pharmaceutical products, carbohydrate additives, thickeners, flavor carriers, suspension stabilizers, food additives, animal feed, animal feed additives, pet food, pet food additives, pet supplies, pet treats, cosmetic additives, sugar substitute, sweeteners, artificial sweeteners, amino acid regulators, acidity regulators, anticaking agents, applications as taste masking agents, disintegrating agents, binders in granulation process, fillers in solid dosage forms, thickening and stabilizing agents, gelling agents, compressibility enhancers, coating agents, drug delivery systems, pharmaceutical coatings process, medical coating applications, topical ophthalmic protectant and lubricant and the like, antifoaming agents, antibacterial agents, anti-aging products, antioxidants, absorption blocking agents, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of carcinogen blocking agents, cellulose vegetable or gelatin capsules for dietary supplements, medications, vitamins, marijuana oils, cannabis oils, hash oils, hemp oils and other types of oils for cancer treatment, pharmaceutical uses and other medical uses, encapsulation products, cholesterol blocking agents, fat blocking agents, caloric blocking agents, blocking sugar absorption, neuromuscular blocking agents, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of food coloring, color retention agents, emulsifiers, natural or artificial flavors, flavor enhancers, flour treatment agent, glazing agents, humectants, tracer gas, preservatives, stabilizers, thickeners, smart packaging and intelligent labeling technology for food, beverages, pharmaceutical and household products, sunscreens, coatings, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of creams or ointments, nanocrystalline (NC) wound dressings, nanocrystalline (NC) silver wound dressings, wound dressings, surgical dressings, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of ultra hard nanocrystalline (NC) coating applications, fiber optic nanocrystalline (NC) coating applications, synthetic nanocrystalline (NC) diamonds, sensor coating applications, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of rubber composites, synthetic rubber, alloys, tires, petroleum-based products, filters, lightweight body armor, ballistic glass, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of thin film and/or textiles, nanocomposites, as well as optionally one or more of biosensing, biomedical applications, treatment for cancer, biocomposites for bone replacement and tooth repair, grafting, antibacterial medical nanocrystalline (NC) coatings, pharmaceutical coating applications, health applications, weight loss applications, viral inhibitor, antiviral ointments and surfaces, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of synthetic fibers, cigarette additives, cigarette ingredients, cellulose cigarette tobacco, cigarette wadding, cigarette filters, cigarette paper, cellulose tobacco products and the like,

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of disposable medical equipment, coatings for medical applications, medical implants, breast implant devices, microchip implants or other types of implants, artificial heart valves, artificial ligaments, hip joints, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of advanced reinforced coating applications, composite materials filter to purify liquids, water purification applications, and the like, filter out blood cells during transfusions, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of providing filters that will trap dangerous chemicals and other toxins in cigarettes and/or other tobacco products.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of implantable microchips, implantable biocompatible device, biosensors, microfluidics, computer chips, flexible screens, flexible electronic displays, flat panel displays, bendable batteries, wearable batteries, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of ultra absorbent aerogels.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of clothing, transportation, components or parts for computers or hand-held portable devices, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of skin tissue repair and other cosmetic or dermatology uses.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of electronics, flexible electronic displays, batteries, catalysis, ceramics, magnetic data storage, telecommunication and data communication components, electronic applications with a higher quality energy storage capacity for use in a variety of industrial and portable consumer electronic products, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of advanced reinforced coating applications, composite materials, kinetic energy penetrators, improved insulation materials, phosphors, tougher and harder cutting tools, elimination of pollutants, high energy density batteries, cell phones, other hand held devices, toys, watches, high power magnets, high sensitivity sensors, automobiles with greater fuel efficiency, aerospace components with enhanced performance characteristics, better and future weapons platforms, longer lasting satellites, ceramic nanocrystalline (NC) coating applications, silicon thin films, electrochromic display devices, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of longer lasting medical implants or devices, household items, microprocessor in athletic shoes, detergents for washing, fabric softener, baseball bats, tennis rackets, motorcycle helmets, automobile bumpers, luggage, and power tool housings can make them simultaneously lightweight, stiff, durable, and resilient, nanoscale additives to or surface treatments of fabrics help them resist wrinkling, staining, and bacterial growth, and provide lightweight ballistic energy deflection in personal body armor, nanoscale thin films on eyeglasses, computer and camera displays, windows, and other surfaces can make them water-repellent, antireflective, self-cleaning, resistant to ultraviolet or infrared light, antifog, antimicrobial, scratch-resistant, or electrically conductive. Nanoscale materials in cosmetic products provide greater clarity or coverage; cleansing; absorption; personalization; and antioxidant, anti-microbial, and other health properties in sunscreens, cleansers, complexion treatments, creams and lotions, shampoos, and specialized makeup, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of nano-engineered materials in the food industry include nanocomposites in food containers to minimize carbon dioxide leakage out of carbonated beverages, or reduce oxygen inflow, moisture outflow, or the growth of bacteria in order to keep food fresher and safer and longer; nanosensors built into plastic packaging to warn against spoiled food; nanosensors for detection of salmonella, pesticides, and other contaminates on food before packaging and distribution; and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of nano-engineered materials in automotive products include high-power rechargeable battery systems; thermoelectric materials for temperature control; lower-rolling-resistance tires; high-efficiency/low-cost sensors and electronics; thin-film smart solar panels; and fuel additives and improved catalytic converters for cleaner exhaust and extended range; nano-engineered materials make superior household products such as degreasers and stain removers; environmental sensors, alert systems, air purifiers and filters; and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of antibacterial cleansers; and specialized paints and sealing products; nanoscale transistors that are faster, more powerful, and increasingly energy-efficient and ability to store computer's memory on a single tiny chip; displays for many new TVs, laptop computers, cell phones, digital cameras, and other devices incorporate nanostructured polymer films known as organic light-emitting diodes, or OLEDs. OLED screens offer brighter images in a flat format, as well as wider viewing angles, lighter weight, better picture density, lower power consumption, and longer lifetimes; other computing and electronic products include flash memory chips for iPod nanos; and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of ultra responsive hearing aids; antimicrobial/antibacterial coatings on mouse/keyboard/cell phone casings; conductive inks for printed electronics for RFID/smart cards/smart packaging; more life-like video games; and flexible displays for e-book readers; and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of gold nanoparticles can optionally be used to detect early-stage Alzheimer's disease; molecular imaging for the early detection where sensitive biosensors constructed of nanoscale components (e.g., nanocantilevers, nanowires, and nanochannels) can recognize genetic and molecular events and have reporting capabilities, thereby offering the potential to detect rare molecular signals associated with malignancy; multifunctional therapeutics where a nanoparticles serves as a platform to facilitate its specific targeting to cancer cells and delivery of a potent treatment, minimizing the risk to normal tissues; microfluidic chip-based nanolabs capable of monitoring and manipulating individual cells and nanoscale probes to track the movements of cells and individual molecules as they move about in their environments; spur the growth of nerve cells, e.g., in damaged spinal cord or brain cells; imaging technology to measure the amount of an antibody-nanoparticles complex that accumulates specifically in plaque in arteries; and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of nano-engineering of steel, concrete, asphalt, and other cementitious materials, and their recycled forms, offers great promise in terms of improving the performance, resiliency, and longevity of highway and transportation infrastructure components while reducing their cost; building and/or construction materials and products, e.g., but not limited to, concrete, reinforced concrete, building materials using plastics, wood, alloys, or polymers, insulation, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of fertilizers, insulation, pesticides, herbicides, fungi, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of batteries, medical prostheses, coatings for medical applications, medical implants, breast implant devices, microchip implants or other types of implants, orthopedic implants, dental implants or other medical products, surgical devices, wound care products, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of disease-fighting and anti-aging products, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of structural components, solar panels, solar cells, silicon thin films, hard chrome nanocrystalline (NC) coating applications, industrial and military applications, protective shielding, weapon applications, sports and/or leisure products, aerospace and transportation structures and/or automotive applications, aviation applications, replacement of hard chromium plating in aircraft manufacturing, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of flexible screens, flexible electronic displays, flat panel displays, bendable batteries, wearable batteries, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of ultra absorbent aerogels, clothing, transportation fuels, biofuels, liquid fuels, chemical, fuel and/or lubrication industries, and the like.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more for smart packaging and intelligent labeling technology for food, beverages, pharmaceutical and household products.

The invention can optionally provide wherein the nanocrystalline (NC) products comprising a combination of two or more of nanocrystalline cellulose (NCC), nanocrystalline (NC) plastics or nanocrystalline (NC) polymers or other nanocrystals of cellulose composites or structures that are optionally combined with other materials for different nanocrystalline (NC) applications, products or uses, and the like, such as, but not limited to, one or more of metals, non-metals, amorphous metals or alloys, copper alloy, cobalt alloy, silver alloy, aluminum, steel, kevlar, cast iron, tungsten, chromium, titanium, mechanical alloying or other types of alloys, composite nanocrystalline (NC) coating agents, structural bulk materials, metals or elements, ultra hard nanocrystalline (NC) coating applications, plastics and/or other materials, metal replacement, automotive products and/or parts, electronics, nanocrystalline (NC) injection molding applications, and/or different nanocrystalline (NC) applications, products or uses, and the like.

The invention can optionally provide wherein the nanocrystalline cellulose (NCC) reflect on their surface or internally specific wavelengths of light or EMF radiation that can optionally reflect on or penetrate one or more of the skin, tissues, cavities, or orifices of the body and provide specific effects that can optionally be therapeutic or diagnostic. Tracking the reflection of these wavelengths from one or more sensors can optionally provide health or medical professionals with a variety of diagnostic or therapeutic information or treatment. For example, they can optionally provide information relating to one or more of changes in the reflection spectrum as a joint is stressed at different angles, whether sutures used to sew up incisions have dissolved, or how much of a drug implanted in a polymer has been delivered to a patient.

The invention can optionally provide for absorption of electromagnetic wave radiation using nanocrystalline magnetic materials to reduce the harmful effects of electromagnetic waves on the human body through electromagnetic shielding and other nearby devices causing them to malfunction.

The invention can optionally provide the nanocrystalline cellulose (NCC) or products and/or other materials that can optionally be combined with other materials, e.g., but not limited to, one or more of plastic, metals, glass, aluminum, steel, kevlar, cast iron, fibers, alloys and/or other composites that can optionally increase strength and/or hardness and/or used for construction applications and multiple of other nanocrystalline (NC) applications, products or uses.

To satisfy the long-felt but unsolved needs identified above, at least one embodiment of the invention is directed towards a method of making or using a nanocrystalline (NC) product comprising one or more of an NC: cellulose material, polymer, or plastic. Such a method optionally comprises one or more of the steps of: providing an at least one nanocrystalline cellulose (NCC), nanocrystalline (NC) polymers, nanocrystalline (NC) plastics, or other nanocrystals of cellulose composites or structures, and adding the at least one nanocrystalline cellulose (NCC), nanocrystalline (NC) polymers, nanocrystalline (NC) plastics, or other nanocrystals of cellulose composites or structures to a substrate, component, or additive in the dry or wet end of a nanocrystalline (NC) product making process, wherein the at least one nanocrystalline cellulose (NCC), nanocrystalline (NC) polymers, nanocrystalline (NC) plastics, or other nanocrystals of cellulose composites or structures is substantially distributed on, near, or adjacent to the surface of the substrate. The at least one nanocrystalline cellulose (NCC), nanocrystalline (NC) polymers, nanocrystalline (NC) plastics, or other nanocrystals of cellulose composites or structures can optionally be distributed with the use of a size press or other suitable manufacturing device component.

The method can optionally include a method for producing nanocrystalline (NC) products comprising a combination of one or more of nanocrystalline cellulose (NCC), nanocrystalline (NC) polymers and/or nanocrystalline (NC) plastics, optionally comprising one or more:

(a) combining at least one nanocrystalline cellulose (NCC), nanocrystalline (NC) polymers, nanocrystalline (NC) plastics, or other nanocrystals of cellulose composites or structures with at least one substrate, component, or additive, in at least a partial dry form, to provide a Nanocrystalline (NC) composition, wherein the at least one NC cellulose material, polymer, or plastic, is substantially distributed on the surface of the substrate, component, or additive, and wherein the at least one nanocrystalline cellulose (NCC), nanocrystalline (NC) polymers, nanocrystalline (NC) plastics, or other nanocrystals of cellulose composites or structures is a branched polymer having at least a first polymer chain extending from a nanocrystalline (NC) cellulose core and at least one branch diverting away from the first polymer chain; and

(b) processing the nanocrystalline (NC) composition using at least vapor processing, solid state processing, liquid processing or other processing methods to form a nanocrystalline (NC) product comprising one or more of a solid, flake, particles, liquid, non-liquid, spray dried, non-spray dried, bulk, cellulose, coating applications, composite material, components, powder, paste, pulp, fibers, foam, gel, resin, wax, wood chips, wood pulp, bamboo pulp, bleached pulp, wood-based fibers, plant fibers, pulp fibers, extract, seeds, encapsulated, grains, tablets or other forms wherein the nanocrystalline (NC) product reflect specific wavelengths that can optionally penetrate one or more of the skin, tissues, cavities, or orifices of the body.

The method can optionally further comprise wherein the nanocrystalline (NC) product comprises a combination of two or more of nanocrystalline cellulose (NCC), nanocrystalline (NC) polymers or nanocrystalline (NC) polymers structures.

The method can optionally further comprise providing the nanocrystalline (NC) product in one or more of bottles, water bottles, caps, engineered wood, furniture, hardwood floors, containers, food or beverage containers, lids, plastics, personal care products, chemicals, pharmaceutical products, carbohydrate additives, thickeners, flavor carriers, suspension stabilizers, food additives, animal feed, animal feed additives, pet food, pet food additives, pet supplies, pet medications or pet treats, cosmetic additives, sugar substitute, sweeteners, artificial sweeteners, amino acid regulators, acidity regulators, anticaking agents, applications as taste masking agents, disintegrating agents, binders in granulation process, fillers in solid dosage forms, thickening and stabilizing agents, gelling agents, compressibility enhancers, coating agents, drug delivery systems, pharmaceutical coatings process, medical coating applications, topical ophthalmic protectant and lubricant and the like, antifoaming agents, antibacterial agents, anti-aging products, antioxidants, absorption blocking agents, carcinogen blocking agents, cellulose vegetable or gelatin capsules for dietary supplements, medications, vitamins, marijuana oils, cannabis oils, hash oils, hemp oils and other types of oils for cancer treatment, pharmaceutical uses and other medical uses, encapsulation products, cholesterol blocking agents, fat blocking agents, caloric blocking agents, blocking sugar absorption, neuromuscular blocking agents, food coloring, color retention agents, emulsifiers, natural or artificial flavors, flavor enhancers, flour treatment agent, glazing agents, humectants, tracer gas, preservatives, stabilizers, thickeners, smart packaging and intelligent labeling technology for food, beverages, pharmaceutical and household products, sunscreens, coatings, creams or ointments, nanocrystalline (NC) wound dressings, nanocrystalline (NC) silver wound dressings, wound dressings, surgical dressings, rubber composites, synthetic rubber, alloys, tires, petroleum-based products, filters, lightweight body armor, ballistic glass, thin films, textiles, and nanocomposites, and the like.

The method can optionally further comprise providing the nanocrystalline (NC) product in one or more of: disposable medical equipment or medical implant, artificial heart valves, artificial ligaments, artificial hip joints, and the like, and other artificial components, advanced reinforced coating applications, composite materials, filter to purify liquids, water purification applications, and the like, filter out blood cells during transfusions, trap dangerous chemicals in cigarettes, implantable microchips, implantable biocompatible device, biosensors, microfluidics, computer chips, flexible screens, flexible electronic displays, flat panel displays, bendable batteries, wearable batteries, ultra absorbent aerogels, clothing, transportation, components or parts for computers or hand-held portable devices, skin tissue repair compositions, electrical or electronic components, batteries, catalysis, ceramics, magnetic data storage, telecommunication and data communication components, building or construction materials and products, industrial materials, insulation, fertilizers, pesticides, herbicides, fungi, batteries, sports or leisure products, aerospace components with enhanced performance characteristics, better and future weapons platforms, longer lasting satellites, ceramic nanocrystalline (NC) coating applications, silicon thin films, electrochromic display devices, automotive products and/or parts, electronics, flexible screens, flexible electronic displays, flat panel displays, bendable batteries, wearable batteries, ultra absorbent aerogels, clothing, transportation fuels, biofuels, liquid fuels, chemical, fuel or lubrication products, metals, non-metals, amorphous metals or alloys, copper alloy, cobalt alloy, silver alloy, metallic materials, metals or elements, ultra hard nanocrystalline (NC) coating applications, construction applications, ceramics, plastics and/or other materials, alloys, copper alloy, cobalt alloy, silver alloy, metal replacement compositions, ceramics, and the like.

The method can optionally further comprise tracking the reflection or absorption of the wavelengths from one or more sensors to provide diagnostic or therapeutic information, wherein the information comprises one or more of changes in a reflection spectrum generated from reflecting the wavelengths off of said skin, joint or tissue.

The method can optionally further comprise wherein the reflection spectrum is compared to health spectrum reflected off of health skin, joints or tissue, and where the change in wavelengths in the diagnostic or therapeutic information indicates a status of treatment, disease or non-healthy condition in the skin, joint or tissue.

The method can optionally further comprise wherein the status is selected from the disease or non-healthy condition of skin, joint or tissue; whether sutures used to sew up incisions have dissolved, or how much of a drug implanted in a polymer has been delivered to a patient.

The method can optionally further comprise wherein the combining step (a) further comprises adding to the composition at least one material selected from a plastic, a form or alloy of metal, a form or alloy of nanocrystalline copper, nanocrystalline aluminum, nanocrystalline steel, kevlar, cast iron, tungsten, chromium, titanium, mechanical alloying or other types of alloys, a fiber, or a composite, wherein the adding results in at least a 10% increase in at least one the tensile strength or hardness of the resulting nanocrystalline (NC) product material.

The method can optionally further comprise wherein the at least one nanocrystalline cellulose (NCC), nanocrystalline (NC) polymers, nanocrystalline (NC) plastics, or other nanocrystals of cellulose composites or structures comprises at least one first branch of the at least one first polymer chain bonded to a nanocrystalline (NC) cellulose core and the first polymer chain is made up of one or more monomers selected from one or more of:
vinyl acetate, acrylic acid, sodium acrylate, ammonium acrylate, methyl acrylate, acrylamide, acrylonitrile, N,N-dimethyl acrylamide, 2-acrylamido-2-methylpropane-1-sulfonic acid, sodium 2-acrylamido-2-methylpropane-1-sulfonate, 3-acrylamidopropyl-trimethyl-ammonium chloride, diallyldimethylammonium chloride, 2-(dimethylamino)ethyl acrylate, 2-(acryloyloxy)-N,N,N-trimethylethanaminium chloride, N,N-dimethylaminoethyl acrylate benzyl chloride quaternary salt, 2-(acryloyloxy)-N,N,N-trimethylethanamninium methyl sulfate, 2-(dimethylamino)ethyl methacrylate, 2-(methacryloyloxy)-N,N,N-trimethylethanaminium chloride, 3-(dimethylamino)propyl methacrylamide, 2-(methacryloyloxy)-N,N,N-trimethylethanaminium methyl sulfate, methacrylic acid, methacrylic anhydride, methyl methacrylate, methacryloyloxy ethyl trimethyl ammonium chloride, 3-methacrylamidopropyl-trimethyl-ammonium chloride, hexadecyl methacrylate, octadecyl methacrylate, docosyl acrylate, n-vinyl pyrrolidone, 2-vinyl pyridine, 4-vinyl pyridine, epichlorohydrin, n-vinyl formamide, n-vinyl acetamide, 2-hydroxyethyl acrylate glycidyl methacrylate, 3-(allyloxy)-2-hydroxypropane-1-sulfonate, 2-(allyloxy)ethanol, ethylene oxide, propylene oxide, 2,3-epoxypropyltrimethylammonium chloride, (3-glycidoxypropyl)trimethoxy silane, epichlorohydrin-dimethylamine, vinyl sulfonic acid sodium salt, sodium 4-styrene sulfonate, caprolactam and any combination thereof;
non-ionic, water-soluble monomers selected from one or more of: acrylamide, methacrylamide, N,N-dimethylacrylamide, N,N-diethylacrylamide, N-isopropylacrylamide, N-vinylformamide, N-vinylmethylacetamide, N-vinyl pyrrolidone, 2-vinyl pyridine, 4-vinyl pyridine, epichlorohydrin, acrylonitrile, hydroxyethyl methacrylate, hydroxyethyl acrylate, hydroxypropyl acrylate, hydroxypropyl methacrylate, hexadecyl methacrylate, octadecyl methacrylate, glycidyl methacrylate, 3-(glycidoxypropyl)trimethoxy silane, 2-allyloxy ethanol, docosyl acrylate, N-t-butylacrylamide, N-methylolacrylamide, epichlorohydrin-dimethylamine, caprolactam, and any combination thereof;
anionic monomers selected from one or more of acrylic acid; methacrylic acid, 2-acrylamido-2-methylpropanesulfonic acid (AMPS), sodium vinyl sulfonate, styrene sulfonate, maleic anhydride, maleic acid, sulfonate itaconate, sulfopropyl acrylate, polymerisable carboxylic or sulphonic acids, crotonic acid, sulfomethylated acrylamide, allylsulfonate, sodium vinyl sulfonate, itaconic acid, acrylamidomethyl butanoic acid, fumaric acid, vinylphosphonic acid, vinylsulfonic acid, vinylsulfonic acid sodium salt, allylphosphonic acid, 3-(allyloxy)-2-hydroxypropane sulfonate, sulfomethyalted acryamide, phosphono-methylated acrylamide, ethylene oxide, propylene oxide, and any salts or combinations thereof; and
cationic monomers selected from one or more of dialkylaminoalkyl acrylates, methacrylates and their quaternary or acid salts.

The method can optionally further comprise wherein at least one second branch of the first polymer chain comprises a different selection of monomers than the at least one first branch of the at least one first polymer chain, the different selection being different in at least one selected from monomer type, or monomer ratio.

The method can optionally further comprise wherein the at least one nanocrystalline cellulose (NCC), nanocrystalline (NC) polymers, nanocrystalline (NC) plastics, or other nanocrystals of cellulose composites or structures increases the dry or wet strength of the substrate, component, or additive.

The method can optionally further comprise wherein the at least one nanocrystalline cellulose (NCC), nanocrystalline (NC) polymers, nanocrystalline (NC) plastics, or other nanocrystals of cellulose composites or structures increases the wet web strength of the substrate, component, or additive.

The method can optionally further comprise wherein the combining step (a) comprises blending the nanocrystalline cellulose (NCC), nanocrystalline (NC) materials, nanocrystalline (NC) components, nanocrystalline (NC) polymers, nanocrystalline (NC) plastics, or other nanocrystals of cellulose composites or structures with a polymer to provide a blend, and adding the blend to the substrate, component, or additive, wherein the blend is substantially distributed on the surface of the substrate, component, or additive, and wherein the at least one nanocrystalline cellulose (NCC), nanocrystalline (NC) polymers, nanocrystalline (NC) plastics, or other nanocrystals of cellulose composites or structures comprises a nanocrystalline (NC) cellulose-core which consists essentially the nanocrystalline (NC) crystallites having a diameter of 5-10 nm.

The method can optionally further comprise wherein the at least one nanocrystalline cellulose (NCC), nanocrystalline (NC) polymers, nanocrystalline (NC) plastics, or other nanocrystals of cellulose composites or structures is combined in step (a) at the wet end and/or in the dry end of the combining.

The method can optionally further comprise wherein the nanocrystalline cellulose (NCC), nanocrystalline (NC) materials, nanocrystalline (NC) components, nanocrystalline (NC) polymers, nanocrystalline (NC) plastics, or other nanocrystals of cellulose composites or structures is added in the combining step (a) as: (i) a coating outside of the substrate, component, or additive; or (ii) dispersed within the substrate, component or additive.

The method can optionally further comprise wherein the nanocrystalline cellulose (NCC), nanocrystalline (NC) materials, nanocrystalline (NC) components, nanocrystalline (NC) polymers, nanocrystalline (NC) plastics, or other nanocrystals of cellulose composites or structures comprises one or more of linear, branched, or cyclic polymers extending from the nanocrystalline (NC) cellulose core or a nanocrystalline (NC) cellulose graft polymer.

The method can optionally further comprise wherein the nanocrystalline (NC) cellulose is selected from one or more of naturally occurring crystals obtained by separating the crystalline cellulose regions from the amorphous cellulose regions of a plant fiber.

The method can optionally further comprise wherein the nanocrystalline (NC) crystallites are 100-500 nm length and comprise between 85% and 97% of the nanocrystalline (NC) cellulose.

The method can optionally further comprise wherein the combining step (a) comprises one or more of:
providing an aqueous mixture comprising partially hydrolyzed forms of the nanocrystalline cellulose (NCC), nanocrystalline (NC) materials, nanocrystalline (NC) components, nanocrystalline (NC) polymers, nanocrystalline (NC) plastics, or other nanocrystals of cellulose composites or structures in a dissolution media;
providing a solution comprising the substrate, component or additive in a polar organic solvent;
combining the mixture with the solution to form a precipitate; and
washing the precipitate with water to remove solvent and dissolution media and produce a wet composite of the nanocrystalline (NC) composition; and
drying the wet composite to produce a dry composite as the nanocrystalline (NC) composition.

The method can optionally further comprise wherein the washing step is carried out continuously or as a batch process selected from one or more of mixing and separating; washing of a cake of the nanocrystalline (NC) composition; dialysis; or combinations thereof.

The method can optionally further comprise wherein the washing step is carried out until the wet composite has a pH between 6 and 7.

The method can optionally further comprise wherein the drying step is carried out at one or more selected from room temperature, heating, cooling; atmospheric pressure, and reduced pressure.

The method can optionally further comprise wherein the dry composite produced is rigid and has (i) a storage modulus of between 1-5 and 20-35 gigapascals, at a temperature of 20 degrees C., or (ii) a storage modulus between 0.1-1 gigapascals and 10-20 gigapascals, at a temperature of 100 degrees Centigrade.

The method can optionally further comprise wherein dry composite is porous and has a density of 0.01 to 10 grams per cubic centimeter and a residual weight of about 1-20% at a temperature of 400 degrees C. and combinations thereof.

The at least one nanocrystalline cellulose (NCC), nanocrystalline (NC) polymers, nanocrystalline (NC) plastics, or other nanocrystals of cellulose composites or structures can optionally be a polymer grafted on to at least one NC core component, compound, or moiety. The at least one nanocrystalline cellulose (NCC), nanocrystalline (NC) polymers, nanocrystalline (NC) plastics, or other nanocrystals of cellulose composites or structures can optionally be a branched or linear polymer having a first polymer chain extending from an NCC core and at least one branch diverting away from the first polymer chain. The branch can optionally be constructed out of one or more different combinations of monomers than the first polymer chain, the different selection being optionally different in one or more of monomer type, monomer ratio, or both. The at least one nanocrystalline cellulose (NCC), nanocrystalline (NC) polymers, nanocrystalline (NC) plastics, or other nanocrystals of cellulose composites or structures can optionally increase the dry or wet strength of the substrate, component, or additive.

The invention can optionally include a method of making a nanocrystalline (NC) composite or product, comprising: (a) providing an aqueous mixture comprising partially hydrolyzed cellulose in a dissolution media; (b) providing a solution comprising a aliphatic polyester in a polar organic solvent; (c) combining the mixture with the solution to form a precipitate; and (d) washing the precipitate with water to remove solvent and dissolution media and form a wet composite; and then (e) drying the wet composite to form a dry composite.

In one or more optional embodiments, the combining step, and the washing step, can optionally be carried out in a form or mold; and the method further comprises the step of: (e) releasing the composite from the form or mold to produce a composite product (optionally having a shape corresponding to the shape of the form or mold), and then optionally (f) cutting or grinding the product to further define the features thereof. Other optional embodiments include a shaped product produced by a process as described herein or known in the art or a particulate nanocrystalline (NC) composite produced by the process described herein.

The method can optionally further comprise wherein the form or alloy of metal is selected from iron or titanium based nanocrystalline magnetic materials that absorb or reflect electromagnetic energy in the range of 10 to 100 kHz that are provided with crystal diameters in the range of 10-15 nm.

The method can optionally further comprise wherein the iron or titanium based nanocrystalline magnetic material is selected from a FeSiBNbCu alloy, dialectric TiO2 powder, or BaTiO3 powder.

The method can optionally further comprise a method for blocking or absorbing electromagnetic (EM) radiation, comprising one or more of:

providing a nanocrystalline (NC) product providing by a method according to claim 25;

blocking or absorbing EM radiation using the nanocrystalline (NC) product.

The method can optionally further comprise wherein said blocking or absorption is used for diagnostic comparison of normal and disease conditions in a mammalian subject using said nanocrystalline (NC) product.

The method can optionally further comprise wherein said blocking or absorption is used for treatment of skin, joint or tissue pathogenic conditions in a mammalian subject using said nanocrystalline (NC) product.

The method can optionally further comprise wherein said nanocrystalline (NC) product is selected from one or more of bottles, water bottles, caps, engineered wood, furniture, hardwood floors, containers, food or beverage containers, lids, plastics, personal care products, chemicals, pharmaceutical products, carbohydrate additives, thickeners, flavor carriers, suspension stabilizers, food additives, animal feed, animal feed additives, pet food, pet food additives, pet supplies, pet medications or pet treats, cosmetic additives, sugar substitute, sweeteners, artificial sweeteners, amino acid regulators, acidity regulators, anticaking agents, applications as taste masking agents, disintegrating agents, binders in granulation process, fillers in solid dosage forms, thickening and stabilizing agents, gelling agents, compressibility enhancers, coating agents, drug delivery systems, pharmaceutical coatings process, medical coating applications, topical ophthalmic protectant and lubricant and the like, antifoaming agents, antibacterial agents, anti-aging products, antioxidants, absorption blocking agents, carcinogen blocking agents, cellulose vegetable or gelatin capsules for dietary supplements, medications, vitamins, marijuana oils, cannabis oils, hash oils, hemp oils and other types of oils for cancer treatment, pharmaceutical uses and other medical uses, encapsulation products, cholesterol blocking agents, fat blocking agents, caloric blocking agents, blocking sugar absorption, neuromuscular blocking agents, food coloring, color retention agents, emulsifiers, natural or artificial flavors, flavor enhancers, flour treatment agent, glazing agents, humectants, tracer gas, preservatives, stabilizers, thickeners, smart packaging and intelligent labeling technology for food, beverages, pharmaceutical and household products, sunscreens, coatings, creams or ointments, nanocrystalline (NC) wound dressings, nanocrystalline (NC) silver wound dressings, wound dressings, surgical dressings, rubber composites, synthetic rubber, alloys, tires, petroleum-based products, filters, lightweight body armor, ballistic glass, thin films, textiles, and nanocomposites, and the like.

The method can optionally further comprise wherein said nanocrystalline (NC) product is selected from one or more of biomedical applications, treatment for cancer, biocomposites for bone replacement and tooth repair, grafting, antibacterial medical nanocrystalline (NC) coatings, pharmaceutical coating applications, health applications, weight loss applications, viral inhibitor, antiviral ointments and surfaces, synthetic fibers, cigarette additives, cigarette ingredients, cellulose cigarette tobacco, cigarette wadding, cigarette filters, cigarette paper, cellulose tobacco products and the like, disposable medical equipment, coatings for medical applications, medical implants, breast implant devices, microchip implants or other types of implants, artificial heart valves, artificial ligaments, hip joints, and the like, advanced reinforced coating applications, composite materials, filter to purify liquids, water purification applications, and the like, filter out blood cells during transfusions, trap dangerous chemicals in cigarettes, implantable microchips, implantable biocompatible device, biosensors, microfluidics, computer chips, flexible screens, flexible electronic displays, flat panel displays, bendable batteries, wearable batteries, ultra absorbent aerogels, clothing, transportation, components or parts for computers or hand-held portable devices, skin tissue repair, electronics, flexible electronic displays, batteries, catalysis, ceramics, magnetic data storage, telecommunication and data communication components, electronic applications with a higher quality energy storage capacity for use in a variety of industrial and portable consumer electronic products, advanced reinforced coating applications, composite materials, kinetic energy penetrators, improved insulation materials, fertilizers, insulation, pesticides, herbicides, fungi, batteries, medical prostheses, coatings for medical applications, medical implants, breast implant devices, microchip implants or other types of implants, orthopedic implants, dental implants or other medical products, surgical devices, wound care products, disease-fighting and anti-aging products, phosphors, tougher and harder cutting tools, elimination of pollutants, high energy density batteries, cell phones, other hand held devices, toys, watches, high power magnets, high sensitivity sensors, automobiles with greater fuel efficiency, aerospace components with enhanced performance characteristics, better and future weapons platforms, longer lasting satellites, ceramic nanocrystalline (NC) coating applications, silicon thin films, electrochromic display devices, longer lasting medical implants or devices, household items, microprocessor in athletic shoes, detergents for washing, fabric softener, baseball bats, tennis rackets, motorcycle helmets, automobile bumpers, luggage, and power tool housings can make them simultaneously lightweight, stiff, durable, and resilient, nanoscale additives to or surface treatments of fabrics help them resist wrinkling, staining, and bacterial growth, and provide lightweight ballistic energy deflection in personal body armor, nanoscale thin films on eyeglasses, computer and camera displays, windows, and other surfaces can make them water-repellent, antireflective, self-cleaning, resistant to ultraviolet or infrared light, antifog, antimicrobial, scratch-resistant, or electrically conductive. Nanoscale materials in cosmetic products provide greater clarity or coverage; cleansing; absorption; personalization; and antioxidant, anti-microbial, and other health properties in sunscreens, cleansers, complexion treatments, creams and lotions, shampoos, and specialized makeup, nano-engineered materials in the food industry include nanocomposites in food containers to minimize carbon dioxide leakage out of carbonated beverages, or reduce oxygen inflow, moisture outflow, or the growth of bacteria in order to keep food fresher and safer and longer; nanosensors built into plastic packaging to warn against spoiled food; nanosensors for detection of salmonella, pesticides, and other contaminates on food before packaging and distribution; nano-engineered materials in automotive products include high-power rechargeable battery systems; thermoelectric materials for temperature control; lower-rolling-resistance tires; high-efficiency/low-cost sensors and electronics; thin-film smart solar panels; and fuel additives and improved catalytic converters for cleaner exhaust and extended range; nano-engineered materials make superior household products such as degreasers and stain removers; environmental sensors, alert systems, air purifiers and filters; antibacterial cleansers; and specialized paints and sealing products; nanoscale transistors that are faster, more powerful, and increasingly energy-efficient and ability to store computer's memory on a single tiny chip; displays for many new TVs, laptop computers, cell phones, digital cameras, and other devices incorporate nanostructured polymer films known as organic light-emitting diodes, or OLEDs. OLED screens offer brighter images in a flat format, as well as wider viewing angles, lighter weight, better picture density, lower power consumption, and longer lifetimes; other computing and electronic products include flash memory chips for iPod nanos; ultra responsive hearing aids; antimicrobial/antibacterial coatings on mouse/keyboard/cell phone casings; conductive inks for printed electronics for RFID/smart cards/smart packaging; more life-like video games; and flexible displays for e-book readers; gold nanoparticles can optionally be used to detect early-stage Alzheimer's disease; molecular imaging for the early detection where sensitive biosensors constructed of nanoscale components (e.g., nanocantilevers, nanowires, and nanochannels) can recognize genetic and molecular events and have reporting capabilities, thereby offering the potential to detect rare molecular signals associated with malignancy; multifunctional therapeutics where a nanoparticles serves as a platform to facilitate its specific targeting to cancer cells and delivery of a potent treatment, minimizing the risk to normal tissues; microfluidic chip-based nanolabs capable of monitoring and manipulating individual cells and nanoscale probes to track the movements of cells and individual molecules as they move about in their environments; spur the growth of nerve cells, e.g., in damaged spinal cord or brain cells; imaging technology to measure the amount of an antibody-nanoparticles complex that accumulates specifically in plaque in arteries; nano-engineering of steel, concrete, asphalt, and other cementitious materials, and their recycled forms, offers great promise in terms of improving the performance, resiliency, and longevity of highway and transportation infrastructure components while reducing their cost; building and/or construction materials and products, e.g., but not limited to, concrete, reinforced concrete, building materials using plastics, wood, alloys, or polymers, insulation, and the like, structural components, fertilizers, insulation, pesticides, herbicides, fungi, batteries, medical prostheses, coatings for medical applications, medical implants, breast implant devices, microchip implants or other types of implants, orthopedic implants, dental implants or other medical products, surgical devices, wound care products, disease-fighting and anti-aging products, solar panels, solar cells, silicon thin films, hard chrome nanocrystalline (NC) coating applications, industrial and military applications, protective shielding, weapon applications, sports and/or leisure products, aerospace and transportation structures and/or automotive applications, aviation applications, replacement of hard chromium plating in aircraft manufacturing, flexible screens, flexible electronic displays, flat panel displays, bendable batteries, wearable batteries, ultra absorbent aerogels, clothing, transportation fuels, biofuels, liquid fuels, chemical, fuel and/or lubrication applications, metals, non-metals, amorphous metals or alloys, copper alloy, cobalt alloy, silver alloy, aluminum, steel, kevlar, cast iron, tungsten, chromium, titanium, mechanical alloying or other types of alloys, composite nanocrystalline (NC) coating agents, structural bulk materials, metals or elements, ultra hard nanocrystalline (NC) coating applications, plastics and/or other materials, metal replacement, automotive products and/or parts, electronics, clothing and/or different nanocrystalline (NC) applications, products or uses, and the like.

The method can optionally further comprise wherein said nanocrystalline (NC) product is selected from one or more of replacement of plastic or glass consumer products, packaged goods and other end use products with nanocrystalline (NC) products, replacement of petroleum-based or glass consumer products, packaged goods and other end use products with nanocrystalline (NC) products.

The method can optionally further comprise wherein said NCC crystals can optionally be designed to adsorb viruses and disable them through the use of antiviral ointments and surfaces providing protection against viruses, spread by mosquitoes, by applying ointment containing nanocrystalline cellulose onto the skin. Nanocrystalline cellulose applied, in a non-liquid form, on hospital door handles could be a viral inhibitor to kill viruses and prevent them from spreading.

The method can optionally further comprise wherein said NCC crystals can optionally be used as a drug carrier for the treatment of cancer or other diseases.

The method can optionally further comprise wherein said NCC crystals can optionally be used to produce synthetic fibers, cigarette additives, cigarette ingredients, cellulose cigarette tobacco, cigarette wadding, cigarette filters and/or cigarette paper.

The method can optionally further comprise wherein said NCC crystals can optionally be used to produce synthetic nanocrystalline (NC) diamonds.

The invention can also optionally include, but it not limited to, using or adding nanocrystalline cellulose (NCC), nanocrystalline (NC) plastics or nanocrystalline (NC) polymers or other nanocrystals of cellulose composites or structures with manganese phosphates are of considerable industrial interesting properties nowadays because of their wide applications in laser host, ceramic, dielectric, electric, magnetic, and catalytic processes, including but not limited to, manganese (III) phosphates such as Manganese dihydrogenphosphate dihydrate (Mn(H2PO4)2.2H2O), MnP3O9, MnPO4.H2O, MnPO4, MnHP2O7 and Mn3(PO4)3, which can be made according to known methods, as known in the art, e.g., Danvirutai et al., Journal of Alloys and Compounds 457 (2008) pp. 75-80, entirely incorporated by reference. The invention can also optionally include compositions and methods using the nanocrystalline cellulose (NCC), nanocrystalline (NC) plastics or nanocrystalline (NC) polymers or other nanocrystals of cellulose composites or structures of the invention for use in fertilizers, pesticides and/or herbicides and/or with micronutrients added to fertilizers, such as insoluble micronutrients, smart macronutrients or smart micronutrients, optionally in applications including combining them with nitrogen-phosphorus-potassium (NPK) fertilizers and coating them on NPK fertilizers and seeds, and also in and used with controlled-release fertilizer of zinc encapsulated by a manganese hollow core shell (Soil Science and Plant Nutrition, v.61, (2), pp. 319-326 (2015)), e.g., macronutrients can include one or more of sources or compounds comprising one or more of calcium, carbon, hydrogen, magnesium, nitrogen, oxygen, phosphorus, potassium, or sulphur; and/or micronutrients can include one or more of sources or compounds comprising one or more of boron, chloride, cobalt, copper, iron, molybdenum, manganese, nickel, silicon, sodium, and/or zinc.

The invention can also include adding using or adding nanocrystalline cellulose (NCC), nanocrystalline (NC) plastics or nanocrystalline (NC) polymers or other nanocrystals of cellulose composites or structures to magnesium chloride, potassium chloride and/or sodium chloride; for use with hydroxyapatite, e.g., one or more of reconstruction of bone or teeth, chromotrography, gas sensors, filter to purify liquids, water purification and/or desalination (e.g., polyvinylidenefluoride-co-hexafluoropropylene (PVDF-HFP) membranes containing different amounts of nanocrystalline cellulose (NCC), as known in the art, e.g., Lalia et. al., Desalination v.332, pp. 134-141 (2014)), fertilizers, and drug carriers, based on properties including one or more of powder properties, e.g., particles size, surface area, and morphology, which improve the properties thereof, (e.g., as known in the art, e.g., Klinkaewnarong et al. Current Applied Physics 10 (2010) 521-525).

Nanocrystalline cellulose (NCC), nanocrystalline (NC) plastics or nanocrystalline (NC) polymers or other nanocrystals of cellulose composites or structures can optionally be used in batteries, e.g., NiMH, or Lithium (Li) batteries or rechargeable batteries or super capacitors, as nanocrytalline metal hydrides, including, but not limited to, one or more of structure, electrochemical and electronic properties of nanocrystalline and polycrystalline TiFe-, LaNi5- and Mg2Ni-type phases, which can optionally be prepared by mechanical alloying (MA) followed by annealing or by induction melting method, respectively. The properties of hydrogen host materials can be modified substantially by alloying to obtain the desired storage characteristics, e.g., respective replacement of Fe in TiFe by Ni and/or by Mg, Cr, Mn, Co, Mo, Zr, or for Li batteries, e.g., LiMn2O4, γ-Fe2O3, fluorine-doped tin oxide and potassium manganese oxyiodide or nanocrystalline solid solutions AlySn1-yO2-y/2 (y=0.57, 0.4) as electrode materials for lithium-ion batteries (e.g., Becker et al. Journal of Power Sources, Volume 229, 1 May 2013, Pages 149-158, which can improve not only the discharge capacity but also the cycle life of these electrodes, e.g., nanocrystalline TiFe0.125Mg0.125Ni(0.75) powder, e.g., cobalt substituting nickel in LaNi4-xMn0.75Al0.25Cox alloy greatly improves the discharge capacity and cycle life of LaNi5 material, e.g., nanocrystalline LaNi3.75Mn0.75Al0.25Co0.25 powder.

Super capacitors and batteries can optionally include nanocrystalline transition metal nitrides (TMN) based on vanadium nitride, that can optionally deliver a specific capacitance of 1,340 F/g when tested at low scan rates of 2 mV/s and 554 F/g when tested at high charging rates of 100 mV/s in the presence of a 1M KOH electrolyte; and/or using nanostructured vanadium nitride and controlled oxidation of the surface at the nanoscale can optionally be in super capacitors used in e.g., cars, camcorders and lawn mowers to industrial backup power systems at hospitals and airports.

Nanocrystalline cellulose (NCC), nanocrystalline (NC) plastics or nanocrystalline (NC) polymers or other nanocrystals of cellulose composites or structures can optionally be used in inverter components and materials such as nanocrystalline soft magnetic materials, e.g., of Fe-based soft magnetic material.

Nanocrystalline cellulose (NCC), nanocrystalline (NC) plastics or nanocrystalline (NC) polymers or other nanocrystals of cellulose composites or structures of the invention can also optionally include nanocomposites fabricated by gelation and electro spinning, which can have advantages for improving mechanical properties of both nanocomposite hydrogels and electrospun nanocomposite fibers/mats, as used in the invention, which can optionally include, as known in the art, including multifunctional properties, nanocomposite hydrogels from CNCs and other stimuli responsive polymers, e.g., nanocomposite hydrogels reinforced with CNCs can include one or more of fast temperature, pH, and salt sensitivity, e.g., for controllable drug delivery systems, pharmaceutical coatings process, medical coating applications, topical ophthalmic protectant and lubricant and the like, and other applications, e.g., hydrophilicity, biodegradability, biocompatibility, low cost, and non-toxicity, e.g., tissue engineering. Electrospun nanocomposite fibers can optionally include improved fabrication, morphology, mechanical and/or thermal properties with designed and improved functional characteristics and properties, such as, but not limited to energy-related materials, sensor, barrier films, and tissue engineering scaffolds, as known in the art.

The foregoing and other objects and aspects of the present invention are explained in greater detail in the drawings herein and the specification set forth below. The disclosures of all United States Patent references cited herein are to be incorporated by reference herein in their entirety.

### DETAILED DESCRIPTION

Alternative embodiments of the invention optionally relate to methods, apparatus, products, and/or systems relating to making or using nanocrystalline (NC) products comprising a combination of one or more of nanocrystalline cellulose (NCC), nanocrystalline (NC) plastics or nanocrystalline (NC) polymers or other nanocrystals of cellulose composites or structures that have been processed into one or more of solid, flake, particles, liquid, non-liquid, spray dried, non-spray dried, bulk, cellulose, coating applications, composite material, components, powder, paste, pulp, fibers, foam, gel, resin, wax, wood chips, wood pulp, bamboo pulp, bleached pulp, wood-based fibers, plant fibers, pulp fibers, extract, seeds, encapsulated, grains, tablets or other forms with vapor processing, solid state processing, liquid processing or other processing methods that can optionally be combined with other materials for different nanocrystalline (NC) applications, products or uses, and the like.

The invention can optionally provide wherein the nanocrystalline (NC) products comprising a combination of two or more of nanocrystalline cellulose (NCC), nanocrystalline (NC) polymers or nanocrystalline (NC) plastics structures or other nanocrystals of cellulose composites or structures that are optionally combined with other materials for different nanocrystalline (NC) applications, products or uses, and the like, such as, but not limited to, one or more of bottles, water bottles, caps, engineered wood, furniture, hardwood floors, replacement of plastic or glass consumer products, packaged goods and other end use products with nanocrystalline (NC) products, replacement of petroleum-based or glass consumer products, packaged goods and other end use products with nanocrystalline (NC) products, containers, food and/or beverage containers, lids, plastics, personal care products, chemicals, cellulose in foods, pharmaceutical products, carbohydrate additives, thickeners, flavor carriers, suspension stabilizers, food additives, animal feed, animal feed additives, pet food, pet food additives, pet supplies, pet treats, cosmetic additives, sugar substitute, sweeteners, artificial sweeteners, amino acid regulators, acidity regulators, anticaking agents, applications as taste masking agents, disintegrating agents, binders in granulation process, fillers in solid dosage forms, thickening and stabilizing agents, gelling agents, compressibility enhancers, coating agents, drug delivery systems, pharmaceutical coatings process, medical coating applications, topical ophthalmic protectant and lubricant and the like, antifoaming agents, antibacterial agents, anti-aging products, antioxidants, absorption blocking agents, carcinogen blocking agents, cellulose vegetable or gelatin capsules for dietary supplements, medications, vitamins, marijuana oils, cannabis oils, hash oils, hemp oils and other types of oils for cancer treatment, pharmaceutical uses and other medical uses, encapsulation products, cholesterol blocking agents, fat blocking agents, caloric blocking agents, blocking sugar absorption, neuromuscular blocking agents, food coloring, color retention agents, emulsifiers, natural or artificial flavors, flavor enhancers, flour treatment agent, glazing agents, humectants, tracer gas, preservatives, stabilizers, thickeners, smart packaging and intelligent labeling technology for food, beverages, pharmaceutical and household products, sunscreens, coatings, creams or ointments, nanocrystalline (NC) wound dressings, nanocrystalline (NC) silver wound dressings, wound dressings, surgical dressings, multiple nanocrystalline (NC) coating applications, synthetic nanocrystalline (NC) diamonds, sensor coating applications, rubber composites, synthetic rubber, alloys, tires, petroleum-based products, filters, lightweight body armor, ballistic glass, thin film and/or textiles, nanocomposites, as well as optionally one or more of biosensing, biomedical applications, treatment for cancer, biocomposites for bone replacement and tooth repair, grafting, antibacterial medical nanocrystalline (NC) coatings, pharmaceutical coating applications, health applications, weight loss applications, viral inhibitor, antiviral ointments and surfaces, synthetic fibers, cigarette additives, cigarette ingredients, cellulose cigarette tobacco, cigarette wadding, cigarette filters, cigarette paper, cellulose tobacco products and the like, disposable medical equipment, coatings for medical applications, medical implants, breast implant devices, microchip implants or other types of implants, artificial heart valves, artificial ligaments, hip joints, and the like, advanced reinforced coating applications, composite materials, filter to purify liquids, water purification applications, and the like, filter out blood cells during transfusions, trap dangerous chemicals in cigarettes, implantable microchips, implantable biocompatible device, biosensors, microfluidics, computer chips, flexible screens, flexible electronic displays, flat panel displays, bendable batteries, wearable batteries, ultra absorbent aerogels, clothing, transportation, components or parts for computers or hand-held portable devices, skin tissue repair, electronics, flexible electronic displays, batteries, catalysis, ceramics, magnetic data storage, telecommunication and data communication components, electronic applications with a higher quality energy storage capacity for use in a variety of industrial and portable consumer electronic products, advanced reinforced coating applications, composite materials, kinetic energy penetrators, improved insulation materials, fertilizers, insulation, pesticides, herbicides, fungi, batteries, medical prostheses, coatings for medical applications, medical implants, breast implant devices, microchip implants or other types of implants, orthopedic implants, dental implants or other medical products, surgical devices, wound care products, disease-fighting and anti-aging products, phosphors, tougher and harder cutting tools, elimination of pollutants, high energy density batteries, cell phones, other hand held devices, toys, watches, high power magnets, high sensitivity sensors, automobiles with greater fuel efficiency, aerospace components with enhanced performance characteristics, better and future weapons platforms, longer lasting satellites, ceramic nanocrystalline (NC) coating applications, silicon thin films, electrochromic display devices, longer lasting medical implants or devices, household items, microprocessor in athletic shoes, detergents for washing, fabric softener, baseball bats, tennis rackets, motorcycle helmets, automobile bumpers, luggage, and power tool housings can make them simultaneously lightweight, stiff, durable, and resilient, nanoscale additives to or surface treatments of fabrics help them resist wrinkling, staining, and bacterial growth, and provide lightweight ballistic energy deflection in personal body armor, nanoscale thin films on eyeglasses, computer and camera displays, windows, and other surfaces can make them water-repellent, antireflective, self-cleaning, resistant to ultraviolet or infrared light, antifog, antimicrobial, scratch-resistant, or electrically conductive. Nanoscale materials in cosmetic products provide greater clarity or coverage; cleansing; absorption; personalization; and antioxidant, anti-microbial, and other health properties in sunscreens, cleansers, complexion treatments, creams and lotions, shampoos, and specialized makeup, nano-engineered materials in the food industry include nanocomposites in food containers to minimize carbon dioxide leakage out of carbonated beverages, or reduce oxygen inflow, moisture outflow, or the growth of bacteria in order to keep food fresher and safer and longer; nanosensors built into plastic packaging to warn against spoiled food; nanosensors for detection of salmonella, pesticides, and other contaminates on food before packaging and distribution; nano-engineered materials in automotive products include high-power rechargeable battery systems; thermoelectric materials for temperature control; lower-rolling-resistance tires; high-efficiency/low-cost sensors and electronics; thin-film smart solar panels; and fuel additives and improved catalytic converters for cleaner exhaust and extended range; nano-engineered materials make superior household products such as degreasers and stain removers; environmental sensors, alert systems, air purifiers and filters; antibacterial cleansers; and specialized paints and sealing products; nanoscale transistors that are faster, more powerful, and increasingly energy-efficient and ability to store computer's memory on a single tiny chip; displays for many new TVs, laptop computers, cell phones, digital cameras, and other devices incorporate nanostructured polymer films known as organic light-emitting diodes, or OLEDs. OLED screens offer brighter images in a flat format, as well as wider viewing angles, lighter weight, better picture density, lower power consumption, and longer lifetimes; other computing and electronic products include flash memory chips for iPod nanos; ultra responsive hearing aids; antimicrobial/antibacterial coatings on mouse/keyboard/cell phone casings; conductive inks for printed electronics for RFID/smart cards/smart packaging; more life-like video games; and flexible displays for e-book readers; gold nanoparticles can optionally be used to detect early-stage Alzheimer's disease; molecular imaging for the early detection where sensitive biosensors constructed of nanoscale components (e.g., nanocantilevers, nanowires, and nanochannels) can recognize genetic and molecular events and have reporting capabilities, thereby offering the potential to detect rare molecular signals associated with malignancy; multifunctional therapeutics where a nanoparticles serves as a platform to facilitate its specific targeting to cancer cells and delivery of a potent treatment, minimizing the risk to normal tissues; microfluidic chip-based nanolabs capable of monitoring and manipulating individual cells and nanoscale probes to track the movements of cells and individual molecules as they move about in their environments; spur the growth of nerve cells, e.g., in damaged spinal cord or brain cells; imaging technology to measure the amount of an antibody-nanoparticles complex that accumulates specifically in plaque in arteries; nano-engineering of steel, concrete, asphalt, and other cementitious materials, and their recycled forms, offers great promise in terms of improving the performance, resiliency, and longevity of highway and transportation infrastructure components while reducing their cost; building and/or construction materials and products, e.g., but not limited to, concrete, reinforced concrete, building materials using plastics, wood, alloys, or polymers, insulation, and the like, structural components, insulation, fertilizers, pesticides, herbicides, fungi, batteries, medical prostheses, coatings for medical applications, medical implants, breast implant devices, microchip implants or other types of implants, orthopedic implants, dental implants or other medical products, surgical devices, wound care products, disease-fighting and anti-aging products, solar panels, solar cells, silicon thin films, hard chrome nanocrystalline (NC) coating applications, industrial and military applications, protective shielding, weapon applications, sports and/or leisure products, aerospace and transportation structures and/or automotive industry, aviation industry, flexible screens, flexible electronic displays, flat panel displays, bendable batteries, wearable batteries, ultra absorbent aerogels, clothing, transportation fuels, biofuels, liquid fuels, chemical, fuel and/or lubrication industries, metals, non-metals, amorphous metals or alloys, copper alloy, cobalt alloy, silver alloy, aluminum, steel, kevlar, cast iron, tungsten, chromium, titanium, mechanical alloying or other types of alloys, composite nanocrystalline (NC) coating agents, structural bulk materials, metals or elements, ultra hard nanocrystalline (NC) coating applications, plastics and/or other materials, metal replacement, automotive products and/or parts, electronics, nanocrystalline (NC) injection molding applications, and/or different nanocrystalline (NC) applications, products or uses, and the like.

The invention can optionally provide wherein the materials reflect specific wavelengths that can optionally penetrate one or more of the skin, tissues, cavities, or orifices of the body. Tracking the reflection of these wavelengths from one or more sensors can optionally provide health or medical professionals with a variety of diagnostic or therapeutic information. For example, they can optionally provide information relating to one or more of changes in the reflection spectrum as a joint is stressed at different angles, whether sutures used to sew up incisions have dissolved, or how much of a drug implanted in a polymer has been delivered to a patient.

The invention can optionally provide for absorption of electromagnetic wave radiation using nanocrystalline magnetic materials to reduce the harmful effects of electromagnetic waves on the human body through electromagnetic shielding and other nearby devices causing them to malfunction.

### DEFINITIONS

The following definitions are provided to determine how terms used in this application, and in particular how the claims, are to be construed. The organization of the definitions is for convenience only and is not intended to limit any of the definitions to any particular category.

In the event that the above definitions or a description stated elsewhere in this application is inconsistent with a meaning (explicit or implicit) which is commonly used, in a dictionary, or stated in a source incorporated by reference into this application, the application and the claim terms in particular are understood to be construed according to the definition or description in this application, and not according to the common definition, dictionary definition, or the definition that is incorporated by reference.

Acetate Tow for cigarette filters is a mesh structure of fibers made from cellulose acetate. It is highly effective at removing toxic substances such as tar and nicotine without spoiling the cigarette's flavor or aroma.

Active Packaging, Intelligent Packaging, and Smart Packaging refer to packaging systems used with foods, pharmaceuticals, and several other types of products. They help extend shelf life, monitor freshness, display information on quality, improve safety, and improve convenience. The terms are closely related. Active packaging usually means having active functions beyond the inert passive containment and protection of the product. Intelligent and smart packaging usually involve the ability to sense or measure an attribute of the product, the inner atmosphere of the package, or the shipping environment. This information can be communicated to users or can trigger active packaging functions. Depending on the working definitions, some traditional types of packaging might be considered as “active” or “intelligent”. More often, the terms are used with new technologically advanced systems: microelectronics, computer applications, nanotechnology, etc.

Aliphatic Polyester as used herein can optionally be any suitable aliphatic polyester, including but not limited to polylactic acid, polyglycolic acid, polycaprolactone, polybutylene succinates, polyhydroxyalkanoates, and combinations thereof. Additional examples include, but are not limited to, those described in U.S. Pat. Nos. 8,008,373; 8,003,721; 8,003,719; and 7,994,078, the disclosures of which are incorporated by reference herein in their entirety.

Amino Acids are biologically important organic compounds composed of amine (—NH₂) and carboxylic acid (—COOH) functional groups, along with a side-chain specific to each amino acid. The key elements of an amino acid are carbon, hydrogen, oxygen, and nitrogen, though other elements are found in the side-chains of certain amino acids. About 500 amino acids are known and can be classified in many ways. They can be classified according to the core structural functional groups' locations as alpha- (α-), beta- (β-), gamma- (γ-) or delta- (δ-) amino acids; other categories relate to polarity, pH level, and side-chain group type (aliphatic, acyclic, aromatic, containing hydroxyl or sulfur, etc.). In the form of proteins, amino acids comprise the second-largest component (water is the largest) of human muscles, cells and other tissues. Outside proteins, amino acids perform critical roles in processes such as neurotransmitter transport and biosynthesis. In biochemistry, amino acids having both the amine and the carboxylic acid groups attached to the first (alpha-) carbon atom have particular importance. They are known as 2-, alpha-, or α-amino acids (generic formula H₂NCHRCOOH in most cases where R is an organic substituent known as a “side-chain”); often the term “amino acid” is used to refer specifically to these. They include the 23 proteinogenic (“protein-building”) amino acids, which combine into peptidechains (“polypeptides”) to form the building-blocks of a vast array of proteins. These are all L-stereoisomers (“left-handed” isomers), although a few D-amino acids (“right-handed”) occur in bacterial envelopes and some antibiotics. Twenty of the proteinogenic amino acids are encoded directly by triplet codons in the genetic code and are known as “standard” amino acids. The other three (“non-standard” or “non-canonical”) are selenocysteine (present in many noneukaryotes as well as most eukaryotes, but not coded directly by DNA), pyrrolysine (found only in some archea and one bacterium) and N-formylmethionine (which is often the initial amino acid of proteins in bacteria, mitochondria, and chloroplasts). Pyrrolysine and selenocysteine are encoded via variant codons; for example, selenocysteine is encoded by stop codon and SECIS element. Codon-tRNA combinations not found in nature can optionally be used to “expand” the genetic code and create novel proteins known as alloproteins incorporating non-proteinogenic amino acids. Many important proteinogenic and non-proteinogenic amino acids also play critical non-protein roles within the body. For example, in the human brain, glutamate (standard glutamic acid) and gamma-amino-butyric acid (“GABA”, non-standard gamma-amino acid) are, respectively, the main excitatory and inhibitory neurotransmitters; hydroxyproline (a major component of the connective tissue collagen) is synthesized from proline; the standard amino acid glycine is used to synthesize porphyrins used in red blood cells; and the non-standard carnitine is used in lipid transport. Nine proteinogenic amino acids are called “essential” for humans because they cannot be created from other compounds by the human body and, so, must be taken in as food. Others may be conditionally essential for certain ages or medical conditions. Essential amino acids may also differ between species. Because of their biological significance, amino acids are important in nutrition and are commonly used in nutritional supplements, fertilizers, and food technology. Industrial uses include the production of drugs, plastics, and chiral catalysts.

Amorphous Metal (also known metallic glass or glassy metal) is a solid metallic material, usually an alloy, with a disordered atomic-scale structure. Most metals are crystalline in their solid state, which means they have a highly ordered arrangement of atoms. Amorphous metals are non-crystalline, and/or have a glass-like structure. But unlike common glasses, such as window glass, which are typically insulators, amorphous metals have good electrical conductivity. There are several ways in which amorphous metals can optionally be produced, including cooling, physical, solid-state reaction, ion irradiation, and/or mechanical alloying. In the past, small batches of amorphous metals have been produced through a variety of quick-cooling methods. For instance, amorphous metal ribbons have been produced by sputtering molten metal onto a spinning metal disk (melt spinning). The rapid cooling, on the order of millions of degrees a second, is too fast for crystals to form and/or the material is “locked” in a glassy state. More recently a number of alloys with critical cooling rates low enough to allow formation of amorphous structure in thick layers (over 1 millimeter) had been produced; these are known as bulk metallic glasses (BMG). Liquid metal sells a number of titanium-based BMGs, developed in studies originally performed at Caltech. More recently, batches of amorphous steel have been produced that demonstrate strengths much greater than conventional steel alloys.

Amorphous Solid. Amorphous solid is a solid, which lacks a crystalline structure. That is, it does not have long range ordered arrangement of atoms, molecules, or ions within the structure. Glass, gels, thin films, plastics and nano structures materials are some examples for amorphous solids. Glass is primarily made with sand (silica/SiO₂), and bases like sodium carbonate, and calcium carbonate. At high temperatures, these materials melt together, and when they are cooled, a rigid glass is formed rapidly. When cooling, the atoms are arranged in a disordered manner to produce glass; thus, it is referred to as amorphous. However, atoms can have a short-range order due to chemical bonding characteristics. Likewise, other amorphous materials can optionally be prepared by rapidly cooling molten material. Amorphous solids don't have a sharp melting point. They liquefy over a broad range of temperature. Amorphous solids like rubber are used in tire manufacturing. Glass and plastics are used in the making of house ware, laboratory equipment etc.

Crystalline Solid. Crystalline solids or crystals have ordered structures and symmetry. The atoms, molecules, or ions in crystals are arranged in a particular manner; thus, have a long-range order. In crystalline solids, there is a regular, repeating pattern; by definition, a crystal is “a homogenous chemical compound with a regular and periodic arrangement of atoms. Examples are halite, salt (NaCl), and quartz (SiO₂). But crystals are not restricted to minerals: they comprise most solid matter such as sugar, cellulose, metals, bones and even DNA.” Crystals are naturally occurring on earth as large crystalline rocks such as quartz, granite. Crystals are formed by living organisms. For example, calcite is produced by mollusks. There are water-based crystals in the form of snow, ice or glaciers. Crystals can be categorized according to their physical and chemical properties. They are covalent crystals (e.g.: diamond), metallic crystals (e.g.: pyrite), ionic crystals (e.g.: sodium chloride) and molecular crystals (e.g. sugar). Crystals can have different shapes and colors. Crystals have an aesthetic value, and it is believed to have healing properties; thus, people use them to make jewelry.

Animal Feed is food given to domestic animals in the course of animal husbandry. There are two basic types, fodder and forage. Used alone, the word “feed” more often refers to fodder.

Fodder. “Fodder” refers particularly to food given to the animals (including plants cut and carried to them), rather than that which they forage for themselves. It includes hay, straw, silage, compressed and pelleted feeds, oils and mixed rations, and sprouted grains and legumes. Feed grains are the most important source of animal feed globally. The amount of grain used to produce the same unit of meat varies substantially.

Forage. “Forage” is plant material (mainly plant leaves and stems) eaten by grazing livestock. Historically, the term forage has meant only plants eaten by the animals directly as pasture, crop residue, or immature cereal crops, but it is also used more loosely to include similar plants cut for fodder and carried to the animals, especially as hay or silage. Nutrition. In agriculture today, the nutritional needs of farm animals are well understood and may be satisfied through natural forage and fodder alone, or augmented by direct supplementation of nutrients in concentrated, controlled form. The nutritional quality of feed is influenced not only by the nutrient content, but also by many other factors such as feed presentation, hygiene, digestibility, and effect on intestinal health. Feed additives provide a mechanism through which these nutrient deficiencies can be resolved effect the rate of growth of such animals and also their health and well-being. Even with all of the benefits of higher quality feed, most of a farm animal's diet still consists of grain-based ingredients because of the higher costs of quality feed. Animals. Bird food, Cat food, Cattle feeding, Dog food, Equine nutrition, Pet food, Pig farming, Poultry feed, Sheep husbandry.

Animal Feed Ingredients. non-limiting examples of animal feed ingredients and feed formulations used in the manufacture of feeds is the most important factor in feed processing, and quality and composition of feeds plays an integral part in the nutrition and up bring of the animals/fish/shrimp to be fed.

Feed Ingredients Glossary: non-limiting examples of food ingredients, include: Barley, Beet Pulp Pellets, Blood Meal □, Bone Meal □, Cassava Leaf Meal □, Copra Meal □, Corn □, Corn Gluten Meal □, Cottonseed Meal, Feather Meal, Fish Meal, □ Fish Silage □, Limestone □, Linseed □, Maize □, Meat Meal, Meat and Bone Meal, Molasses, □ Oat Groats, □ Oil Cakes, □ Palm Kernel Cake, □ Palm Oil Sludge □, Peanut Meals, Poultry Feathers, □ Poultry by-products.

Rapeseed Meal, Rice □, Rice bran, □ Rice Husk, □ Rice Polishings, Rye, □ Sago □, Sesame Cake, □ Shrimp meal, □ Skim Milk Powder (dried), □ Squid Meal, □ Sorghum, □ Soybean Meal, □ Starches, □ Sunflower Meal, □ Sweet Potato, □ Tapioca, □ Wheat □, Wheat Germ Meal □, Wheat Gluten, □ Whey Dried, □ Yeast Brewers, □ Yeast, Sugar Cane.

Artificial Ingredient usually refers to an ingredient which is artificial or man-made, such as: Artificial flavor, Food additive, food coloring, preservative, sugar substitute, artificial sweetener.

Bamboo Pulp is a tribe of flowering perennial evergreen plants in the grass family Poaceae, subfamily Bambusoideae, tribe Bambuseae; although, the forestry services and departments of many countries where bamboo is utilized as a building material consider bamboo to be a forestry product, and it is specifically harvested as a tree exclusively for the wood it produces, which in many ways is a wood superior in strength and resilience to other natural, fibrous building materials. In bamboos, the internodal regions of the stem are hollow and the vascular bundles in the cross section are scattered throughout the stem instead of in a cylindrical arrangement. The dicotyledonous woody xylem is also absent. The absence of secondary growth wood causes the stems of monocots, even of palms and large bamboos, to be columnar rather than tapering. Bamboos are some of the fastest-growing plants in the world, due to a unique rhizome-dependent system. Certain species of bamboo can grow 35 inches within a 24-hour period, at a rate of 0.00003 km/h (a growth of approximately 1 millimeter (or 0.02 inches) every 2 minutes). Bamboos are of notable economic and cultural significance in South Asia, Southeast Asia and East Asia, being used for building materials, as a food source, and as a versatile raw product. Bamboo has a higher compressive strength than wood, brick or concrete and a tensile strength that rivals steel.

Battery Types can include, but not limited to, (a) primary cells or non-rechargeable batteries, alkaline battery, aluminum-air battery, aluminum-ion battery, atomic battery, bendable battery, betavoltaics, optoelectric nuclear battery, nuclear micro-battery, bunsen cell, chromic acid cell (Poggendorff cell), cell phone battery, Clark cell, Daniell cell, Dry cell, Earth battery, flexible battery, Frog battery, Galvanic cell, Grove cell, Leclanché cell, Lemon battery, Lithium battery, Lithium air battery, Mercury battery, Molten salt battery, Nickel oxyhydroxide battery, Oxyride battery, Pulvermacher's chain, Reserve battery, Silver-oxide battery, Solid-state battery, Voltaic pile, wearable battery, Weston cell, Zinc-air, battery, Zinc-carbon battery, Zinc chloride battery; (b) Secondary cells or rechargeable batteries: Flow battery, Vanadium redox battery, Zinc-bromine battery, Zinc-cerium battery, Fuel cell, Lead-acid battery, Deep cycle battery, VRLA battery, AGM battery, Gel battery, Lithium air battery, Lithium-ion battery, Beltway battery, Lithium ion manganese oxide battery (IMR), Lithium ion polymer battery, Lithium iron phosphate battery, Lithium-sulfur battery, Lithium-titanate battery, Molten salt battery, Nickel-cadmium battery, Nickel-cadmium battery vented cell type, Nickel hydrogen battery, Nickel-iron battery, Nickel metal hydride battery, Low self-discharge NiMH battery, Nickel-zinc battery, Organic radical battery, Polymer-based battery, Polysulfide bromide battery, Potassium-ion battery, Rechargeable alkaline battery, Rechargeable fuel battery, Silicon air battery, Silver-zinc battery, Silver calcium battery, Sodium-ion battery, Sodium-sulfur battery, Sugar battery, Super iron battery, Ultra Battery; and/or (c) Batteries by application: e.g., Backup battery, Battery (vacuum tube), Battery pack, Battery room, Biobattery, Button cell, Car battery, CMOS battery, Common battery, Commodity cell, Electric vehicle battery, Home battery, Business battery, Laptop battery, Smart battery, Solar battery, Flow battery, Inverter battery, Lantern battery, Nanobatteries, Nanowire battery, Local battery, Polapulse battery, Photoflash battery, Smart battery system, Thin film rechargeable lithium battery, Traction battery, Watch battery, Water-activated battery, Wet cell, and/or Zamboni pile.

Plastics are derived from renewable biomass sources, such as vegetable fats and/or oils, corn starch, pea starch or microbiota. Plastic can optionally be made from agricultural byproducts and/or also from used plastic bottles or plastic water bottles or other types of bottles or other containers using microorganisms. Common plastics, such as fossil-fuel plastics (also called petro based polymers), are derived from petroleum. Production of such plastics tends to require more fossil fuels and/or to produce more greenhouse gases than the production of biobased polymers (plastics). Some, but not all, plastics are designed to biodegrade. Plastics can optionally break down in either anaerobic or aerobic environments, depending on how they are manufactured. Plastics can optionally be composed of starches, cellulose, polymers, and/or a variety of other materials.

Biosensor is an analytical device, used for the detection of an analyte, that combines a biological component with a physicochemical detector; the sensitive biological element (e.g. tissue, microorganisms, organelles, cell receptors, enzymes, antibodies, nucleic acids, etc.), a biologically derived material or biomimetic component that interacts (binds or recognizes) the analyte under study. The biologically sensitive elements can optionally be created by biological engineering, the transducer or the detector element (works in a physicochemical way; optical, piezoelectric, electrochemical, etc.) that transforms the signal resulting from the interaction of the analyte with the biological element into another signal (i.e., transduces) that can be more easily measured and quantified, biosensor reader device with the associated electronics or signal processors that are primarily responsible for the display of the results in a user-friendly way) This sometimes accounts for the most expensive part of the sensor device, however it is possible to generate a user-friendly display that includes transducer and sensitive element (see Holographic Sensor). The readers are usually custom-designed and manufactured to suit the different working principles of biosensors.

Blocking Agent is an agent that inhibits a biologic action, such as movement of an ion across the cell membrane, passage of a neural impulse, or interaction with a specific receptor.

Blocking Absorption of Dietary Cholesterol. Dietary cholesterol is optionally obtained from foods derived from animal sources that are rich in fat content. A healthy adult only needs to ingest about 30% of the daily cholesterol requirement. Obtaining more than this amount from dietary cholesterol can lead to increased cholesterol levels and serious health risks. Dietary cholesterol is absorbed within the lumen of the small intestine. Bile salts produced from cholesterol in the liver interact with phospholipids to produce a biliary micelle that is transported via bile into the lumen. Dietary cholesterol in the lumen is easily incorporated into these micelles and together with the already present biliary cholesterol can now be absorbed into the enterocytes that make up the walls of the lumen. The micelles enter the cell by a channel know as Niemann-Pick C1 Like 1 protein (NPC1L1). Once in the cells the cholesterol can either be pumped back out into the lumen or it can be esterified for transport within chylomicrons. Preventing the absorption of this dietary cholesterol has become a key area in cholesterol related research. Plant sterols and stanols have been shown to be effective inhibitors of cholesterol absorption. Ingested as part of a normal diet, plant sterols and stanols are very similar in structure to cholesterol. They actually have a stronger binding affinity than cholesterol to the biliary micelles that aid in absorption. Because of this the sterols and stanols can displace cholesterol from the micelles thus preventing its absorption. Recently, inhibitors that block the absorption of the biliary micelles into the enterocytes have also been used to block the uptake of dietary cholesterol.

Breast Implant is a prosthesis used to change the size, form, and texture of a woman's breast; in plastic surgery, breast implants are applied for post-mastectomy breast reconstruction; for correcting congenital defects and deformities of the chest wall; and for aesthetic breast augmentation. There are three general types of breast implant devices, defined by their filler material: saline solution, silicone gel, and composite filler. The saline implant has an elastomer silicone shell filled with sterile saline solution; the silicone implant has an elastomer silicone shell filled with viscous silicone gel; and the alternative composition implants featured miscellaneous fillers, such as oil, polypropylene, et cetera. In surgical practice, for the reconstruction of a breast, the tissue expander device is a temporary breast prosthesis used to form and establish an implant pocket for emplacing the permanent breast implant. For the correction of male breast defects and deformities, the pectoral implant is the breast prosthesis used for the reconstruction and the aesthetic repair of a man's chest wall.

Carboxylated Nanocrystalline Cellulose (CNCC). Specialty paper (e.g. cigarette paper and battery diaphragm paper) requires extremely high strength properties. The addition of strength agents plays an important role in increasing strength properties of paper. Nanocrystalline cellulose (NCC), or other cellulose whiskers, has the potential to enhance the strength properties of paper via improving inter-fibers bonding. This paper was to determine the potential of using carboxylated nanocrystalline cellulose (CNCC) to improve the strength properties of paper made of cellulosic fiber or poly (vinyl alcohol) (PVA) fiber. The results indicated that the addition of CNCC can effectively improve the strength properties. At a CNCC dosage of 0.7%, the tear index and tensile index of the cellulosic paper reached the maximum of 12.8 mN m²/g and 100.7 N m/g, respectively. More importantly, when increasing the CNCC dosage from 0.1 to 1.0%, the tear index and tensile index of PVA fiber paper were increased by 67.29%, 22.55%, respectively.

Carcinogen is any substance, radionuclide, or radiation that is an agent directly involved in causing cancer. This may be due to the ability to damage the genome or to the disruption of cellular metabolic processes. Several radioactive substances are considered carcinogens, but their carcinogenic activity is attributed to the radiation, for example gamma rays and alpha particles, which they emit. Common examples of non-radioactive carcinogens are inhaled asbestos, certain dioxins, and tobacco smoke. Although the public generally associates carcinogenicity with synthetic chemicals, it is equally likely to arise in both natural and synthetic substances. Carcinogens are not necessarily immediately toxic, thus their effect can be insidious. Cancer is any disease in which normal cells are damaged and do not undergo programmed cell death as fast as they divide via mitosis. Carcinogens may increase the risk of cancer by altering cellular metabolism or damaging DNA directly in cells, which interferes with biological processes, and induces the uncontrolled, malignant division, ultimately leading to the formation of tumors. Usually, severe DNA damage leads to apoptosis, but if the programmed cell death pathway is damaged, then the cell cannot prevent itself from becoming a cancer cell. There are many natural carcinogens. Aflatoxin B₁, which is produced by the fungus Aspergillus flavus growing on stored grains, nuts and peanut butter, is an example of a potent, naturally occurring microbial carcinogen. Certain viruses such as hepatitis and human papilloma virus have been found to cause cancer in humans. The first one shown to cause cancer in animals is Rous sarcoma virus, discovered in 1910 by Peyton Rous. Other infectious organisms which cause cancer in humans include some bacteria (e.g. Helicobacter pylori) and helminths (e.g. Opisthorchis viverrini and Clonorchis sinensis). Dioxins and dioxin-like compounds, benzene, kepone, EDB, and asbestos have all been classified as carcinogenic. In the 1930s, industrial smoke and tobacco smoke were identified as sources of dozens of carcinogens, including benzo[a]pyrene, tobacco-specific nitrosamines such as nitrosonornicotine, and reactive aldehydes such as formaldehyde—which is also a hazard in embalming and making plastics. Vinyl chloride, from which PVC is manufactured, is a carcinogen and thus a hazard in PVC production. Co-carcinogens are chemicals that do not necessarily cause cancer on their own, but promote the activity of other carcinogens in causing cancer. After the carcinogen enters the body, the body makes an attempt to eliminate it through a process called biotransformation. The purpose of these reactions is to make the carcinogen more water-soluble so that it can be removed from the body. However, in some cases, these reactions can also convert a less toxic carcinogen into a more toxic carcinogen. DNA is nucleophilic, therefore soluble carbon electrophiles are carcinogenic, because DNA attacks them. For example, some alkenes are toxicated by human enzymes to produce an electrophilic epoxide. DNA attacks the epoxide, and is bound permanently to it. This is the mechanism behind the carcinogenicity of benzo[a]pyrene in tobacco smoke, other aromatics, aflatoxin and mustard gas. A carcinogen is a substance that is capable of causing cancer in humans or animals. If a substance is known to promote or aggravate cancer, but not necessarily cause cancer, it may also be called a carcinogen. Though there are many things that are believed to cause cancer, a substance is only considered carcinogenic if there is significant evidence of its carcinogenicity. A carcinogen may act on deoxyribonucleic acid (DNA), causing dangerous changes, or it may work to increase the rate of cell division. This change in cell division may work to increase the probability of DNA changes. Some carcinogens promote the development of cancer in other ways as well. It is important to note that carcinogens don't lead to cancer after every exposure. Some cause cancerous changes following high-level, prolonged exposure, while others may cause damage at lower levels and shorter exposure periods.

Cannabis is a genus of flowering plants that includes sativa, Cannabis, and Cannabis ruderalis. Indigenous to Central and South Asia, cannabis has long been used for fiber, seeds and oils, and for medicinal and recreational purposes. Cannabis is commonly referred to as marijuana, among other names, when as a psychoactive drug or as medicine. Tetrahydrocannabinol (THC) is the principal psychoactive constituent (cannabinoid) and is only found in the female cannabis plant. Cannabis oil is a thick, sticky, resinous substance made up of cannabinoids, such as THC and CBD, that is extracted from the cannabis plant (Cannabis sativa or Cannabis indica). Cannabis oil is a cannabis based product obtained by separating the resins from cannabis flowers using a solvent extraction process. Cannabis oil can optionally be known as marijuana oil, full extract cannabis oil (FECO), hash oil, dabs, shatter, or wax. Cannabis oil is the most potent of three main cannabis products, which are the actual cannabis flower (marijuana), resin (hashish), and oil (cannabis oil). Cannabis oil is the most concentrated form of the three main cannabis products. That is what makes cannabis oil the most potent.

Cannabis Oil is not to be confused with “ordinary” hemp oil because the two differ greatly, especially in the amount of THC available in the plant from which the oil is derived from. So before you run out and get some of that wicked hemp oil, be sure you know exactly what you're looking for because most of the hemp oils out there have no nutritional value whatsoever, let alone medicinal value. Cannabis Oil. Pure cannabis oil has taken a revolutionary role in cancer research. The oil is derived from female flowers of an indica cannabis plant. Depending on what you are after, you can make cannabis oil out of any female plant currently available in nature.

Cancer Causing Ingredients. Non-limiting examples of cancer-causing chemicals, include, but not limited to: Acesulfame-K, also known as Acesulfame-potassium or “Sunnette” is an artificial sweetener. It has not been adequately tested for human consumption. The FDA approved this additive even though the tests done to determine it's safety did not meet the FDA standards and caused cancer in lab animals, which increases the probability that it will also cause cancer in humans.

Artificial Colors, or FD&C Colors, are mostly are derived from coal tar, which is a carcinogen. Over the years, many FD&C colors have been banned because of their harmful effects. And it is likely that more will be banned in the future. Some of the worst FD&C colors include: Green #3, Blue #1, Blue #2 & Yellow #6, which cause allergic reactions and cancer in lab animals. Red #3 is a carcinogen, which may interfere with nerve transmission in the brain and causes genetic damage. It is banned in cosmetics, but allowed in food, and it's especially harmful to children. Yellow #5 causes allergic reactions in those sensitive to aspirin. It may be life threatening. Citrus Red #2 is a known carcinogen. Its only allowed use is to color orange skins. So, if you use orange zest in some of your recipes, you may be ingesting carcinogens. Any color with “lake” after it means that aluminum has been added to the color to make it insoluble.

BHA & BHT are widely used as preservatives, stabilizers and anti-aging products, antioxidants. BHA is known to cause cancer in humans. Both BHA and BHT are toxic to the liver and kidneys. BHT may react with other ingested substances to cause the formation of carcinogens. BHT is banned in England.

Potassium Bromate is used to treat flour to give bread and baked goods a sponge-like quality. It is probably not used in California because it might require a cancer warning on the label. Outside of California, “unbromated” breads do not contain potassium bromate. It is also used in toothpaste, mouth washes and gargles. It is a carcinogen, mutagen and highly toxic. It is banned worldwide, except in the U.S. and Japan.

Carrageenan is a seaweed derivative used in a wide variety of foods and cosmetics. In its native form, it has not been classified as a carcinogen, but in it's degraded or broken down form it has been classified as a possible human carcinogen by the International Agency for Research on Cancer (IARC).

Nitrates and nitrites are found primarily in processed meats. They combine with stomach acids and chemicals in foods to form nitrosamines, which are powerful carcinogens.

Olestra has not been shown to cause cancer. However, it robs the body of carotenoids, which are known to have a protective effect against cancer. Studies have shown a 40%-50% drop in blood carotenoids after consuming only 3-8 grams of olestra in a day, equivalent to 6-16 chips. It also may causes severe gastrointestinal cramping and diarrhea, which may last for extended periods of time.

Propyl Gallate is used as an antioxidant in fats, oils, candy and a variety of processed foods. It is a suspected carcinogen and is known to cause kidney, liver and gastrointestinal problems. It can cause allergic reactions in those with asthma and sensitivity to aspirin. It has not been adequately tested.

Saccharin, or Sweet 'N Low, is an artificial sweetener that is known to cause cancer. Because of pressure from the food industry, in 2000, saccharin was removed from the list of cancer-causing chemicals, in spite of the fact that studies still show that it causes cancer in lab animals.

Cancer-Causing Substances in the Environment. Cancer is caused by changes to certain genes that alter the way our cells function. Some of these genetic changes occur naturally when DNA is replicated during the process of cell division. But others are the result of environmental exposures that damage DNA. Non-limiting examples of cancer-causing substances in the environment include, but are not limited to the exposure of substances, such as the chemicals in tobacco smoke, or radiation, such as ultraviolet rays from the sun. People can avoid some cancer-causing exposures, such as tobacco smoke and the sun's rays. But others are harder to avoid, especially if they are in the air we breathe, the water we drink, the food we eat, or the materials we use to do our jobs. Scientists are studying which exposures may cause or contribute to the development of cancer. Understanding which exposures are harmful, and where they are found, may help people to avoid them. The substances listed below are among the most likely carcinogens to affect human health. Simply because a substance has been designated as a carcinogen, however, does not mean that the substance will necessarily cause cancer. Many factors influence whether a person exposed to a carcinogen will develop cancer, including the amount and duration of the exposure to substances such as, Aflatoxins, Aristolochic Acids, Arsenic, Asbestos, Benzene, Benzidine, Beryllium, 1,3-Butadiene, Cadmium, Coal Tar and Coal-Tar Pitch, Coke-Oven Emissions, Crystalline Silica (respirable size), Erionite, Ethylene Oxide, Formaldehyde, Hexavalent Chromium Compounds, Indoor Emissions from the Household Combustion of Coal, Mineral Oils: Untreated and Mildly Treated, Nickel Compounds, Radon, Secondhand Tobacco Smoke (Environmental Tobacco Smoke), Soot, Strong Inorganic Acid Mists Containing Sulfuric Acid, Thorium, Vinyl Chloride, Wood Dust, etc.

Carcinogens. Non-limiting examples of carcinogens, include, but not limited to: Group 1. Acetaldehyde (from consuming alcoholic beverages), Acheson process, occupational exposure associated with Acid mists, strong inorganic, Aflatoxins, Alcoholic beverages, Aluminum production, 4-Aminobiphenyl, Areca nut, Aristolochic acid (and plants containing it), Arsenic and inorganic arsenic compounds, Asbestos (all forms) and mineral substances (such as talc or vermiculite) that contain asbestos, Auramine production, Azathioprine, Benzene, Benzidine and dyes metabolized to benzidine, Benzo[a]pyrene, Beryllium and beryllium compounds, Betel quid, with or without tobacco, Bis(chloromethyl)ether and chloromethyl methyl ether (technical-grade), Busulfan, 1,3-Butadiene, Cadmium and cadmium compounds, Chlorambucil, Chlornaphazine, Chromium (VI) compounds, Clonorchis sinensis (infection with), also known as the Chinese liver fluke, Coal, indoor emissions from household combustion, Coal gasification, Coal-tar distillation, Coal-tar pitch, Coke production, Cyclophosphamide, Cyclosporine, 1,2 Dichloropropane, Diethylstilbestrol, Engine exhaust, diesel, Epstein-Barr virus (infection with), Erionite, Estrogen postmenopausal therapy, Estrogen-progestogen postmenopausal therapy (combined), Estrogen-progestogen oral contraceptives (combined) (Note: There is also convincing evidence in humans that these agents confer a protective effect against cancer in the endometrium and ovary), Ethanol in alcoholic beverages, Ethylene oxide, Etoposide, Etoposide in combination with cisplatin and bleomycin, Fission products, including strontium-90, Fluoro-edenite fibrous amphibole, Formaldehyde, Haematite mining (underground), Helicobacter pylori (infection with), Hepatitis B virus (chronic infection with), Hepatitis C virus (chronic infection with), Human immunodeficiency virus type 1 (HIV-1) (infection with), Human papilloma virus (HPV) types 16, 18, 31, 33, 35, 39, 45, 51, 52, 56, 58, 59 (infection with) (Note: The HPV types that have been classified as carcinogenic to humans can differ by an order of magnitude in risk for cervical cancer), Human T-cell lymphotropic virus type I (HTLV-1) (infection with), Ionizing radiation (all types), Iron and steel founding (workplace exposure), Isopropyl alcohol manufacture using strong acids, Kaposi sarcoma herpes virus (KSHV), also known as human herpes virus 8 (HHV-8) (infection with), Leather dust, Magenta production, Melphalan, Methoxsalen (8-methoxypsoralen) plus ultraviolet A radiation, also known as PUVA, 4,4′-Methylenebis (chloroaniline) (MOCA), Mineral oils, untreated or mildly treated, MOPP and other combined chemotherapy including alkylating agents, 2-Naphthylamine, Neutron radiation, Nickel compounds, N′-Nitrosonornicotine (NNN) and 4-(N-Nitrosomethylamino)-1-(3-pyridyl)-1-butanone (NNK), Opisthorchis viverrini (infection with), also known as the Southeast Asian liver fluke, Outdoor air pollution and the particulate matter in it, Painter (workplace exposure as a), 3,4,5,3′,4′-Pentachlorobiphenyl (PCB-126), 2,3,4,7,8-Pentachlorodibenzofuran, Phenacetin (and mixtures containing it), Phosphorus-32, as phosphate, Plutonium, Polychlorinated biphenyls (PCBs), dioxin-like, with a Toxicity Equivalency Factor according to WHO (PCBs 77, 81, 105, 114, 118, 123, 126, 156, 157, 167, 169, 189), Radioiodines, including iodine-131, Radionuclides, alpha-particles-emitting, internally deposited (Note: Specific radionuclides for which there is sufficient evidence for carcinogenicity to humans are also listed individually as Group 1 agents), Radionuclides, beta-particles-emitting, internally deposited (Note: Specific radionuclides for which there is sufficient evidence for carcinogenicity to humans are also listed individually as Group 1 agents), Radium-224 and its decay products, Radium-226 and its decay products, Radium-228 and its decay products, Radon-222 and its decay products, Rubber manufacturing industry, Salted fish (Chinese-style), Schistosoma haematobium (infection with), Semustine (methyl-CCNU), Shale oils, Silica dust, crystalline, in the form of quartz or cristobalite, Solar radiation, Soot (as found in workplace exposure of chimney sweeps), Sulfur mustard, Tamoxifen (Note: There is also conclusive evidence that tamoxifen reduces the risk of contralateral breast cancer in breast cancer patients), 2,3,7,8-Tetrachlorodibenzo-para-dioxin, Thiotepa, Thorium-232 and its decay products, Tobacco, smokeless, Tobacco smoke, secondhand, Tobacco smoking, ortho-Toluidine, Treosulfan, Trichloroethylene, Ultraviolet (UV) radiation, including UVA, UVB, and UVC rays, Ultraviolet-emitting tanning devices, Vinyl chloride.

National Toxicology Program 13th Report on Carcinogens “Known to be human carcinogens”. Aflatoxins, Alcoholic beverage consumption, 4-Aminobiphenyl, Analgesic mixtures containing phenacetin, Aristolochic acids, Arsenic and inorganic arsenic compounds, Asbestos, Azathioprine, Benzene, Benzidine, Beryllium and beryllium compounds, Bis(chloromethyl) ether and technical-grade chloromethyl methyl ether, 1,3-Butadiene, 1,4-Butanediol dimethylsulfonate (also known as busulfan), Cadmium and cadmium compounds, Chlorambucil, 1-(2-Chloroethyl)-3-(4-methylcyclohexyl)-1-nitrosourea (MeCCNU), Chromium hexavalent compounds, Coal tar pitches, Coal tars, Coke oven emissions, Cyclophosphamide, Cyclosporin A, Diethylstilbestrol (DES), Dyes metabolized to benzidine, Erionite, Estrogens, steroidal, Ethylene oxide, Formaldehyde, Hepatitis B virus, Hepatitis C virus, Human papilloma viruses: some genital-mucosal types. Melphalan, Methoxsalen with ultraviolet A therapy (PUVA), Mineral oils (untreated and mildly treated), Mustard gas, 2-Naphthylamine, Neutrons, Nickel compounds, Oral tobacco products, Radon, Silica, crystalline (respirable size), Solar radiation, Soots, Strong inorganic acid mists containing sulfuric acid, Sunlamps or sunbeds, exposure to Tamoxifen, 2,3,7,8-Tetrachlorodibenzo-p-dioxin (TCDD); “dioxin”, Thiotepa, Thorium dioxide, Tobacco smoke, environmental, Tobacco, smokeless, Tobacco smoking, o-Toluidine, Vinyl chloride, Ultraviolet (UV) radiation, broad spectrum, Wood dust, X-radiation and gamma radiation.

Probable Carcinogens. International Agency for Research on Cancer Group 2A: Probably carcinogenic to humans, Acrylamide, Adriamycin (doxorubicin), Androgenic (anabolic) steroids, Art glass, glass containers, and press ware (manufacture of), Azacitidine, Biomass fuel (primarily wood), emissions from household combustion, Bischloroethyl nitrosourea (BCNU), also known as carmustine, Captafol, Carbon electrode manufacture, Chloral, Chloral hydrate, Chloramphenicol, alpha-Chlorinated toluenes (benzal chloride, benzotrichloride, benzyl chloride) and benzoyl chloride (combined exposures), 1-(2-Chloroethyl)-3-cyclohexyl-1-nitrosourea (CCNU), 4-Chloro-ortho-toluidine, Chlorozotocin, Cisplatin, Cobalt metal with tungsten carbide, Creosotes, Cyclopenta[cd]pyrene, Diazinon, Dibenz[a,j]acridine, Dibenz[a,h]anthracene, Dibenzo[O]pyrene, Dichloromethane (methylene chloride), Diethyl sulfate, Dimethylcarbamoyl chloride, 1,2-Dimethylhydrazine, Dimethyl sulfate, Epichlorohydrin, Ethyl carbamate (urethane), Ethylene dibromide, N-Ethyl-N-nitrosourea, Frying, emissions from high-temperature, Glycidol, Glyphosate, Hairdresser or barber (workplace exposure as), Human papillomavirus (HPV) type 68 (infection with), Indium phosphide, IQ (2-Amino-3-methylimidazo[4,5-f]quinoline), Lead compounds, inorganic, Malaria (caused by infection with Plasmodium falciparum), Malathion, Mate, hot, Merkel cell polyomavirus (MCV), 5-Methoxypsoralen, Methyl methanesulfonate, N-Methyl-N′-nitro-N-nitrosoguanidine (MNNG), N-Methyl-N-nitrosourea, Nitrate or nitrite (ingested) under conditions that result in endogenous nitrosation, 6-Nitrochrysene, Nitrogen mustard, 1-Nitropyrene, N-Nitrosodiethylamine, N-Nitrosodimethylamine, 2-Nitrotoluene, Non-arsenical insecticides (workplace exposures in spraying and application of), Petroleum refining (workplace exposures in), Pioglitazone, Polybrominated biphenyls (PBBs), Procarbazine hydrochloride, 1,3-Propane sultone, Shiftwork that involves circadian disruption, Styrene-7,8-oxide, Teniposide, Tetrachloroethylene (perchloroethylene), Tetrafluoroethylene, Trichloroethylene, 1,2,3-Trichloropropane, Tris(2,3-dibromopropyl) phosphate, Vinyl bromide (Note: For practical purposes, vinyl bromide should be considered to act similarly to the human carcinogen vinyl chloride.), Vinyl fluoride (Note: For practical purposes, vinyl fluoride should be considered to act similarly to the human carcinogen vinyl chloride.)

National Toxicology Program 13th Report on Carcinogens “Reasonably anticipated to be human carcinogens”. Acetaldehyde, 2-Acetylaminofluorene, Acrylamide, Acrylonitrile, Adriamycin® (doxorubicin hydrochloride), 2-Aminoanthraquinone, o-Aminoazotoluene, 1-Amino-2,4-dibromoanthraquinone, 1-Amino-2-methylanthraquinone, 2-Amino-3,4-dimethylimidazo[4,5-f]quinoline (MeIQ), 2-Amino-3,8-dimethylimidazo[4,5-f]quinoxaline (MeIQx), 2-Amino-3-methylimidazo[4,5-f]quinoline (IQ), 2-Amino-1-methyl-6-phenylimidazo[4,5-b]pyridine (PhIP), Amitrole, o-Anisidine and its hydrochloride, Azacitidine (5-Azacytidine®, 5-AzaC), Basic Red 9 Monohydrochloride, Benz[a]anthracene, Benzo[b]fluoranthene, Benzo[j]fluoranthene, Benzo[k]fluoranthene, Benzo[a]pyrene, Benzotrichloride, 2, 2-bis-(bromoethyl)-1,3-propanediol (technical grade), Bromodichloromethane, 1-Bromopropane, Butylated hydroxyanisole (BHA), Captafol, Carbon tetrachloride, Ceramic fibers (respirable size), Chloramphenicol, Chlorendic acid, Chlorinated paraffins (C₁₂, 60% chlorine), Chloroform, 1-(2-chloroethyl)-3-cyclohexyl-1-nitrosourea, Bis(chloroethyl) nitrosourea, 3-Chloro-2-methylpropene, 4-Chloro-o-phenylenediamine, Chloroprene, p-Chloro-o-toluidine and p-chloro-o-toluidine hydrochloride, Chlorozotocin, Cisplatin, Cobalt sulfate, Cobalt-tungsten carbide: powders and hard metals, p-Cresidine, Cumene, Cupferron, Dacarbazine, Danthron (1,8-dihydroxyanthraquinone), 2,4-Diaminoanisole sulfate, 2,4-Diaminotoluene, Diazoaminobenzene, Dibenz[a,h]acridine, Dibenz[a,j]acridine, Dibenz[a,h]anthracene, 7H-Dibenzo[c,g]carbazole, Dibenzo[a,e]pyrene, Dibenzo[a,h]pyrene, Dibenzo[a,i]pyrene, Dibenzo[a,l]pyrene, 1,2-Dibromo-3-chloropropane, 1,2-Dibromoethane (ethylene dibromide), 2,3-Dibromo-1-propanol, Tris (2,3-dibromopropyl) phosphate, 1,4-Dichlorobenzene, 3,3′-Dichlorobenzidine and 3,3′-dichlorobenzidine dihydrochloride, Dichlorodiphenyltrichloroethane (DDT), 1,2-Dichloroethane (ethylene dichloride), Dichloromethane (methylene chloride), 1,3-Dichloropropene (technical grade), Diepoxybutane, Diesel exhaust particulates, Diethyl sulfate, Diglycidyl resorcinol ether, 3,3′-Dimethoxybenzidine, 4-Dimethylaminoazobenzene, 3,3′-Dimethylbenzidine, Dimethylcarbamoyl chloride, 1,1-Dimethylhydrazine, Dimethyl sulfate, Dimethylvinyl chloride, 1,6-Dinitropyrene, 1,8-Dinitropyrene, 1,4-Dioxane, Disperse blue 1, Dyes metabolized to 3,3′-dimethoxybenzidine, Dyes metabolized to 3,3′-dimethylbenzidine, Epichlorohydrin, Ethylene thiourea, Ethyl methanesulfonate, Furan, Glass wool fibers (inhalable), Glycidol, Hexachlorobenzene, Hexachlorocyclohexane isomers, Hexachloroethane, Hexamethylphosphoramide, Hydrazine and hydrazine sulfate, Hydrazobenzene, Indeno[1,2,3-cd]pyrene, Iron dextran complex, Isoprene, Kepone® (chlordecone), Lead and lead compounds, Lindane, hexachlorocyclohexane, 2-Methylaziridine (propylenimine), 5-Methylchrysene, 4,4′-Methylenebis(2-chloroaniline), 4-4′-Methylenebis(N,N-dimethyl)benzenamine, 4,4′-Methylenedianiline and its dihydrochloride salt, Methyleugenol, Methyl methanesulfonate, N-methyl-N′-nitro-N-nitrosoguanidine, Metronidazole, Michler's ketone [4,4′-(dimethylamino) benzophenone], Mirex, Naphthalene, Nickel, metallic, Nitrilotriacetic acid, o-Nitroanisole, Nitrobenzene, 6-Nitrochrysene, Nitrofen (2,4-dichlorophenyl-p-nitrophenyl ether), Nitrogen mustard hydrochloride, Nitromethane, 2-Nitropropane, 1-Nitropyrene, 4-Nitropyrene, N-nitrosodi-n-butylamine, N-nitrosodiethanolamine, N-nitrosodiethylamine, N-nitrosodimethylamine, N-nitrosodi-n-propylamine, N-nitroso-N-ethylurea, 4-(N-nitrosomethylamino)-1-(3-pyridyl)-1-butanone, N-nitroso-N-methylurea, N-nitrosomethylvinylamine, N-nitrosomorpholine, N-nitrosonornicotine, N-nitrosopiperidine, N-nitrosopyrrolidine, N-nitrososarcosine, o-Nitrotoluene, Norethisterone, Ochratoxin A, 4,4′-Oxydianiline, Oxymetholone, Pentachlorophenol and by-products of its synthesis, Phenacetin, Phenazopyridine hydrochloride, Phenolphthalein, Phenoxybenzamine hydrochloride, Phenytoin and phenytoin sodium, Polybrominated biphenyls (PBBs), Polychlorinated biphenyls (PCBs), Polycyclic aromatic hydrocarbons (PAHs), Procarbazine and its hydrochloride, Progesterone, 1,3-Propane sultone, beta-Propiolactone, Propylene oxide, Propylthiouracil, Reserpine, Riddelliine, Safrole, Selenium sulfide, Streptozotocin, Styrene, Styrene-7,8-oxide, Sulfallate, Tetrachloroethylene (perchloroethylene), Tetrafluoroethylene, Tetranitromethane, Thioacetamide, 4,4′-Thiodianaline, Thiourea, Toluene diisocyanates, Toxaphene, Trichloroethylene, 2,4,6-Trichlorophenol, 1,2,3-Trichloropropane, Tris(2,3-dibromopropyl) phosphate, Ultraviolet A radiation, Ultraviolet B radiation, Ultraviolet C radiation, Urethane, Vinyl bromide, 4-Vinyl-1-cyclohexene diepoxide, Vinyl fluoride.

Carcinogens in Foods. There are many carcinogens in food that is consumed by most Americans today. It is important that the general public be aware of these poisons so that they can make rational decisions when choosing food.

Fish. Fish are extremely sensitive to pesticides. They sicken or die at very low concentrations, much lower than for most living organisms. Concentrations as low as two parts per trillion of DDT were found to cause problems in the Great Lakes. The consumer can be harmed by eating fish that has been poisoned but not killed outright. Fish have been known to concentrate these poisons 2,000-fold over the amounts in the water where they were found.

Shellfish. In recent years, state and federal agencies that control oyster beds and their care have been pouring materials into the sandbanks to protect oysters from their enemies, such as starfish and other sea creatures. These materials are made of insecticide, and a chemical (orthodichlorobenzene) combined with sand. Sea animals, venturing into the treated sandbank, perish from the poisons in different ways.

Hatchery-Raised Trout. The feeds developed for trout were similar to those previously used for poultry. When the pellets were fed to baby chicks, the birds developed cancer. Later rainbow trout, raised in hatcheries, were given this feed in hopes of achieving maximum weight gains in the shortest period of time before being released into streams. In the early 1960s, hatchery-raised rainbow trout that were fed this pelleted feed developed liver cancer in what leading cancer specialists considered epidemic proportions. In some hatcheries, 100% of the trout were affected. The outbreak seemed related to a cancer-inducing ingredient, still not completely identified, but present in the fat fraction of the feed.

Fresh Fish. Fresh fish may be refrigerated in crushed ice containing preservatives such as sodium benzoate, sodium nitrite, hydrogen peroxide, ozone, or chlorine to inhibit spoilage. In recent years, cases of illness and deaths were traced to excessive amounts of sodium nitrite added to fish by sellers' who hoped to prolong even further the shelf life of their products.

Eggs. Eggs are vehicles for residues of a wide range of chemicals present in the diet and environment of laying hens. Antibiotics in feed may more than double the egg laying in low-producing hens. There is also pressure to include antibiotics in the drinking water of layers as well. Feed medicated with antibiotics must be withheld from birds when they are laying. But even when this recommendation has been followed, antibiotics have been detected. Although the FDA has set “zero” or “negligible residue” tolerance levels for pesticides in eggs, there is no assurance that this food is uncontaminated. Poultry management and poultry feed may both contribute to pesticide residues in eggs.

Drugs. Drugs may be used with laying hens. A tranquilizer, used in conjunction with antibiotics in layer feed, was advertised as boosting egg production since it “calms birds, reduces blood pressure and heart rate, increases respiratory rate.” Experimentally, hens fed aspirin laid more eggs. Another drug has been found to be effective in reducing “laying slump”; at the same time it cuts feeding costs. Poultry Arsenic. Since 1950, small amounts of arsenic as arsanilic acid have been incorporated into poultry feed to stimulate early maturation, increase efficiency of feed utilization, produce more eggs, “improve” skin coloring and feathering, and yield more profits. Currently 90% of all commercial chickens are raised with arsenic in their feed. The arsenic-containing feed must be discontinued long enough before slaughter for the birds to eliminate most—but not all—of it from their meat. Even though arsenic is listed as a carcinogen for man, the FDA allows tolerance residues of 0.5 ppm for it in chicken and turkey tissues, and twice that amount in the byproducts of these birds. The liver is the detoxifying organ of animal and man. Dr. Manuel Schreiber, FDA toxicologist, stated that dangerous accumulations of arsenic have been found in chicken livers. Another group of “anti-infective” agents, the bacteriostats, are incorporated routinely in poultry feed to control the growth of “undesirable” bacteria. These include drugs, which can result in dermatitis in man when applied to the skin, and others, which are toxic. Little is known about the general effects of these materials, when eaten frequently in small amounts.

Caponettes. Hormones in poultry production have been used even longer than with livestock. The estrogenic female sex hormones, especially stilbestrol, were first used to caponize birds chemically. The use of stilbestrol was extended to include the treatment of all types of table poultry of both sexes, being highly profitable to the poultrymen. It put weight on birds quickly, and could even give old birds the appearance of youth, with plumper, more attractive flesh. Since the cancer-inciting nature of stilbestrol was established, the FDA was forced to take action. The agency chose a course least upsetting to the economics of poultrymen, by persuading the industry to “voluntarily” discontinue the use of stilbestrol implants.

Pesticides. In 1965, the USDA tested 2,600 poultry samples in every federally-inspected plant throughout the nation, and found all birds contaminated with pesticide residues. No one section of the country was better than another. Primary sources of infection were traced to sprayed grain and animal tallows in the feed and to poor husbandry practices. No seizures were made, nor did the USDA divulge specific results, such as the most common contaminant or levels of pesticides found.

Cancerous Chickens. Until 1970, the USDA's policy had been to condemn the entire carcass of the chicken if any organs or sections of the bird showed signs of leukosis. The poultry industry considered this to be an economic hardship and it pressured for lowering the standards for condemning birds. The USDA appointed a panel of veterinarians and animal-disease specialists to review the problem. Although the panel recommended continuing the policy, of condemning birds whose internal organs show active signs of leukosis, it suggested that chickens bearing cancer be allowed on the market if they “do not look too repugnant.” The USDA endorsed the proposals. Officials from the agency said that if tumors were detected on the wing of a bird, the wing could be cut off and used in products such as hot dogs, while the rest of the bird could be cut up as chicken—all without posing a threat to human health.

Hamburger. Toxins are prevalent in ground beef. This popular food item offers many opportunities for economic frauds, such as additives and illegal extenders. It may be adulterated with coal-tar colors, cochineal, and sodium nitrite or benzoate of soda. Dr. Freese at the National Institute of Health has strongly recommended that sodium nitrite be banned from use in foods. In the human stomach, sodium nitrite is converted to nitrous acid, which is mutagenic in a variety of lower organisms. Sodium sulfur is another additive, can mask the smell of deteriorating meat, and give it a fresh-meat redness. Such meat is injurious, especially if eaten rare. Sodium sulfite is a poison that destroys vitamin B, and is capable of causing considerable damage to the digestive system and other organs. Yet tested samples of ground beef purchased as ready-chopped hamburger, or sold at hot dog stands, cafeterias, and restaurants, frequently shows adulteration with this chemical. Hamburger meat served in restaurants often contains sodium nicotinate to preserve its bright red color. Although this chemical is illegal in some municipalities, 37 states permit its use. Several outbreaks of poisoning have been traced to this additive. Eating grilled or pan-fried hamburgers may result in cancer.

Hot Dogs. Preservatives similar to those in ground beef may also be used in frankfurters. Other, additives may also be present, such as anti-aging products, antioxidants to retard rancidity, or tenderizers. A coal-tar color (Red No. 1) was commonly used in the casings of frankfurters until banned by the FDA after this material produced liver damage in experimental animals. Casings are still dyed with artificial colors, and proof of their safety is not conclusive. Although regulations prohibit the use of coloring if it penetrates the produce, on occasion dyes on frankfurters have been found to penetrate as much as one fourth of an inch into the meat.

Pork. Results of a nationwide survey revealed that “whether the sausage came from a federally-inspected packing plant or from the meat grinder of a local butcher, it was often sour or rancid and frequently contaminated with an overabundance of bacteria. Some of it was contaminated with filth. Not one sample, out of the packages tested for quality and flavor, could be judged really outstanding.” Pigs raised commercially are diseased. Many die before the farmer gets a chance to market them. Those that do survive are sick and toxic. These toxins are transferred to the people who eat the pork.

Other Toxins in Meat. Federal Label. Labels for federally-inspected, canned, packaged, or frozen meat must list all the ingredients, common name of the produce, name and address of the processor or distributor, mark of approval and accurate weight. However, meat can be processed legally with sodium nitrate, sodium nitrite, sodium ascorbate, and many other chemicals sanctioned by the FDA. Presently in the United States, up to 500 ppm of sodium nitrate, and up to 200 ppm of sodium nitrite are permitted in certain meats and meat products (and in certain fish and fish products, poultry, and wild game). Far lower limits are set in Europe. “Nitrites should be immediately reduced or eliminated as food preservatives, especially on meat and fish.” This recommendation was made jointly in February of 1970, by Dr. Samuel S. Epstein of the Children's Cancer Research Foundation of Boston and Dr. William Lijinsky of the college of medicine at the University of Nebraska. Noting that nitrosamines—which include nitrites—can produce mutagenic changes, the researchers suspect that by similar pathogenic processes, these agents are carcinogenic and teratogenic as well. Stilbestrol. Medications in feed are used to increase weight rapidly. The most sensational gains are achieved by adding hormones and hormone-like substances to the feed. Stilbestrol is used extensively. Currently, it is estimated that 80% to 85% of all beef cattle are being fed on feed containing stilbestrol. It is also used in the feed for sheep and lambs. Stilbestrol has been acknowledged by scientists as a potent carcinogen, and has been labeled “biological dynamite.” Quantities of stilbestrol as small as 2 ppb are toxic in diets of experimental mice. Cancers in these test animals have been induced by daily doses as low as 7/100,000,000 of a gram (one fourth of a hundred-millionth of an ounce).

Unhealthy Animals. An average of 10% to 30% of beef livers at slaughterhouses are condemned because of abscesses. USDA records showed that during a one-year period, Americans ate millions of pounds of beef from cattle that had “cancer eye” or similar tumorous disorders. The diseased parts were merely cut out and the remainders of the carcasses were permitted to be marketed. Agriculture officials claim that such localized tumors pose no threat to human beings eating meat from other portions of such animals. A government report showed that more than 10% of the 30.1 million cattle carcasses approved by federal inspection underwent some post-mortem cutting for removal of diseased parts. Another report showed that 2,400,000 cattle whose cancerous or tubercular livers were discarded had the rest of their carcasses sent on to be processed for food.

Sugar. The refined-carbohydrate diet is blamed by Dr. Denis F. Burkitt as the single most important cause of large-bowel cancers, occurring on a worldwide scale when people forsake their traditional dietary habits and consume large amounts of refined carbohydrates. Avoid sugar, advises a famous English nutritionist, and you are less likely to become fat, run into nutritional deficiencies, have a heart attack, develop diabetes or dental decay or a duodenal, ulcer, and possibly reduce your chances of getting gout, dermatitis, some forms of cancer, and in general increase your life span. It is important to realize that by the mere omission of a single common food, and beverage ingredient such as sugar, so many benefits may result, and that its excessive use can contribute, at least in part, to so many desperate conditions and diseases.

Brown Sugar. The commercial brown sugar color isn't from molasses residue. Virgin sugar is rinsed to remove molasses residue, then put into a centrifuge where it is separated from the crystals. This is melted, filtered and boiled repeatedly with animal-bone charcoal to concentrate and form crystals. Molasses is added back to sugar to achieve its brown color. Dr. W. C. Heuper, M.D. in an experimental study for Cancer Research warns that sugar manufactured with this animal-bone charcoal process may be carcinogenic (cancerous).

Fats and Oils. Hydrogenation. How is the liquid oil or soft fat hardened? It is exposed o a high temperature and placed under pressure. Hydrogen is then bubbled through the oil in the presence of nickel, platinum, or some other catalyst. The hydrogen atoms combine with the carbon atoms, and the produce becomes saturated or hardened. There is no assurance that nickel, if used as the catalyst, leaves no residue in the product. This element, even in minute quantities in the diet, is suspected of being a carcinogen. As evidence has mounted revealing the menace of hydrogenation, some shortening processors have attempted to change their methods to avoid economic losses. They proclaim their products “high in unsaturates” but adding that they “stay freshly sweet at room temperature.” These products may contain anti-aging products, antioxidants, in addition to emulsifiers, defoamers, and artificial colors and natural or artificial flavors. Artificial anti-aging products, antioxidants in fats are considered possible carcinogens.

Heated Oils. Prolonged heating and reheating at high temperatures produce harmful substances suspected as cancer-inciting or oils. Deep-fat frying is favored in many short-order diners and also in “good” restaurants for economy, speed, and convenience. It is a method also used extensively in processed foods like potato chips, doughnuts, baked goods, and serve-and-heat dishes, as well as in many homes. Consumers Research recommends that deep-fat frying should be avoided. Also be shunned are all burned fatty foods and charcoal-broiled meats. All charred, blackened, or burned portions of meats or other fatty foods are carcinogenic.

Caffeine. Caffeine has been shown to result in genetic and chromosomal changes in animals, bacteria, and higher plants. A retrospective study showed that men who drank cola beverages containing caffeine have a significant increase in bladder cancer compared to noncola drinkers. Other studies' which implicate coffee and caffeine as mutagenic include discussion of genetic effects and effects on chromosomes of human lymphocytes.

Alcoholic Beverages. Consumption of alcoholic beverages entails an increased risk of developing cancer of the mouth, larynx, pharynx, and esophagus according to the International Agency for Research on Cancer of the World Health Organization. The evidence is that the increase in cancer of the esophagus is proportional to the amount of ethanol consumed. In all four cancer sites, the role of tobacco is also important, and the ill effects of these two factors—drinking alcoholic beverages and smoking tobacco—tend to multiply when they act together. Ethanol may act as a co-carcinogen by enhancing the role of other cancer-causing agents. Ethanol is an excellent solvent for chemicals that are themselves cancer-causing agents, such as polycyclic hydrocarbons and nitrosamines. The presence of these chemicals has been detected in some commonly-consumed alcoholic beverages drunk in areas where esophageal cancer is common.

Carboxymethyl Cellulose (CMC) or other cellulose gum is a cellulose derivative with carboxymethyl groups (—CH₂—COOH) bound to some of the hydroxyl groups of the glucopyranose monomers that make up the cellulose backbone. It is often used as its sodium salt, sodium carboxymethyl cellulose.

Catalytic Properties of Nanocrystalline. The gas-condensation technique is used to produce the nanocrystalline (NC) WO₃₋ₓ, Pt/WO₃₋ₓ, and Pd/WO₃₋ₓ powders under different atmosphere and pressure. HRTEM images show that a coherently bonded interface exists between Pt or Pd and WO₃₋ₓ. The nanocrystal WO₃₋ₓ, Pt/WO₃₋ₓ, and Pd/WO₃₋ₓ grow into a needle shape with a plate inside when these as-evaporated powders are compacted and sintered at 900° C. for 2 h. The plate grows preferentially in {220} plane along the <0011> direction. However, the mean particles size of nanophase Pt and Pd increases only from <10 nm to 30 nm and 50 nm, respectively. The results of CO oxidation show that nanophase Pt/WO₃₋ₓ, powders have better catalytic effects on converting CO to CO₂ than nanophase WO₃₋ₓ and Pd/WO₃₋ₓ powders.

Cellulose is an organic compound and/or a polysaccharide consisting of a linear chain of several hundred to many thousand/ors of β(1→4) linked D-glucose units. Cellulose is an important structural component of the primary cell wall of green plants, many forms of algae and/or the oomycetes. Some species of bacteria secrete it to form biofilms. Cellulose is the most abundant organic polymer on Earth. The cellulose content of cotton fiber is 90%, that of wood is 40%-50% and/or that of dried hemp is approximately 45%. Cellulose is mainly used to produce paper board and/or paper. Smaller quantities are converted into a wide variety of derivative products such as cellophane and/or rayon. Conversion of cellulose from energy crops into biofuels such as cellulosic ethanol is under investigation as an alternative fuel source.

Cellulose Acetate is the acetate ester of cellulose. Cellulose acetate can optionally be used as a film base in photography, as a component in some coatings, and as a frame material for eyeglasses. Cellulose acetate can optionally be used as a synthetic fiber in the manufacture of cigarette filters and playing cards.

Cellulose for Industrial Use is mainly obtained from wood pulp and/or cotton. Cellulose, one of the world's most abundant, natural and/or renewable polymer resources, is widely present in various forms of biomasses, such as trees, plants, tunicate and/or bacteria. Cellulose molecule consists of β-1, 4-D-linked glucose chains with molecular formula of (C₆H₁₀O₅)ₙ (n ranging from 10,000 to 15,000) through a acetal oxygen covalently bonding C1 of one glucose ring and/or C4 of the adjoining ring. In plant cell walls, approximately 36 individual cellulose molecule chains connect with each other through hydrogen bonding to form larger units known as elementary fibrils, which are packed into larger micro fibrils with 5-50 nm in diameter and/or several micrometers in length. These micro fibrils have disordered (amorphous) regions and/or highly ordered (crystalline) regions. In the crystalline regions, cellulose chains are closely packed together by a strong and/or highly intricate intra- and/or intermolecular hydrogen-bond network, while the amorphous domains are regularly distributed along the microfibrils. When lignocellulosic biomass are subjected to pure mechanical shearing, and/or a combination of chemical, mechanical and/or enzymatic treatment, the amorphous regions of cellulose microfibrils are selectively hydrolyzed under certain conditions because they are more susceptible to be attacked in contrast to crystalline domains. Consequently, these microfibrils break down into shorter crystalline automotive products and/or parts, electronics with high crystalline degree, which are generally referred to as cellulose nanocrystals (CNCs). CNCs are also named as microcrystals, whiskers, nano products, microcrystallites, nanofibers, or nanofibrils in the liturautes, all of which are called “cellulose nanocrystals.”

Cellulose is a complex carbohydrate, or polysaccharide, consisting of 3,000 or more glucose units. The basic structural component of plant cell walls, cellulose comprises about 33% of all vegetable matter (90% of cotton and 50% of wood are cellulose) and is the most abundant of all naturally occurring organic compounds. Nondigestible by man, cellulose is a food for herbivorous animals (e.g., cows, horses) because they retain it long enough for digestion by microorganisms present in the alimentary tract; protozoans in the gut of insects such as termites also digest cellulose. Of great economic importance, cellulose is processed to produce papers and fibers and is chemically modified to yield substances used in the manufacture of such items as plastics, photographic films, and rayon. Other cellulose derivatives are used as adhesives, explosives, thickening agents for foods, and in moisture-proof coatings.

Cellulose [selú-lōs] is a carbohydrate forming the skeleton of most plant structures and plant cells. It is the most abundant polysaccharide in nature and is the source of dietary FIBER, preventing constipation by adding bulk to the stool. Good sources in the diet are vegetables, cereals, and fruits.

Absorbable cellulose (oxidized cellulose) an absorbable oxidation product of cellulose, applied locally to stop bleeding. Cellulose sodium phosphate an insoluble, nonabsorable cation exchange resin prepared from cellulose;
it binds calcium and is used to prevent formation of calcium-containing KIDNEY STONES.

Cellulose Fibers are fibers made with ether or esters of cellulose, which can optionally be obtained from the bark, wood or leaves of plants, or from a plant-based material. Besides cellulose, these fibers are compound of hemicellulose and/or lignin, and/or different percentages of these components are responsible for different mechanical properties observed. The main applications of cellulose fibers are in textile industry, as chemical filter, and/or fiber-reinforcement composite, due to their similar properties to engineered fibers, being another option for biocomposites and/or polymer composites. Cellulose in Medicine. Cellulose is a complex carbohydrate that is composed of glucose units, forms the main constituent of the cell wall in most plants and is important in the manufacturing of numerous products, such as pharmaceuticals.

Cellulose in Science. Cellulose is a carbohydrate that is a polymer composed of glucose units and that is the main component of the cell walls of most plants. It is insoluble in water and used to make paper, cellophane, textiles, explosives and other products.

Cellulose Nanocrystals (CNC) are cellulose-based nanoparticles that can be extracted by acid hydrolysis from a wide variety of natural source materials (e.g., trees, annual plants, tunicates, algae, bacteria). These rod-like or whisker-shaped particles (3-20 nm wide, 50-2000 nm long) have a unique combination of characteristics: high axial stiffness (˜150 GPa), high tensile strength (estimated at 7.5 GPa), low coefficient of thermal expansion (˜1 ppm/K), thermal stability up to ˜300° C., high aspect ratio (10-100), low density (˜1.6 g/cm3), lyotropic liquid crystalline behavior, and shear thinning rheology in CNC suspensions. The exposed —OH groups on CNC surfaces can be readily modified to achieve different surface properties and have been used to adjust CNC self-assembly and dispersion for a wide range of suspensions and matrix polymers and to control interfacial properties in composites (e.g., CNC-CNC and CNC-matrix). This unique set of characteristics results in new capabilities compared to more traditional cellulose based particles (wood flakes, plant fibers, pulp fibers, etc.). and the development of new composites that can take advantage of CNCs' enhanced mechanical properties, low defects, high surface area to volume ratio, and engineered surface chemistries. CNCs have been successfully added to a wide variety of natural and synthetic polymers and have been shown to modify composite properties (mechanical, optical, thermal, barrier). Additionally, CNCs are a particularly attractive nanoparticles because they have low environmental, health, and safety risks, are inherently renewable, sustainable, and carbon-neutral like the sources from which they are extracted, and have the potential to be processed in industrial-scale quantities at low costs.

Processing of Cellulose Nanocrystals. Although there are many variants of the process to isolate CNCs from a given cellulose source material, this process generally occurs in two primary stages. The first stage is a purification of the source material (plants, tunicates, algae, bacteria, etc.) to remove most of the non-cellulose components in the biomass. These include lignin, hemicellulose, fats and waxes, proteins, and inorganic contaminants. The second stage uses an acid hydrolysis process to deconstruct the “purified” cellulose material into its crystalline components. This is accomplished by preferentially removing the amorphous regions of the cellulose microfibrils. The resulting whisker-like particles (3-20 nm wide, 50-2000 nm long) are ˜100% cellulose, are highly crystalline (62%-90%, depending on cellulose source material and measurement method), and have been referred to in the literature as cellulose nanocrystals (CNCs), nanocrystalline cellulose (NCC), and cellulose nanowhiskers (CNW) to name a few. The variations in CNC characteristics. Transmission electron microscopy (TEM) image of CNCs extracted from microcrystalline cellulose. Cellulose Nanocrystals 10 Production and Applications of Cellulose Nanomaterials.

Cellulose Insulation. The word cellulose comes from the French word for a living cellule and glucose, which is sugar. Building insulation is low-thermal-conductivity material used to reduce building heat loss and gain, and reduce noise transmission. Cellulose insulation is plant fiber used in wall and roof cavities to insulate, draught proof and reduce noise.

Cellulase. Human cannot digest cellulose because we don't have the necessary enzymes. Cellulolysis is the process of breaking cellulose. Since they are made of glucose molecules, cellulose can be broken down into glucose by hydrolysis. First, the last molecule is broken down into smaller polysaccharides, which are known as cellodextrins. Finally, these are broken down to glucose. Though humans cannot digest cellulose, some mammals like cows, sheeps, goats, and horses can digest cellulose. These animals are known as ruminants. They have this capability due to a bacteria living in their digestive tract. These symbiotic bacteria possesses enzymes to break down cellulose by anaerobic metabolism. These enzymes are known as cellulases. Further cellulase enzymes are produced by fungi and protozoans, to catalyze cellulolysis. Five types of cellulases are there in this class of enzymes. Endocellulase, exocellulase, cellobiase, oxidative cellulases, and cellulose phosphorylases are those five types.

Classification of Cellulose-Based Polymers. CELLULOSE. Pure cellulose is available in different forms in the market with very different mechanical and pharmaceutical properties. The difference between various forms of cellulose is related to the shape, size and degree of crystallinity of their particles (fibrous or agglomerated).

Microcrystalline Cellulose (MCC) is the most known cellulose, which extensively used in pharmaceutical industries. MCC grades are multifunctional pharmaceutical excipients, which can optionally be used as compressibility enhancer, binder in wet and dry granulation processes, thickener and viscosity builder in liquid dosage forms and free-flowing agents in solid dosage forms. Mechanical properties of MCC grades are greatly influenced by their particles size and degree of crystallization. In recent years the new grades of MCC are prepared with improved pharmaceutical characteristics such as silisified MCC (SMCC) and second-generation MCC grades or MCC type II (MCC-II). These grades are prepared by co-processing of cellulose with other substances such as colloidal silicon dioxide or by special chemical procedures. Other types of available pure cellulose are powdered cellulose (PC) and low crystallinity powdered cellulose (LCPC). Regenerated cellulose is one of the other forms of processed cellulose, which produced by chemical processing on natural cellulose. In the first step, cellulose dissolves in alkali and carbon disulfide to make a solution called “viscose”. Viscose reconverted to cellulose by passing through a bath of dilute sulfuric acid and sodium sulphate. Reconverted cellulose passed through several more baths for sulfur removing, bleaching and adding a plasticizer (glycerin) to form a transparent film called cellophane. Cellophane has several applications in pharmaceutical packaging due to its suitable characteristics such as good compatibility, durability, transparency and elasticity.

CELLULOSE ETHER DERIVATIVES. Cellulose ethers are high molecular weight compounds produced by replacing the hydrogen atoms of hydroxyl groups in the anhydroglucose units of cellulose with alkyl or substituted alkyl groups. The commercially important properties of cellulose ethers are determined by their molecular weights, chemical structure and distribution of the substituent groups, degree of substitution and molar substitution (where applicable). These properties generally include solubility, viscosity in solution, surface activity, thermoplastic film characteristics and stability against biodegradation, heat, hydrolysis and oxidation. Viscosity of cellulose ether solutions is directly related with their molecular weights. Examples of mostly used cellulose ethers are: Methyl cellulose (MC), Ethyl cellulose (EC), Hydroxyethyl cellulose (HEC), Hydroxypropyl cellulose (HPC), hydroxypropylmethyl cellulose (HPMC), carboxymethyl cellulose (CMC) and sodium carboxymethyl cellulose (NaCMC).

CELLULOSE ESTER DERIVATIVES. Cellulose esters are generally water insoluble polymers with good film forming characteristics. Cellulose esters are widely used in pharmaceutical controlled release preparations such as osmotic and enteric-coated drug delivery systems, pharmaceutical coatings process, medical coating applications, topical ophthalmic protectant and lubricant and the like. These polymers are often used with cellulose ethers concurrently for preparation of micro-porous delivery membranes. Cellulose esters categorized in organic and inorganic groups. Organic cellulose esters are more important in pharmaceutical industries. Various types of organic cellulose esters have been used in commercial products or in pharmaceutical investigations such as cellulose acetate (CA), cellulose acetate phthalate (CAP), Cellulose acetate butyrate (CAB), Cellulose acetate trimelitate (CAT), hydroxupropylmethyl cellulose phthalate (HPMCP) and so on (Heinämäki et al., 1994). The most available formulations in market which made by these polymers are enteric coated dosage forms which are usually produced applying acid resistant polymeric coats containing phthalate derivatives of cellulose esters especially cellulose acetate phthalate (Lecomte et al., 2003; Liu & Williams III, 2002). Inorganic cellulose esters such as cellulose nitrate and cellulose sulphate are less important than organic cellulose esters in pharmaceutical industries. Cellulose nitrate or pyroxylin is a transparent compound with good film forming ability but rarely applied alone in pharmaceutical formulations due to its very low solubility in currently used pharmaceutical solvents as well as their very high flammability. The use of pure cellulose nitrate in drug formulations only limited to one topical anti-wart solution named collodion that made with 4% w/v concentration in diethyl ether/ethanol mixture as solvent. Cellulose nitrate/cellulose acetate mixture is also exploited to prepare micro-porous membrane filters used in pharmaceutical industries. Applications of cellulose and its derivatives in pharmaceutical industries

APPLICATION IN BIOADHESIVE AND MUCOADHESIVE DRUG DELIVERY SYSTEMS. Bioadhesives and mucoadhesives are drug containing polymeric films with ability of adhering to biological membranes after combining with moisture or mucus compounds. Bioadhesives were developed in mid 1980s as a new idea in drug delivery and nowadays they have been accepted as a promising strategies to prolong the residence time and to improve specific localization of drug delivery systems, pharmaceutical coatings process, medical coating applications, topical ophthalmic protectant and lubricant and the like on various biological membranes (Lehr, 2000; Grabovac et al., 2005; Movassaghian et al., 2011). In compared with tablets, these dosage forms have higher patient compliance due to their small size and thickness. Other advantage of these drug delivery systems, pharmaceutical coatings process, medical coating applications, topical ophthalmic protectant and lubricant and the like is their potential to prolong residence time at the site of drug absorption and thus they can reduce the dosing frequency in controlled release drug formulations. These dosage forms can also intensify the contact of their drug contents with underlying mucosal barrier and improve the epithelial transport of drugs across mucus membranes especially in the case if poorly absorbed drugs (Ludwig, 2005; Lehr, 2000). Some special polymers can optionally be in these formulations with epithelial permeability modulation ability by loosening the tight intercellular junctions. Some of these polymers also can act as proteolytic enzymes inhibitor in orally used adhesive formulations of sensitive drugs (Lehr, 2000). Bioadhesives considered as novel drug delivery systems, pharmaceutical coatings process, medical coating applications, topical ophthalmic protectant and lubricant and the like. These dosage forms are formulated to use on the skin and mucus membranes of gastrointestinal, ear, nose, eye, rectum and vagina. The main excipients of these formulations are adhesive and film-former polymer(s). Adhesive polymers are synthetic, semi synthetic or natural macromolecules with capability of attaching to skin or mucosal surfaces. Very different types of polymers have been used as bioadhesive polymers. Synthetic polymers such as acrylic derivatives, carbopols and polycarbophil, natural polymers such as carageenan, pectin, acacia and alginates and semi-synthetic polymers like chitosan and cellulose derivatives are used in bioadhesive formulations (Deshpande et al., 2009; Grabovac et al., 2005). Cellulose derivatives especially cellulose ethers are widely used in bioadhesives. There are used in various types of these formulations such as buccal, ocular, vaginal, nasal and transdermal formulations alone or with combination of other polymers. More recently used cellulose ethers in bioadhesives include nonionic cellulose ethers such as ethyl cellulose (EC), hydroxyethyl cellulose, hydoxypropyl cellulose (HPC), methyl cellulose (MC), carboxymethyl cellulose (CMC) or hydroxylpropylmethyl cellulose (HPMC) and anionic ether derivatives like sodium carboxymethyl cellulose (NaCMC). Ability of polymer to take up water from mucus and pH of target place are important factors determining the adhesive power of polymers. Some bioadhesive polymers such as polyacrylates show very different adhesion ability in various pH values thus the selection of adhesive polymer should be made based on the type of bioadhesive preparation. One advantage of cellulose ethers such as NaCMC and HPC is lesser dependency of adhesion time and adhesion force of them to pH of medium in compared with polyacrylate and thiolated bioadhesive polymers (Grabovac et al., 2005). Cellulose ethers, alone or their mixtures with other polymers, have been studied in oral (Deshpande et al., 2009; Venkatesan et al., 2006), buccal (Perioli et al 2004), ocular (Ludwig, 2005), vaginal (Karasulu et al., 2004) and transdermal (Sensoy et al., 2009) bioadhesives. In some studies, other groups of adhesive polymers or polysaccharides are used with cellulose ethers to improve their adhesion characteristics such as adhesion time and adhesion force. Concurrent use of polyvinyl pyrrolidone (PVP), hydroxypropyl beta cyclodextrin, polycarbophil, carbopol(s), pectin, dextran and mannitol with HPMC, HEC or NaCMC have been reported in the literatures. (Karavas et al., 2006).

APPLICATION IN PHARMACEUTICAL COATING PROCESSES. Solid dosage forms such as tablets, pellets, pills, beads, spherules, granules and microcapsules are often coated for different reasons such as protection of sensitive drugs from humidity, oxygen and all of inappropriate environmental conditions, protection against acidic or enzymatic degradation of drugs, odor or taste masking or making site or time specific release characteristics in pharmaceuticals to prepare various modified release drug delivery systems, pharmaceutical coatings process, medical coating applications, topical ophthalmic protectant and lubricant and the like such as sustained release, delayed release, extended release, immediate release, pulsatile release or step-by-step release dosage forms (Barzegar jalali et al., 2007; Gafourian et al., 2007). Both ether and ester derivatives of cellulose are widely used as coating of solid pharmaceuticals. Cellulose ethers are generally hydrophil and convert to hydrogel after exposing to water. Although, some of the cellulose ethers e.g. ethyl cellulose are insoluble in water but majority of them such as methyl, hydroxypropyl and hydroxylpropylmethyl cellulose are water soluble. Both of soluble and insoluble cellulose ethers can absorb water and form a gel. After exposing of these coated dosage forms with water, the coating polymers form to hyrogel and gradually dissolve in water until disappear but the insoluble cellulose ether coatings remain as a viscose gel around tablets and drug release is performed by diffusion of drug molecules within this layer. These two types of dosage forms called dissolution-controlled and diffusion-controlled drug delivery systems, pharmaceutical coatings process, medical coating applications, topical ophthalmic protectant and lubricant and the like, respectively. Despite cellulose ethers, the cellulose esters are generally water insoluble or water soluble in a distinct pH range. These polymers like cellulose acetate (CA), cellulose acetate phthalate (CAP) and cellulose acetate butyrate (CAB) do not form gel in presence of water and they are widely used for preparing of pH sensitive and semi-permeable micro-porous membranes. These membranes are employed for wide variety of controlled release coating of pharmaceuticals especially in enteric or osmotic drug delivery devices. These polymers are benefited to make different cellulosic membrane filters applied in pharmaceutical industries.

APPLICATION IN EXTENDED RELEASE (ER) SOLID DOSAGE FORMS. Extended release pharmaceuticals refer to dosage forms that allow a twofold or greater reduction in frequency of the drug administration in comparison with conventional dosage forms. These formulations can be made as coated or matrix type. Coated ER formulations are generally made with water insoluble polymeric film coating with or without gel-forming ability. The dominant mechanism of drug release in coated ERs is diffusion whereas in matrix type of ERs, erosion of matrix is the main mechanism of drug release. The most used cellulosic polymer in these modified release dosage forms is ethyl cellulose. Ethyl cellulose is completely insoluble in water, glycerin and propylene glycol and soluble in some organic solvents such as ethanol, methanol, toluene, chloroform and methyl acetate. Aqueous dispersions of ethyl cellulose such as Surelease® (Colorcon) or Aquacoat® (FMC Polymer) or its organic solutions can optionally be used for coating of extended release formulations. After ingestion of these formulations, an insoluble viscose gel is forming around the tablet, which doesn't allow to drug to freely release from dosage form. Drug molecules should pass across this barrier by diffusion mechanism to enter the bulk dissolution medium and thus the release duration is extended much more than the same uncoated conventional formulation. Larger solid pharmaceuticals like tablets can be coated with rotating pan coaters whereas the smaller types as pills, beads or granules are coated with fluidized bed or air-suspension coater equipments. Because of water insolubility of EC, it is often used in conjunction with water soluble polymers such as MC and HPMC in aqueous coating liquids (Frohoff-Hülsmann et al., 1999a, 1999b). EC solutions in organic solvents such as ethanol can be thickened by HPMC or HPC (Rowe, 1986; Larsson et al., 2010). Water soluble cellulosic polymers with higher amounts can optionally be used as pore former in micro-porous types of extended release and enteric systems. Using of plasticizers is necessary for achieving acceptable coating of pharmaceuticals by these polymers. EC is compatible with commonly used plasticizers such as dibutyl phthalate, diethyl phthalate, dicyclohexyl phthalate, butyl phthalyl butyl glycolate, benzyl phthalate, butyl stearate and castor oil. Other plasticizers such as triacetin, cholecalciferol and α-tocopherol also have been used in EC film coats (Arwidson et al., 1990; Kangarlou et al., 2008). The molecular weights of ECs are in a wide range and different grades of them are existed from 4 to 350 (Colorcon official website). Concentration of 5% w/v from these EC grades in toluene/ethanol mixture at 25° C. can produce about 3 to 380 cp viscosity.

IN EXTENDED RELEASE POLYMERIC MATRICES. Matrices are very simple and efficient systems for controlling drug release from dosage forms. Production of these systems is less time consuming and no needs to special or sophisticated equipments. Majority of ER matrices are made by a simple mixing of drug, polymer(s) and filler followed by one or two stage compaction process. Polymeric matrices as drug delivery systems, pharmaceutical coatings process, medical coating applications, topical ophthalmic protectant and lubricant and the like are very important in developing of modified release dosage forms. In these devices, the drug is dispersed either molecularly or in particulate form within a polymeric network. The main types of drug delivery matrices included swellable and hydrophilic monolithic, erosion controlled and non-erodible matrices (Roy et al., 2002). The use of hydrophilic matrices has become extremely popular in controlling the release rate of drugs from solid dosage forms due to their attractiveness in the case of economic and process development points of view (Conti et al., 2007). During the last two decades, hydrophilic swellable polymers have been widely used for preparation of controlled release matrix tablet formulations. Although various types of rate controlling polymers have been used in hydrophilic matrices, cellulose derivatives especially cellulose ethers are probably the most frequently encountered in pharmaceutical literatures and the most popular polymers in formulation of commercially available oral controlled release matrices. They good compressibility characteristics so they are easily converted to matrices by direct compression technique. In contact with an aqueous liquid, i.e., dissolution medium or gastrointestinal fluid, the hydrophilic polymers present in the matrix swell and a viscose gelatinous layer formed in outer surface of matrix. This layer controls the drug release from matrix. Drug molecules can release out of system by diffusion across this layer. Viscosity of the gel layer is a critical rate-controlling factor in drug release rate from matrices. Erosion of polymeric matrices also can influence the release of the drug from system. Increasing viscosity of the gel, gives rise to increase the resistance against polymer erosion and drug diffusion resulting in reduction of the drug release rate. Various types of cellulose derivatives have been used in formulation of hydrophilic polymeric matrices such as HPMC, NaCMC, CMC, HEC, HPC and EC with different molecular weights (Barzegar-jalali et al., 2010; Javadzadeh et al., 2010; adibkia et al., 2011; Asnaashari et al., 2011). Both of soluble and insoluble cellulose ethers can optionally be in hydrophilic polymeric matrices due to their hydrophilic nature and ability of them to forming gel in aqueous media. The highest swelling power and hydration rate among cellulose ethers is related to HEC (Sa\check{s}a et al., 2006) but the mostly used cellulose ether is hydrophilic matrices is HPMC due to its excellent swelling properties, good compressibility and fast hydration in contact with water (Ferrero et al., 2008, 2010; Nerurcar et al., 2005). For achieving the good release characteristics, mixtures of various cellulose ethers or mixtures of different grades of a distinct polymer with different ratios can optionally be based on the intended release rate of controlled release system (Chopra et al., 2007). Some specialized hydrophilic matrices can be made with cellulose ethers for special purposes for example, HPMC matrices with alkalizing buffers like sodium citrate for protection of acid labile drugs have been investigated (Pygall et al., 2009).

APPLICATION IN OSMOTIC DRUG DELIVERY SYSTEMS. In recent years, considerable attention has been focused on development of novel drug delivery systems, pharmaceutical coatings process, medical coating applications, topical ophthalmic protectant and lubricant and the like (NDDS). Among various NDDS available in the market, oral controlled release (CR) systems hold the major market share because of their advantages over others. Majority of oral CR systems fall in the category of matrices, reservoirs and osmotic devices. Among various types of CR systems, osmotic devices are considered as novel CR systems (J. Shokri et al., 2008a). These formulations utilize osmotic pressure as energy source and driving force for delivery of drugs. Some physiological factors such as pH, presence of food and gastrointestinal motility may affect drug release from conventional CR systems (matrices and reservoirs), whereas, drug release from oral osmotic systems is independent of these factors to a large extent. A classic osmotic device basically consists of an osmotically active core surrounded by a semi-permeable membrane (SPM) and a small orifice drilled through SPM using LASER or mechanical drills. In fact, this system is really a coated tablet with an aperture, which acts as drug delivery port. This type of devices is called monolithic or elementary osmotic pumps (EOPs). The more sophisticated osmotic devices have bi-layer (push-pull systems) or tri-layer (sandwich osmotic pumps) cores consisted of an osmotically active drug layer and polymeric layer(s) in one or two sides. Some of osmotic systems called asymmetric membrane or controlled porosity osmotic pumps have not any orifice in their SPM (wang et al., 2005). In these devices, water soluble polymers are used in their SPM as pore formers. Pore formers dissolve after exposing of dosage form to aqueous media and numerous micro pores are created in SPM for drug delivery reason. When an osmotic tablet exposed to an aqueous environment, water pumps from outside into the system due to the great osmotic pressure difference between two sides of SPM. Pumping of water into the system increases the inner hydrostatic pressure leading the saturated drug solution to flow through the small drug delivery orifice or micro pores (in the case of asymmetric membrane devices). Because of high difference of osmotic pressure between two sides of SPM, the osmotic pressure gradient remain constant and thus, the release rate of drug from these devices is almost constant and independent to environmental conditions. EOPs are the most commercially important osmotic devices so that more than 240 patents have been devoted. Procardia XL® and Adalat CR (nifedipine), Acutrium® (phenylpropanolamine), Minipress XL® (prazocine) and Volmax® (salbutamol) are examples of EOPs available in the market (J. Shokri et al., 2008a; Nokhodchi et al., 2008).

IN SPM FORMULATION OF OSMOTIC SYSTEMS. As noted earlier, each osmotic delivery systems, pharmaceutical coatings process, medical coating applications, topical ophthalmic protectant and lubricant and the like, is consisted of two main components included osmotically active core and semi-permeable membrane (SPM). Cellulose acetate (CA) is the mostly used polymer in formulation of SPM in all types of osmotic drug delivery devices. This polymer is the most important cellulose ester derivative with good film forming ability and mechanical characteristics for using in osmotic systems. CA is insoluble in water in both acidic and alkaline conditions. The CA films are only permeable to small molecules such as water while larger molecules like organic drugs can not pass through them. Plasticizers are used in SPM composition for improving the flexibility and mechanical properties of membrane. Various types of plasticizers have been used in formulation of osmotic pharmaceuticals such as castor oil, low and medium molecular weights polyethylenglycols (PEGs), sorbitol, glycerin, propylene glycol, triacetine, ethylene glycol monoacetate, diethyl phthalate, diethyl tartrate and trimethyl phosphate (J. Shokri et al., 2008a, 2008b; Prabakaran et al., 2004; Makhija & Vavia, 2003; Liu et al., 2000a, 2000b; Okimoto et al., 1999). Generally, the mixture of hydrophilic and hydrophobic plasticizers is used for producing the intended drug release characteristics. In controlled porosity osmotic pumps (CPOPs), the additional components such as pore formers are needed. The most efficient pore formers are hydrophilic polymers with high water solubility properties. Water soluble cellulose ether derivatives can optionally be used as pore former in SPM of these devices. Low molecular weight grades of these polymers are suitable for this purpose due to their faster dissolution rate and lower viscosities. Low molecular weight MCs and HPMCs have been used as pore former in CPOP formulations. Central cores are coated with a coating formulation containing SPM components such as film former (CA), pore former(s) and plasticizer(s) dissolved or dispersed in a suitable liquid base. Acetone/ethanol mixtures are generally used as solvent system to dissolve cellulose acetate in coating liquid (J. Shokri et al., 2008a; Nokhodchi et al., 2008; M. H. Shokri et al., 2011). In some studies, cellulose acetate is used as fine particles suspended in an aqueous medium for coating of osmotic cores (Liu et al., 2000b). Ethyl cellulose (EC) and ethylhydroxyl propyl cellulose also have been used as SPM of osmotic devices in some studies but permeability of these membranes is lower than CA membranes. In these formulations, hydrophilic cellulose ether derivatives such as HPMC have been used for improving SPM permeability (Marucci et al., 2010; Wang et al., 2005; Hjärtstam et al., 1990).

IN CENTRAL CORE OF OSMOTIC SYSTEMS. Central core of an osmotic pump is generally a simple compressed tablet basically consisted of the active drug(s), osmotically active agent(s), hydrophilic polymer(s) and other commonly used ingredients such as filler, compressibility enhancer, free flowing agent and lubricant. In one compartment devices (EOPs and controlled porosity OP), these polymers mixed with other ingredients and compressed to a tablet whereas in two layered (Push-Pull systems), or tri layered (Sandwich systems) cores, these polymers compressed in one or two separated layer in one or both sides of drug layer (J. Shokri et al., 2008b; Kumaravelrajan et al., 2010). These polymers should have high water uptake and swelling capacity. Cellulose derivatives play an important role in core formulations of osmotic devices. Water soluble cellulose ethers commonly used as core polymers due to their hydrophilicity and good swelling properties. Most currently used polymers for this purpose are MC, HEC, HPC and HPMC with various molecular weights. After exposing of system to water, water move into the system due to great osmotic pressure difference between outer and inner part of device. This water is imbibed to polymer(s) and causes swelling of them. Swelling of core polymer(s) produce the driving force for ejecting the drug solution from drug release orifice with constant rate (J. Shokri et al., 2008a, 2008b; Prabakaran et al., 2004; Makhija & Vavia, 2003; Liu et al., 2000a, 2000b). Among cellulose ethers, different grades of HPMC have been used more than others in core formulation. Microcrystalline cellulose (MCC) has also frequently used the core formulations as compressibility enhancer. MCC is one of the most compressibility enhancers that widely used in direct compression as well as wet granulation techniques for preparing various types of tablets, pellets and pills.

APPLICATION IN ENTERIC COATED SOLID DOSAGE FORM. Enteric coated solid dosage forms are the main groups of delayed release drug delivery systems, pharmaceutical coatings process, medical coating applications, topical ophthalmic protectant and lubricant and the like which designed for releasing of their drug(s) content in the lower parts of gastrointestinal tract such as small intestine and colon. Enteric dosage forms can be considered as a type of oral site specific pharmaceuticals that initiate drug release after passing from stomach. Enteric oral dosage forms are suitable for formulation of acid-labile drugs or drugs with irritancy potential for inner protective layer of stomach such as non-steroidal anti inflammatory drugs (NSAIDs). The commonly used materials in enteric coated formulations are pH-dependent polymers containing carboxylic acid groups. These polymers remain un-ionized in low pH conditions like environment of stomach and become ionized with increasing of pH toward natural and light alkaline zone similar to the small intestine condition (Liu et al., 2011). These polymers also should have the good film forming properties to produce smooth coats with good integrity. Various polymers have been used for production of enteric coated dosage forms such as Eudragit® polymers and pH-dependent cellulose derivatives. Cellulose derivatives which commonly used as enteric coating polymers include cellulose acetate phthalate (CAP), cellulose acetate trimelitate (CAT), hydroxypropylmethyl cellulose phthalate (HPMCP), carboxymethylethyl cellulose (CMEC) and hydroxypropylmethyl cellulose acetate succinate (HPMCAP) (Williams III & Liu, 2000). Apart from the main enteric polymer, the type and amount o plasticizer(s) is very important for achieving uniform, smooth and resistant enteric films. Some of mostly used plasticizers in enteric coated formulations are diethyl phthalate, glyceryl triacetate, glyceryl monocaprylate and triethyl citrate (Williams III & Liu, 2000; Gosh et al., 2011). In some cases, hydrophilic cellulose ether derivatives especially HPMC are used with enteric polymer for improving the film forming and plasticity of main enteric polymer. HPMC is also used in enteric coating process as pre-coating or sub-coating polymer due to its very good film forming properties and suitable polymer-to-polymer adhesion with enteric coating polymers especially with cellulose ester derivatives such as CAP, HPMCP, HPMCAS, CMEC and CAT (Williams III & Liu, 2000). Three commercially available enteric coating preparations included solid forms of enteric polymers which should be dissolved in suitable organic solvent mixture before coating process, ready-to-use organic enteric coating solutions and aqueous polymeric dispersions. Aqueous nanodispersions of enteric coating polymers such as HPMCP have also been investigated for improving physicochemical and mechanical characteristics of coating (Kim et al., 2003).

APPLICATION AS COMPRESSIBILITY ENHANCERS. More than 80% of all dosage forms available or administered to man are tablets. The main reason of this great popularity is the advantages of tablets over other forms of pharmaceuticals. Ease of manufacturing, convenience dosing and greater stability in compared with liquid or semisolid dosage forms are some of these advantages. Two common ways for tablet manufacturing are compression and molding. Except of a few cases, tablets are made by compression technique. The simplest and fastest kind of compression is named direct compression method in which the drug and all of other excipients are mixed and compressed in one-stage process with proper compression force to form tablet. This method commonly used for tableting of medium to high potency drugs where the drug content in them is less than 30% of formulation (Jivari et al., 2000). In the other cases with higher amounts of low compactable drugs, dry or wet granulation techniques are used for preparing tablets. In dry granulation method, compression of ingredients are performed in two or multi-stage process to improve compressibility of the ingredients. Slugging and roller compaction techniques used for initial compression of powder mixtures before final tableting process. One of the common difficulties in direct compression and dry granulation is low compactability of the drug content especially when the drug amount is higher than 30% of formulation. In these cases, an efficient compressibility enhancer can help to achieving a good tablet with pharmaceutically accepted characteristics. Although, all of the cellulose based polymers are good compactable, however special grades of microcrystalline cellulose exhibit excellent compatibility. These grades can significantly improve compressibility of low compactable powder mixtures so they are widely used as compressibility enhancers in tablet manufacturing by direct compression and dry granulation methods. Various grades of MCC have different fundamental properties including their morphology, particles size, surface area, porosity and density (Rojas & Kumar, 2011). These physicochemical properties poses the different characteristics to them for example, smaller particles size MCC grades have good compressibility and poor flow ability whereas the larger particles size grades have poor compressibility and excellent flow ability. Particles size of MCC varies from 20 to 270 micrometer based on the manufacturer and type of application. MCC is available in three public brand names including Avicel® (FMC Polymer), VIVAPUR®/EMCOCEL® (JRS Pharma) and TABULOSE® (Blanver). The effects of size, shape and porosity of MCC particles on flow ability and compatibility have also been investigated by several researchers (Johansson et al., 2001). Various types of MCCs are extensively used in direct compression and dry granulation methods especially in roller compaction for preparing compressed tablets or pellets (Strydom et al., 2011; Bultmann, 2002). Microcrystalline cellulose type II (MCC-II) was recently introduced as new pharmaceutical excipients. MCC-II has a fibrous structure with lower compactability than MCC grades and suitable for using in rapid disintegrating dosage forms (Rojas et al., 2011; Reus-Medina & Kumar, 2006). In recent years, the new methods have been established for improving mechanical characteristics of MCCs. One of these innovative methods is lubricating or silisfying for improving compactability of low compressible grades of MCCs such as MCC-II or large particles size MCC grades. In this method, amorphous silicon dioxide (SiO2) is used as companion excipient for co-processing with low compressible MCC grades. Cellulose/SiO2 ratio is 98:2 and resulted product is called lubricated or silisified microcrystalline cellulose (SMCC). This method can optionally be used for both MCC-I or MCC-II for production SMCC-I or SMCC-II (Rojas & Kumar, 2011; Van Veen et al., 2005). SMCC-I have excellent compaction properties and less stickiness to the lower punches over MCC-I or MCC-I/SiO2 physical mixtures (Rojas & Kumar, 2011). SMCC-II has also better mechanical properties especially higher compactability than MCC-II without detriment if it's self-disintegrating characteristics. SMCC-I grades are commercially available under trade name of ProSolv® (JRS Pharma) but SMCC-II is not commercialized yet. Apart from MCC, other forms of cellulose are existed such as powdered cellulose (PC) and low crystallinity powdered cellulose (LCPC). LCPC and MCC have agglomerated and PC has fibrous structure. PC applications in pharmaceutical industries is similar that MCC. It is widely used in direct compression formulation and in dry granulation by either slugging or roller compaction methods. LCPC is a new direct compression cellulose excipient, which is prepared by controlled decrystallization and depolymerization of cellulose with phosphoric acid (Rojas & Kumar, 2011). LCPC was shown superior tableting properties than direct compression grades of MCC like Avicel®PH-101 (Kothari et al., 2002).

APPLICATION AS GELLING AGENTS. Gels are semisolid systems consisting of dispersions of very small particles or large molecules in an aqueous liquid vehicle rendered jellylike by the addition of a gelling agent. In recent decades, synthetic and semi-synthetic macromolecules are mostly used as gelling agents in pharmaceutical dosage forms. Some of these agents include: carbomers, cellulose derivatives and natural gums. Cellulose derivatives such as HPMC and CMC are the most popular gelling agents used in drug formulations. These polymers are less sensitive for microbial contamination than natural gelling agents such as tragacanth, acacia, sodium algininate, agar, pectin and gelatin. Cellulose derivatives generally dissolve better in hot water (except MC grades) and their mechanisms of jellification is thermal. For preparing gel, powder of these polymers with suitable amount initially dispersed in cold water by using mechanical mixture and then, the dispersion is heated to about 60-80° C. and gradually cooled to normal room temperature to form a gel (except MC grades). The resulted gels from these polymers are single-phase gels. Adding of electrolytes in the low concentrations increase the viscosity of these gels by salting out mechanism and higher concentrations (above 3-4%) can precipitate the polymer and breakup the gel system (Allen, et al., 1995). Maximum stability and transparency of the gels prepared by these polymers is about neutral range (pH=7-9) and acidic pHs can precipitate them from gel system. Minimum gel-forming concentrations of cellulose derivatives are different based on the type and the molecular weights of them but the medium range is about 4-6% w/v. The type of cellulose derivative in pharmaceutical gels can significantly affect drug release from gel formulations (Tas, et al., 2003). These gels also can optionally be used as the base of novel drug delivery systems, pharmaceutical coatings process, medical coating applications, topical ophthalmic protectant and lubricant and the like such as liposomal formulations (Gupta, et al., 2012).

APPLICATION AS THICKENING AND STABILIZING AGENTS. Cellulose derivatives are extensively used for thickening of pharmaceutical solutions and disperse systems such as emulsions and suspensions (Adibkia et al., 2007a, 2007b). Furthermore, these polymers can increase viscosity of non-aqueous pharmaceutical solution likes organic-based coating solutions. Viscosity enhancing of drug solutions poses many advantages such as improving consuming controllability and increasing residence time of drugs in topical and mucosal solutions which lead to improve bioavailability of topical, nasal or ocular preparations (Grove et al., 1990; Adibkia et al., 2007a, 2007b). It has been revealed that viscosity enhancement, in some cases, can increase absorption of some poorly-absorb drugs like insulin from oral dosage forms (Mesiha, M. & Sidhom, M.). Cellulose ethers in concentrations lower than minimum gel-forming amounts are used as thickening agents or viscosity builder. These polymers play an important role in stabilizing of pharmaceutical disperse systems especially in suspensions and coarse emulsions. By increasing the viscosity of suspension, based on the stock's equation, the sedimentation rate of dispersant decreased and thus, the uniformity of dispersion after shaking of product will improve. In the case of emulsions, these polymers can increase the shelf life and their resistance against mechanical and thermal shocks. Among cellulose derivatives, cellulose ethers especially their higher molecular weight grades are more suitable for using as viscosity enhancer and stabilizer for liquid pharmaceutical disperse systems such as suspensions and emulsions. There is a direct proportionality between viscosity of cellulose ether solutions and molecular weights of them.

APPLICATION AS FILLERS IN SOLID DOSAGE FORMS. Cellulose and related polymers are commonly used in solid dosage forms like tablets and capsules as filler. Various forms of cellulose have been used in pharmaceutical preparations as multifunctional ingredients thus; they are concerned as precious excipients for formulation of solid dosage forms. Cellulose and its derivatives have many advantages in using as filler in solid pharmaceuticals such as their compatibility with the most of other excipients, pharmacologically inert nature and indigestibility by human gastrointestinal enzymes. These polymers do not cause any irritancy potential on stomach and esophagus protective mucosa. Various forms of pure cellulose and cellulose ether derivatives can optionally be used as filler in these formulations.

APPLICATION AS BINDERS IN GRANULATION PROCESS. Binders are the essential components of solid drug formulations made by wet granulation process. In wet granulation process, drug substance is combined with other excipients and processed with the use of a solvent (aqueous or organic) with subsequent drying and milling to produce granules. Cellulose and some derivatives have excellent binding effects in wet granulation process. A number of MCC grades such as PH-101 are widely used as binder in wet granulation. Other cellulose derivatives such as MC, HPMC and HPC have good binding properties in wet granulation. Low substituted cellulose ethers such as low substituted HPC (L-HPC) also used as binder in wet granulation process (Desai et al., 2006; Wan & Prasad, 1988). Even though, low substituted cellulose ethers have lower water solubility compared with normal grades, however they have very good binding efficacy. Cross-linked cellulose (CLC) and cross-linked cellulose derivatives such as cross-linked NaCMC can optionally be used as excellent binders in pharmaceuticals as well (Chebli & Cartilier, 1998).

APPLICATION AS DISINTEGRATING AGENTS. Solid oral dosage forms such as tablets undergo several steps before systemic absorption of the drug. Disintegration is the first step immediately after administration of oral dosage forms that breakup the dosage forms into the smaller fragments in an aqueous environment. Converting of solid dosage forms to smaller fragments, increase the available surface area and promote a more rapid release of the drug substances from dosage forms. The earliest known disintegrant is Starch. Corn Starch or Potato Starch was recognized as being the ingredient in tablet formulations responsible for disintegration as early as 1906. Due to low compressibility of starch, pre-gelatinized starch was invented for using as disintegrant. Pre-gelatinized starch and MCC are two main types of classic disintegrants. In recent years, the classic disintegrants have been gradually replaced with the newer ones called super disintegrants. Super disintegrants can acts in lower concentrations than starch and have not detriment effect on compressibility and flow ability of formulations. Three main groups of these excipients are: modified starches like sodium starch glycolate (Primogel®, Explotab®) with 4%-6% effective concentration, cross-linked polyvinyl pyrrolidones like crospovidone (Polyplasdone XL, Kollidon CL) with 2%-4% effective concentration and modifies cellulose like cross-linked sodium carboxymethyl cellulose or croscarmellose (Ac-Di-Sol™ and Nymcel) with 2%-4% effective concentration in wet granulation process. Modified cellulose compounds are very efficient disintegrants and additionally, can accelerate the dissolution rate of drugs in aqueous environment (Chebli & Cartilier, 1998).

APPLICATION AS TASTE MASKING AGENTS. There are numerous drugs with unfavorable tastes. The most prevalent unpleasant taste of the drugs is bitter taste. Unpleasant-tasting dosage forms leads to lack of patient compliance of oral drug preparations. Various tastes are feeling by taste buds on the tongue. Taste buds are onion-shaped structures containing between 50 to 100 taste cells. Chemicals from food or oral ingested medicine are dissolved by the saliva and enter via the taste pore. They either interact with surface proteins known as taste receptors or with pore-like proteins called ion channels. These interactions cause electrical changes within the taste cells that trigger them to send chemical signals that translate into neurotransmission to the brain. Salt and sour responses are of the ion channel type of responses, while sweet and bitter are surface protein responses. Taste masking is an important consideration in formulation of oral dosage forms especially in the case of high dose, poorly tasting drugs. Improving the taste of liquid dosage forms is more important because of better sensitivity and faster stimulation of taste receptors by liquids in compared than solids. Taste masking in solid dosage forms can be performed by coating (in the case of tablets, pellets, pills or coarse granules) or micro-coating (in the case of fine granules, powders or microcapsules) of them by a gastro-soluble polymeric coating. These coats can prevent from contacting of the drug with taste buds without detriment of release characteristics of the drug formulations in gastrointestinal tract. Soluble cellulose ether derivatives are suitable for this purpose. These polymers like HPMC, HEC, MC and HPC are water soluble and have very good film forming properties. Some grades of MCC also can improve tooth-feel such as Avicel® CE-15. These coats can produce additional benefits in drug formulations such as protection of the active ingredients against moisture, oxygen of the air and light due to their barrier effects. Masking of the taste in liquid dosage forms especially in drug solutions is more sophisticated. In these cases test receptor blockers, flavoring agents and viscosity enhancers are simultaneously needed.

Cellulose Triacetate, also known simply as triacetate, CTA and TAC, is manufactured from cellulose and a source of acetate esters, typically acetic anhydride. Triacetate is typically used for the creation of fibers and film. It is similar chemically to cellulose acetate, with the distinguishing characteristics being that in triacetate, according to the Federal Trade Commission definition, at least “92 percent of the hydroxyl groups are acetylated.” During the manufacture of triacetate the cellulose is completely acetylated whereas in regular cellulose acetate or other cellulose, it is only partially acetylated. Triacetate is significantly more heat resistant than cellulose acetate.

Chemical Vapor Deposition (CVD) is a type of chemical process used to produce high quality, high-performance, solid materials. The process is often used in the semiconductor industry to produce thin films. In typical CVD, the wafer (substrate) is exposed to one or more volatile precursors, which react and/or decompose on the substrate surface to produce the desired deposit. Frequently, volatile by-products are also produced, which are removed by gas flow through the reaction chamber. Microfabrication processes widely use CVD to deposit materials in various forms, including: monocrystalline, polycrystalline, amorphous, and epitaxial. These materials include: silicon, carbon fiber, carbon nanofibers, fluorocarbons, filaments, carbon nanotubes, SiO₂, silicon-germanium, tungsten, silicon carbide, silicon nitride, silicon oxynitride, titanium nitride, and various high-k dielectrics. CVD is also used to produce synthetic nanocrystalline (NC) diamonds.

Coating Applications and Thin Films can optionally include, but not limited to, being applied to structural bulk materials in order to improve the desired properties of the surface, such as wear resistance, friction, corrosion resistance and others, yet keeping the bulk properties of the material unchanged. A typical example is nitriding and carbonitriding of steel parts for engines and other machines at relatively low temperatures of about 500° C. in order to increase the hardness of the surface and reduce wear. Modern nanostructured coating applications and thin films for structural and functional applications, which were developed during the past 10-15 years, are used mainly for wear protection of machining tools and for the reduction of friction in sliding parts. One distinguishes between nanocrystalline (NC) coating applications and thin films, where a few nanometers thin layers of two different materials are deposited subsequently, and nanocomposites, which are, in the optimum case, isotropic. The ultra hard (NC) nanocomposites, such as nc-(Ti₁₋ₓAlx)N/a-Si₃N₄ (nc- and a-stand for nanocrystalline and X-ray amorphous, respectively), show superior cutting performance as compared with conventional, state-of-the art hard coating applications (Ti₁₋ₓAlx)N that presently dominate the applications for dry machining. The costs of their large-scale industrial production are comparable with those of the conventional coating applications. Also, the heterostructures and multilayer coating applications are successfully applied on industrial scale. Low-friction nanostructured coating applications consisting of a hard transition-metal carbide or nitride in combination with a solid lubricant, such as diamond-like carbon (DLC), MoS₂, WS₂ and others that combine with a high hardness and low friction. They are applied in a variety of bearings and sliding parts operating without liquid lubricants, which is an important advantage particularly in a hostile environment, and when the movable parts have to stop and go very frequently, e.g. in the textile industry. The recent development of nanocomposites consisting of a hard transition-metal nitride or carbide in combination with soft and ductile metal is likely to find numerous applications in a variety of machine parts. The hardness of these coating applications varies between about 13 and 30 GPa depending on the composition. When deposited under energetic ion bombardment and temperatures below about 350° C., an enhancement of the hardness up to about 50 GPa was found, in a similar way as for hard transition-metal nitrides (e.g. 100 GPa for TiAlVN and 80 GPa for TiN). However, this hardness enhancement is of a little use because, upon annealing to ≧500° C., these coating applications soften. Unfortunately, these nanocomposites were often confused with the thermally highly stable ultra hard (NC) nanocomposites prepared according to the generic design principle. The nanocrystalline (NC) coating applications should be subdivided into multilayers and superlattices. When a 3-4 μm thick monolytic layer of a hard ceramic material, such as TiN, is replaced by a stack of 20-100 nm thin multilayers of TiN and another hard nitride, boride or carbide, the resistance against brittle failure strongly increases because the crack cannot propagate through the whole layer. Usually, also an increase of the hardness above that of the rule-of-mixtures is found. Similar enhancement was found also in metallic multilayers, for example Fe/Cu and Ni/Cu and Ni₃Al/Ni, and in metal/nitride multilayers, for example Ti/TiN. The majority of hard protecting coating applications applied to machining tools nowadays are such multilayers.

Coatings Applications can optionally include, but not limited to, the nanocrystalline metal oxides require dispersion into a liquid medium, such as a solvent, or blended directly into the resin system. As produced, the metal oxide powders disperse well in aqueous environments wherein hydrogen bonding is sufficiently strong to disrupt the loose agglomerates and provide stable dispersions of the primary crystalline particles. The affinity of nanocrystalline powders for aqueous environments is often sufficient to allow the powders to be used in many waterborne coating formulations. However, because the powders do not disperse well in non-aqueous media, several specialized surface treatments have been developed that reduces particles agglomerates and yield stable dispersions in hydrocarbon solvents. Such treatments also prevent reagglomeration and thus enable the oxides to be used in a variety of solvent borne coating applications.

Colloidal Gold is a sol or colloidal suspension of submicrometre-size nanoparticles of gold in a fluid, usually water. The liquid is usually either an intense red color (for particles less than 100 nm) or blue/purple (for larger particles). Due to the unique optical, electronic, and molecular-recognition properties of gold nanoparticles, they are the subject of substantial research, with applications in a wide variety of areas, including electron microscopy, electronics, nanotechnology, and materials science. The properties of colloidal gold nanoparticles, and thus their applications, depend strongly upon their size and shape. For example, rod like particles have both transverse and longitudinal absorption peak, and anisotropy of the shape affects there self-assembly.

Coconut Oil or Copra Oil is a dietary supplement and edible oil extracted from the kernel or meat of matured coconuts harvested from the coconut palm (Cocos nucifera). It has various applications in food, medicine, and industry. Because of its high saturated fat content it is slow to oxidize and, thus, resistant to rancidification, lasting up to two years without spoiling. Many health organizations advise against the consumption of high amounts of coconut oil due to its high levels of saturated fat. Production. Dry Process. Coconut oil can be extracted through “dry” or “wet” processing. Dry processing requires the meat to be extracted from the shell and dried using fire, sunlight, or kilns to create copra. The copra is pressed or dissolved with solvents, producing the coconut oil and a high-protein, high-fiber mash. The mash is of poor quality for human consumption and is instead fed to ruminants; there is no process to extract protein from the mash. A portion of the oil extracted from copra is lost to the process of extraction. Wet Process. The all-wet process uses raw coconut rather than dried copra, and the protein in the coconut creates an emulsion of oil and water. The more problematic step is breaking up the emulsion to recover the oil. This used to be done by prolonged boiling, but this produces a discolored oil and is not economical; modern techniques use centrifuges and pre-treatments including cold, heat, acids, salts, enzymes, electrolysis, shock waves, or some combination of them. Despite numerous variations and technologies, wet processing is less viable than dry processing due to a 10%-15% lower yield, even compared to the losses due to spoilage and pests with dry processing. Wet processes also require investment of equipment and energy, incurring high capital and operating costs. Proper harvesting of the coconut (the age of a coconut can be 2 to 20 months when picked) makes a significant difference in the efficacy of the oil-making process. Copra made from immature nuts is more difficult to work with and produces an inferior product with lower yields. Conventional coconut oil uses hexane as a solvent to extract up to 10% more oil than just using rotary mills and expellers. The oil is then refined to remove certain free fatty acids, in order to reduce susceptibility to rancidification. Other processes to increase shelf life include using copra with a moisture content below 6%, keeping the moisture content of the oil below 0.2%, heating the oil to 130-150° C. (266-302° F.) and adding salt or citric acid. Virgin coconut oil (VCO) can be produced from fresh coconut milk, meat, or residue. Producing it from the fresh meat involves removing the shell and washing, then either wet-milling or drying the residue, and using a screw press to extract the oil. VCO can optionally be extracted from fresh meat by grating and drying it to a moisture content of 10%-12%, then using a manual press to extract the oil. Producing it from coconut milk involves grating the coconut and mixing it with water, then squeezing out the oil. The milk can optionally be fermented for 36-48 hours, the oil removed, and the cream heated to remove any remaining oil. A third option involves using a centrifuge to separate the oil from the other liquids. Coconut oil can optionally be extracted from the dry residue left over from the production of coconut milk. A thousand mature coconuts weighing approximately 1,440 kilograms (3,170 lb) yield around 170 kilograms (370 lb) of copra from which around 70 litres (15 imp gal) of coconut oil can be extracted.

RBD. RBD stands for “refined, bleached, and deodorized.” RBD oil is usually made from copra (dried coconut kernel). The dried copra is placed in a hydraulic press with added heat and the oil is extracted. This yields up practically all the oil present, amounting to more than 60% of the dry weight of the coconut. This “crude” coconut oil is not suitable for consumption because it contains contaminants and must be refined with further heating and filtering. Another method for extraction of a “high-quality” coconut oil involves the enzymatic action of alpha-amylase, polygalacturonases, and proteases on diluted coconut paste. Unlike virgin coconut oil, refined coconut oil has no coconut taste or aroma. RBD oil is used for home cooking, commercial food processing, and cosmetic, industrial, and pharmaceutical purposes.

Hydrogenation. RBD coconut oil can be processed further into partially or fully hydrogenated oil to increase its melting point. Since virgin and RBD coconut oils melt at 24° C. (76° F.), foods containing coconut oil tend to melt in warm climates. A higher melting point is desirable in these warm climates, so the oil is hydrogenated. The melting point of hydrogenated coconut oil is 36-40° C. (97-104° F.). In the process of hydrogenation, unsaturated fats (monounsaturated and polyunsaturated fatty acids) are combined with hydrogen in a catalytic process to make them more saturated. Coconut oil contains only 6% monounsaturated and 2% polyunsaturated fatty acids. In the partial hydrogenation process, some of these are transformed into trans fatty acids.

Fractionation. Fractionated coconut oil provides fractions of the whole oil so that its different fatty acids can be separated for specific uses. Lauric acid, a 12-carbon chain fatty acid, is often removed because of its high value for industrial and medical purposes. The fractionation of coconut oil can optionally be used to isolate caprylic acid and capric acid, which are medium-chain triglycerides, as these are used for medical applications, special diets and cosmetics, sometimes also being used as an carrier oil for fragrances.

Copper is a chemical element with symbol Cu (from Latin: cuprum) and atomic number 29. It is a ductile metal with very high thermal and electrical conductivity. Pure copper is soft and malleable; a freshly exposed surface has a reddish-orange color. It is used as a conductor of heat and electricity, a building material, and a constituent of various metal alloys, copper alloy, cobalt alloy, and silver alloy. The metal and its alloys, copper alloy, cobalt alloy, silver alloy or other types of alloys have been used for thousands of years. In the Roman era, copper was principally mined on Cyprus, hence the origin of the name of the metal as cyprium (metal of Cyprus), later shortened to cuprum. Its compounds are commonly encountered as copper(II) salts, which often impart blue or green colors to minerals such as azurite and turquoise and have been widely used historically as pigments. Architectural structures built with copper corrode to give green verdigris (or patina). Decorative art prominently features copper, both by itself and as part of pigments. Copper is essential to all living organisms as a trace dietary mineral because it is a key constituent of the respiratory enzyme complex cytochrome. In molluscs and crustacea copper is a constituent of the blood pigment hemocyanin, which is replaced by the iron-complexed hemoglobin in fish and other vertebrates. The main areas where copper is found in humans are liver, muscle and bone. Copper compounds are used as bacteriostatic substances, fungicides, and wood preservatives.

Crop Yield (also known as “agricultural output”) refers to both the measure of the yield of a crop per unit area of land cultivation, and the seed generation of the plant itself (e.g. if three grains are harvested for each grain seeded, the resulting yield is in a ratio of 1:3). The ratio 1:3 is considered by agronomists as the minimum required to sustain human life. One of the three seeds must be set aside for the next planting season, the remaining two either consumed by the grower, or one for human consumption and the other for livestock feed. The higher the surplus, the more livestock can be established and maintained, thereby increasing the physical and economic well-being of the farmer and his family. This, in turn, resulted in better stamina, better over-all health, and better, more efficient work. In addition, the more the surplus the more draft animals—horse and oxen—could be supported and harnessed to work, and manure, the soil thereby easing the farmer's burden. Increased crop yields meant few hands were needed on farm, freeing them for industry and commerce.

Crystal is a crystal or crystalline solid is a solid material whose constituent atoms, molecules, or ions are arranged in an orderly repeating pattern extending in all three spatial dimensions. This orderly repeating pattern is called a “crystal lattice.” Essentially these molecules are arranged in an orderly formation. In other words a crystal is in-formation. Computers store and/or transmit information on silicone crystals. Crystals, which are basically, sand/or in formation and/or so can optionally hold memory. Crystalline water or structured water is in-formation and/or so can optionally also store and/or transmit information.

Crystalline Forms. Each of the Earth's minerals has a crystalline form. Synthetic nanocrystalline (NC) diamonds are crystalline carbon; emeralds are crystalline beryllium; and/or rubies are crystalline corundum. The difference between corundum and/or a ruby is the way the molecules are organized or structured (see images of corundum and/or ruby at right). Each crystal has a specific structural pattern. Minerals form crystals when circumstances (for example: heat and/or pressure) cause the molecules to form a repeating pattern. Most people know that extreme pressure is required to form a diamond. Pressure forces molecules to arrange themselves in a different configuration to withstand/or the pressure. Structural organization changes the characteristics of the substance. Some of these changes are obvious—like the visible difference between carbon and/or a diamond. It's all a matter of organization.

Crystalline Silicon (c-Si) is an umbrella term for the crystalline forms of silicon encompassing multicrystalline silicon (multi-Si) and monocrystalline silicon (mono-Si), the two dominant semiconducting materials used in photovoltaic technology for the production of solar using semiconducting materials, that are assembled into a solar panel and part of a solar cell or photovoltaic system to generate solar power from sunlight. In electronics, the term crystalline silicon typically refers to monocrystalline form silicon, as the sole material used for producing microchips, containing much lower impurity levels than those required for solar cells. Production of semiconductor grade silicon involves a chemical purification to produce hyper pure polysilicon followed by a recrystallization process to grow monocrystalline silicon. The cylindrical boules are then cut into wafers for further processing. Solar cells made of crystalline silicon are often called conventional, traditional, or first generation solar cells, as they were developed in the 1950s and remained the most common type up to the present time. Because they are produced from about 160 μm thick solar wafers—slices from bulks of solar grade silicon—they are sometimes called wafer-based solar cells. Solar cells made from c-Si are single-junction cells and are generally more efficient than their rival technologies, which are the second-generation thin-film solar cells, the most important being CdTe, CIGS, and amorphous silicon (a-Si). Amorphous silicon is an allotropic variant of silicon, and amorphous means “without shape” to describe its non-crystalline form.

Crystal Structure. In mineralogy and crystallography, a crystal structure is a unique arrangement of atoms, ions or molecules in a crystalline liquid or solid. It describes a highly ordered structure, occurring due to the intrinsic nature of its constituents to form symmetric patterns. The crystal lattice can be thought of as an array of ‘small boxes’ infinitely repeating in all three spatial directions. Such a unit cell is the smallest unit of volume that contains all of the structural and symmetry information to build-up the macroscopic structure of the lattice by translation. Patterns are located upon the points of a lattice, which is an array of points repeating periodically in three dimensions. The lengths of the edges of a unit cell and the angles between them are called the lattice parameters. The symmetry properties of the crystal are embodied in its space group. A crystal's structure and symmetry play a role in determining many of its physical properties, such as cleavage, electronic band structure, and optical transparency.

Dairy Products or Milk Products is a food produced from the milk of mammals. Dairy products are usually high energy-yielding food ingredients and food products. A production plant for the processing of milk is called a dairy or a dairy factory. Apart from breastfed infants, the human consumption of dairy products is sourced primarily from the milk of cows, water buffaloes, goats, sheep, yaks, horses, camels, domestic buffaloes, and other mammals. Dairy products are commonly found in European, Middle Eastern, and Indian cuisine, whereas aside from Mongolian cuisine they are little-known in traditional East Asian cuisine.

Types of Dairy Products. Milk after optional homogenization, pasteurization, in several grades after standardization of the fat level, and possible addition of the bacteria Streptococcus lactis and Leuconostoc citrovorum, Crème fraîche, slightly fermented cream, Clotted cream, thick, spoonable cream made by heating milk, Single cream, double cream and whipping cream, Smetana, Central and Eastern European variety of sour cream, Cultured milk resembling buttermilk, but uses different yeast and bacterial cultures, Kefir, fermented milk drink from the Northern Caucasus, Kumis/Airag, slightly fermented mares' milk popular in Central Asia, Powdered milk (or milk powder), produced by removing the water from (usually skim) milk, Whole milk products, Buttermilk products, Skim milk, Whey products, High milk-fat and nutritional products (for infant formulas), Cultured and confectionery products, Condensed milk, milk which has been concentrated by evaporation, with sugar added for reduced process time and longer life in an opened can, Khoa, milk which has been completely concentrated by evaporation, used in Indian cuisine including gulab jamun, peda, etc.), Evaporated milk, (less concentrated than condensed) milk without added sugar, Ricotta, acidified whey, reduced in volume, Infant formula, dried milk powder with specific additives for feeding human infants, Baked milk, a variety of boiled milk that has been particularly popular in Russia, Butter, mostly milk fat, produced by churning cream, Buttermilk, the liquid left over after producing butter from cream, often dried as livestock feed, Ghee, clarified butter, by gentle heating of butter and removal of the solid matter, Smen, a fermented, clarified butter used in Moroccan cooking, Anhydrous milkfat (clarified butter), Cheese, produced by coagulating milk, separating from whey and letting it ripen, generally with bacteria and sometimes also with certain molds, Curds, the soft, curdled part of milk (or skim milk) used to make cheese, Paneer, Whey, the liquid drained from curds and used for further processing or as a livestock feed, Cottage cheese, Quark, Cream cheese, produced by the addition of cream to milk and then curdled to form a rich curd or cheese, Fromage frais, Casein are Caseinates, sodium or calcium salts of casein Milk protein concentrates and isolates, Whey protein concentrates and isolates, reduced lactose whey, Hydrolysates, milk treated with proteolytic enzymes to alter functionality, Mineral concentrates, byproduct of demineralizing whey, Yogurt, milk fermented by Streptococcus salivarius ssp. thermophilus and Lactobacillus delbrueckii ssp. bulgaricus sometimes with additional bacteria, such as Lactobacillus acidophilus, Ayran, Lassi, Indian subcontinent, Leben, Clabber, milk naturally fermented to a yogurt-like state, Gelato, slowly frozen milk and water, lesser fat than ice cream, Ice cream, slowly frozen cream, milk, artificial flavors and emulsifying additives (dairy ice cream), Ice milk, low-fat version of ice cream, Frozen custard, Frozen yogurt, yogurt with emulsifiers. Other. Viili. Kajmak, Filmjölk, Piimä, Vla, Dulce de Leche, Skyr, Junket, milk solidified with rennet. Health. Dairy products can cause health issues for individuals who have lactose intolerance or a milk allergy. Additionally dairy products including cheese, ice cream, milk, butter, and yogurt can contribute significant amounts of cholesterol and saturated fat to the diet. Diets high in fat and especially in saturated fat can increase the risk of heart disease and can cause other serious health problems.

Dangerous Food Additives BHT (Butylated Hydroxytoluene). non-limiting examples of dangerous food additives, include, but not limited to this common additive used to prevent oxidation in a wide variety of foods and cosmetics is listed by the National Toxicology Program (NTP) in 2005 as “reasonably anticipated to be a human carcinogen” on the basis of experimental findings in animals. It is also used in jet fuels, rubber petroleum products, and transformer oil and embalming fluid. As if this were not enough, the Material Safety Data Sheet (MSDS) warns that BHT should not be allowed to enter the environment, can cause liver damage, and is harmful to aquatic organisms. High Fructose Corn Syrup (HFCS): Loaded with “unbound” fructose and glucose molecules, studies have shown that the reactive carbonyl molecules can cause tissue damage that may lead to obesity, diabetes, and also heart disease. So much for this “Strong Heart Anti-aging products, antioxidants” cereal recipe! HFCS is made from genetically modified corn and processed with genetically modified enzymes. To make matters worse, studies have recently revealed that nearly half of tested samples of HFCS contained mercury. Yellow #5: Almost all colorants approved for use in food are derived from coal tar and may contain up to 10 ppm of lead and arsenic. Also, and not surprisingly, most coal tar colors could potentially cause cancer Soybean Oil: More than half of all soybeans crops grown in the US are genetically-modified (GMO) representing a meteoric rise since 1996, when only 7% were GMO soybeans. Genetically modified crops not only pose environmental dangers. There is a growing concern (and mounting scientific evidence) that genetic engineering of food plant seeds may have an unexpected and negative impact on human health. Propylene Glycol Alginate (E405): this food thickener, stabilizer, and emulsifier is derived from alginic acid esterified and combined with propylene glycol. Bear in mind that even though propylene glycol is used as a food additive, it has many industrial uses including automotive antifreezes and airport runway de-icers. Polysorbate 60: short for polyoxyethylene-(20)-sorbitan monostearate this emulsifier is widely used in the food industry. Made of made of corn, palm oil and petroleum, this gooey mix can't spoil, so it often replaces dairy products in baked goods and other liquid products. Enriched Flour: these pretzels are made with enriched flour. But don't let the attractive description mislead you: like most highly processed cellulose in foods, enriched flour is devoid of nutrients and more often than not it is also bleached. Since the wheat germ and bran are removed from this type of flour, the body treats it as a refined starch. The “enrichment” itself is made using toxic ingredients. For example, iron is added back into enriched flour. Unfortunately, food makers use a metallic form of iron that your body can barely absorb and should not be ingested. Textured soy protein concentrate, carrageenan, maltodextrin, disodium inosinate, disodium guanylate, modified cornstarch: All of these are basically different names to hide ingredients that either contain Monosodium Glutamate (MSG) or form MSG during processing. It is very important for your bone health and your overall health that you avoid these acidifying chemicals that can cause a variety of undesirable side effects, besides accelerate your bone loss. MSG and its related products have been linked skin rashes, nausea, migraine headaches, heart irregularities, and even seizures. Deoxyribonucleic Acid (/di, [symbol] ksi,raIb-o.nju:,kleI. -ik′æsId/; DNA) is a molecule that encodes the genetic instructions used in the development and functioning of all known living organisms and many viruses. DNA is a nucleic acid; alongside proteins and carbohydrates, nucleic acids compose the three major macromolecules essential for all known forms of life. Most DNA molecules consist of two polymer strands coiled around each other to form a double helix. The two DNA strands are known as polynucleotides since they are composed of simpler units called nucleotides. Each nucleotide is composed of a nitrogen-containing nucleobase—either guanine (G), adenine (A), thymine (T), or cytosine (C)—as well as a monosaccharide sugar called deoxyribose and a phosphate group. The nucleotides are joined to one another in a chain by covalent bonds between the sugar of one nucleotide and the phosphate of the next, resulting in an alternating sugar-phosphate backbone. According to base pairing rules (A with T and C with G), hydrogen bonds bind the nitrogenous bases of the two separate polynucleotide strands to make double-stranded DNA. DNA is well-suited for biological information storage. The DNA backbone is resistant to cleavage, and both strands of the double-stranded structure store the same biological information. Biological information is replicated as the two strands are separated. A significant portion of DNA (more than 98% for humans) is non-coding, meaning that these sections do not serve as patterns for protein sequences. The two strands of DNA run in opposite directions to each other and are therefore anti-parallel. Attached to each sugar is one of four types of nucleobases (informally, bases). It is the sequence of these four nucleobases along the backbone that encodes biological information. Under the genetic code, RNA strands are translated to specify the sequence of amino acids within proteins. These RNA strands are initially created using DNA strands as a template in a process called transcription. Within cells, DNA is organized into long structures called chromosomes. During cell division these chromosomes are duplicated in the process of DNA replication, providing each cell its own complete set of chromosomes. Eukaryotic organisms (animals, plants, fungi, and protists) store most of their DNA inside the cell nucleus and some of their DNA in organelles, such as mitochondria or chloroplasts. In contrast, prokaryotes (bacteria and archaea) store their DNA only in the cytoplasm. Within the chromosomes, chromatin proteins such as histones compact and organize DNA. These compact structures guide the interactions between DNA and other proteins, helping control, which parts of the DNA are transcribed. Scientists use DNA as a molecular tool to explore physical laws and theories, such as the ergodic theorem and the theory of elasticity. The unique material properties of DNA have made it an attractive molecule for material scientists and engineers interested in micro- and nano-fabrication. Among notable advances in this field are DNA origami and DNA-based hybrid materials. The obsolete synonym “desoxyribonucleic acid” may occasionally be encountered, for example, in pre-1953 genetics.

Different Types of Plastic Bottles. These types of bottles, water bottles, caps are present in almost all parts of the world and are in abundance. Many manufacturers use these bottles, water bottles, caps, to contain soft-drinks, oils and many other household liquid, non-liquids. Plastic is an ideal material for manufacturers to make bottles, water bottles, caps, engineered wood, furniture, and floors from as it is cheap and easily available. The process to manufacturing plastic bottles, water bottles, caps, is inexpensive thus not rendering manufacturers much production cost. However, using plastic bottles, water bottles, caps, engineered wood, can have harmful effect on the environment if not disposed off properly. Plastic is not a biodegradable material thus it can cause pollution if disposed off simply into the environment. Despite the advantages, there are some disadvantages as well to using plastic bottles, water bottles, caps, engineered wood, furniture, and floors.

Different Types of Coating Applications is a covering that is applied to the surface of an object, usually referred to as the substrate. The purpose of applying the coating may be decorative, functional, or both. The coating itself may be an all-over coating, completely covering the substrate, or it may only cover parts of the substrate. An example of all of these types of coating is a product label on many drinks, plastic bottles, plastic water bottles one side has an all-over functional coating (the adhesive) and the other side has one or more decorative coating applications in an appropriate pattern (the printing) to form the words and images. Paints and lacquers are coating applications that mostly have dual uses of protecting the substrate and being decorative, although some artist's paints are only for decoration, and the paint on large industrial pipes is presumably only for the function of preventing corrosion. Functional coating applications may be applied to change the surface properties of the substrate, such as adhesion, wettability, corrosion resistance, or wear resistance. In other cases, e.g. semiconductor device fabrication (where the substrate is a wafer), the coating adds a completely new property such as a magnetic response or electrical conductivity and forms an essential part of the finished product. A major consideration for most coating processes is that the coating is to be applied at a controlled thickness, and a number of different processes are in use to achieve this control, ranging from a simple brush for painting a wall, to some very expensive machinery applying coating applications in the electronics industry. A further consideration for ‘non-all-over’ coating applications is that control is needed as to where the coating is to be applied. A number of these non-all-over coating processes are printing processes. Many industrial coating processes involve the application of a thin film of functional material to a substrate, such as paper, fabric, film, foil, or sheet stock. If the substrate starts and ends the process wound up in a roll, the process may be termed “roll-to-roll” or “web-based” coating. A roll of substrate, when wound through the coating machine, is typically called a web. Coating applications may be applied as liquid, non-liquids, gases or solids.

Different Types of Fertilizers can include, but are not limited to the following: Soil amendments are made further comprising at least one fertilizers to the soil but there are different types of fertilizers. There is bulky organic fertilizer such as cow manure, bat guano, bone meal, organic compost and green manure crops. And then there is also chemical fertilizer, which is also referred to as inorganic fertilizer and is made up with different formulations to suit a variety of specified uses. Though many governments and agricultural departments go to great lengths to increase the supply of organic fertilizers, such as bulky organic manures and composting materials, there are just not enough of these fertilizers available to meet the existing and future fertilizer needs. Compared to organic compost, chemical or inorganic fertilizers also have the added advantage of being less bulky. Being less bulky makes chemical fertilizer easier to transport, both overland and from the soil into the plants itself, because they get to be available to the plant relatively quickly when incorporated as part of the plant-food constituents. Chemical fertilizer usually comes in either granular or powder form in bags and boxes, or in liquid formulations in bottles. The different types of chemical fertilizers are usually classified according to the three principal elements, namely Nitrogen (N), Phosphorous (P) and Potassium (K), and may, therefore, be included in more than one group. Organic and Inorganic Chemical Nitrogenous Fertilizer Types. This type of fertilizer is divided into different groups according to the manner in which the Nitrogen combines with other elements. These groups are: Sodium Nitrates, Ammonium Sulphate and ammonium salts, Chemical compounds that contains Nitrogen in amide form, and Animal and plant by products. Sodium Nitrates are also known as Chilates or Chilean nitrate. The Nitrogen contained in Sodium Nitrate is refined and amounts to 16%. This means that the Nitrogen is immediately available to plants and as such is a valuable source of Nitrogen in a type of fertilizer. When one makes a soil amendment using Sodium Nitrates as a type of fertilizer in the garden, it is usually as a top- and side-dressing. Particularly when nursing young plants and garden vegetables. In soil that is acidic Sodium Nitrate is quite useful as a type of fertilizer. However, the excess use of Sodium Nitrate may cause deflocculation. Ammonium Sulphate. This fertilizer type comes in a white crystalline salt form, containing 20% to 21% ammonia cal nitrogen. It is easy to handle and it stores well under dry conditions. However, during the rainy season, it sometimes, forms lumps. Though this fertilizer type is soluble in water, its nitrogen is not readily lost in drainage, because the ammonium ion is retained by the soil particles. Ammonium sulphate may have an acid effect on garden soil. Over time, the long-continued use of this type of fertilizer will increase soil acidity and thus lower the yield. The application of Ammonium sulphate fertilizer can be done before sowing, at sowing time, or even as a top-dressing to the growing crop. Ammonium Nitrate. This fertilizer type also comes in white crystalline salts. Ammonium Nitrate salts contains 33% to 35% nitrogen, of which half are nitrate nitrogen and the other half in the ammonium form. As part of the ammonium form, this type of fertilizer cannot be easily leached from the soil. This fertilizer is quick-acting, but highly hygroscopic thus making it unfit for storage. On a note of caution: Ammonium Nitrate also has an acid effect on the soil, in addition this type of fertilizer can be explosive under certain conditions, and, should thus be handled with care. Nitro Chalk′ is the trade name of a product formed by mixing ammonium nitrate with about 40% lime-stone or dolomite. This fertilizer is granulated, non-hazardous and less hygroscopic. The lime content of this fertilizer type makes it useful for application to acidic garden soils. Ammonium Sulphate Nitrate. This fertilizer type is available as a mixture of ammonium nitrate and ammonium sulphate and is recognizable as a white crystal or as dirty-white granules. This fertilizer contains 26% nitrogen, three-fourths of it in the ammoniac form and the remainder (i.e. 6.5%) as nitrate nitrogen. Ammonium Sulphate Nitrate is non-explosive, readily soluble in water and is very quick-acting. Because this type of fertilizer keeps well, it is very useful for all crops. Though it can also render garden soil acidic, the acidifying effects is only one-half of that of ammonium sulphate on garden soil. Application of this fertilizer type can be done before sowing, at sowing time or as a top-dressing, but it should not be applied along the seed. Ammonium Chloride. This fertilizer type comes in a white crystalline compound, which contains a good physical condition and 26% ammoniac nitrogen. In general, Ammonium Chloride is similar to ammonium sulphate in action. Urea. This type of fertilizer usually is available to the public in a white, crystalline, organic form. It is a highly concentrated nitrogenous fertilizer and fairly hygroscopic. This also means that this fertilizer can be quite difficult to apply. Urea is also produced in granular or pellet forms and is coated with a non-hygroscopic inert material. It is highly soluble in water and therefore, subject to rapid leaching. It is, however, quick-acting and produces quick results. When applied to the soil, its nitrogen is rapidly changed into ammonia. Similar to ammonium nitrate, urea supplies nothing but nitrogen and the application of Urea as fertilizer can be done at sowing time or as a top-dressing, but should not be allowed to come into contact with the seed. Ammonia. This fertilizer type is a gas that is made up of about 80% of nitrogen and comes in a liquid form as well because under the right conditions regarding temperature and pressure, Ammonia becomes liquid (anhydrous ammonia). Another form, ‘aqueous ammonia’, results from the absorption of Ammonia gas into water, in which it is soluble. Ammonia is used as a fertilizer in both these forms. The anhydrous liquid form of Ammonia can be applied by introducing it into irrigation water, or directly into the soil from special containers. Not really suitable for the home gardener as this renders the use of ammonia as a fertilizer very expensive. Organic Nitrogenous Fertilizers. Organic Nitrogenous fertilizer is the type of fertilizer that includes plant and animal by-products. These by-products can be anything from oil cakes, to fish manure and even to dried blood. The Nitrogen available in organic nitrogenous fertilizer types first has to be converted before the plants can use it. This conversion occurs through bacterial action and is thus a slow process. Oil-cakes contain not only nitrogen but also some phosphoric and potash, besides a large quantity of organic matter. This type of fertilizer is used in conjunction with quicker-acting chemical fertilizers. Then there is also blood meal which contains 10% to 12% highly available Nitrogen as well as 1% to 2% Phosphoric acid. Blood meal, used in much the same way as oilcakes, makes for a quick remedy and can effectively be used on all types of soil as a type of fertilizer. Fish meal, which can be dried fish, fish-meal or even powder, is extracted in areas where fish oil is extracted. The resulting residue is used as a fertilizer type. Obviously depending on the type of fish used, the available Nitrogen can be between 5% and 8% and the Phosphoric content can be from 4% to 6%. Fish meal also constitutes a fast-acting fertilizer type, which is suitable for most soil types and crops. Organic and Inorganic Chemical Phosphate Fertilizer Types. The Phosphate fertilizers are categorized as natural phosphates, either treated or processed, and also by products of phosphates and chemical phosphates. Rock Phosphate. As a type of fertilizer, rock phosphate occurs as natural deposits in some countries. This fertilizer type has its advantages and disadvantages. The advantage is that with adequate rainfall this fertilizer results in a long growing period, which can enhance crops. Powdered phosphate fertilizer is an excellent remedy for soils that are acidic and has a phosphorous deficiency and requires soil amendments. However, the disadvantage is that although phosphate fertilizer such as rock phosphate contains 25% to 35% phosphoric acid, the phosphorous is insoluble in water. It has to be pulverized to be used as a type of fertilizer before rendering satisfactory results in garden soil. Thus it is not surprising that Rock Phosphate is used to manufacture superphosphate, which makes the Phosphoric acid water soluble. Superphosphate. Superphosphate is a fertilizer type that most gardeners are familiar with. As a fertilizer type one can get superphosphate in three different grades, depending on the manufacturing process. The following is a short description of the different superphosphate fertilizer grades: Single Superphosphate containing 16% to 20% phosphoric acid; Dicalcium Phosphate containing 35% to 38% phosphoric acid; and Triple Superphosphate containing 44% to 49% phosphoric acid. Triple superphosphate is used mostly in the manufacture of concentrated mixed fertilizer types. The greatest advantage to be had of using Superphosphate as a fertilizer is that the phosphoric acid is fully water soluble, but when Superphosphate is applied to the soil, it is converted into soluble phosphate. This is due to precipitation as calcium, iron or aluminum phosphate, which is dependent on the soil type to which the fertilizer is added, be it alkaline or acidic garden soil. All garden soil types can benefit from the application of Superphosphate as a fertilizer. Used in conjunction with an organic fertilizer, it should be applied at sowing or transplant time. Slag. Basic slag is a by-product of steel mills and is used as a fertilizer to a lesser extent than Superphosphate. Slag is an excellent fertilizer that can be to amend soils that are acidic because of its alkaline reaction. For slag application to be an effective fertilizer it has to be pulverized first. Bonemeal. Bone meal as a fertilizer type needs no introduction. Bone-meal is used as a phosphate fertilizer type and is available in two types: raw and steamed. The raw bone-meal contains 4% organic Nitrogen that is slow acting, and 20% to 25% phosphoric acid that is not soluble in water. The steamed bone-meal on the other hand has all the fats, greases, nitrogen and glue-making substances removed as a result of high pressure steaming. But it is more brittle and can be ground into a powder form. In powder form this fertilizer is of great advantage to the gardener in that the rate of availability of the phosphoric acid depends on its pulverization. This fertilizer is particularly suitable as a soil amendment for acid soil and should be applied either at sowing time or even a few days prior to sowing. Organic and Inorganic Chemical Potassium Fertilizer Types. Chemical Potassium fertilizer should only be added when there is absolute certainty that there is a Potassium Deficiency in your garden soil. Potassium fertilizers also work well in sandy garden soil that responds to their application. Crops such as chilies, potato and fruit trees all benefit from this type of fertilizer since it improves the quality and appearance of the produce. There are basically two different types of potassium fertilizers: Muriate of Potash (Potassium chloride) and Sulphate of Potash (Potassium sulphate). Both muriate of potash and sulphate of potash are salts that make up part of the waters of the oceans and inland seas as well as inland saline deposits. Muriate of potash or potassium chloride is a gray crystal type of fertilizer that consists of 50% to 60% potash. All the potash in this fertilizer type is readily available to plants because it is highly soluble in water. Even so, it does not leach away deep into the soil since the potash is absorbed on the colloidal surfaces. Sulphate Of Potash. Sulphate of potash is a fertilizer type manufactured when potassium chloride is treated with magnesium sulphate. It dissolves readily in water and can be applied to the garden soil at any time up to sowing. Some gardeners prefer using sulphate of potash to muriate of potash. Different Types of Fertilizers. The different types of fertilizers with all its specifications and cautions that should be kept in mind should not detract us from the joys of gardening. The different types of chemical and organic fertilizers that are usually commercially available in most countries can be categorized further into: Complete Inorganic Fertilizers:—these types of inorganic fertilizers contain all three major macronutrients, Nitrogen (N), Phosphorous (P) and Potassium (K). On the containers you will find that these macronutrients are depicted as a ratio, e.g. 2:3:2 (22). Complete inorganic fertilizers are usually applied at a rate of 60 g/m² or roughly 4 tablespoons per square meter. Special Purpose Fertilizer:—these types of fertilizer are formulated especially to target certain plants' requirements or certain soil deficiencies. Of the examples that come to mind here are the Blue Hydrangea Food, and straight fertilizer that are made up of one particular plant nutrient for example lawn fertilizer. Liquid Fertilizers:—these types of fertilizer come in a variety of formulations and even include organic fertilizer, complete fertilizer as well as special purpose fertilizer. Some examples of liquid fertilizer are Nitrosol and African Violet Food. Slow-Release Fertilizer:—these types of fertilizer are formulated to release their nitrogen at a steady pace. On the packs of this fertilizer that are available commercially it will usually be depicted as 3:1:5 (SR) where the SR indicates slow-release. Fertilizer with Insecticide:—these types of fertilizer that are prepared and combined with an insecticide. One such example is Wonder 4:1:1 (21)+Karbaspray. The reason why there are so many different types of chemical fertilizers in different formulations is because different plants require different nutrients and different pH levels in the soil. However, organic fertilizers have more diversity, and these types of fertilizers do not burn plant roots, get into ground water, or affect surrounding growth as is the case when using the different types of chemical fertilizer and NPK amendments.

Different Types of Metals. Non-limiting partial list of examples of metals include, but not limited to, the alkali metals, alkaline earth metals, transition metals, basic metals, and rare earth elements. Hydrogen in its metallic state (considered a nonmetal), Lithium, Sodium, Potassium, Rubidium, Cesium, Francium, Beryllium, Magnesium, Sodium, Calcium, Strontium, Barium, Radium, Aluminum, Gallium, Indium, Tin, Thallium, Lead, Bismuth, Element 113-Ununtrium, Flerovium, Element 115, Ununpentium, Livermorium, Scandium, Titanium, Vanadium, Chromium, Manganese, Iron, Cobalt, Nickel, Copper, Zinc, Yttrium, Zirconium, Niobium, Molybdenum, Technetium, Ruthenium, R hodium, Palladium, Silver, Cadmium, Lanthanum, Hafnium, Tantalum, Tungsten, Rhenium, Osmium, Iridium, Platinum, Gold, Mercury, Actinium, Rutherfordium, Dubnium, Seaborgium, Bohrium, Hassium, Meitnerium, Darmstadtium, Roent genium, Copernicium, Cerium, Praseodymium, Neodymium, Promethium, Samarium, Europium, Gadolinium, Terbium Dysprosium, Holmium, Erbium, Thulium, Ytterbium, Lutetium, Thorium. Protactinium, Uranium Neptunium, Plutonium, Americium, Curium, Berkelium, Californium, Einsteinium, Fermium, Mendelevium, Nobelium and Lawrencium.

Different Types of Plastics. Non-limiting partial list of examples of plastics can be divided into two major categories: Thermoset or thermosetting plastics. Once cooled and hardened, these plastics retain their shapes and cannot return to their original form. They are hard and durable. Thermosets can be used for auto parts, aircraft parts and tires. Examples include polyurethanes, polyesters, epoxy resins and phenolic resins. Thermoplastics. Less rigid than thermosets, thermoplastics can soften upon heating and return to their original form. They are easily molded and extruded into films, fibers and packaging. Examples include polyethylene (PE), polypropylene (PP) and polyvinyl chloride (PVC). Polyethylene Terephthalate (PET or PETE): John Rex Whinfield invented a new polymer in 1941 when he condensed ethylene glycol with terephthalic acid. The condensate was polyethylene terephthalate (PET or PETE). PET is a thermoplastic that can be drawn into fibers (like Dacron) and films (like Mylar). It's the main plastic in Ziplock™ food storage bags. Polystyrene (Styrofoam): Polystyrene is formed by styrene molecules. The double bond between the CH2 and CH parts of the molecule rearranges to form a bond with adjacent styrene molecules, thereby producing polystyrene. It can form a hard impact-resistant plastic for furniture, cabinets (for computer monitors and TVs), glasses and utensils. When polystyrene is heated and air blown through the mixture, it forms Styrofoam. Styrofoam is lightweight, moldable and an excellent insulator. Polyvinyl Chloride (PVC) is a thermoplastic that is formed when vinyl chloride (CH2═CH—Cl) polymerizes. When made, it's brittle, so manufacturers add a plasticizer liquid to make it soft and moldable. PVC is commonly used for pipes and plumbing because it's durable, can't be corroded and is cheaper than metal pipes. Over long periods of time, however, the plasticizer may leach out of it, rendering it brittle and breakable. Polytetrafluoroethylene (Teflon): Teflon was made in 1938 by DuPont. It's created by polymerization of tetrafluoroethylene molecules (CF2=CF2). The polymer is stable, heat-resistant, strong, and resistant to many chemicals and has a nearly frictionless surface. Teflon is used in plumbing tape, cookware, tubing, and waterproof coating applications, films and bearings. Polyvinylidine Chloride (Saran): Dow makes Saran resins, which are synthesized by polymerization of vinylidine chloride molecules (CH2=CCl2). The polymer can be drawn into films and wraps that are impermeable to food odors. Saran wrap is a popular plastic for packaging foods. Polyethylene, LDPE and HDPE: The most common polymer in plastics is polyethylene, which is made from ethylene monomers (CH2=CH2). The first polyethylene was made in 1934. This is called a low-density polyethylene (LDPE) because it will float in a mixture of alcohol and water. In LDPE, the polymer strands are entangled and loosely organized, so it's soft and flexible. It was first used to insulate electrical wires, but today it's used in films, wraps, plastic bottles, caps, plastic water bottles and other bottles, disposable gloves and garbage bags. In the 1950s, Karl Ziegler polymerized ethylene in the presence of various metals. The resulting polyethylene polymer was composed of mostly linear polymers. This linear form produced tighter, denser, more organized structures and is now called high-density polyethylene (HDPE). HDPE is a harder plastic with a higher melting point than LDPE, and it sinks in an alcohol-water mixture. HDPE was first introduced in the hula hoop, but today it's mostly used in containers. Polypropylene (PP): In 1953, Karl Ziegler and Giulio Natta, working independently, prepared polypropylene from propylene monomers (CH2=CHCH3) and received the Nobel Prize in Chemistry in 1963. The various forms of polypropylene have different melting points and hardnesses. Polypropylene is used in car trim, battery cases, bottles, water bottles and other bottles, tubes, filaments, etc. Acetal. non-limiting partial list of examples of plastics including, Acetal, which is a thermoplastic that was introduced in 1956. It is widely recognized as a potential replacement for die-cast metals because it is very rigid, yet not brittle. Acetal has a high melting point, is resistant to fatigue, and very strong. Currently, acetal is used to create cams, bearings, gears, bushings, housings, and conveyors. In addition, acetal is used in automotive seat belt components and door handles, shaver cartridges, in the moving parts in appliances and business machines, in gas tank caps, in plumbing fixtures, and in zippers. Acrylic. Acrylics became a part of the plastics family in 1936 and were used in World War II as aircraft canopies. Acrylics are known for being rigid, hard, and transparent. It is particularly useful in products that will be exposed to sunlight or other weather elements for periods of time because it is very resistant to sunlight and weathering. Today, acrylics are used in outdoor signs, lighting diffusers, washbasins, automobile tail lights, sinks, tables, safety shields, and skylights. Acrylics are also used for large enclosures, such as swimming pools and room dividers. Acrylonitrile-Butadiene-Styrene (ABS). This thermoplastic, which was introduced in 1948, is made by combining acrylonitrile, butadiene, and styrene. As a result, it draws upon the strengths of each. ABS is very durable against impact and has a high mechanical strength. Therefore, it is commonly used in automotive parts, appliances, business machines, pipes, and telephone components. Alkyds. Developed in 1926, alkyds were originally used in paints, enamels, lacquers, and other coatings used for refrigerators, automobiles with greater fuel efficiency, aerospace components with enhanced performance characteristics, better and future weapons platforms, longer lasting satellites, ceramic nanocrystalline (NC) coating applications, silicon thin films, electrochromic display devices, longer lasting coatings for medical applications, medical implants, breast implant devices, microchip implants or other types of implants and stoves. Today, this is still the main purpose for alkyds, though alkyd compounds are now also used as a molding material. In this capacity, it is used for encapsulation of capacitors and resistors, in circuit breaker insulation, in housings, in coal forms, in cases, and in switchgear components. This is large, resin due to the fact that alkyds have excellent dielectric strength as well as heat resistance. Cellulosics. Cellulosics has been around since 1868 when it was invented by John Wesley Hyatt. Several variations of the original cellulosics have been introduced since the early 1900s. Today, it is found in appliance housings, toys, knobs, handles, smart packaging and intelligent labeling technology for food, beverages, pharmaceutical and household products, and automotive parts. Coumarone-Indene. Coumarone-indene is mixed with other products to meet commercial applications. They are mostly used as extenders and plasticizers, as processing aids, and in asphalt floor tile. Diallyl Phthalate (DP). DAP can be made in both polymeric and monomeric form. As a polymer, it is used to produce casting resins, thermosetting molding powders, and laminates. Epoxy. Epoxies are used in numerous ways. In combination with glass fibers, it is capable of producing composites that are of high strength and that are heat resistant. This composite is typically used for filament wound rocket motor casings in missiles, in aircraft components, and in tanks, pipes, tooling jigs, pressure vessels, and fixtures. Epoxies are also found in gymnasium floors, industrial equipment, sealants, and protective coatings in appliances. Fluoropolymer. Fluoropolymers are inert to most chemicals and resistant to high temperatures. They also have low coefficients of friction and have superb dielectric properties. Therefore, fluoropolymers are used in electronics, as well as in pipe and in chemical processing equipment. It is also found in the non-stick coatings used for cookware. Malamine-Formaldehyde. Malamine-formaldehyde is used in many household goods, including dinnerware. This plastic is very easy to color and is very hard. Nitrile Resins. Nitrile resins were first developed in the late 1960s. They are resistant to flavor, aroma, and the transmission of gas. Therefore, they are ideal for packaging. Nylon. Nylon first appeared in 1939 when its fiber was used in the production of nylon stockings. Nylon is found in more than just stockings, however, such as in electronics, automotive parts, and in packaging. Petroleum Resins. Petroleum resins are used in printing inks, adhesives, surface coatings, and rubber compounding. It is optionally obtained as the byproduct from distilled petroleum streams. Phenolic. Phenolic plastics are thermosetting resins used in potting compounds, casting resins, and laminating resins. They can be used for electrical purposes and are a popular binder for holding together plies of wood for plywood. Polyamide-Imide. Polyamide-imide is used in the automotive, aerospace, and heavy equipment industries. Polyarylates. Polyarylates are used in appliance, automotive, and electrical applications such as outdoor lighting because they are resistant to heat. Polybutylene. Polybutylene is a thermoplastic that is resistant to creep, chemicals, and cracking, while being very flexible. It is typically used in packaging film and pipe.

Polycarbonate. Polycarbonate is a thermoplastic that was first developed in 1957. It was originally created as a means of competing against die-cast metals. Polycarbonates are tough, strong, and rigid, yet ductile. They can be maintained over a wide range of loading rates and temperatures and are excellent electrical insulators. They are transparent and, therefore, are often used in the creation of bottles, water bottles, caps, engineered wood, furniture, and floors. They are also used for electrical purposes, glazing, and appliances. In addition, they can be processed in numerous ways, including extrusion, injection molding, rotational molding, and blow molding. Polyethylene. Polyethylene came to the forefront during World War II, when it was used for underwater cable coating. It was then used as an insulating material for other military purposes, such as radar cable. After the war was over, it was put to commercial use and has become one of the most popular forms of plastic. In fact, it was the first plastic in the United States to sell more than billion pounds a year. It remains the most popular plastic in the country, being found in drums, containers, pipe, toys, housewares, shopping bags, trash bags, garment bags, packaging films, gasoline tanks, and coatings. Polyimides. Polymides are a thermoset plastic that first appeared in the 1960s. They are typically used in laminates, enamels, gears, adhesives, bushings, covers, valve seats, piston rings, and solutions such as laminating varnish. Polyphenylene Oxide, Modified. Polyphenylene Oxide is an engineered thermoplastic used in business machine parts, automotive parts, appliances, and electronics.
Polyphenylene Sulfide. Polyphenylene sulfide is heat and chemical resistant. It also has a good retention of mechanical properties at high temperatures and is very stiff. Therefore, they are often used in automotive and electronic parts. Polypropylene. Polypropylene is a highly used thermoplastic that was first developed in Europe and brought to the United States in 1957. It is fairly rigid, has a low density, excellent chemical resistance, barrier properties, and has a heat distortion temperature of 150 to 200 degrees Fahrenheit. In addition, it is very simple to process. It is most often used in automotive parts, smart packaging and intelligent labeling technology for food, beverages, pharmaceutical and household products, carpeting, and appliances. Polystyrene. Polystyrene was first created in 1845, but was not put into commercial production until 1925. Now, polystyrene is one of the most used thermoplastics, with the foamed version being used in protective smart packaging and intelligent labeling technology for food, beverages, pharmaceutical and household products, foam containers and cups, and building insulation. It is also used in toys, automotive parts, housewares, wall tiles, appliance parts, television and radio housings, floats, furniture, and luggage. Polyurethane. Polyurethanes have been around since 1954 and are very versatile. In fact, they are available in rubbers, adhesives, sealants, coatings, and flexible or rigid foams. Most are considered to be thermosets, though some are thermoplastics. The foam version is created by reacting polyols and isocyanates, which are then introduced to a blowing agent. The foams can be made to be rigid, flexible, or tough, depending on the purpose. The foam polyurethanes have excellent thermal insulating properties and, therefore, are used in building insulation. In addition, they have good dimensional stability and compressive strength, making them ideal for use in trucks, refrigerators, and boats for floatation purposes. They can be very cushiony with energy-absorbing properties and durability. Therefore, they are also used as carpet underlay, in furniture, in automobile seating, in bedding, in smart packaging and intelligent labeling technology for food, beverages, pharmaceutical and household products, and as safety padding. Polyurethanes also have protective qualities, making them great for use as coatings for metals, wood, rubber, concrete, leather, paper, and plastic. Their toughness and resistance to abrasion also make them ideal for gaskets and seals, printing rolls, drive and conveyor belts, cable insulation, and solid tires. Polyvinyl Acetate (PVAc) and Other Vinyls. Polyvinyl acetate, which is a thermoplastic, is used to create solid vinyl acetate. It is typically used in paints, adhesives, coatings, and packaging. Polyvinyl Chloride. Polyvinyl Chloride, commonly referred to as PVC or vinyl, was first invented in Germany around 1910. It didn't become a useful product in the United States, however, until the late 1920s. It became particularly useful during World War II when it was used as a substitute for rubber, which was in short supply. Polyvinyl Chloride is resistant to abrasion and is both weather and chemical resistant. Today, it is commonly found in upholstery, wall coverings, flooring, siding, pipe, and even apparel. In fact, vinyl is perhaps the best known of all plastics. Styrene Acrylonitrile. Styrene Acrylonitrile is typically used in housewares an din the interior trim and instrument panels of automobiles with greater fuel efficiency, aerospace components with enhanced performance characteristics, better and future weapons platforms, longer lasting satellites, ceramic nanocrystalline (NC) coating applications, silicon thin films, electrochromic display devices, longer lasting medical implants. Styrene Butadiene Latexes and Other Styrene Copolymers. Styrene Butadiene Latexes are commonly found in coatings, paints, and floor polishes. Sulfone Polymers. Sulfone Polymers are found in automotive parts and electronics. Thermoplastic Polyster (Saturated). Thermoplastic Polyster compounds were introduced in the 1970s and are hard, crystalline, strong, and tough. They are commonly used in soda bottles, water bottles, caps, engineered wood, furniture, and floors, as well as in magnetic tape for video, audio, and computers. They are also used in X-ray film, strapping, labels, and packaging. Unsaturated Polyester. Unsaturated Polyeters are very different from saturated thermoplastic polyester. These thermosets are found in fiberglass reinforced plastics and were first used in the United States during World War II. They are also used in fishing poles, luggage, and in electrical applications. Urea-Formaldehyde. Developed in 1929, Urea-Formaldehyde is scratch resistant, chemical resistant, heat resistant, hard, and contains good electrical qualities. The molding compounds of Urea-Formaldehyde are used in rigid decorative and electrical products, while the liquid, non-liquid resins are used in laminates and in chemically resistant coatings.

Dietary Supplement is intended to provide nutrients that may otherwise not be consumed in sufficient quantities. Supplements as generally understood include vitamins, minerals, fiber, fatty acids, or amino acids, among other substances. U.S. authorities define dietary supplements as cellulose in foods, while elsewhere they may be classified as drugs or other products. There are more than 50,000 dietary supplements available. More than half of the U.S. adult population (53%-55%) consume dietary supplements with most common ones being multivitamins. These products are not intended to prevent or treat any disease and in some circumstances are dangerous, according to the U.S. National Institutes of Health. For those who fail to consume a balanced diet, the agency says that certain supplements “may have value.” Most supplements should be avoided, and usually people should not eat micronutrients except people with clearly shown deficiency. Those people should first consult a doctor. An exception is vitamin D, which is recommended in Nordic countries due to weak sunlight. Definition. According to the United States Food and Drug Administration (FDA), dietary supplements are products which are not pharmaceutical drugs, food additives like spices or preservatives, or conventional food, and which also meet any of these criteria: The product is intended to supplement a person's diet, despite it not being usable as a meal replacement. The product is or contains a vitamin, dietary element, herb used for herbalism or botanical used as a medicinal plant, amino acid, any substance which contributes to other food eaten, or any concentrate, metabolite, ingredient, extract, or combination of these things. The product is labeled as a dietary supplement.

Vitamins. Vitamin is an organic compound required by an organism as a vital nutrient in limited amounts. An organic chemical compound (or related set of compounds) is called a vitamin when it cannot be synthesized in sufficient quantities by an organism, and must optionally be obtained from the diet. Thus, the term is conditional both on the circumstances and on the particular organism. For example, ascorbic acid (vitamin C) is a vitamin for humans, but not for most other animals. Supplementation is important for the treatment of certain health problems but there is little evidence of benefit when used by those who are otherwise healthy.

Dietary Element. Dietary elements, commonly called “dietary minerals” or “minerals”, are the chemical elements required by living organisms, other than the four elements carbon, hydrogen, nitrogen, and oxygen present in common organic molecules. The term “dietary mineral” is archaic, as the substances it refers are chemical elements rather than actual minerals.

Herbal Medicine. Herbal medicine is the use of plants for medicinal purposes. Plants have been the basis for medical treatments through much of human history, and such traditional medicine is still widely practiced today. Modern medicine recognizes herbalism as a form of alternative medicine, as the practice of herbalism is not strictly based on evidence gathered using the scientific method. Modern medicine, does, however, make use of many plant-derived compounds as the basis for evidence-tested pharmaceutical drugs, and phytotherapy works to apply modern standards of effectiveness testing to herbs and medicines that are derived from natural sources. The scope of herbal medicine is sometimes extended to include fungal and bee products, as well as minerals, shells and certain animal parts.

Amino Acids and Proteins. Amino acids are biologically important organic compounds composed of amine (—NH₂) and carboxylic acid (—COOH) functional groups, along with a side-chain specific to each amino acid. The key elements of an amino acid are carbon, hydrogen, oxygen, and nitrogen, though other elements are found in the side-chains of certain amino acids. Amino acids can be divided into three categories: essential amino acids, non-essential amino acids, and conditional amino acids. Essential amino acids cannot be made by the body, and must be supplied by food. Non-essential amino acids are made by the body from essential amino acids or in the normal breakdown of proteins. Conditional amino acids are usually not essential, except in times of illness, stress, or for someone challenged with a lifelong medical condition.

Essential Fatty Acids. Essential fatty acids, or EFAs, are fatty acids that humans and other animals must ingest because the body requires them for good health but cannot synthesize them. The term “essential fatty acid” refers to fatty acids required for biological processes but does not include the fats that only act as fuel.

Bodybuilding Supplements. Bodybuilding supplements are dietary supplements commonly used by those involved in bodybuilding and athletics. Bodybuilding supplements may be used to replace meals, enhance weight gain, promote weight loss or improve athletic performance. Among the most widely used are vitamin supplements, protein drinks, branched-chain amino acids (BCAA), glutamine, essential fatty acids, meal replacement products, creatine, weight loss products and testosterone boosters. Supplements are sold either as single ingredient preparations or in the form of “stacks”—proprietary blends of various supplements marketed as offering synergistic advantages. While many bodybuilding supplements are also consumed by the general public their salience and frequency of use may differ when used specifically by bodybuilders. Contraindications. Dietary supplements for healthy cell growth may include a combination of Spirulina mixed with natural yeast, natural vitamin C powder, L Lysine, organic Lecithin, organic coconut oil and purified water.

Digestion and Absorption of Food. The gastrointestinal (GI) system includes the gastrointestinal tract (mouth, pharynx, esophagus, stomach, small intestine, large intestine) and accessory organs (salivary gland, liver, gallbladder, pancreas) that secrete substances into the tract via connecting ducts. GI system breaks down particles of ingested food into molecular forms by enzymes (digestion) that are then transferred to the internal environment (absorption). Functions of GI organs. The GI tract begins at the mouth, where digestion begins with chewing. Saliva containing mucus and the enzyme amylase is secreted from 3 pairs of salivary glands, located in the head. Mucus moistens the food and amylase partially digests polysaccharides (starches). Food then reaches the stomach through the pharynx and esophagus. The stomach is the sac that stores and digests food macromolecules into a solution called chyme. Glands lining the stomach secrete hydrochloric acid that dissolves food particles and protein-digesting enzymes, called pepsin. Final stages of digestion and most of the nutrient absorption occur in next portion of the tract: the small intestine. The small intestine is divided into 3 segments—duodenum, jejunum, and ileum. The pancreas is a gland located behind the stomach. From its exocrine portion it secretes (1) digestive enzymes and (2) a fluid rich in HCO₃₋ ions to neutralize the acid from stomach. The liver secretes bile. Bile contains HCO₃₋ ions and bile salts to solubilize fats. Bile reaches the gall bladder through hepatic ducts and is stored in the gall bladder between meals. During a meal, bile is secreted from the gland by smooth muscle contraction and reaches the duodenum portion of the small intestine by the common bile duct. Monosaccharides, amino acids and mineral salts are absorbed by transporter-mediated processes while fatty acid water diffuse passively. Undigested material is passed to large intestine, where it is temporarily stored and concentrated by reabsorption of salts and water. Finally, contractions of rectum, the last part of large intestine, expel the feces through the anus. Structure of GI Tract Wall The luminar surface is covered by a single layer of epithelium containing exocrine and endocrine cells. The exocrine cells disintegrate and discharge into the lumen, releasing their enzymes. The epithelia with an underlying layer of connective tissue (lamnia propia) and muscle (muscularis mucosa) are called mucosa. Below the mucosa is a layer of inner circular and outer longitudinal smooth muscle called muscularis externa, which provides the forces for moving and mixing the GI contents. The outermost layer of the tube is made up of connective tissue called serosa. The luminar surface of the tube is highly convoluted into projections called villi and microvilli; both of which increase total surface area for absorption. The center of each villus has a single blunt-ended lymphatic vessel called lacteal. Venous drainage from the intestine transports absorbed materials to the liver for processing via the hepatic portal vein. Digestion and Absorption. Carbohydrate. Digestion begins in the mouth by salivary amylase and completed in the small intestine by pancreatic amylase. Monosaccharides, such as glucose, galactose and fructose, are produced by the breakdown of polysaccharides and are transported to the intestinal epithelium by facilitated diffusion or active transport. Facilitated diffusion moves the sugars to the bloodstream. Protein. Proteins are broken down to peptide fragments by pepsin in the stomach, and by pancreatic trypsin and chemotrypsin in the small intestine. The fragments are then digested to free amino acids by carboxypeptidase from the pancreas and aminopeptidase from the intestinal epithelium. Free amino acids enter the epithelium by secondary active transport and leave it by facilitated diffusion. Small amounts of intact proteins can enter interstitial fluid by endo- and exocytosis. Fat. Fat digestion occurs by pancreatic lipase in small intestine. A monoglyceride and two fatty acids are produced in the digestive process. Large lipid droplets are first broken down into smaller droplets, by a process called emulsification. Emulsification is driven by mechanical disruption (by contractile activity of GI tract) and emulsifying agents (amphipathic bile salts). Pancreatic colipase binds the water-soluble lipase to the lipid substrate. Digested products and bile salts form amphipathic micelles. These micelles keep the insoluble products in soluble aggregates from which small amounts are released and absorbed by epithelial cells via diffusion. Free fatty acids and monoglycerides then recombine into triacylglycerols at the smooth ER, are processed further in the Golgi and enter the interstitial fluid as droplets called chylomicrons, which are then taken up by the lacteals in the intestine. Vitamins. Fat-soluble vitamins are absorbed and stored along with fats. Most water-soluble vitamins are absorbed by diffusion or mediated transport. Vitamin B₁₂, because of its large size and charged nature, first binds to a protein, called intrinsic factor, which is secreted by the stomach epithelium, and is then absorbed by endocytosis. Water. The stomach absorbs some water but most is absorbed at small intestine by diffusion. Regulation of GI Processes. Control mechanisms of the GI system regulate conditions in the lumen of the tract. Reflexes are initiated by: (1) Distension of wall by volume of luminal contents, (2) Chyme osmolarity, (3) Chyme pH, (4) Chyme concentrations of specific products. Neural Regulation of the GI tract. Impulses to the GI muscles and exocrine glands are supplied by enteric nervous system, the local nervous system of GI tract, which allows local, short reflexes, independent of CNS. Long reflexes through the CNS are possible via sympathetic and parasympathetic nerves, which also innervate the GI tract. Hormonal Regulation. Endocrine cells are scattered throughout GI epithelia and surface of these cells is exposed to the lumen. Chemical substances in the chyme stimulate them to release hormones into blood. Phases of GI control. Each phase is named according to where the receptor for a reflex is located. Bile Secretion. Bile contains bile salts, which solubilize fats, and bicarbonate ions, which in turn are used to neutralize stomach acids. Bile salts, secreted by hepatocytes (liver cells) enter the GI tract and are reabsorbed by transporters in the intestine and are returned to the liver via the portal vein. This recycling pathway is called the entero-hepatic circulation. The sphincter of Oddi controls the entry of the bile duct into the duodenum. When the sphincter is closed, secreted bile is shunted into the gallbladder. The presence of fat in the intestine releases CCK, which relaxes the sphincter to discharge bile salts into the duodenum. Small Intestine. The most common motion of the small intestine is stationary contraction and relaxation, called segmentation. Segmentation results in little net movement. The chyme is mixed and brought into contact with the intestine wall and then moved slowly toward the large intestine. The movements are initiated by pacemaker cells in the smooth muscle layer. After most of the materials are absorbed, segmentation is replaced by peristaltic activity called migrating motility complex, which moves any undigested material to the large intestine. The candidate intestinal hormone, motilin, initiates migrating motility. Large Intestine. The large intestine consists of 3 parts: the cecum, colon and rectum. The primary function is to store and concentrate fecal material for elimination. Chyme enters the cecum through the ileocecal sphincter, which relaxes and opens as a result of the gastroileal reflex. Na⁺ is absorbed along with water. K⁺ and HCO₃₋ ions are secreted into the lumen. Undigested polysaccharides (fiber) are metabolized to short-chain fatty acids by the residing bacteria and these are then absorbed by diffusion. A small amount of vitamin K is also produced and absorbed. Bacterial metabolism produces a mixture of gases, called flatus. Motility and Defecation. Regular contractions of the circular smooth muscle produce a slow rhythmic segmentation movement. The undigested material moves slowly in order to provide resident bacteria time to grow and multiply. Following a meal, there is a wave of intense contraction, called mass movement. The internal anal sphincter is made of smooth muscle and closes the anus, while the external anal sphincter is made of skeletal muscle and is under voluntary control. Both sphincters regulate the anal opening and closing. Mass movement of fecal material into the anus initiates the defecation reflex, which is mediated by mechanoreceptors. The two sphincters open to expel the feces. If defecation is delayed, rectal contents are driven back into colon by reverse peristalsis until the next mass movement. Pathophysiology of the GI Tract Ulcers. Ulcers are eroded areas of gastric surface and breaks in the mucosal barrier, which expose the underlying tissue to corrosive action of acid and pepsin. Damage to underlying blood vessels may cause bleeding. Gallstones. Excessive secretion of water insoluble cholesterol in bile results in formation of crystals, called gallstones, which can close the opening of gallbladder or the bile duct. If a stone prevents bile from entering the intestine fat digestion and absorption decreases. If a stone blocks the entry of the pancreatic duct, it prevents pancreatic enzymes from entering the intestine, thus preventing the digestion of other nutrients. A blocked bile duct inhibits further secretion of bile, resulting in accumulation of bilirubin in tissues, producing a yellowish coloration called jaundice. Jaundice is common in newborns and is rectified by sunlight exposure. Lactose Intolerance. Lactose intolerance results from a lack of the enzyme lactase, which digests lactose, the sugar in milk. The lack of lactase results in the incomplete digestion of lactose to glucose and galactose. Constipation and Diarrhea. Constipation is the absence of defecation due to decreased motility of the large intestine. This results in excess absorption of water from feces, making it hard to expel. Dietary fiber, which is not digested in small intestine, can produce distension and increase motility. Diarrhea results from decreased fluid absorption, or increased fluid secretion resulting in increased luminal fluid, which in turn, causes distension and increased motility. Diarrhea results in decreased blood volume, loss of water and other nutrients.

Dissolution Media as used herein can optionally be any suitable dissolution media. In general, such a media breaks or disrupts the hydrogen bonding between individual cellulose chains and substantially isolates individual cellulose chains by surrounding them with ions and solvent molecules. Examples of dissolution media include, but are not limited to, acid solutions such as sulfuric acid, nitric acid, phosphoric acid, organic solvents, ionic liquids, basic solutions (e.g., NaOH, NaOH/Urea solutions) LiCl/DMAc solutions, and the like, including suitable combinations thereof.

DNA Sequencing is the process of determining the precise order of nucleotides within a DNA molecule. It includes any method or technology that is used to determine the order of the four bases—adenine, guanine, cytosine, and thymine—in a strand of DNA. The advent of rapid DNA sequencing methods has greatly accelerated biological and medical research and discovery. Knowledge of DNA sequences has become indispensable for basic biological research, and in numerous applied fields such as medical diagnosis, biotechnology, forensic biology, virology and biological systematics. The rapid speed of sequencing attained with modern DNA sequencing technology has been instrumental in the sequencing of complete DNA sequences, or genomes of numerous types and species of life, including the human genome and other complete DNA sequences of many animal, plant, and microbial species. The first DNA sequences were obtained in the early 1970s by academic researchers using laborious methods based on two-dimensional chromatography. Following the development of fluorescence-based sequencing methods with automated analysis, DNA sequencing has become easier and orders of magnitude faster.

Recombinant DNA (rDNA) molecules are DNA molecules formed by laboratory methods of genetic recombination (such as molecular cloning) to bring together genetic material from multiple sources, creating sequences that would not otherwise be found in biological organisms. Recombinant DNA is possible because DNA molecules from all organisms share the same chemical structure. They differ only in the nucleotide sequence within that identical overall structure. Recombinant DNA is the general name for taking a piece of one DNA, combining it with another strand of DNA. Recombinant DNA molecules are sometimes called chimeric DNA, because they are usually made of material from two different species, like the mythical chimera. R-DNA technology uses palindromic sequences and leads to the production of sticky and blunt ends. The DNA sequences used in the construction of recombinant DNA molecules can originate from any species. For example, plant DNA may be joined to bacterial DNA, or human DNA may be joined with fungal DNA. In addition, DNA sequences that do not occur anywhere in nature may be created by the chemical synthesis of DNA, and incorporated into recombinant molecules. Using recombinant DNA technology and synthetic DNA, literally any DNA sequence may be created and introduced into any of a very wide range of living organisms. Proteins that can result from the expression of recombinant DNA within living cells are termed recombinant proteins. When recombinant DNA encoding a protein is introduced into a host organism, the recombinant protein is not necessarily produced. Expression of foreign proteins requires the use of specialized expression vectors and often necessitates significant restructure by foreign coding sequence.

Recombinant DNA differs from genetic recombination in that the former results from artificial methods in the test tube, while the latter is a normal biological process that results in the remixing of existing DNA sequences in essentially all organisms. Creating recombinant DNA. Molecular cloning is the laboratory process used to create recombinant DNA. It is one of two widely used methods, along with polymerase chain reaction (PCR) used to direct the replication of any specific DNA sequence chosen by the experimentalist. The fundamental difference between the two methods is that molecular cloning involves replication of the DNA within a living cell, while PCR replicates DNA in the test tube, free of living cells. Formation of recombinant DNA requires a cloning vector, a DNA molecule that replicates within a living cell. Vectors are generally derived from plasmids or viruses, and represent relatively small segments of DNA that contain necessary genetic signals for replication, as well as additional elements for convenience in inserting foreign DNA, identifying cells that contain recombinant DNA, and, where appropriate, expressing the foreign DNA. The choice of vector for molecular cloning depends on the choice of host organism, the size of the DNA to be cloned, and whether and how the foreign DNA is to be expressed. The DNA segments can be combined by using a variety of methods, such as restriction enzyme/ligase cloning or Gibson assembly. In standard cloning protocols, the cloning of any DNA fragment essentially involves seven steps: (1) Choice of host organism and cloning vector, (2) Preparation of vector DNA, (3) Preparation of DNA to be cloned, (4) Creation of recombinant DNA, (5) Introduction of recombinant DNA into the host organism, (6) Selection of organisms containing recombinant DNA, and (7) Screening for clones with desired DNA inserts and biological properties. These steps are described in some detail in a related article (molecular cloning). Expression of recombinant DNA. Following transplantation into the host organism, the foreign DNA contained within the recombinant DNA construct may or may not be expressed. That is, the DNA may simply be replicated without expression, or it may be transcribed and translated so that a recombinant protein is produced. Generally speaking, expression of a foreign gene requires restructuring the gene to include sequences that are required for producing an mRNA molecule that can be by the host's translational apparatus (e.g. promoter, translational initiation signal, and transcriptional terminator). Specific changes to the host organism may be made to improve expression of the ectopic gene. In addition, changes may be needed to the coding sequences as well, to optimize translation, make the protein soluble, direct the recombinant protein to the proper cellular or extracellular location, and stabilize the protein from degradation. Properties of organisms containing recombinant DNA. In most cases, organisms containing recombinant DNA have apparently normal phenotypes. That is, their appearance, behavior and metabolism are usually unchanged, and the only way to demonstrate the presence of recombinant sequences is to examine the DNA itself, typically using a polymerase chain reaction (PCR) test. Significant exceptions exist, and are discussed below. If the rDNA sequences encode a gene that is expressed, then the presence of RNA and/or protein products of the recombinant gene can be detected, typically using RT-PCR or western hybridization methods. Gross phenotypic changes are not the norm, unless the recombinant gene has been chosen and modified so as to generate biological activity in the host organism. Additional phenotypes that are encountered include toxicity to the host organism induced by the recombinant gene product, especially if it is over-expressed or expressed within inappropriate cells or tissues. In some cases, recombinant DNA can have deleterious effects even if it is not expressed. One mechanism by which this happens is insertional inactivation, in which the rDNA becomes inserted into a host cell's gene. In some cases, researchers use this phenomenon to “knock out” genes to determine their biological function and importance. Another mechanism by which rDNA insertion into chromosomal DNA can affect gene expression is by inappropriate activation of previously unexpressed host cell genes. This can happen, for example, when a recombinant DNA fragment containing an active promoter becomes located next to a previously silent host cell gene, or when a host cell gene that functions to restrain gene expression undergoes insertional inactivation by recombinant DNA. Applications of recombinant DNA technology.

Recombinant DNA is widely used in biotechnology, medicine and research. Today, recombinant proteins and other products that result from the use of rDNA technology are found in essentially every western pharmacy, doctor's or veterinarian's office, medical testing laboratory, and biological research laboratory. In addition, organisms that have been manipulated using recombinant DNA technology, as well as products derived from those organisms, have found their way into many farms, supermarkets, home medicine cabinets, and even pet shops, such as those that sell GloFish and other genetically modified animals. The most common application of recombinant DNA is in basic research, in which the technology is important to most current work in the biological and biomedical sciences. Recombinant DNA is used to identify, map and sequence genes, and to determine their function. rDNA probes are employed in analyzing gene expression within individual cells, and throughout the tissues of whole organisms. Recombinant proteins are widely used as reagents in laboratory experiments and to generate antibody probes for examining protein synthesis within cells and organisms. Many additional practical applications of recombinant DNA are found in industry, food production, human and veterinary medicine, agriculture, and bioengineering. Non-limiting examples are identified below.

Recombinant Chymosin. Found in rennet, is an enzyme required to manufacture cheese. It was the first genetically engineered food additive used commercially. Traditionally, processors obtained chymosin from rennet, a preparation derived from the fourth stomach of milk-fed calves. Scientists engineered a non-pathogenic strain (K-12) of E. coli bacteria for large-scale laboratory production of the enzyme. This microbiologically produced recombinant enzyme, identical structurally to the calf derived enzyme, costs less and is produced in abundant quantities. Today about 60% of U.S. hard cheese is made with genetically engineered chymosin. In 1990, FDA granted chymosin “generally-recognized-as-safe” (GRAS) status based on data showing that the enzyme was safe.

Recombinant Human Insulin. Almost completely replaced insulin obtained from animal sources (e.g. pigs and cattle) for the treatment of insulin-dependent diabetes. A variety of different recombinant insulin preparations are in widespread use. Recombinant insulin is synthesized by inserting the human insulin gene into E. coli, or yeast (saccharomyces cerevisiae), which then produces insulin for human use.

Recombinant Human Growth Hormone (HGH, somatotropin). Administered to patients whose pituitary glands generate insufficient quantities to support normal growth and development. Before recombinant HGH became available, HGH for therapeutic use was obtained from pituitary glands of cadavers. This unsafe practice led to some patients developing Creutzfeldt-Jakob disease. Recombinant HGH eliminated this problem, and is now used therapeutically. It has also been misused as a performance enhancing drug by athletes and others. Drug Bank entry.

Recombinant Blood Clotting Factor VIII. A blood-clotting protein that is administered to patients with forms of the bleeding disorder hemophilia, who are unable to produce factor VIII in quantities sufficient to support normal blood coagulation. Before the development of recombinant factor VIII, the protein was obtained by processing large quantities of human blood from multiple donors, which carried a very high risk of transmission of blood borne infectious diseases, for example HIV and hepatitis B. Drug Bank entry.

Recombinant Hepatitis B Vaccine. Hepatitis B infection is controlled through the use of a recombinant hepatitis B vaccine, which contains a form of the hepatitis B virus surface antigen that is produced in yeast cells. The development of the recombinant subunit vaccine was an important and necessary development because hepatitis B virus, unlike other common viruses such as polio virus, cannot be grown in vitro. Vaccine information from Hepatitis B Foundation.

Diagnosis of infection with HIV. Each of the three widely used methods for diagnosing HIV infection has been developed using recombinant DNA. The antibody test (ELISA or western blot) uses a recombinant HIV protein to test for the presence of antibodies that the body has produced in response to an HIV infection. The DNA test looks for the presence of HIV genetic material using reverse transcription polymerase chain reaction (RT-PCR). Development of the RT-PCR test was made possible by the molecular cloning and sequence analysis of HIV genomes. HIV testing page from US Centers for Disease Control (CDC).

Golden Rice. A recombinant variety of rice that has been engineered to express the enzymes responsible for β-carotene biosynthesis. This variety of rice holds substantial promise for reducing the incidence of vitamin A deficiency in the world's population. Golden rice is not currently in use, pending the resolution of regulatory issues. Gelatin or Gelatine (from Latin: gelatus meaning “stiff”, “frozen”) is a translucent, colourless, brittle (when dry), flavourless foodstuff, derived from collagen obtained from various animal by-products. It is commonly used as a gelling agent in food, pharmaceuticals, photography, and cosmetic manufacturing. Substances containing gelatin or functioning in a similar way are called gelatinous. Gelatin is an ineversiblyhydrolyzed form of collagen. It is found in most gummy candy as well as other products such as marshmallows, gelatin dessert, and some ice cream, dip and yogurt. Household gelatin comes in the form of sheets, granules, or powder. Instant types can be added to the food as they are; others need to be soaked in water beforehand.

Vegetable or Gelatin Capsules. In the manufacture of pharmaceuticals, encapsulation refers to a range of dosage forms—techniques used to enclose medicines—in a relatively stable shell known as a capsule, allowing them to, for example, be taken orally or be used as suppositories. The two main types of capsules are: Hard-shelled capsules, which are typically made using gelatin and contain dry, powdered ingredients or miniature pellets made by e.g. processes of extrusion or spheronisation. These are made in two halves: a lower-diameter “body” that is filled and then sealed using a higher-diameter “cap”. Soft-shelled capsules, primarily used for oils and for active ingredients that are dissolved or suspended in oil. Both of these classes of capsules are made from aqueous solutions of gelling agents like: Animal protein, mainly gelatin; Plant polysaccharides or their derivatives like carrageenans and modified forms of starch and cellulose. Other ingredients can be added to the gelling agent solution like plasticizers such as glycerin or sorbitol to decrease the capsule's hardness, coloring agents, preservatives, disintegrants, lubricants and surface treatment. Since their inception, capsules have been viewed by consumers as the most efficient method of taking medication. For this reason, producers of drugs such as OTC analgesics wanting to emphasize the strength of their product developed the “caplet” or “capsule-shaped tablet” in order to tie this positive association to more efficiently-produced tablet pills.

Herbicide-Resistant Crops. Commercial varieties of important agricultural crops (including soy, maize/corn, sorghum, canola, alfalfa and cotton) have been developed that incorporate a recombinant gene that results in resistance to the herbicide glyphosate (trade name Roundup), and simplifies weed control by glyphosate application. These crops are in common commercial use in several countries.

Insect-Resistant Crops. Bacillus thuringeiensis is a bacterium that naturally produces a protein (Bt toxin) with insecticidal properties. The bacterium has been applied to crops as an insect-control strategy for many years, and this practice has been widely adopted in agriculture and gardening. Recently, plants have been developed that express a recombinant form of the bacterial protein, which may effectively control some insect predators. Environmental issues associated with the use of these transgenic crops have not been fully resolved.

Electromagnetic Shielding is the practice of reducing the electromagnetic field in a space by blocking the field with barriers made of conductive or magnetic materials. Shielding is typically applied to enclosures to isolate electrical devices from the ‘outside world’, and to cables to isolate wires from the environment through which the cable runs. Electromagnetic shielding that blocks radio frequency electromagnetic radiation is also known as RF shielding. The shielding can reduce the coupling of radio waves, electromagnetic fields and electrostatic fields. A conductive enclosure used to block electrostatic fields is also known as a Faraday cage. The amount of reduction depends very much upon the material used, its thickness, the size of the shielded volume and the frequency of the fields of interest and the size, shape and orientation of apertures in a shield to an incident electromagnetic field.

Electromagnetic Wave Absorption and Nanocrystalline Magnetic Materials is used in high-frequency electronic and communication devices has led to a rise in the amount of electromagnetic (EM) waves, causing harmful effects on human body and other nearby devices to malfunction. As concern about the effect of EM wave grows, the devices are required to have electromagnetic compatibility (EMC). Fe-based nanocrystalline magnetic materials such as Finemet alloys have excellent soft magnetic properties including large saturation magnetization and high relative permeability in the high frequency range. One application of the Finemet type alloy is an EM wave absorber, which absorbs the generated EM waves to transform into heats. FeSiBNbCu alloys exhibit excellent soft magnetic properties when nanocrystalline bcc-Fe(Si) phases that was formed by the crystallization annealing were embedded uniformly in the amorphous matrix. Numerous studies have been made on the effect of grain size of crystalline bcc-Fe(Si) phase on the magnetic properties of FeSiBNbCu alloy, in which the optimum magnetic properties can be acquired when the grain size is controlled to the range 10˜15 nm.

Electrostatic Spray Assisted Vapor Deposition (ESAVD) is a technique (developed by a company called IMPT) to deposit both thin and thick layers of a coating onto various substrates. In simple terms chemical precursors are sprayed across an electrostatic field towards a heated substrate, the chemicals undergo a controlled chemical reaction and are deposited on the substrate as the required coating. Electrostatic spraying techniques were developed in the 1950s for the spraying of ionized particles on to charged or heated substrates. ESAVD (branded by IMPT as Layatec) is used for many applications in many markets including: Thermal barrier coating applications for jet engine turbine blades, various thin layers in the manufacture of flat panel displays and photovoltaic panels, electronic components, batteries, catalysis, ceramics, magnetic data storage, telecommunication and data communication components, biomedical coating applications, glass coating applications (such as self-cleaning), corrosion protection coating applications. The process has advantages over other techniques for layer deposition (Plasma, Electron-Beam) in that it does not require the use of any vacuum, electron beam or plasma so reduces the manufacturing costs. It also uses less power and raw materials making it more environmentally friendly. Also the use of the electrostatic field means that the process can coat complex 3D parts easily.

Entomopathogenic Fungus is a fungus or fungi that can act as a parasite of insects and kills or seriously disables them. Typical Life Cycle of Fungus. These fungi usually attach to the external body surface of insects in the form of microscopic spores (usually asexual, mitosporic spores also called conidia). Under the right conditions of temperature and (usually high) humidity, these spores germinate, grow as hyphae and colonize the insect's cuticle; eventually they bore through it and reach the insects' body cavity (hemocoel). Then, the fungal cells proliferate in the host body cavity, usually as walled hyphae or in the form of wall-less protoplasts (depending on the fungus involved). After some time the insect is usually killed (sometimes by fungal toxins) and new propagules (spores) are formed in or on the insect if environmental conditions are again right. High humidity is usually required for sporulation. Groups. The entomopathogenic fungi include taxa from several of the main fungal groups and do not form a monophyletic group. Many common and/or important entomopathogenic fungi are in the order Hypocreales of the Ascomycota: the asexual (anamorph) phases Beauveria, Metarhizium, Nomuraea, Paecilomyces=Isaria, Hirsutella and the sexual (teleomorph) state Cordyceps; others (Entomophthora, Zoophthora, Pandora, Entomophaga) belong in the order Entomophthorales of the Zygomycota. Related fungi attack and kill other invertebrates (e.g. nematodes). Pest Control. Since they are considered natural mortality agents and environmentally safe, there is worldwide interest in the use and manipulation of entomopathogenic fungi for biological control of insects and other arthropod pests. In particular, the asexual phases of Ascomycota (Beauveria spp., Lecanicillium spp., Metarhizium spp., Paecilomyces spp. and others) are under intense scrutiny due to the traits favoring their use as biological insecticides. Production. Most entomopathogenic fungi can be grown on artificial media. However, some require extremely cplex media; others, like Beauveria bassiana and exploitable species in the genus Metarhizium, can be grown on starch-rich substrates like cereal grains (rice, wheat). Virulence. The Entomophthorales are often reported as causing high levels of mortality (epizootics) in nature. These fungi are highly virulent. The anamorphic Ascomycota (Metarhizium, Beauveria etc.) are reported as causing epizootics less frequently in nature. Also important are their properties regarding specificity (host range), storage, formulation, and application.

Feed Additives is a food supplements for farm animals that cannot get enough nutrients from regular meals that the farmers provide and include vitamins, amino acids, fatty acids, and minerals. In some cases if an animal does not have some specific nutrition in its diet it may not grow properly. The nutritional values of animal feeds are influenced not only by their nutrient content, but also by many other factors. These include feed presentation, hygiene, digestibility, and effect on intestinal health. Even with all of the benefits of higher quality feed, most of a farm animal's diet still consists of maize, wheat and soybean meal because of the higher costs of quality feed.

Fiber (or fibre; from the Latin fibra) is a natural or synthetic string or used as a component of coating applications, composite materials, or, when matted into sheets, used to make products such as paper, papyrus, or felt. Fibers are often used in the manufacture of other materials. The strongest engineering materials often incorporate fibers, for example carbon fiber and/or ultra-high-molecular-weight polyethylene. Synthetic fibers can optionally often be produced very cheaply and/or in large amounts compared to natural fibers, but for clothing natural fibers can optionally give some benefits, such as comfort, over their synthetic counter automotive products and/or parts, electronics.

Flash Memory is an electronic non-volatile computer storage medium that can be electrically erased and reprogrammed. Introduced by Toshiba in 1984, flash memory was developed from EEPROM (electrically erasable programmable read-only memory). There are two main types of flash memory, which are named after the NAND and NOR logic gates. The individual flash memory cells exhibit internal characteristics similar to those of the corresponding gates. Whereas EPROMs had to be completely erased before being rewritten, NAND type flash memory may be written and read in blocks (or pages), which are generally much smaller than the entire device. NOR type flash allows a single machine word (byte) to be written—to an erased location—or read independently. The NAND type is primarily used in memory cards, USB flash drives, solid-state drives (those produced in 2009 or later), and similar products, for general storage and transfer of data. NAND or NOR flash memory is also often used to store configuration data in numerous digital products, a task previously made possible by EEPROM or battery-powered static RAM. One significant disadvantage of flash memory is the finite number of read/write cycles in a specific block. Example applications of both types of flash memory include personal computers, PDAs, digital audio players, digital cameras, mobile phones, synthesizers, video games, scientific instrumentation, industrial robotics, medical electronics, and so on. In addition to being non-volatile, flash memory offers fast read access times, as fast as dynamic RAM, although not as fast as static RAM or ROM. Its mechanical shock resistance helps explain its popularity over hard disks in portable devices, as does its high durability, being able to withstand high pressure, temperature, immersion in water, etc. Although flash memory is technically a type of EEPROM, the term “EEPROM” is generally used to refer specifically to non-flash EEPROM, which is erasable in small blocks, typically bytes. Because erase cycles are slow, the large block sizes used in flash memory erasing give it a significant speed advantage over non-flash EEPROM when writing large amounts of data. As of 2013, flash memory costs much less than byte-programmable EEPROM and has become the dominant memory type wherever a system requires a significant amount of non-volatile, solid-state storage.

Food Additives are substances further comprising one or more added to food to preserve flavor or enhance its taste and appearance. Some additives have been used for centuries; for example, preserving food by pickling (with vinegar), salting, as with bacon, preserving sweets or using sulfur dioxide as with wines. With the advent of processed foods in the second half of the 20th century, many more food additives have been introduced, of both natural and artificial origin. Non-limiting examples of food additives, include, but not limited to: Acids. Food acids are added to make natural or artificial flavors “sharper,” and also act as preservatives and anti-aging products, antioxidants. Common food acids include vinegar, citric acid, tartaric acid, malic acid, fumaric acid, and lactic acid. Acidity Regulators. Acidity regulators are used to change or otherwise control the acidity and alkalinity of foods. Anticaking Agents. Anticaking agents keep powders such as milk powder from caking or sticking. Antifoaming Agents. Antifoaming agents reduce or prevent foaming in foods. Anti-Aging Products, antioxidants. Anti-aging products, antioxidants such as vitamin C act as preservatives by inhibiting the effects of oxygen on food, and can be beneficial to health. Bulking Agents. Bulking agents such as starch are additives that increase the bulk of a food without affecting its nutritional value. Food Coloring. Colorings are added to food to replace colors lost during preparation, or to make food look more attractive. Color Retention Agents. In contrast to colorings, color retention agents are used to preserve a food's existing color.

Emulsifiers. Emulsifiers allow water and oils to remain mixed together in an emulsion, as in mayonnaise, ice cream, and homogenized milk.

Natural or Artificial Flavors. Natural or artificial flavors are additives that give food a particular taste or smell, and may be derived from natural ingredients or created artificially.

Flavor Enhancers. Flavor enhancers enhance a food's existing natural or artificial flavors. They may be extracted from natural sources (through distillation, solvent extraction, maceration, among other methods) or created artificially. Flour Treatment Agents. Flour treatment agents are added to flour to improve its color or its use in baking. Glazing Agents. Glazing agents provide a shiny appearance or protective coating to foods. Humectants. Humectants prevent foods from drying out. Tracer Gas. Tracer gas allow for package integrity testing to prevent foods from being exposed to atmosphere, thus guaranteeing shelf life.

Presentatives. Preservatives prevent or inhibit spoilage of food due to fungi, bacteria and other microorganisms. Stabilizers. Stabilizers, thickeners and gelling agents, like agar or pectin (used in jam for example) give foods a firmer texture. While they are not true emulsifiers, they help to stabilize mullions. Sweeteners. Sweeteners are added to foods for flavoring. Sweeteners other than sugar are added to keep the food energy (calories) low, or because they have beneficial effects for diabetes mellitus and tooth decay and diarrhea.
Thickeners. Thickeners are substances which, when added to the mixture, increase its viscosity without substantially modifying its other properties. Caffeine and other GRAS (generally recognized as safe) additives such as sugar and salt are not required to go through the regulation process.

Food Additives, include, but not limited to, Abietic acid, Acacia vera, Acacia, Acesulfame potassium—artificial sweetener, Acesulfame, Acetic acid—acidity regulator, Acetic acid esters of mono- and diglycerides of fatty acids—emulsifier, Acetylated distarch adipate—thickener, vegetable gum, Acetylated distarch phosphate—thickener, vegetable gum, Acetylated oxidized starch—thickener, vegetable gum, Acetylated starch—thickener, vegetable gum, Acid treated starch—thickener, vegetable gum, Adipic acid—food acid, Agar—thickener, vegetable gum, stabilizer, gelling agent, Alcohol, Alfalfa, Alginic acid—thickener, vegetable gum, stabilizer, gelling agent, emulsifier, Alitame—artificial sweetener, Alkaline treated starch—thickener, vegetable gum, Alkanet—color (red), Allspice, Allura red AC—color (FDA: FD&C Red #40), Almond oil—used as a substitute for olive oil. Also used as an emollient, Aluminium—color (silver), Aluminum ammonium sulfate—mineral salt, Aluminum potassium sulfate—mineral salt, Aluminum silicate—anticaking agent, Aluminum sodium sulfate—mineral salt, Aluminum sulfate—mineral salt, Amaranth—color (red) (FDA: Red #2) Note that amaranth dye is unrelated to the amaranth plant, Amaranth oil—high in squalene and unsaturated fatty acids—used in food and cosmetic industries, Amchur (mango powder), Ammonium acetate—preservative, acidity regulator, Ammonium adipates—acidity regulator, Ammonium alginate—thickener, vegetable gum, stabilizer, gelling agent, emulsifier, Ammonium bicarbonate—mineral salt, Ammonium carbonate—mineral salt, Ammonium chloride—mineral salt, Ammonium ferric citrate—food acid, Ammonium fumarate—food acid, Ammonium hydroxide—mineral salt, Ammonium lactate—food acid. Ammonium malate—food acid, Ammonium phosphates—mineral salt, Ammonium phosphatides—emulsifier, Ammonium polyphosphates—anticaking agent, Ammonium sulfate—mineral salt, improving agent, Amylases—flour treatment agent, Angelica (Angelica archangelic), Anise, Annatto—color, Anthocyanins—color, Apricot oil—a cooking oil from certain cultivars, Arabinogalactan—thickener, vegetable gum, Argan oil—a food oil from Morocco that has also attracted recent attention in Europe, Argon—propellant, Rocket (Arugula), Asafoetida, Ascorbic acid (Vitamin C)—antioxidant (water soluble), Ascorbyl palmitate—antioxidant (fat soluble), Ascorbyl stearate—antioxidant (fat soluble), Aspartame—artificial sweetener, Astaxanthin—color, Avocado oil—used a substitute for olive oil. Also used in cosmetics and skin care products, Azodicarbonamide—flour bleaching agent. Also used in the production of foamed plastics and the manufacture of gaskets. Banned as a food additive in Australia and Europe, Azorubine—color (red) (FDA: Ext D&C Red #10), B, Babassu oil—similar to, and used as a substitute for coconut oil, Baking powder—leavening agent; includes acid and base, Baking soda—food base, Balm, lemon, Balm oil, Balsam of Peru—used in food and drink for flavoring, Barberry, Barley flour, Basil (Ocimum basilicum), Basil extract, Bay leaves, Beeswax—glazing agent, Beet red—color (red), Beetroot red—color (red), Ben oil—extracted from the seeds of the moringa oleifera. High in behenic acid. Extremely stable edible oil. Also suitable for biofuel, Bentonite—anticaking agent, Benzoic acid—preservative, Benzoyl peroxide—flour treatment agent, Berebere, Bergamot—in Earl Grey tea, Beta-apo-8′-carotenal (C 30)—color, Beta-apo-8′-carotenic acid ethyl ester—color, Betanin—color (red), Biphenyl—preservative, Bison grass (Hierochloe odorata), Bixin—color, Black 7984—color (brown and black), Black cardamom-, Black cumin, Blackcurrant seed oil—used as a food supplement, because of high content of omega-3 and omega-6 fatty acids. Also used in cosmetics, Black limes, Pepper (black, white, and green), Black PN—color (brown and black), Bleached starch—thickener, vegetable gum, Bolivian Coriander (Porophyllum ruder ale), Bone phosphate—anticaking agent, Borage (Borago officials), Borage seed oil—similar to blackcurrant seed oil—used primarily medicinally, Borax—preservative, Boric acid—preservative, Brilliant Black BN— color (brown and black), Brilliant blue FCF—color (FDA: FD&C Blue #1), Brilliant Scarlet 4R—color (FDA: Ext D&C Red #8), Brominated vegetable oil—emulsifier, stabilizer, Brown FK—color (brown and black) Bush tomato, Butane—propellant, Butylated hydroxyanisole (BHA)—antioxidant (fat soluble), Butylated hydroxytoluene (BHT)—antioxidant (fat soluble), C, Cacao shell, Cachou extract, Cactus root extract, Cadinene, Caffeine—stimulant, Cajeput oil, Calamus, Calcium 5′-ribonucleotides—flavor enhancer, Calcium acetate—preservative, acidity regulator, Calcium alginate—thickener, vegetable gum, stabilizer, gelling agent, emulsifier, Calcium ascorbate—antioxidant (water soluble), Calcium aluminosilicate (calcium aluminium silicate)—anticaking agent, Calcium ascorbate (Vitamin C), Calcium benzoate—preservative, Calcium bisulfite—preservative, antioxidant, Calcium carbonates—color (white), anticaking agent, stabilizer, Calcium chloride—mineral salt, Calcium citrates—food acid, firming agent, Calcium diglutamate—flavor enhancer, Calcium disodium EDTA—preservative, Calcium ferrocyanide—anticaking agent, Calcium formate—preservative, Calcium fumarate—food acid, Calcium gluconate—acidity regulator, Calcium guanylate—flavor enhancer, Calcium hydrogen sulfite—preservative, antioxidant, Calcium hydroxide—mineral salt, Calcium inosinate—flavor enhancer, Calcium lactate—food acid, Calcium lactobionate—stabilizer, Calcium malates—food acid, Calcium oxide—mineral salt, Calcium pantothenate (Vitamin B₅), Calcium peroxide, Calcium phosphates—mineral salt, anticaking agent, firming agent, Calcium polyphosphates—anticaking agent, Calcium propionate—preservative, Calcium salts of fatty acids—emulsifier, stabilizer, anticaking agent, Calcium silicate—anticaking agent, Calcium sorbate—preservative, Calcium stearoyl lactylate—emulsifier, Calcium sulfate—flour treatment agent, mineral salt, sequestrant, improving agent, firming agent, Calcium sulfite—preservative, antioxidant, Calcium tartrate—food acid, emulsifier, False flax oil, considered as a food or fuel oil, Chamomile, Candelilla wax—glazing agent, Candle nut, Canola oil/Rapeseed oil, one of the most widely used cooking oils, from a (trademarked) cultivar of rapeseed, Canthaxanthin—color, Caper (Capparis spinosa), Capsanthin—color, Capsorubin—color, Carrageenan—A family of linear sulphated polysaccharides extracted from red seaweeds, Caramel I (plain)—color (brown and black), Caramel II (Caustic Sulfite process)—color (brown and black), Caramel III (Ammonia process)—color (brown and black), Caramel IV (Ammonia sulfite process)—color (brown and black), Caraway, Carbamide—flour treatment agent, Carbon black—color (brown and black), Carbon dioxide—acidity regulator, propellant, Cardamom, Carmines—color (red), Carmoisine—color (red) (FDA: Ext D&C Red #10), Carnauba wax—glazing agent, Carob Pod, Carob pod oil/Algaroba oil, used medicinally, Carotenes—color, Alpha-carotene—color, Beta-carotene—color. Gamma-carotene—color, Carrageenan—thickener, vegetable gum, stabilizer, gelling agent, emulsifier, Carrot oil, Cashew oil—somewhat comparable to olive oil. May have value for fighting dental cavities, Cassia, Catechu extract, Celery salt, Celery seed, Wheat germ oil—used as a food supplement, and for its “grainy” flavor. Also used medicinally. Highly unstable, Chalk—color (white), anticaking agent, stabilizer, Chervil (Anthriscus cerefolium), Chicory, Chicory Root Extract—High in Inulin, Chile pepper, Chili powder, Chives (Allium schoenoprasum), Chlorine dioxide—flour treatment agent, Chlorine—flour treatment agent, Chlorophylls and Chlorophyllins—color (green), Chocolate Brown HT—color, Choline salts and esters—emulsifier, Chrysoine resorcinol—color (red), Cicely (Myrrhis odorata), Sweet cicely (Myrrhis odorata), Cilantro (see Coriander) (Coriandrum sativum), Cinnamon, Cinnamon oil—used for flavoring, Citranaxanthin—color, Citric acid—food acid, Citric acid esters of mono- and diglycerides of fatty acids—emulsifier, Citrus red 2—color (red), Cloves, Cochineal—color (red), Coconut oil—a cooking oil, high in saturated fat—particularly used in baking, Sage (Salvia officinalis), Copper complexes of chlorophylls—color (green), Coriander, Coriander seed oil—used medicinally. Also used as a flavoring agent in pharmaceutical uses and food industries, Corn oil—one of the most common, and inexpensive cooking oils, Corn syrup, Cottonseed oil—a major food oil, often used in industrial food processing, Cress, Crocetin—color, Crocin—color, Crosslinked Sodium carboxymethylcellulose—emulsifier, Cryptoxanthin—color, Cumin, Cumin oil/Black seed oil—used as a flavor, particularly in meat products. Also used in veterinary medicine, Cupric sulfate—mineral salt, Curcumin—color (yellow and orange), Curry powder, Curry leaf (Murraya koenigii), Cyanocobalamin (Vitamin B12), Cyclamates—artificial sweetener, Cyclamic acid—artificial sweetener, beta-cyclodextrin—emulsifier, Lemongrass (Cymbopogon citratus, C. flexuosus, and other species), D, Damiana (Turnera aphrodisiaca, T. diffusa), Dandelion leaf, Dandelion Root, Dandelion (Taraxacum officinale), Decanal dimethyl acetal, Decanal, Decanoic acid, Dehydroacetic acid—preservative, Delta-tocopherol (synthetic)—antioxidant, Devil's claw (Harpagophytum procumbens) medicinal, Dextrin roasted starch—thickener, vegetable gum, Diacetyltartaric acid esters of mono- and diglycerides of fatty acids—emulsifier, Dicalcium diphosphate—anticaking agent, Dilauryl thiodipropionate—antioxidant, Dill seed, Dill (Anethum graveolens), Dimethyl dicarbonate—preservative, Dimethylpolysiloxane—emulsifier, anticaking agent, Dioctyl sodium sulfosuccinate—emulsifier, Diphenyl—preservative, Diphosphates—mineral salt, emulsifier, Dipotassium guanylate—flavor enhancer, Dipotassium inosinate—flavor enhancer, Disodium 5′-ribonucleotides—flavor enhancer, Disodium ethylenediaminetetraacetate—antioxidant, preservative, Disodium guanylate—flavor enhancer, Disodium inosinate—flavor enhancer, Distarch phosphate—thickener, vegetable gum, Distearyl thiodipropionate—antioxidant, Dl-alpha-tocopherol (synthetic)—antioxidant, Dodecylgallate—antioxidant, E, Echinacea, EDTA—Antioxidant, Chelating Agent, Egg, Egg yolk, Egg white, Elderberry, Eleutherococcus senticosus, Enzymatically hydrolyzed Carboxymethyl cellulose—emulsifier, Enzyme treated starch—thickener, vegetable gum, Epazote (Chenopodium ambrosioides), Epsom salts—mineral salt, acidity regulator, firming agent, Erythorbin acid—antioxidant, Erythrosine—color (red) (FDA: FD&C Red #3), Erythritol—artificial sweetener, Ethanol (alcohol), Ethyl maltol—flavor enhancer, Ethyl methyl cellulose—thickener, vegetable gum, emulsifier, Ethylparaben (ethyl para-hydroxybenzoate)—preservative, Ethylenediamine tetraacetic acid, Evening primrose oil—used as a food supplement for its purported medicinal properties, F, Fantesk, Farnesol, Fast green FCF—color (FDA: FD&C Green #3), Fat, Flavoxanthin—color, Fennel (Foeniculum vulgare), Fenugreek, Ferric ammonium citrate—food acid, Ferrous gluconate—color retention agent, Ferrous lactate, File powder, Five-spice powder (Chinese), Fo-ti-tieng, Formaldehyde—preservative, Formic acid—preservative, Fructose, Fumaric acid—acidity regulator, G, Galangal, Galangal root, Galbanum oil, Gallic acid, Gamma-tocopherol (synthetic)—antioxidant, Garam masala, Garlic extract, Garlic, Garlic oil, Gelatin/gelatine—Gelling agent, emulsifier, Gellan gum—thickener, vegetable gum, stabilizer, emulsifier, Ginger, Ginger oil, Ginger root, Ginseng, Glacial Acetic acid—preservative, acidity regulator, Glucitol, Gluconate, Glucono delta-lactone—acidity regulator, Glucose oxidase—antioxidant, Glucose syrup—sweetener, Glutamate, Glutamic acid—flavor enhancer, Gluten, Glycerin—humectant, sweetener, Glycerol, Glycerol ester of wood rosin—emulsifier, Glyceryl distearate—emulsifier, Glyceryl monostearate—emulsifier, Glycine—flavor enhancer, Glyoxylic acid, Gold—color (gold), Grains of paradise, Grape color extract, Grape seed oil—suitable for cooking at high temperatures. Also used as a salad oil, and in cosmetic additives, sugar substitute, sweeteners, artificial sweeteners, anticaking agent, Green S—color (green), Green tea, Guanylic acid—flavor enhancer, Guar gum—thickener, vegetable gum, stabilizer, Guaranine, Gum arabic/Gum acacia/E414—thickener, vegetable gum, stabilizer, emulsifier, Gum guaicum—preservative, H, Haw bark, Hazelnut oil—used for its flavor. Also used in skin care, because of its slight astringent nature, Heliotropin, Helium—propellant, Hemlock oil, Hemp oil—a high quality food oil, Heptyl p-hydroxybenzoate—preservative, Hesperidin, Hexamine (hexamethylene tetramine)—preservative, Hexyl acetate, High fructose corn syrup, Horseradish, Hydrochloric acid—acidity regulator, Hydroxypropyl cellulose—thickener, vegetable gum, emulsifier, Hydroxypropyl distarch phosphate—thickener, vegetable gum, Hydroxypropyl methylcellulose—thickener, vegetable gum, emulsifier, Hydroxypropyl starch—thickener, vegetable gum, Hyssop (Hyssopus officinalis), I, Indanthrene blue RS—color (blue), Indigo carmine—color (blue) (FDA: FD&C Blue #2), Indigotine—color (blue) (FDA: FD&C Blue #2), Indole, Inosinate, Inosinic acid—flavor enhancer, Inositol, Insoluble fiber, Intense sweeteners, Inulin, Invert sugar, Invertase, Iron ammonium citrate, Iron, Iron oxides and hydroxides—color, Isobutane—propellant, Isomalt—humectant, Isopropyl citrates—antioxidant, preservative, J, Jasmine, Jamaican jerk spice, Jasmine absolute, Jiaogulan (Gynostemma pentaphyllum), Juniper, Juniper berry, Juniper berry oil—used as a flavor. Also used medicinally, including traditional medicine, Juniper extract, K, Kaffir Lime Leaves (Citrus hystrix, C. papedia), Kaolin—anticaking agent, Kapok seed oil—used as an edible oil, and in soap production, Karaya gum—thickener, vegetable gum, stabilizer, emulsifier, Kelp, Kokam, Kola nut extract, Konjac—thickener, vegetable gum, Konjac glucomannate—thickener, vegetable gum, Konjac gum—thickener, vegetable gum, L, L-cysteine—flour treatment agent, Lactic acid—acidity regulator, preservative, antioxidant, Lactic acid esters of mono- and diglycerides of fatty acids—emulsifier, Lactitol—humectant, Lactose, Lactylated fatty acid esters of glycerol and propylene glycol—emulsifier, Larch gum, Lard, Latolrubine—color, Laurel berry, Laurel leaf oil, Lavender (Lavandula spp.), Lavender oil, Lecithins—antioxidant, Emulsifier, Lecithin citrate—preservative, Lemon, Lemon balm (Melissa officinalis), Lemon extract, Lemon juice, Lemon Myrtle (Backhousia citriodora), Lemon oil, Lemon verbena (Lippia citriodora), Lemongrass Oil, Leucine—flavor enhancer, Licorice, Lipases—flavor enhancer, Lithol Rubine BK—color, Litholrubine—color, Locust bean gum—thickener, vegetable gum, stabilizer, gelling agent, emulsifier, Long pepper, Lovage (Levisticum officinale), L(+)-Tartaric acid—food acid, Lutein—color, Lycopene—color, Lysine, Lysozyme—preservative, M, Macadamia oil—used as an edible oil. Also used as a massage oil, Mace, Magnesium, Magnesium carbonate—anticaking agent, mineral salt, Magnesium chloride—mineral salt, Magnesium citrate—acidity regulator, Magnesium diglutamate—flavor enhancer, Magnesium hydroxide—mineral salt, Magnesium lactate—food acid, Magnesium oxide—anticaking agent, Magnesium phosphates—mineral salt, anticaking agent, Magnesium salts of fatty acids—emulsifier, stabilizer, anticaking agent, Magnesium silicate—anticaking agent, Magnesium stearate—emulsifier, stabilizer, Magnesium sulfate—mineral salt, acidity regulator, firming agent, Mahlab, Malabathrum, Malic acid—acidity regulator, Malt extract—flavor enhancer, Maltitol—humectant, stabilizer, Maltodextrin—carbohydrate sweetener, Maltol—flavor enhancer, Maltose, Mandarin oil-leavening agent, Manganese, Mannitol—humectant, anticaking agent, sweetener, Margarine, Marjoram (Origanum majorana), Mastic, Meadowfoam seed oil—highly stable oil, with over 98% long-chain fatty acids. Competes with rapeseed oil for industrial applications, Mega-purple—a Kosher food additive made from grapes, Mentha arvensis oil/Mint oil, used in flavoring toothpastes, mouthwashes and pharmaceuticals, as well as in aromatherapy and other medicinal applications, Metatartaric acid—food acid, emulsifier, Methionine, Methyl butyrate, Methyl disulfide, Methyl ethyl cellulose—thickener, vegetable gum, emulsifier, Methyl hexenoate, Methyl isobutyrate, Methylcellulose—thickener, emulsifier, vegetable gum, Methylparaben (methyl para-hydroxybenzoate)—preservative, Methyltheobromine, Microcrystalline cellulose—anticaking agent, Milk thistle (Silybum), Milk, Mint (Mentha spp.), Mixed acetic and tartaric acid esters of mono- and diglycerides of fatty acids—emulsifier, Modified starch, Molasses extract, Molybdenum, Bergamot (Monarda didyma), Mono- and diglycerides of Fatty acids—emulsifier, Monoammonium glutamate—flavor enhancer, Monopotassium glutamate—flavor enhancer, Monosodium glutamate (MSG)—flavor enhancer, Monostarch phosphate—thickener, vegetable gum, Montanic acid esters—humectant, Mullein (Verbascum thapsus), Mustard, Mustard oil (essential oil), containing a high % age of allylisothiocyanate or other isothiocyanates, depending on the species of mustard, Mustard oil (pressed)—used in India as a cooking oil. Also used as a massage oil, Mustard plant, Mustard seed, N, Natamycin—preservative, Neohesperidin dihydrochalcone—artificial sweetener, Niacin (vitamin B₃)—color retention agent nicotinic acid (vitamin B₃)—color retention agent, Nicotinamide (vitamin B₃)—color retention agent, Nigella (Kolanji, Black caraway), Nisin—preservative, Nitrogen—propellant, Nitrous oxide—propellant, Norbixin—color, Nutmeg, O, Octyl gallate—antioxidant, Evening primrose (Oenothera biennis et al.), Okra oil (Hibiscus seed oil)—from the seed of the Hibiscus esculentus. Composed predominantly of oleic and lanoleic acids, Oleomargarine, Olive oil—used in cooking—cosmetics—soaps and as a fuel for traditional oil lamps, Orange GGN—color (orange), Orange oil—like lemon oil—cold pressed rather than distilled. Consists of 90% d-Limonene. Used as a fragrance, in cleaning products and in flavoring cellulose in foods, Orcein—color (red), Orchil—color (red), Oregano (Origanum vulgare, O. heracleoticum, and other species), Oregano oil—contains thymol and carvacrol—making it a useful fungicide, Orris root, Orthophenyl phenol—preservative, Oxidized polyethylene wax—humectant, Oxidizedo starch—thickener, vegetable gum, Oxystearin—antioxidant, sequestrant, P, Palm oil—the most widely produced tropical oil. Also used to make biofuel, Panax quinquefolius, Ponch phoran, Pandan leaf, Pantothenic acid (Vitamin B₅), Papain—A cysteine protease hydrolase enzyme present in papaya (Carica papaya) and mountain papaya (Vasconcellea cundinamarcensis), Paprika red, Paprika, Paprika extract, Paraffins—glazing agent, Parsley (Petroselinum crispum), Patent blue V—color (blue), Peanut oil/Ground nut oil—mild-flavored cooking oil, Pecan oil—valued as a food oil, but requiring fresh pecans for good quality oil, Pectin—vegetable gum, emulsifier, Perilla seed oil—high in omega-3 fatty acids. Used as an edible oil, for medicinal purposes, in skin care products and as a drying oil, Phosphated distarch phosphate—thickener, vegetable gum, Phosphoric acid—food acid, Phytic acid—preservative, Pigment Rubine—color, Pimaricin—preservative, Pine needle oil, Pine seed oil—an expensive food oil, used in salads and as a condiment, Pistachio oil—strongly flavored oil, particularly for use in salads, Prune kernel oil—marketed as a gourmet cooking oil, Poly vinyl pyrrolidone, Polydextrose—humectant, Polyethylene glycol 8000—antifoaming agent, Polyglycerol esters of fatty acids—emulsifier, Polyglycerol polyricinoleate—emulsifier, Polymethylsiloxane—antifoaming agent, Polyoxyethylene (40) stearate—emulsifier, Polyoxyethylene (8) stearate—emulsifier, stabilizer, Polyphosphates—mineral salt, emulsifier, Polysorbate 20—emulsifier, Polysorbate 40—emulsifier, Polysorbate 60—emulsifier, Polysorbate 65—emulsifier, Polysorbate 80—emulsifier, Polyvinylpolypyrrolidone—color stabilizer, Pomegranate seeds, Ponceau 4R—color (FDA: Ext D&C Red #8), Ponceau 6R—color, Ponceau SX—color, Poppy seed, Poppyseed oil—used for cooking, moisturizing skin, and in paints, varnishes and soaps, Potassium acetates—preservative, acidity regulator, Potassium adipate—food acid, Potassium alginate—thickener, vegetable gum, stabilizer, gelling agent, emulsifier, Potassium aluminium silicate—anticaking agent, Potassium ascorbate—antioxidant (water soluble), Potassium benzoate—preservative, Potassium bicarbonate—mineral salt, Potassium bisulfite—preservative, antioxidant, Potassium bromate—flour treatment agent, Potassium carbonate—mineral salt, Potassium chloride—mineral salt, Potassium citrates—food acid, Potassium ferrocyanide—anticaking agent, Potassium fumarate—food acid, Potassium gluconate—stabilizer, Potassium hydrogen sulfite—preservative, antioxidant, Potassium hydroxide—mineral salt Potassium lactate—food acid, Potassium malate—food acid, Potassium metabisulfite—preservative, antioxidant, Potassium nitrate—preservative, color fixative, Potassium nitrite—preservative, color fixative, Potassium phosphates—mineral salt, Potassium propionate—preservative, Potassium salts of fatty acids—emulsifier, stabilizer, anticaking agent, Potassium sodium tartrate—food acid, Potassium sorbate—preservative, Potassium sulfate—mineral salt, seasoning, Potassium sulfite—preservative, antioxidant, Potassium tartrates—food acid, Powdered Cellulose—anticaking agent, Primrose (Primula)—candied flowers, tea, Processed Eucheuma seaweed—thickener, vegetable gum, stabilizer, gelling agent, emulsifier, Propane-1,2-diol alginate—thickener, vegetable gum, stabilizer, emulsifier, Propionic acid—preservative, Propyl gallate—antioxidant, Propylene glycol—humectant, Propylene glycol alginate—thickener, vegetable gum, stabilizer, emulsifier, Propylene glycol esters of fatty acids—emulsifier, Propylparaben (propyl para-hydroxybenzoate)—preservative, Pumpkin seed oil—a specialty cooking oil, Pulegone, Purslane, Pyridoxine hydrochloride (Vitamin B₆), Q, Quatre épices, Quillaia extract—humectant, Quinoa oil—similar in composition and use to corn oil, Quinoline Yellow WS—color (yellow and orange) (FDA: D&C Yellow #10), R, Ramtil oil—pressed from the seeds of the one of several species of genus Guizotia abyssinica (Niger pea) in India and Ethiopia. Used for both cooking and lighting, Ras-el hanout, Raspberry (leaves), Red 2G—color, Refined microcrystalline wax—glazing agent, Rhodoxanthin—color, Riboflavin (vitamin B₂)—color (yellow and orange), Rice bran oil—suitable for high temperature cooking, AsiaRosemary (Rosmarinus officinalis), Rubixanthin—color, S, Saccharin—artificial sweetener, Safflower oil—a flavorless and colorless cooking oil, Safflower, Saffron—color, Saigon Cinnamon, Salad Burnet (Sanguisorba minor or Poterium sanguisorba), Salt, Sandalwood—color, Savory (Satureja hortensis, S. montana), Scarlet GN—color, Sesame oil—used as a cooking oil, and as a massage oil, particularly in India, Sesame seed, Shellac—glazing agent, Silicon dioxide—anticaking agent, Silver—color (silver), Luohanguo, Sodium acetate—preservative, acidity regulator, Sodium adipate—food acid, Sodium alginate—thickener, vegetable gum, stabilizer, gelling agent, emulsifier Sodium aluminium phosphate—acidity regulator, emulsifier, Sodium aluminosilicate (sodium aluminium silicate)—anticaking agent, Sodium ascorbate—antioxidant (water soluble), Sodium benzoate—preservative, Sodium bicarbonate—mineral salt, Sodium bisulfite (sodium hydrogen sulfite)—preservative, antioxidant, Sodium carbonate—mineral salt, Sodium carboxymethylcellulose—emulsifier, Sodium citrates—food acid, Sodium dehydroacetate—preservative, Sodium erythorbate—antioxidant, Sodium erythorbin—antioxidant, Sodium ethyl para-hydroxybenzoate—preservative, Sodium ferrocyanide—anticaking agent, Sodium formate—preservative, Sodium fumarate—food acid, Sodium gluconate—stabilizer, Sodium hydrogen acetate—preservative, acidity regulator, Sodium hydroxide—mineral salt, Sodium lactate—food acid, Sodium malates—food acid, Sodium metabisulfite—preservative, antioxidant, bleaching agent, Sodium methyl para-hydroxybenzoate—preservative, Sodium nitrate—preservative, color fixative, Sodium nitrite—preservative, color fixative, Sodium orthophenyl phenol—preservative, Sodium propionate—preservative, Sodium propyl para-hydroxybenzoate—preservative, Sodium sorbate—preservative, Sodium stearoyl lactylate—emulsifier, Sodium succinates—acidity regulator, flavor enhancer, Sodium salts of fatty acids—emulsifier, stabilizer, anticaking agent, Sodium sulfite—mineral salt, preservative, antioxidant, Sodium sulfite—preservative, antioxidant, Sodium tartrates—food acid, Sodium tetraborate—preservative, Sorbic acid—preservative, Sorbitan monolaurate—emulsifier, Sorbitan monooleate—emulsifier, Sorbitan monopalmitate—emulsifier, Sorbitan monostearate—emulsifier, Sorbitan tristearate—emulsifier, Sorbitol—humectant, emulsifier, sweetener, Sorbol, Sorrel (Rumex spp.), Soybean oil—accounts for about half of worldwide edible oil production, Spearmint oil—often used in flavoring mouthwash and chewing gum, among other applications, Star anise, Star anise oil—highly fragrant oil using in cooking. Also used in perfumery and soaps, has been used in toothpastes, mouthwashes, and skin creams, Starch sodium octenylsuccinate—thickener, vegetable gum, Stearic acid—anticaking agent, Stearyl tartarate—emulsifier, Succinic acid—food acid, Sucralose—artificial sweetener, Sucroglycerides—emulsifier, Sucrose acetate isobutyrate—emulsifier, stabilizer, Sucrose esters of fatty acids—emulsifier, Sugar, Sulfur dioxide—preservative, antioxidant, Sulfuric acid—acidity regulator, Sumac, Sunflower oil—a common cooking oil, also used to make biodiesel, Sunset Yellow FCF—color (yellow and orange) (FDA: FD&C Yellow #6) Sweet basil, Sweet woodruff, Szechuan pepper (Xanthoxylum piperitum), T, Talc—anticaking agent, Tamarind, Tanacetum balsamita/Costmary, Tandoori masala, Tannins—color, emulsifier, stabilizer, thickener, Tansy, Tara gum—thickener, vegetable gum, stabilizer, Tarragon (Artemisia dracunculus), Tartaric acid esters of mono- and diglycerides of fatty acids—emulsifier, Tartrazine—color (yellow and orange) (FDA: FD&C Yellow #5), Camellia oil/Tea oil, widely used in southern China as a cooking oil. Also used in making soaps, hair oils and a variety of other products, Tert-butylhydroquinone—antioxidant, Tetrahydrocannabinol—flavor enhancer, potent anti-carcinogen, Thaumatin—flavor enhancer, artificial sweetener, Theine, Thermally oxidized soya bean oil—emulsifier, Thiabendazole—preservative, Thiamine (Vitamin B1), Thiodipropionic acid—antioxidant, Thyme, stannous chloride—color retention agent, antioxidant, Titanium dioxide—color (white), Tocopherol (Vitamin E), Tocopherol concentrate (natural)—antioxidant, Tragacanth—thickener, vegetable gum, stabilizer, emulsifier, Triacetin—humectant, Triammonium citrate—food acid, Triethyl citrate—thickener, vegetable gum, Trimethylxanthine, Triphosphates—mineral salt, emulsifier sodium phosphates—Mineral Salt, Turmeric—color (yellow and orange), V, Vanilla (Vanilla planifolia), Vegetable carbon—color (brown and black), Vinegar, Violaxanthin—color, Vitamin A (Retinol), Vitamin B₁ (Thiamine), Vitamin B₂ (Riboflavin), Vitamin B₅ (Pantothenic acid), Vitamin B₆ (Pyrodoxine), Vitamin B₁₂ (Cyanocobalamin), Vitamin C (Ascorbic acid), Vitamin D (Calciferol), Vitamin E (Tocopherol), Vitamin K (Potassium), W, Walnut oil—used for its flavor, also used by Renaissance painters in oil paints, Wasabi, Water, Wattleseed, X, Xanthan gum—thickener, vegetable gum, stabilizer, Xylitol—humectant, stabilizer, Y, Yellow 2G—color (yellow and orange), Yucca extract, Z, Zeaxanthin—color, Zinc acetate—flavor enhancer.

Hash Oil (also known as hashish oil, butane honey oil, BHO, wax, shatter, crumble, honey oil, dabs, budder, liquid cannabis) is a resinoid obtained by solvents, carbon dioxide, nitrogen, and hyperbaric extraction of dried female cannabis flowers, as distinct from hemp flowers as hemp is the name for “industrial” cannabis or other cellulose vegetable or gelatin capsules for dietary supplements, medications, vitamins, marijuana plant without significant thc, the main active cannabanoid. Hash oil may contain much psychoactive cannabinoids, depending on the plant's mix of essential oils and cannabinoids. Hash oil extracted with butane or supercritical carbon dioxide has become popular in recent years.

Hemp Oil. Hemp oil is usually derived from male cannabis plants that have up to 0.3% of THC in them. Hemp oil is quite nutritious as it contains essential fatty acids such as omega-3 and omega-6, both of which can be found in salmon and fish as well.

Unhealthy Foods. Non-limiting examples of unhealthy foods to avoid, include, but not limited to, Genetically-Modified Organisms (GMOs). It goes without saying that GMOs have no legitimate place in any cancer-free diet, especially now that both GMOs and the chemicals used to grow them have been shown to cause rapid tumor growth. But GMOs are everywhere, including in most food derivatives made from conventional corn, soybeans, and canola. However, you can avoid them by sticking with certified organic, certified non-GMO verified, and locally-grown foods that are produced naturally without biotechnology. Processed Meats. Most processed meat products, including lunch meats, bacon, sausage, and hot dogs, contain chemical preservatives that make them appear fresh and appealing, but that can also cause cancer. Both sodium nitrite and sodium nitrate have been linked to significantly increasing the risk of colon and other forms of cancer, so be sure to choose only uncured meat products made without nitrates, and preferably from grass-fed sources. Microwave Popcorn. They might be convenient, but those bags of microwave popcorn are lined with chemicals that are linked to causing not only infertility but also liver, testicular, and pancreatic cancers. The U.S. Environmental Protection Agency (EPA) recognizes the perfluorooctanoic acid (PFOA) in microwave popcorn bag linings as “likely” carcinogenic, and several independent studies have linked the chemical to causing tumors. Similarly, the diacetyl chemical used in the popcorn itself is linked to causing both lung damage and cancer. Soda Pop. Like processed meats, soda pop has been shown to cause cancer as well. Loaded with sugar, food chemicals, and colorings, soda pop acidifies the body and literally feeds cancer cells. Common soda pop chemicals like caramel color and its derivative 4-methylimidazole (4-MI) have also specifically been linked to causing cancer. ‘Diet’ Beverages. Even worse than conventional sugar-sweetened soda pop, though, is “diet” soda pop and various other diet beverages and foods. A recent scientific review issued by the European Food Safety Authority (EFSA) of more than 20 separate research studies found that aspartame, one of the most common artificial sweeteners, causes a range of illnesses including birth defects and cancer. Sucralose (Splenda), saccharin and various other artificial sweeteners have also been linked to causing cancer. Refined ‘White’ Flours. Refined flour is a common ingredient in processed cellulose in foods, but its excess carbohydrate content is a serious cause for concern. A study published in the journal Cancer Epidemiology, Mile Markers, and Prevention found that regular consumption of refined carbohydrates was linked to a 220% increase in breast cancer among women. High-glycemic foods in general have also been shown to rapidly raise blood sugar levels in the body, which directly feeds cancer cell growth and spread. Refined Sugars. The same goes for refined sugars, which tend to rapidly spike insulin levels and feed the growth of cancer cells. Fructose-rich sweeteners like high-fructose corn syrup (HFCS) are particularly offensive, as cancer cells have been shown to quickly and easily metabolize them in order to proliferate. And since cookies, cakes, pies, sodas, juices, sauces, cereals, and many other popular, mostly processed, food items are loaded with HFCS and other refined sugars, this helps explain why cancer rates are on the rise these days. Conventional Apples, Grapes, and Other ‘Dirty’ Fruits. Many people think they are eating healthy when they buy apples, grapes, or strawberries from the store. But unless these fruits are organic or verified to be pesticide-free, they could be a major cancer risk. The Environmental Working Group (EWG) found that up to 98% of all conventional produce, and particularly the type found on its “dirty” fruits list, is contaminated with cancer-causing pesticides. Farmed Salmon. Farmed salmon is another high-risk cancer food, according to Dr. David Carpenter, Director of the Institute for Health and the Environment at the University of Albany. According to his assessment, farmed salmon not only lacks vitamin D, but it is often contaminated with carcinogenic chemicals, PCBs (polychlorinated biphenyls), flame retardants, pesticides, and antibiotics. Hydrogenated Oils. They are commonly used to preserve processed foods and keep them shelf-stable. But hydrogenated oils alter the structure and flexibility of cell membranes throughout the body, which can lead to a host of debilitating diseases such as cancer. Some manufacturers are phasing out the use of hydrogenated oils and replacing them with palm oil and other safer alternatives, but trans fats are still widely used in processed food.

Unhealthy Foods. non-limiting examples of unhealthy foods to avoid, include, but not limited to, GMO foods have been among us for decades, and it seems as if they are only growing in number. There are certain foods that you should definitely avoid if you're trying to get GMOs out of your life. Here are some of the top offenders that unless otherwise labeled, will most likely contain genetically modified organisms of some sort. Corn. Pick up an ear of conventional corn and chances are it's going to contain at least some GMOs in it. Corn is the largest crop grown in America, and some of the biggest providers of corn have altered them so they'll grow better. GMO corn has been engineered to that it will ward off insects and battle back against herbicides. These are not the kind of chemicals you′d want to knowingly put in your body, yet there's no way to avoid them if you're eating corn that has been modified to inherently contain them. It would be easy enough to avoid GMOs from corn, but so many products are derived from corn, and corn is used to make High Fructose Corn Syrup, which is found in hundreds of items throughout the supermarket, that this is easily the one GMO food to put your target on and eliminate from your diet first. Tomatoes. Tomatoes often make health news headlines for their healthy benefits, but they are also a food you'll want to watch out for if you're trying to sidestep the consumption of GMOs. You'll have to seek out organic tomatoes, because most of the tomatoes on store shelves are not organic. The price may be a bit higher, but it's worth it to get the good things tomatoes contain, without the bad. Yes, tomatoes are one of the healthiest foods you can eat, containing lycopene that benefits the body in a number of ways, perhaps most importantly by preventing cancer. However, these benefits are compromised when the tomato also contains herbicides and pesticides built right into it. Potatoes. Potatoes are grown in abundance in the United States, as they are used by fast food companies and prepared food companies to make French fries, hash browns, and more. Potatoes are also grown to be fed to livestock, which in turn ends up on our plate in the form of meat. You may be thinking that because potatoes grown underground they will contain fewer pesticides and herbicides. Unfortunately, when these chemicals are placed right into the seed itself there's nothing you can do to avoid it. Such is the case with GMO potatoes. Squash/Zucchini. While it's possible for many of the conventionally grown vegetables you see in the produce section to contain GMOs, there's an even higher likelihood that squash will contain them. The reason these are so frequently treated with GMOs is because they're especially susceptible to viruses that can wipe out an entire crop. To give them more of a fighting chance, food conglomerates have made it so that the seeds contain antiviral elements to them so they can be more resilient once planted. The end result is that medications, for lack of a better word, enter into our bodies. Soybeans. Soybeans are the second-largest crop grown in America, and it shouldn't come as a surprise that when a food is grown in such large quantities, it has probably been tinkered with genetically. However, the use of GMOs in soybeans is greater than in other crops, which makes it especially tricky to find non-GMO soybeans. Perhaps you don't eat soybeans directly, but you may find that you're using soybean oil, or other soy-derived products. Unless these are labeled differently, they will likely be conventional and have a high chance of being a GMO food. Sugar Beets. Sugar beets are often used to produce sugar, which may look innocent enough on a food label, but you always have to consider how the sugar was made. Unless it says that it was made with pure cane sugar, or that it is 100% organic, it is suspect for being a GMO food. Farmed Salmon. With so many health experts recommending that you eat salmon multiple times per week, it's important to note that you should be buying wild caught salmon, and not farm-raised salmon. The difference is staggering, and eating farm raised salmon is not going to give you the same sort of benefits. Conventional Meat. Because cows, chickens, and other animals humans eat are now being fed unnatural diets based on corn and other grains that have been genetically modified, they become a source of GMOs. Conventional Milk. Conventional milking cows are fed a steady diet of GMO-laced feed, as well as pumped full of antibiotics and other drugs to try to keep them healthy in spite of their living conditions. Conventional Juice. Juice is supposed to be good for you, but the conventional juice you'll find in the store has most probably been sweetened with either High Fructose Corn syrup, or another sweetener that is made from GMO foods. Juice still has a reputation of being a healthy food, but it only takes a minute to read the labels of the best-selling juice brands to realize that any good that can come from the juice has been stripped away and replaced with ingredients that make the Nutrition Information read more like a soda label. Pre-Made Foods. Just about every pre-packaged, processed food is going to contain GMOs, unless it's labeled as organic. Many Types of Oil. Two of the most readily available oils, vegetable oil and canola oil, are almost always going to have GMOs in them, because the vegetables and canola that are used are GMO foods. Soybean oil is another oil that is likely to contain GMOs as we've already seen how the majority of soybeans have them. Organic soybean oil would be one way to go, but there are several more options available to you. Sodas. Sodas are sweetened with High Fructose Corn Syrup, made from corn that has been genetically modified.

Unhealthy Foods. non-limiting examples of unhealthy foods to avoid, include, but not limited to, Agave. Many people believe agave is a “healthy” sweetener because it is “natural” and marketed as being low-glycemic. In fact, agave is a highly processed sweetener. The chemical process for manufacturing agave nectar is nearly the same as the corn refiners using in making high-fructose corn syrup from corn starch. Using the agave glucose and inulin found in the plant's roots, manufacturers subject it to a chemical enzymatic (using genetically modified enzymes) process that converts it into nearly pure fructose (70% or higher). Considering that HFCS contains only 55% fructose and it is currently wreaking havoc on Americans' health, imagine what agave will do.

Corn-Fed Beef. Most of the beef you find in a grocery store comes from corn-fed cattle. In fact, unless it is labeled as “grass-fed,” you can be pretty certain that the beef is, indeed, corn-fed. Cattle are ruminants. That is, they naturally survive on grasses, which their bodies are equipped to digest. Many ranchers have switched to feeding cattle corn and other grain-based diets because the feed is cheap and fattens the cattle quickly. Unfortunately, cattle cannot digest grains effectively, and feeding on corn makes them sick. Corn creates an acidic environment in the cows' stomachs, and much like humans consuming acidic cellulose in foods, this can cause all kinds of health problems for cattle, including the growth of E. coli 0157:H7, which can prove fatal to humans. Corn also makes the meat much fattier, containing higher levels of dangerous saturated fats than grass-fed beef, which is high in omega-3 fatty acids. Corn-fed cattle are fed a nutritionally inferior diet and they are thus, nutritionally inferior as food.

Genetically Modified Organisms (GMO). GMOs are now present in 75% to 80% of conventional processed food in the U.S., according to the Grocery Manufacturers Association. This information does not appear on food labels, so you never know if the foods eat are GMO unless you get them directly from the farmer and ask. Since our country has such a short history of eating GMOs, it amounts to a large public health experiment with you as the subject. Independent rodent studies, show startling health effects in rats fed GMO foods including smaller organs, damaged immune systems, decreased immunity, liver atrophy, and many others. Non-Organic Corn In the documentary movie, King Corn, two recent college graduates set out to farm an acre of corn, following it from planting all the way to market. Corn is the major ingredient in the processed Western diet. The two use genetically modified seeds and powerful herbicides. Farmed Salmon. Often called Atlantic Salmon, farmed salmon may be contaminated with dangerous levels of PCBs (polychlorinated biphenyl). These harmful chemicals penetrate the fat of the farm raised salmon (which is especially fatty), and have many negative effects on human health including nervous and endocrine system disruption, increased risks of cancer, immunosuppression, and reproductive problems. Crowded farms can also attract parasites and lethal fish diseases. Farming salmon presents a danger to the environment as well. Farmed salmon that have escaped the farms have become invasive species that compete with and diminish wild populations of fish and contaminate the gene pool. Farms can also release toxins into surrounding waters. Shrimp from around the world is unhealthy and loaded with chemicals. Shrimp from outside the US may be high in antibiotics banned in the United States, such as chloramephnicol, which can cause aplastic anemia. Shrimp from the United States isn't much better. It is very low quality and contains the highest levels of toxins in seafood. Cupcakes. Store bought cupcake is filled with ingredients hazardous to your health. Polyunsaturated fats can cause inflammation and heart disease. Dairy is full of toxic hormones. Wheat is very likely genetically modified and contains gluten, which is very difficult to digest. Sugar has no nutritive value and raises blood glucose levels, stimulating the release of insulin as well as the formation of advanced glycation end products (AGE's), which can damage skin collagen and lead to wrinkles, among many other issues. High-Fructose Corn Syrup (HFCS). HFCS is an inexpensive substitute for real sugar and is used primarily to sweeten beverages, including soft drinks. The American Heart Association identifies sugar-sweetened beverages as the main source of added sugars in our diet, suggesting that liquid calories are more likely to lead to weight gain than calories obtained from solid foods. HFCS, made from yellow dent corn, has been shown to promote increased belly fat and insulin resistance—not to mention the long list of chronic diseases that result directly. The fructose in high-fructose corn syrup goes directly to the liver, where it converts to fat and can ultimately lead to heart disease. New research shows that fructose (like the fructose in HFCS) causes cancer cells to metastasize in a way that other sugars don't, proving that there is a difference between fructose and other sugars. All sugars can lead to health problems, but high-fructose corn syrup is worse in terms of cancer risk. In 2006, the U.S. government gave the corn industry $4,920,813,719 in subsidies, allowing them to sell their crops very cheaply and still make a profit. It's no wonder food manufacturers prefer to use this sweetener over real sugar. Side effects include: Heart disease □-Insulin Resistance (the step before type 2 diabetes) □-Increased belly fat □-Obesity. Trans Fats. Vegetable oils are hydrogenated to transform them from a liquid to a solid fat, which is done to create a desired consistency and to increase the shelf life of foods. Trans fats raise your triglyceride and low density lipoprotein (LDL, the bad cholesterol) levels, which not only increases your risk of heart attack, but has been linked to prostate cancer, breast cancer, Alzheimer's disease, diabetes and obesity. Most experts agree there is no safe limit of ingestion. It is estimated that trans fats cause at least 30,000 deaths each year. Even if you already check labels for “trans fats,” you could be ingesting small amounts in your foods because the FDA allows food manufacturers to state “0 trans fat” on labels as long as the food contains less than 0.5 grams trans fat per serving (in Canada, that number is less than 0.2 grams per serving). Partially hydrogenated oils □-Hydrogenated oils (Note: If a product states “Fully hydrogenated oil” then it is not a trans fat). □-Shortening □-DATEM □-Mono and di-glycerides.

Artificial Flavors. Artificial flavors are additives designed to mimic the taste of natural ingredients. They are used to make processed food taste good because processing removes much of the flavor. When you see “artificial flavors” on a food label, it could mean a single unnatural additive or a blend of hundreds of chemicals. Strawberry flavor, for example, contains 49 man-made chemical ingredients and the typical artificial butter flavor is made of 100 different man-made chemicals! They are cellulose in foods, and are known to cause allergic and behavioral reactions. Unfortunately the FDA does not require flavor companies to disclose ingredients as long as all the ingredients have been deemed “Generally Recognized as Safe (GRAS).” This protects the proprietary formulas of the companies that produce artificial natural flavors, but it allows for many chemicals to be hidden under the word flavor on the ingredients list.

Monosodium Glutamate (MSG). Monosodium glutamate, or MSG, is an artificial flavor found in thousands of processed cellulose in foods, from fast food, to chips, to soup. While the FDA has classified MSG as “generally recognized as safe,” there have been numerous consumer complaints related to adverse reactions to foods containing MSG, including swelling, facial numbness, heart palpitations, nausea and weakness. Glutamate, an amino acid, occurs naturally in many cellulose in foods, but it's also a component of MSG. The problem arises when flavor-enhancing compounds called free glutamates are added to foods. They act as excitatory neurotransmitters, causing the nerves in the brain to fire rapidly and repeatedly. While this stimulation heightens our sense of taste, it can also cause a variety of symptoms, including impaired memory, perception, cognition, and motor skills. MSG can be hidden on a food label under many different names including: Yeast extract, autolyzed yeast extract, hydrolyzed vegetable protein, vegetable powder, and many more.

Artificial Colors. The use of artificial colors has increased 50% since the 1990's, and the bright hues are found in everything from cereals to cosmetics, candy to pharmaceutical drugs. According to the Center for Science in the Public Interest, chemical food dyes are made from known carcinogens. Artificial colors make foods look pretty, but they're deceptive. Pediatricians and parents have long complained about artificial dyes as they have been linked to hyperactivity, attention deficit disorder (ADD) and attention deficit/hyperactivity disorder (ADHD). Artificial dyes can even affect the behavior of children who don't have behavioral disorders. In the U.S. the type of artificial dye must be listed on the label (i.e. red #40, blue #1, yellow #5). However if you live in Canada, food manufacturers are not required to list the type of food coloring used in their products. On a food label, you will simply see them listed as “color.” Artificial Sweeteners. Many of us use artificial sweeteners in place of sugar to lower our caloric intake. However, while it is recommended that sugar be avoided, replacing that sugar with artificial sweeteners is just as bad for you. Acesulfame potassium (also known as ace-K and sold under the brand names Sunett and Sweet One) is a calorie-free sweetener, and while early studies indicated it may cause cancer in animals, little research has been done since it was approved in 1988. Aspartame, the sweetener in Equal and NutraSweet, is found in more than 5,000 products. The body converts aspartame to formaldehyde, a carcinogen that's used in embalming and to treat lumber. Aspartame has been linked to numerous adverse effects, including headaches, dizziness, mood changes, convulsions and memory loss, and the FDA has received more complaints related to aspartame than any other food additive. Neotame is chemically similar to aspartame, but there have been no long-term studies to ensure its safety. Saccharin, in Sweet′N Low, was the first commercial artificial sweetener, and it's been shown to cause cancer in animals. Finally, Sucralose, sold under the name Splenda, is 600 times sweeter than sugar, and study shows it may cause leukemia in mice. Preservatives. Prepared foods are packed with preservatives to prolong their shelf life (they prevent oxidation and slow rancidity). But these chemicals can have a detrimental effect our health, and many are allergens and/or possible carcinogens. The preservatives found to cause the most harm include: BHA (butylated hydroxyanisole) and BHT (butylated hydroxytoluene): Known to affect kidney and liver function, and a possible carcinogen. Polysorbate 60, 65 and 80: Shown to affect the immune system and have caused severe anaphylactic shock—a potentially lethal allergic reaction. Several studies have also linked polysorbate 80 to infertility. Sodium Benzoate: Linked to allergic reactions and is a carcinogen. Sulfites: Used in dried fruit, wine, flavored vinegars, sausages and other foods. Sulfites are common allergens and have been linked to headaches, bowel irritability, behavioral problems and rashes. Asthmatics need to be particularly careful about sulfites as they can cause sudden constriction of the airways. TBHQ (tertiary butylhyroquinone): A petroleum-based food additive, TBHQ has been associated with nausea, vomiting and tinnitus, and has been linked to cancer. Potassium Sorbate: Linked to DNA damage. Nitrates: Used to cure meats. When combined with stomach acids, nitrates produce nitrosamines, which have been linked to cancer. Food Processing is the transformation of raw ingredients, by physical or chemical means into food, or of food into other forms. Food processing combines raw food ingredients to produce marketable food ingredients or food products that can be easily prepared and served by the consumer. Food processing typically involves activities such as mincing and macerating, liquefaction, emulsification, and cooking (such as boiling, broiling, frying, or grilling); pickling, pasteurization, and many other kinds of preservation; and canning or other packaging. (Primary-processing such as dicing or slicing, freezing or drying when leading to secondary products are also included).

Frequencies non-limiting partial list of examples of optional frequencies include: 01=174 Hz02=285 HzUt=396 HzRe=417 HzMi=528 HzFa=639 HzSol=741 HzLa=852 Hz09=963 Hz. The numerical values of the Solfeggio Frequencies are generated by starting with the vector 1, 7, 4 and/or adding the vector 1, 1, 1 MOD 9. Each higher frequency is found by adding 1, 1, 1 MOD 9 to the previous lower frequency. The final frequency, when 1, 1, 1 is added to is, returns the frequency to the lowest tone 1, 7, 4.Ut=396 Hz which reduces to 9 (reducing numbers: 3+9=12=1+2=3; 3+6=9) Re=417 Hz which reduces to 3Mi=528 Hz which reduces to 6Fa=639 Hz which reduces to 9Sol=741 Hz which reduces to 3La=852 Hz which reduces to 6. The belief the frequency assigned to Mi for “Miracles,” 528 Hz, is said by proponents of the idea to be the exact frequency used by genetic engineers throughout the world to repair DNA. The Ancient “Solfeggio frequencies” are cyclic variation of the numbers 369, 147 and/or 258. It is claimed that each frequency has specific spiritual and/or physical healing properties. It is also claimed that they are part of a process that can optionally assist you in creating the possibility of life without stress, illness, and/or sickness. Other non-limiting partial list of examples of frequencies include 7.83 Hz, 126.22 Hz, 136.1 Hz, 144 Hz and/or 528 Hz.

Frequency Modulation is a means of communication between EMFID biomagnetic sensors tag communications and/or a reader; the data is contained in changes between the two frequencies of the carrier wave sent out by the reader.

Germination is the process by which a plant grows from a seed. The most common example of germination is the sprouting of a seedling from a seed of an angiosperm or gymnosperm. In addition, however, the growth of a sporeling from a spore, such as the growth of hyphae from fungal spores, is also germination. Thus, germination can be thought of in a general sense as anything expanding into greater being from a small existence or germ, a method that is commonly used by many seed germination projects.

Genetic Engineering, also called genetic modification is the direct manipulation of an organism's genome using biotechnology. New DNA may be inserted in the host genome by first isolating and copying the genetic material of interest using molecular cloning methods to generate a DNA sequence, or by synthesizing the DNA, and then inserting this construct into the host organism. Genes may be removed, or “knocked out”, using a nuclease. Gene targeting is a different technique that uses homologous recombination to change an endogenous gene, and can optionally be used to delete a gene, remove exons, add a gene, or introduce point mutations. An organism that is generated through genetic engineering is considered to be a genetically modified organism (GMO). The first GMOs were bacteria in 1973 and GM mice were generated in 1974. Insulin-producing bacteria were commercialized in 1982 and genetically modified food has been sold since 1994. Glofish, the first GMO designed as a pet, was first sold in the United States December in 2003. Genetic engineering techniques have been applied in numerous fields including research, agriculture, industrial biotechnology, and medicine. Enzymes used in laundry detergent and medicines such as insulin and human growth hormone are now manufactured in GM cells, experimental GM cell lines and GM animals such as mice or zebrafish are being used for research purposes, and genetically modified crops have been commercialized.

Genetically Modified Contamination (GMO Contamination). Farmers need to be able to provide customers with a choice between GMO (genetically modified organisms), non-GMO, and organic crops and products. Since different types of agriculture are practiced on adjoining fields, suitable measures during planting, cultivation, harvest, transport, storage, and processing are needed in order to prevent the accidental mixing of GMO and non-GMO materials. Contamination may result from seed impurities, wind or insect-borne crosspollination, volunteer or feral plants, and/or inadequate harvest and handling practices. Producers of GMO crops, including herbicide resistant canola, soybeans, alfalfa, sugar beets, and corn; insecticidal (Bt) corn, sweet corn and cotton; and industrial crops, such as alpha-amylase corn for ethanol, have a responsibility to implement best management practices (BMPs) to minimize genetic drift and other forms of contamination, which can negatively impact organic, identity preserved (IP), and other non-GMO producers. Organic, transitional, IP, and other non-GMO crop farmers also need to implement BMPs to minimize risks of GMO contamination.

Genetically Modified Foods (or GM Foods) are foods produced from organisms that have had specific changes introduced into their DNA using the methods of genetic engineering. These techniques allow for the introduction of new traits as well as greater control over traits than previous methods such as selective breeding and mutation breeding. Commercial sale of genetically modified foods began in 1994, when Calgene first marketed its Flavr Savr delayed-ripening tomato. Most food modifications have primarily focused on cash crops in high demand by farmers such as soybean, corn, canola, and cotton seed oil. These have been engineered for resistance to pathogens and herbicides and for better nutrient profiles. GM livestock have been developed, although as of November 2013 none were on the market. There is broad scientific consensus that food on the market derived from GM crops poses no greater risk to human health than conventional food.

Genetically Modified Crops (GMCs, GM crops, or biotech crops) are plants used in agriculture, the DNA of which has been modified using genetic engineering techniques. In most cases the aim is to introduce a new trait to the plant, which does not occur naturally in the species. Examples in food crops include resistance to certain pests, diseases, or environmental conditions, reduction of spoilage, or resistance to chemical treatments (e.g. resistance to a herbicide), or improving the nutrient profile of the crop. Examples in non-food crops include production of pharmaceutical agents, biofuels, and other industrially useful goods, as well as for bioremediation. Farmers have widely adopted GM technology. Between 1996 and 2011, the total surface area of land cultivated with GM crops had increased by a factor of 94, from 17,000 square kilometers (4,200,000 acres) to 1,600,000 km² (395 million acres). 10% of the world's crop lands were planted with GM crops in 2010. As of 2011, 11 different transgenic crops were grown commercially on 395 million acres (160 million hectares) in 29 countries. There is broad scientific consensus that food on the market derived from GM crops poses no greater risk to human health than conventional food. GM crops also provide a number of ecological benefits. However, opponents have objected to GM crops per se on several grounds, including environmental concerns, whether food produced from GM crops is safe, whether GM crops are needed to address the world's food needs, and economic concerns raised by the fact these organisms are subject to intellectual property law.

Genetically Modified Ingredients. non-limiting examples of genetically modified ingredients or processed foods that often have hidden GM sources (unless they organic or declared non-GMO). The following are ingredient are ingredients that may be made from GMOs. Aspartame (also called Amino Sweet®, NutraSweet®, Equal Spoonful®, Canderel®, BeneVial®, E951, baking powder, canola oil (rapeseed oil), canola oil (rapeseed oil), caramel color, cellulose, citric acid, cobalamin (Vitamin B12), colorose, condensed milk, confectioners sugar, corn flour, corn masa, corn meal, corn oil, corn sugar, corn syrup, cornstarch, cottonseed oil, cyclodextrin, cottonseed oil, cyclodextrin, cystein, dextrin, dextrose, diacetyl, diglyceride, erythritol, Equal, food starch, fructose (any form), fructose (any form), glucose, glutamate, glutamic acid, glycerides, glycerin, glycerol, glycerol, glycerol monooleate, glycine, hemicellulose, high fructose corn syrup (HFCS), hydrogenated starch, hydrolyzed vegetable protein, inositol, inverse syrup, inversol, invert sugar, isoflavones, lactic acid, lecithin, leucine. Lysine, malitol, malt, malt syrup, malt extract, maltodextrin, maltose, mannitol, methylcellulose, milk powder, milo starch, modified food starch. modified starch, mono and diglycerides, monosodium glutamate (MSG), Nutrasweet, oleic acid, Phenylalanine, phytic acid, protein isolate, shoyu, sorbitol, soy flour, soy isolates, soy lecithin, soy milk, soy oil, soy protein, soy protein isolate, soy sauce, starch, stearic acid, sugar (unless specified as cane sugar), tamari, tempeh, teriyaki marinades, textured vegetable protein, threonine, tocopherols (vitamin E), tofu, trehalose, triglyceride, vegetable fat, vegetable oil, vitamin B12, vitamin E, whey, whey powder, xanthan gum.

Genetically Modified Foods have been shown to cause harm to humans, animals, and the environmental, and despite growing opposition, more and more foods continue to be genetically altered. It's important to note that steering clear from these foods completely may be difficult, and you should merely try finding other sources than your big chain grocer. If produce is certified USDA-organic, it's non-GMO (or supposed to be!) Also, seek out local farmers and booths at farmer's markets where you can be ensured the crops aren't GMO.

Top 10 Worst GMO Foods that you should not eat, include, but not limited to: 1. Corn: If you've watched any food documentary, you know corn is highly modified. “As many as half of all U.S. farms growing corn for Monsanto are using genetically modified corn,” and much of it is intended for human consumption. Monsanto's GMO corn has been tied to numerous health issues, including weight gain and organ disruption. 2. Soy: Found in tofu, vegetarian products, soybean oil, soy flour, and numerous other products, soy is also modified to resist herbicides. As of now, biotech giant Monsanto still has a tight grasp on the soybean market, with approximately 90% of soy being genetically engineered to resist Monsanto's herbicide Roundup. In one single year, 2006, 96.7 million pounds of glyphosate was sprayed on soybeans alone. 3. Sugar: According to Natural News, genetically-modified sugar beets were introduced to the U.S. market in 2009. Like others, they've been modified by Monsanto to resist herbicides. Monsanto has even had USDA and court-related issues with the planting of its sugar beets, being ordered to remove seeds from the soil due to illegal approval. 4. Aspartame: Aspartame is a toxic additive used in numerous food ingredients and food products, and should be avoided for numerous reasons, including the fact that it is created with genetically modified bacteria. 5. Papayas: This one may come as a surprise to all of you tropical-fruit lovers. GMO papayas have been grown in Hawaii for consumption since 1999. Though they can't be sold to countries in the European Union, they are welcome with open arms in the U.S. and Canada. 6. Canola: One of the most chemically altered foods in the U.S. diet, canola oil is optionally obtained from rapeseed through a series of chemical actions. 7. Cotton: Found in cotton oil, cotton originating in India and China in particular has serious risks. 8. Dairy: Your dairy products contain growth hormones, with as many as one-fifth of all dairy cows in America are pumped with these hormones. In fact, Monsanto's health-hazardous rBGH has been banned in 27 countries, but is still in most US cows. If you must drink milk, buy organic. 9. and 10. Zucchini and Yellow Squash: Closely related, these two squash varieties are modified to resist viruses. The dangers of some of these foods are well-known. The Bt toxin being used in GMO corn, for example, was recently detected in the blood of pregnant women and their babies. But perhaps more frightening are the risks that are still unknown.

Genetically Modified Organisms (GMO) Foods that have been approved by the Food and Drug Administration (FDA) include: Soybeans, Corn, Canola, Plum, Papaya, Alfalfa, Sugar beet, Wheat, Rice, Cantaloupe, Flax, Tomato, Potato, Radicchio, Squash, etc.

Germination is the process by which a plant grows from a seed. The most common example of germination is the sprouting of a seedling from a seed of an angiosperm or gymnosperm. In addition, however, the growth of a sporeling from a spore, such as the growth of hyphae from fungal spores, is also germination. Thus, germination can be thought of in a general sense as anything expanding into greater being from a small existence or germ, a method that is commonly used by many seed germination projects.

Genetically Modified Organism (GMO) is any organism whose genetic material has been altered using genetic engineering techniques. GMOs are the source of genetically modified foods and are also widely used in scientific research and to produce goods other than food. The term GMO is very close to the technical legal term, ‘living modified organism’, defined in the Cartagena Protocol on Biosafety, which regulates international trade in living GMOs (specifically, “any living organism that possesses a novel combination of genetic material obtained through the use of modern biotechnology”).

Growth Hormones (GH) is primarily associated with linear growth in childhood, it continues to have important metabolic functions in adult life. Adult GH deficiency (AGHD) is a distinct clinical entity, and GH replacement in AGHD can improve body composition, strength, aerobic capacity, and mood, and may reduce vascular disease risk. While there are some hormone-related side effects, the balance of benefits and risks is generally favorable, and several countries have approved GH for clinical use in AGHD. GH secretion declines progressively and markedly with aging, and many age-related changes resemble those of partial AGHD. This suggests that replacing GH, or stimulating GH with GH-releasing hormone or a GH secretagogue could confer benefits in normal aging similar to those observed in AGHD—in particular, could reduce the loss of muscle mass, strength, and exercise capacity leading to frailty, thereby prolonging the ability to live independently. However, while most GH studies have shown body composition effects similar to those in AGHD, functional changes have been much less inconsistent, and older adults are more sensitive to GH side effects. Preliminary reports of improved cognition are encouraging, but the overall balance of benefits and risks of GH supplementation in normal aging remains uncertain.

Herbicides, also commonly known as weed killers, are pesticides used to kill unwanted plants. Selective herbicides kill specific targets, while leaving the desired crop relatively unharmed. Some of these act by interfering with the growth of the weed and are often synthetic mimics of natural plant hormones. Herbicides used to clear waste ground, industrial sites, railways and railway embankments are not selective and kill all plant material with which they come into contact. Smaller quantities are used in forestry, pasture systems, and management of areas set aside as wildlife habitat. Some plants produce natural herbicides, such as the genus Juglans (walnuts), or the tree of heaven; such action of natural herbicides, and other related chemical interactions, is called allelopathy. Herbicides are widely used in agriculture and landscape turf management. In the U.S, they account for about 70% of all agricultural pesticide use. Modern, intensively managed agricultural systems have an intrinsic reliance on the use of herbicides and other pesticides. Some high-yield varieties of crop species are not very tolerant of competition from weeds. Therefore, if those crops are to be successfully grown, herbicides must be used. Many studies have indicated the shorter-term benefits of herbicide use. For example, studies of the cultivation of maize in Illinois have demonstrated that the average reduction of yield was 81% in unweeded plots, while a 51% reduction was reported in Minnesota. Yields of wheat and barley can be reduced by 25%-50% as a result of competition from weeds. To reduce these important, negative influences of weeds on agricultural productivity, herbicides are commonly applied to agricultural fields. As noted above, the herbicide must be toxic to the weeds, but not to the crop species. Non-limiting examples of herbicides include:

Chlorophenoxy Acid Herbicides. Chlorophenoxy acid herbicides cause toxicity to plants by mimicking their natural hormone-like auxins, and thereby causing lethal growth abnormalities. These herbicides are selective for broad-leaved or angiosperm plants, and are tolerated by monocots and conifers at the spray rates normally used. These chemicals are moderately persistent in the environment, with a half-life in soil typically measured in weeks, and a persistence of a year or so. The most commonly used compounds are 2,4-D (2,4-Dichlorophenoxyacetic acid); 2,4,5-T (2,4,5-Trichlorophenoxyacetic acid); MCPA (2-Methyl-4-chlorophenoxyacetic acid); and silvex [2-(2,4,5-Trichlorophenoxy)-propionic acid]. Triazine Herbicides. Triazine herbicides are mostly used in corn agriculture, and sometimes as soil sterilants. These chemicals are not very persistent in surface soils, but they are mobile and can cause a contamination of groundwater. Important examples of this class of chemicals are: atrazine [2-Chloro-4-(ethylamino)-6-(isopropylamino)s-triazine]; cynazine [2-(4-Chloro-6-ethylamino-5-triazin-2-ylamino)-2-methylpropionitrile]; hexazinone [3-Cyclohexyl-6-(dimethyl-amino)-1-methyl-1,3,5-triazine-2,4(1H,3H)-dione]; metribuzin [4-Amino-6-tert-butyl-3-(methylthio)-as-triazin-5(4H)-one]; and simazine [2-chloro-4,6-bis-(ethyl-amino)-s-triazine]. Organic Phosphorus Herbicides. Organic phosphorus herbicides are few, but they include the commonly used chemical, glyphosate (N-phosphonomethyl-glycine). Glyphosate has a wide range of agricultural uses, and it is also an important herbicide in forestry. To kill plants, glyphosate must be taken up and transported to perennating tissues, such as roots and rhizomes, where it interferes with the synthesis of certain amino acids. Because glyphosate can potentially damage many crop species, its effective use requires an understanding of seasonal changes in the vulnerability of both weeds and crop species to the herbicide. Glyphosate is not mobile in soils, has a moderate persistence, and is not very toxic to animals. Recently, varieties of certain crops, notably the oilseed canola, have been modified through genetic engineering (transgenics) to be tolerant of glyphosate herbicide. Previously, there were no effective herbicides that could be applied to canola crops to reduce weed populations, but now glyphosate can optionally be used for this purpose. However, this has become controversial because many consumers do not want to eat foods made from transgenic crops. Hybrid. In biology a hybrid is mix of two animals or plants of different breeds, varieties, species or genera. Using genetics terminology, it may be defined as follows. In general usage, hybrid is synonymous with heterozygous: any offspring resulting from the breeding of two genetically distinct individuals, a genetic hybrid carries two different alleles of the same gene, a structural hybrid results from the fusion of gametes that have differing structure in at least one chromosome, as a result of structural abnormalities, a numerical hybrid results from the fusion of gametes having different haploid numbers of chromosomes a permanent hybrid is a situation where only the heterozygous genotype occurs, because all homozygous combinations are lethal. From a taxonomic perspective, hybrid refers to: Offspring resulting from the interbreeding between two animal species or plant species. Hybrids between different subspecies within a species (such as between the Bengal tiger and Siberian tiger) are known as intra-specific hybrids. Hybrids between different species within the same genus (such as between lions and tigers) are sometimes known as interspecific hybrids or crosses. Hybrids between different genera (such as between sheep and goats) are known as intergeneric hybrids. Extremely rare interfamilial hybrids have been known to occur (such as the guinea fowl hybrids). No interordinal (between different orders) animal hybrids are known. The third type of hybrid consists of crosses between populations, breeds or cultivars within a single species. This meaning is often used in plant and animal breeding, where hybrids are commonly produced and selected, because they have desirable characteristics not found or inconsistently present in the parent individuals or populations. This flow of genetic material between populations is often called hybridization.

Hydroxypropyl Cellulose (HPC) is a derivative of cellulose with both water solubility and organic solubility. It is used as a topical ophthalmic protectant and lubricant.

Ingredient is a substance that forms part of a mixture (in a general sense). For example, in cooking, recipes specify which ingredients are used to prepare a specific dish. Many commercial products contain a secret ingredient that is purported to make them better than competing products. In the pharmaceutical industry, an active ingredient is that part of a formulation that yields the effect required by the customer. National laws usually require prepared food ingredients to display a list of ingredients, and specifically require that certain additives be listed. In most developed countries, the law requires that ingredients be listed according to their relative weight in the product. If an ingredient itself consists of more than one ingredient (such as the cookie pieces which are a part of “cookies and cream” flavor ice cream), then that ingredient is listed by what % age of the total product it occupies, with its own ingredients displayed next to it in brackets. The term constituent is often chosen when referring to the substances that constitute the tissue of living beings such as plants and people, because the word ingredient in many minds connotes a sense of human agency (that is, something that a person combines with other substances), whereas the natural products present in living beings were not added by any human agency but rather occurred naturally (“a plant doesn't have ingredients”). Thus all ingredients are constituents, but not all constituents are ingredients.

Injection Molding is a manufacturing process in the U.S.A. for producing parts by injecting material into a mold. Injection molding can be performed with a host of materials, including metals, glasses, elastomers, confections, and most commonly thermoplastic and thermosetting polymers. Material for the part is fed into a heated barrel, mixed, and forced into a mold cavity, where it cools and hardens to the configuration of the cavity. After a product is designed, usually by an industrial designer or an engineer, molds are made by a mold maker (or tool maker) from metal, usually either steel or aluminum, and precision-machined to form the features of the desired part. Injection molding is widely used for manufacturing a variety of parts, from the smallest components to entire body panels of cars. Advances in 3D printing technology, using photopolymers, which do not melt during the injection molding of some lower temperature thermoplastics, can optionally be used for some simple injection molds. Parts to be injection molded must be very carefully designed to facilitate the molding process; the material used for the part, the desired shape and features of the part, the material of the mold, and the properties of the molding machine must all be taken into account. The versatility of injection molding with nanocrystalline (NC) materials is facilitated by this breadth of design considerations and possibilities.

Lecithin is a dietary supplement and generic term to designate any group of yellow-brownish fatty substances occurring in animal and plant tissues composed of phosphoric acid, choline, fatty acids, glycerol, glycolipids, triglycerides, and phospholipids (e.g., phosphatidylcholine, phosphatidylethanolamine, and phosphatidylinositol)

Lyocell refers to a type of fabric that is made from the cellulose of previously treated wood pulp. With its natural origins and/or chemical processing, some people classify lyocell fabric as somewhere in between natural fibers and/or synthetic fibers. Lyocell fabric is also commonly referred to “Tencel” fabric, but the latter is actually the brand/or name of this particular fabric classification.

L Lysine (abbreviated as Lys or K) is a dietary supplement and an α-amino acid with the chemical formula HO₂CCH(NH₂)(CH₂)₄NH₂. It is an essential amino acid for humans. Lysine's codons are AAA and AAG. Lysine is a base, as are arginine and histidine. The ∈-amino group often participates in hydrogen bonding and as a general base in catalysis. (The ∈-amino group (NH₃⁺) is attached to the fifth carbon from the α-carbon, which is attached to the carboxyl (C═OOH) group). Common posttranslational modifications include methylation of the ∈-amino group, giving methyl-, dimethyl-, and trimethyllysine. The latter occurs incalmodulin. Other posttranslational modifications at lysine residues includeacetylation, sumoylation, and ubiquitination. Collagen contains hydroxylysine, which is derived from lysine by lysyl hydroxylase. O-Glycosylation of hydroxylysine residues in the endoplasmic reticulum or Golgi apparatus is used to mark certain proteins for secretion from the cell. In opsins like rhodopsin and the visual opsins (encoded by the genes OPN1SW, OPN1MW, and OPN1LW), retinaldehyde forms a Schiff base with a conserved lysine residue, and interaction of light with the retinylidene group causes signal transduction in color vision (See visual cycle for details). Deficiencies may cause blindness, as well as many other problems due to its ubiquitous presence in proteins.

Macrofibril or Microfibril is a very fine fibril, or fiber-like strand, consisting of glycoproteins and cellulose. It is usually, but not always, used as a general term in describing the structure of protein fiber, e.g. hair and sperm tail. Its most frequently observed structural pattern is the 9+2 pattern in which two central protofibrils are surrounded by nine other pairs. Cellulose inside plants is one of the examples of non-protein compounds that are using this term with the same purpose. Cellulose microfibrils are laid down in the inner surface of the primary cell wall. As the cell absorbs water, its volume increases and the existing microfibrils separate and new ones are formed to help increase cell strength.

Magnetic Field is the magnetic influence of electric currents and/or magnetic materials. The magnetic field at any given point is specified by both a direction and/or a magnitude (or strength); as such it is a vector field The term is used for two distinct but closely related fields denoted by the symbols B and/or H, where H is measured in units of amperes per meter (symbol: A·m⁻¹ or A/m) in the SI. B is measured in teslas (symbol: T) and/or newtons per meter per ampere (symbol: N·m⁻¹·A⁻¹ or N/(m·A)) in the SI. B is most commonly defined in terms of the Lorentz force it exerts on moving electric charges. Magnetic fields are produced by moving electric charges and/or the intrinsic magnetic moments of elementary products associated with a fundamental quantum property, their spin. In special relativity, electric and/or magnetic fields are two interrelated aspects of a single object, called the electromagnetic tensor; the split of this tensor into electric and/or magnetic fields depends on the relative velocity of the observer and/or charge. In quantum physics, the electromagnetic field is quantized and/or electromagnetic interactions result from the exchange of photons.

Medical Implants are being used in every organ of the human body include artificial hips, heart pacemaker, breast implant devices, spine screws, rods and artificial discs, metal screws, pins, plates and rods, artificial knees, coronary stents, ear tubes, artificial eye lenses, etc. Coatings for medical applications, medical implants, breast implant devices, microchip implants or other types of implants such as coatings for medical applications, medical implants, breast implant devices, microchip implants or other types of implants, orthopedic implants and heart valves, are made of titanium and stainless steel alloys, primarily because they are biocompatible The implant material market has evolved over the years starting from vanadium steel and PTFE to the usage of shape memory alloys and resorbabales. Unfortunately, in some cases these metal alloys may wear out within the lifetime of the patient. Metallic Materials, Polymeric Materials and Ceramic Materials are used. Nanocrystalline zirconium oxide (zirconia) is hard, wear resistant, bio-corrosion resistant and bio-compatible. Nanocrystalline (NC) materials present an attractive alternative material for medical implants. This and other nanoceramics can optionally be made as strong, light aerogels by sol-gel techniques. Nanocrystalline (NC) silicon carbide is another candidate material for artificial heart valves primarily because of its low weight, high strength and inertness.

Metals (from Greek μ\acute{∈}ταλλov métallon, “mine, quarry, metal” is a material (an element, compound, or alloy) that is typically hard, opaque, shiny, and/or has good electrical and/or thermal conductivity. Metals are generally malleable—that is, they can optionally be hammered or pressed permanently out of shape without breaking or cracking—as well as fusible (able to be fused or melted) and/or ductile (able to be drawn out into a thin wire). About 91 of the 118 elements in the periodic table are metals (some elements appear in both metallic and/or non-metallic forms). The meaning of “metal” differs for various communities. For example, astronomers use the blanket term “metal” for convenience to collectively describe all elements other than hydrogen and/or helium (the main components of stars, which in turn form most of the visible matter in the universe). Thus, in astronomy and/or physical cosmology, the metallicity of an object is the proportion of its matter made up of chemical elements other than hydrogen and/or helium) In addition, many elements, coating applications, plastics and/or compounds that are not normally classified as metals become metallic under high pressures; these are formed as metallic allotropes of non-metals, amorphous metals.

Microcrystalline Cellulose (MCC) is known in the art as typically a purified, partially depolymerized cellulose that is prepared by treating alpha cellulose, in the form of a pulp manufactured from fibrous plant material, with mineral acids. See, e.g., U.S. Pat. No. 4,744,987. It is a generally white, odorless, tasteless, relatively free flowing powder that is generally insoluble in water, organic solvents, dilute alkalis and dilute acids. U.S. Pat. No. 2,978,446 to Battista et al. and U.S. Pat. No. 3,146,168 to Battista describe microcrystalline cellulose and its manufacture; the latter patent concerns microcrystalline cellulose (MCC) for pharmaceutical applications. Microcrystalline Cellulose (MCC) can optionally include a term for refined wood pulp and is used as a texturizer, an anticaking agent, a fat substitute, an emulsifier, an extender, and a bulking agent in food production. The most common form is used in vitamin supplements or tablets. It is also used in plaque assays for counting viruses, as an alternative to carboxymethylcellulose. In many ways' cellulose makes the ideal excipient. A naturally occurring polymer, it is composed of glucose units connected by a 1-4 beta glycosidic bond. These linear cellulose chains are bundled together as microfibril spiralled together in the walls of plant cell. Each microfibril exhibits a high degree of three-dimensional internal bonding resulting in a crystalline structure that is insoluble in water and resistant to reagents. There are, however, relatively weak segments of the microfibril with weaker internal bonding. These are called amorphous regions; some argue that they are more accurately called dislocations, because of the single-phase structure of microfibrils. The crystalline region is isolated to produce microcrystalline cellulose. Microcrystalline Cellulose (MCC) can optionally include free-flowing crystalline powder (a non-fibrous microparticles). It is insoluble in water, dilute acids and most organic solvents, but slightly soluble in the alkali solution of 20%. It has a wide range of uses in the pharmaceutical excipients and can be directly used for tabletting of dry powder. It is widely used as pharmaceutical excipients, flow aids, fillers, disintegrating agents, anti-sticking agents, adsorbents, and capsule diluents. Microcrystalline cellulose (MCC) is a pure product of cellulose depolymerization, an odorless and tasteless crystalline powder prepared from the natural cellulose. In the pharmaceutical industry, the MCC products can optionally be used as pharmaceutical excipients and disintegrating agents of tablets; in the food industry, MCC can optionally be used as an important base material in functional foods and is an ideal health food additive; in the paint industry, MCC can optionally be used as thickeners and emulsifiers of water-based coating applications by using its thixotropic and thickening properties; in cosmetic additives, sugar substitute, sweeteners, artificial sweeteners, amino acid regulators, acidity regulators, anticaking agents, applications as taste masking agents, disintegrating agents, binders in granulation process, fillers in solid dosage forms, thickening and stabilizing agents, gelling agents, compressibility enhancers, coating agents, drug delivery systems, pharmaceutical coatings process, medical coating applications, topical ophthalmic protectant and lubricant and the like, antifoaming agents, antibacterial agents, anti-aging products, antioxidants, absorption blocking agents, carcinogen blocking agents, cellulose geltain capsules for dietary supplements, medications, vitamins, marijuana oils, cannabis oils, hash oils, hemp oils and other types of oils for cancer treatment, pharmaceutical uses and other medical uses, encapsulation products, cholesterol blocking agents, fat blocking agents, caloric blocking agents, blocking sugar absorption, neuromuscular blocking agents, food coloring, color retention agents, emulsifiers, natural or artificial flavors, flavor enhancers, flour treatment agent, glazing agents, humectants, tracer gas, preservatives, stabilizers, thickeners, smart packaging and intelligent labeling technology for food, beverages, pharmaceutical and household products, sunscreens, coatings, MCC has a combination of filler, thickening and emulsifying effects in itself, and has very good emulsifying capacity for the oily substance. Thus it can be seen that MCC has a wide range of uses, and that the domestic demand for the product will continue to increase.

Microfluidics is a multidisciplinary field intersecting engineering, physics, chemistry, biochemistry, nanotechnology, and biotechnology, with practical applications to the design of systems in which small volumes of fluids will be handled. Microfluidics emerged in the beginning of the 1980s and is used in the development of inkjet print heads, DNA chips, lab-on-a-chip technology, micro-propulsion, and micro-thermal technologies. It deals with the behavior, precise control and manipulation of fluids that are geometrically constrained to a small, typically sub-millimeter, and scale. Typically, micro means one of the following features: small volumes (μL, nL, pL, fL), small size, low energy consumption, and effects of the micro domain. Typically fluids are moved, mixed, separated or otherwise processed. Numerous applications employ passive fluid control techniques like capillary forces. In some applications external actuation means are additionally used for a directed transport of the media. Examples are rotary drives applying centrifugal forces for the fluid transport on the passive chips.

Active Microfluidics refers to the defined manipulation of the working fluid by active (micro) components such as micro pumps or micro valves. Micro pumps supply fluids in a continuous manner or are used for dosing. Micro valves determine the flow direction or the mode of movement of pumped liquids. Often processes, which are normally carried out in a lab, are miniaturized on a single chip in order to enhance efficiency and mobility as well as reducing sample and reagent volumes.

Microchip Implant is a human microchip implant with an identifying integrated circuit device or RFID transponder encased in silicate glass with GPS tracking or satellite tracking, which is implanted in the body of a human being or pet. A subdermal implant typically contains a unique ID number that can be linked to information contained in an external database, such as personal identification, medical history, medications, allergies, and contact information.

Microcrystalline Cellulose (MCC) is particularly used because it contains cellulose which is perhaps the most widely used fillers. Celluloses are biocompatible, chemically inert and have good-tablet forming and disintegrating properties. They are therefore used also as dry binders and disintegrants in tablets. Microcrystalline Cellulose is prepared by hydrolysis of cellulose is followed by spray drying. The particles thus formed are aggregates of smaller cellulose fibers. Hence, aggregates of different particles size can be prepared which have different flowablities. The flow properties of the material are generally good, and the direct compression characteristics are excellent. MCC is a unique diluent for producing cohesive compacts. The material also acts as a disintegrating agent. Microcrystalline cellulose (MCC) can be combined with other materials such as lubricants or disintegrants.

Monocrystalline Silicon (or “single-crystal silicon”, “single-crystal Si”, “mono c-Si”, or just mono-Si) is the base material for silicon chips used in virtually all electronic equipment today. Mono-Si also serves as photovoltaic, light-absorbing material in the manufacture of solar cells. It consists of silicon in which the crystal lattice of the entire solid is continuous, unbroken to its edges, and free of any grain boundaries. Mono-Si can be prepared intrinsic, consisting only of exceedingly pure silicon, or doped, containing very small quantities of other elements added to change its semiconducting properties. Most silicon monocrystals are grown by the Czochralski process into ingots of up to 2 meters in length and weighing several hundred kilogrammes. These cylinders are then sliced into thin wafers of a few hundred microns for further processing. Single-crystal silicon is perhaps the most important technological material of the last few decades—the “silicon era”, because its availability at an affordable cost has been essential for the development of the electronic devices on which the present day electronic and informatic revolution is based. Monocrystalline silicon differs from other allotropic forms, such as the non-crystallineamorphous silicon—used in thin-film solar cells, and polycrystalline silicon, that consists of small crystals, also known as crystallites.

Monomer is a molecule that may bind chemically to other molecules to form a polymer. The term “monomeric protein” may also be used to describe one of the proteins making up amultiprotein complex. The most common natural monomer is glucose, which is linked by glycosidic bonds into polymers such as cellulose and starch, and is over 77% of the mass of all plant matter. Most often the term monomer refers to the organic molecules which form synthetic polymers, such as, for example, vinyl chloride, which is used to produce the polymer polyvinyl chloride (PVC). The process by which monomers combine end to end to form a polymer is called polymerization. Molecules made of a small number of monomer units, up to a few dozen, are called oligomers. Monomers are the building blocks of more complex molecules, called polymers.

Nanocrystalline DNA Interface. Hybrid nanocomposites can electronically link TiO2 nanoparticles to DNA oligonucleotides can optionally be used to link biomolecules with inorganic components was achieved by using bridging enediol ligands, such as dopamine (DA), which facilitate hole transfer across the interface, establishing efficient crosstalk between the biomolecule and metal oxide nanoparticles. The inherent programmability of oligonucleotides builds recognition properties into the hybrid system, allowing selective binding of nanoparticles to targeted molecules. The inorganic nanoparticles are inherently photoresponsive and therefore serve as a source of photogenerated charges that act as reporters of the electronic properties of the biomolecules. These photoactive bioinorganic TiO2/DA/DNA triads are capable of complex photo chemistries such as light induced manipulation of biomolecules and their switching functions. Consequently, light induced extended charge separation in these systems was found to be a fingerprint of DNA oligonucleotide hybridization.

Nanocrystalline MgFe₂O₄ Particles for Cancer Cure. Nanocrystalline magnesium ferrites (MgFe₂O₄) can optionally be used as a drug carrier for the treatment of cancer or other diseases. The cytotoxic effects of MgFe₂O₄ nanoparticles in various concentrations (25, 50, 100, 200, 400, and 800 μg/mL) against MCF-7 human breast cancer cells were analyzed. MTT assay findings suggest the increased accumulation of apoptotic bodies with the increasing concentration of MgFe₂O₄ nanoparticles in a dose-dependent manner. Flow cytometry analysis shows that MgFe₂O₄ nanoparticles in 800 μg/mL concentration are more cytotoxic compared to vehicle-treated MCF-7 cells and suggests their potential utility as a drug carrier in the treatment of cancer or other diseases.

Nanocrystalline Cellulose (NCC) are solid-state systems constituting crystals of sizes less than 100 nm in at least one dimension. The understanding of the extraordinary behavior of nanostructured materials requires detailed studies of the correlations between the processing, structure, and properties. These studies rely on the identification and development of appropriate processing methods and suitable characterization methods and analytical tools for the nanocrystalline cellulose (NCC). This review has shown that PVD and CVD methods have the capability of producing nanophase materials. However, most of these vapor processing techniques involve the use of a vacuum system and sophisticated deposition chamber. Therefore, the drawbacks of these vapor processing techniques are the high production costs and the difficulty of fabricating nanophase materials cost effectively in large quantity. The recent development of novel and cost-effective vapor processing methods, especially those based on the aerosol and flame synthesis methods, offer cheaper alternatives to the conventional CVD and PVD techniques and may widen the scope of commercial applications of vapor processing of nanostructured materials.

Nanocrystalline Cellulose (NCC) or Nanocrystalline (NC) Materials can be prepared in several ways. Methods are typically categorized based on the phase of matter the material transitions through before forming the nanocrystalline final product.

Solid-State Processing. Solid-state processes do not involve melting or evaporating the material and are typically done at relatively low temperatures. Examples of solid-state processes include mechanical alloying using a high-energy ball mill and certain types of severe plastic deformation processes.

Liquid Processing. Nanocrystalline metals can be produced by rapid solidification from the liquid using a process such as melt spinning. This often produces an amorphous metal, which can be transformed into an NC metal by annealing above the crystallization temperature.

Vapor-Phase Processing. Thin films of nanocrystalline (NC) materials can be produced using vapor deposition processes such as MOCVD. Solution processing. Some metals, particularly nickel and nickel alloys, can be made into nanocrystalline foils using electrodeposition.

Nanocrystalline Cellulose (NCC) Material or Nanocrystalline (NC) Materials are a polycrystalline material with a crystallite size of only a few nanometers. These materials fill the gap between amorphous materials without any long range order and conventional coarse-grained materials. Definitions vary, but nanocrystalline material is commonly defined as a crystallite (grain) size below 100 nm. Grain sizes from 100-500 nm are typically considered “ultrafine” grains. The grain size of a NC sample can be estimated using x-ray diffraction. In materials with very small grain sizes, the diffraction peaks will be broadened. This broadening can be related to a crystallite size using the Scherrer equation (applicable up to ˜50 nm), a Williamson-Hall plot, or more sophisticated methods such as the Warren-Averbach method or computer modeling of the diffraction pattern. The crystallite size can be measured directly using transmission electron microscopy.

Nanocrystalline Cellulose (NCC) or Nanocrystalline (NC) Materials are single or multi-phase polycrystalline solids with a grain size of a few nanometers (1 nm=10-9 m=10 Å), typically less than 100 nm. Since the grain sizes are so small, a significant volume of the microstructure in nanocrystalline materials is composed of interfaces, mainly grain boundaries, i.e., a large volume fraction of the atoms resides in grain boundaries. Consequently, nanocrystalline materials exhibit properties that are significantly different from, and often improved over, their conventional coarse-grained polycrystalline counterparts. Materials with microstructural features of nanometric dimensions are referred to in the literature as nanocrystalline materials (a very generic term), nanocrystals, nanostructured materials, nanophase materials, nanometer-sized crystalline solids, or solids with nanometer-sized microstructural features. Nanostructured solids is perhaps the most accurate description, even though nanocrystalline materials will be the appropriate term if one is dealing with solids with grains made up of crystals. Nanocrystalline materials can be classified into different categories depending on the number of dimensions in which the material has nanometer modulations. Thus, they can be classified into (a) layered or lamellar structures, (b) filamentary structures, and (c) equiaxed nanostructured materials. A layered or lamellar structure is a one dimensional (1D) nanostructure in which the magnitudes of length and width are much greater than the thickness that is only a few nanometers in size. One can also visualize a two-dimensional (2D) rod-shaped nanostructure that can be termed filamentary and in this the length is substantially larger than width or diameter, which are of nanometer dimensions. The most common of the nanostructures, however, is basically equiaxed (all the three dimensions are of nanometer size) and are termed nanostructured crystallites (three-dimensional [3D] nano structures). The nanostructured materials may contain crystalline, quasicrystalline, or amorphous phases and can be metals, ceramics, polymers, or composites. If the grains are made up of crystals, the material is called nanocrystalline. On the other hand, if they are made up of quasicrystalline or amorphous (glassy) phases, they are termed nanoquasicrystals and nanoglasses, respectively. Nanocrystalline materials can be synthesized either by consolidating small clusters or breaking down the bulk material into smaller and smaller dimensions. Synthesized nano crystalline materials can optionally be with the inert gas condensation technique to produce nanocrystalline powder particles and consolidated them in situ into small disks under ultra-high vacuum (UHV) conditions. Since then a number of techniques have been developed to prepare nanostructured materials starting from the vapor, liquid, or solid states. Nanostructured materials have been synthesized in recent years by methods including inert gas condensation, mechanical alloying, spray conversion processing, severe plastic deformation, electrodeposition, rapid solidification from the melt, physical vapor deposition, chemical vapor processing, co-precipitation, sol-gel processing, sliding wear, spark erosion, plasma processing, auto-ignition, laser ablation, hydrothermal pyrolysis, thermophoretic forced flux system, quenching the melt under high pressure, biological templating, sonochemical synthesis, and devitrification of amorphous phases. Actually, in practice any method capable of producing very fine grain-sized materials can optionally be used to synthesize nanocrystalline materials. The grain size, morphology, and texture can be varied by suitably modifying/controlling the process variables in these methods. Each of these methods has advantages and disadvantages and one should choose the appropriate method depending upon the requirements. If a phase transformation is involved, e.g., liquid to solid or vapor to solid, then steps need to be taken to increase the nucleation rate and decrease the growth rate during formation of the product phase. In fact, it is this strategy that is used during devitrification of metallic glasses to produce nano crystalline materials.

Nanomaterials are experiencing a rapid development in recent years due to their existing and/or potential applications in a wide variety of technological areas such as electronics, flexible electronic displays, batteries, catalysis, ceramics, magnetic data storage, telecommunication and data communication components, etc. To meet the technological demands in these areas, the size of the materials should be reduced to the nanometer scale. For example, the miniaturization of functional electronic devices demands the placement or assembly of nanometer scale components into well-defined structures. As the size reduces into the nanometer range, the materials exhibit peculiar and interesting mechanical and physical properties, e.g. increased mechanical strength, enhanced diffusivity, higher specific heat and electrical resistivity compared to conventional coarse grained counterparts. Nanomaterials can be classified into nanocrystalline (NC) materials and nanoparticles. The former are polycrystalline bulk materials with grain sizes in the nanometer range (less than 100 nm), while the latter refers to ultrafine dispersive particles with diameters below 100 nm. Nanoparticles are generally considered as the building blocks of bulk nanocrystalline (NC) materials.

Nanocrystalline Silver (NCS) has proven to be an important wound dressing particularly in chronic infected wounds. However, debate still rages around its use in the case of partially epithelialized wounds, particularly when these are non-infected. Much of the debate has revolved around seemingly contradictory research publications that blurred the use of NCS in these clinical situations, primarily based on reported cytotoxic effects of NCS on cell lines in vitro. MMPs, in particular MMP-9 (gelatinase) have been demonstrated to be pivotal in the progression from keratinocyte cleavage, to migration and re-epithelialisation. High levels promote increases in TNF-α; IL-8 and TGFβ, all associated with exaggerated ongoing inflammation and chronicity. Low levels impede the process of keratinocyte migration. Thus, as in so many clinical situations, a balance of MMP level is extremely important. NCS has been demonstrated to decrease these undesirable high levels of MMP-9 making it an ideal dressing for chronic infected wounds, acute inflamed wounds and burn wounds of all types which are associated with protracted raised MMP-9 levels. The converse applies too—NCS used in a situation of minimal inflammation may undesirably decrease the low levels of MMP-9 and adversely affect epithelialisation. NCS would be contra-indicated in conjunction with cell lines in vitro, cell cultured lines in vivo and integrated artificial matrices with added cell lines.

Nanocrystalline Silver Dressings is a term referring to the emergence of multi-drug-resistant strains of bacteria and represents a particular challenge in the field of wound management. Nanocrystalline silver has both antimicrobial and anti-inflammatory properties. Nanocrystalline silver dressings may optionally possess the physical properties to act as a barrier to the transmission of methicillin-resistant Staphylococcus aureus (MRSA) in the laboratory setting and in a clinical setting. MRSA suspension and colony culture experiments were performed showing that nanocrystalline silver dressings act as potent and sustained antimicrobial agents, efficiently inhibiting MRSA penetration. Subsequently, a double-center clinical trial was initiated using nanocrystalline silver dressings as a cover for 10 MRSA colonized wounds in a total of seven patients. By delineating the MRSA load on the upper side of the dressing and the wound bed each time the dressing was changed (i.e. after 1, 24, 48 and 72 h), nanocrystalline silver dressings were found to provide a complete, or almost complete, barrier to the penetration/spread of MRSA in 95% of readings. In addition, 67% of all wound observations showed a decrease in the MRSA load with an eradication rate of 11%.

Nanocellulose is a term referring to nano-structured cellulose. This may be either cellulose nanofibers (CNF) also called microfibrillated cellulose (MFC), nanocrystalline cellulose (NCC), or bacterial nanocellulose, which refers to nano-structured cellulose produced by bacteria. CNF is a material composed of nanosized cellulose fibrils with a high aspect ratio (length to width ratio). Typical lateral dimensions are 5-20 nanometers and longitudinal dimension is in a wide range, typically several micrometers. It is pseudo-plastic and exhibits the property of certain gels or fluids that are thick (viscous) under normal conditions, but flow (become thin, less viscous) over time when shaken, agitated, or otherwise stressed. This property is known as thixotropy. When the shearing forces are removed the gel regains much of its original state. The fibrils are isolated from any cellulose-containing source including wood-based fibers (pulp fibers) through high-pressure, high temperature and high velocity impact homogenization, grinding or microfluidization (see manufacture below). Nanocellulose can also optionally be obtained from native fibers by an acid hydrolysis, giving rise to highly crystalline and rigid nanoparticles (often referred to as CNC or nanowhiskers), which are shorter (100s to 1000 nanometers) than the nanofibrils obtained through the homogenization, microfluiodization or grinding routes. The resulting material is known as nanocrystalline cellulose (NCC).

Nanocellulose is a unique and promising natural material extracted from native cellulose that has gained much attention for its use as biomedical material because of its remarkable physical properties, special surface chemistry and excellent biological properties (biocompatibility, biodegradability and low toxicity). Three different types of nanocellulose, viz. cellulose nanocrystals (CNC), cellulose nanofibrils (CNF) and bacterial cellulose (BC), are optionally used at the molecule level in biomedical applications (e.g. tissue bioscaffolds for cellular culture; drug excipient and drug delivery; and immobilization and recognition of enzyme/protein) as well as at the level of macroscopic materials (e.g. blood vessel and soft tissue substitutes; skin and bone tissue repair materials; and antimicrobial materials). Nanocellulose can optionally be used as a low calorie replacement for carbohydrate additives used as thickeners, flavor carriers and suspension stabilizers in a wide variety of food ingredients, food products and is useful for producing fillings, crushes, chips, wafers, soups, gravies, puddings, etc.

Nanocrystalline Cellulose (NCC) is an emerging renewable nanomaterial that holds promise in many different nanocrystalline (NC) applications, such as in personal care, chemicals, cellulose in foods, pharmaceuticals, etc. By appropriate modification of NCC, various functional nanomaterials with outstanding properties, or significantly improved physical, chemical, biological, as well as electronic properties can be developed. The nanoparticles are stabilised in aqueous suspension by negative charges on the surface, which are produced during the acid hydrolysis process. NCC suspensions can form a chiral nematic ordered phase beyond a critical concentration, i.e. NCC suspensions transform from an isotropic to an anisotropic chiral nematic liquid crystalline phase. Due to its nanoscale dimension and intrinsic physicochemical properties, NCC is a promising renewable biomaterial that can optionally be used as a reinforcing component in high performance nanocomposites. Many new nanocomposite materials with attractive properties were obtained by the physical incorporation of NCC into a natural or synthetic polymeric matrix. Simple chemical modification on NCC surface can improve its dispersability in different solvents and expand its utilisation in nano-related applications, such as drug delivery, protein immobilisation, and inorganic reaction template.

Nanocrystalline Synthetic (NC) Diamonds. Diamond properties are significantly affected by crystallite size. High surface to volume fractions result in enhanced disorder, sp² bonding, hydrogen content and scattering of electrons and phonons. Most of these properties are common to all low dimensional materials, but the addition of carbon allotropes introduces sp² bonding, a significant disadvantage over systems such as amorphous silicon. Increased sp² bonding results in enhanced disorder, a significantly more complex density of states within the bandgap, reduction of Young's modulus, increased optical absorption etc. At sizes below 10 nm, many diamond particles and film properties deviate substantially from that of bulk diamond, mostly due not only to the contribution of sp² bonding, but also at the extreme low dimensions due to size effects. Despite these drawbacks, nano-diamond films and particles are powerful systems for a variety of applications and the study of fundamental science. Knowledge of the fundamental properties of these materials allows a far greater exploitation of their attributes for specific applications. This review attempts to guide the reader between the various nanocrystalline diamond forms and applications, with a particular focus on thin films grown by chemical vapor deposition.

Nanocrystalline (NC) Silicon (nc-Si), sometimes also known as microcrystalline silicon (μc-Si), is a form of porous silicon. It is an allotropic form of silicon with paracrystalline structure—is similar to amorphous silicon (a-Si), in that it has an amorphous phase. Where they differ, however, is that nc-Si has small grains of crystalline silicon within the amorphous phase. This is in contrast to polycrystalline silicon (poly-Si), which consists solely of crystalline silicon grains, separated by grain boundaries. The difference comes solely from the grain size of the crystalline grains. Most materials with grains in the micrometer range are actually fine-grained polysilicon, so nanocrystalline (NC) silicon is a better term. The term nanocrystalline (NC) silicon refers to a range of materials around the transition region from amorphous to microcrystalline phase in the silicon thin film. The crystalline volume fraction (as measured from Raman spectroscopy) is another criterion to describe the materials in this transition zone. nc-Si has many useful advantages over a-Si, one being that if grown properly it can have a higher electron mobility, due to the presence of the silicon crystallites. It also shows increased absorption in the red and infrared wavelengths, which make it an important material for use in a-Si solar cells. One of the most important advantages of nanocrystalline (NC) silicon, however, is that it has increased stability over a-Si, one of the reasons being because of its lower hydrogen concentration. Although it currently cannot attain the mobility that poly-Si can, it has the advantage over poly-Si that it is easier to fabricate, as it can be deposited using conventional low temperature a-Si deposition techniques, such as PECVD, as opposed to laser annealing or high temperature CVD processes, in the case of poly-Si.

Nanocrystalline Thin-Film Solar Cell (TFSC) is also called a thin-film photovoltaic cell (TFPV), is a second generation solar cell that is made by depositing one or more thin layers, or thin film (TF) of photovoltaic material on a substrate, such as glass, plastic or metal. Thin-film solar cells are commercially used in several technologies, including cadmium telluride (CdTe), copper indium gallium diselenide (CIGS), and amorphous and other thin-film silicon (a-Si, TF-Si). Film thickness varies from a few nanometers (nm) to tens of micrometers (μm), much thinner than thin-film's rival technology, the conventional, first-generation crystalline silicon solar cell (c-Si), that uses silicon wafers of up to 200 μm. This allows thin film cells to be flexible, lower in weight, and have less drag. It is used in building integrated photovoltaics and as semi-transparent, photovoltaic glazing material that can be laminated onto windows. Other commercial applications use rigid thin film solar panels (sandwiched between two panes of glass) in some of the world's largest photovoltaic power stations. Thin-film has always been cheaper but less efficient than conventional c-Si technology. However, they significantly improved over the years, and lab cell efficiency for CdTe and CIGS are now beyond 21%, outperforming multicrystalline silicon, the dominant material currently used in most solar PV systems. Despite these enhancements, market-share of thin-film never reached more than 20% in the last two decades and has been declining in recent years to about 9% of worldwide photovoltaic production in 2013. Other thin-film technologies, that are still in an early stage of ongoing research or with limited commercial availability, are often classified as emerging or third generation photovoltaic cells and include, organic, dye-sensitized, and polymer solar cells, as well as quantum dot, copper zinc tin sulfide, nanocrystal, micromorph and perovskite solar cells.

Nanocrystalline Soft Magnetic Materials. Nanocrystalline structures offer a new opportunity for tailoring soft magnetic materials. The most prominent example are devitrified glassy FeCuNbSiB alloys which reveal a homogeneous ultrafine grain structure of bcc-FeSi with grain sizes of typically 10-15 nm and random texture. Owing to the small grain size the local magneto-crystalline anisotropy is randomly averaged out by exchange interaction so that there is only a small anisotropy net-effect on the magnetization process. Moreover the structural phases present lead to low or vanishing saturation magnetostriction, which minimizes magneto-elastic anisotropies. Both the suppressed magnetocrystalline anisotropy and the low magnetostriction provide the basis for the superior soft magnetic properties comparable to those of permalloys or near zero-magnetostrictive Co-base amorphous alloys but at a higher saturation induction. Like in other soft magnetic material the hysteresis loop can be tailored by uniaxial anisotropies induced by magnetic field annealing.

Nanocrystalline ZnO Thin Film. Nanocrystalline ZnO thin film can optionally be used as filters to purify liquids for water purification and making saltwater drinkable via evaporation of Zn metal on a glass sheet following by calcination (oxidation) process for photocatalytic purification of water. The influences of calcination parameters such as temperature and time on the surface morphology and phase structure of ZnO films were investigated by scanning electron microscopy (SEM) and X-ray diffraction (XRD), respectively. The analysis of XRD patterns indicated that the growth of ZnO nano-structure was controlled by calcination time and temperature. Optimum ZnO nano-fibers can be formed uniformly after 2 h of oxidation at 550° C. Nanostructured ZnO catalyst exhibited a significantly greater superiority for the photodegradation of 2,4,6-Trichlorophenol (TCP) as a model pollutant in water over photolysis via irradiation with UV of 254 nm wavelength.

Nanocellulose Dimensions and Crystallinity can optionally include an ultrastructure of cellulose derived from various sources has been extensively studied. Techniques such as transmission electron microscopy (TEM), scanning electron microscopy (SEM), atomic force microscopy (AFM), wide angle X-ray scattering (WAXS), small incidence angle X-ray diffraction and solid state C cross-polarization magic angle spinning (CP/MAS) nuclear magnetic resonance (NMR) spectroscopy have been used to characterize nanocellulose morphology. These methods have typically been applied for the investigation of dried nanocellulose morphology. Although a combination of microscopic techniques with image analysis can provide information on nanocellulose fibril widths, it is more difficult to determine nanocellulose fibril lengths because of entanglements and difficulties in identifying both ends of individual nanofibrils. It has been reported that nanocellulose suspensions may not be homogeneous and that they consist of various structural components, solar panels, solar cells, silicon thin films, including cellulose nanofibrils and nanofibril bundles. Most methods have typically been applied to investigation of dried nanocellulose dimensions, although a study was conducted where the size and size-distribution of enzymatically pre-treated nanocellulose fibrils in a suspension was studied using cryo-TEM. The fibrils were found to be rather mono-dispersed mostly with a diameter of ca. 5 nm although occasionally thicker fibril bundles were present. It should be noted that, some newly published results indicated that by combining ultrasonication with an “oxidation pretreatment,” cellulose microfibrils with a lateral dimension that belows 1 nm is observed by AFM. The lower end of the thickness dimension is around 0.4 nm, which is related to the thickness of a cellulose monolayer sheet. The aggregate widths can be determined by CP/MAS NMR developed by Innventia AB, Sweden, which also has been demonstrated to work for nanocellulose (enzymatic pre-treatment). An average width of 17 nm has been measured with the NMR-method, which corresponds well with SEM and TEM. Using TEM, values of 15 nm have been reported for nanocellulose from carboxymethylated pulp. However, also thinner fibrils can be detected. Wågberg et al. reported fibril widths of 5-15 nm for a nanocellulose with a charge density of about 0.5 meq./g. The group of Isogai reported fibril widths of 3-5 nm for TEMPO-oxidized cellulose having a charge density of 1.5 meq./g. The influence of cellulose pulp chemistry on the nanocellulose microstructure has been investigated using AFM to compare the microstructure of two types of nanocellulose prepared at Innventia AB (enzymatically pre-treated nanocellulose and carboxymethylated nanocellulose). Due to the chemistry involved in producing carboxymethylated nanocellulose, it differs significantly from the enzymatically pre-treated one. The carboxymethylation pre-treatment makes the fibrils highly charged and, hence, easier to liberate, which results in smaller and more uniform fibril widths (5-15 nm) compared to the enzymatically pre-treated nanocellulose, where the fibril widths were 10-30 nm. The degree of crystallinity and the cellulose crystal structure of nanocellulose were also studied at the same time. The results clearly showed the nanocellulose exhibited cellulose crystal I organization and that the degree of crystallinity was unchanged by the preparation of the nanocellulose. Typical values for the degree of crystallinity were around 63%.

Viscosity. The unique rheology of nanocellulose dispersions was recognized by the early investigators. The high viscosity at low nanocellulose concentrations makes nanocellulose very interesting as a non-caloric stabilizer and gellant in food applications, the major field explored by the early investigators. The dynamic rheological properties were investigated in great detail and revealed that the storage and loss modulus were independent of the angular frequency at all nanocellulose concentrations between 0.125% to 5.9%. The storage modulus values are particularly high (104 Pa at 3% concentration) compared to results for other cellulose nanowhiskers (102 Pa at 3% concentration). There is also a particular strong concentration dependence as the storage modulus increases 5 orders of magnitude if the concentration is increased from 0.125% to 5.9%. Nanocellulose gels are also highly shear thinning (the viscosity is lost upon introduction of the shear forces). The shear-thinning behavior is particularly useful in a range of different coating applications.

Mechanical Properties. Crystalline cellulose has interesting mechanical properties for use in material applications. Its tensile strength is about 500 MPa, similar to that of aluminum. Its stiffness is about 140-220 GPa, comparable with that of Kevlar and better than that of glass fiber, both of which are used commercially to reinforce plastics. Films made from nanocellulose have high strength (over 200 MPa), high stiffness (around 20 GPa) and high strain (12%). Its strength/weight ratio is 8 times that of stainless steel.

Barrier Properties. In semi-crystalline polymers, the crystalline regions are considered to be gas impermeable. Due to relatively high crystallinity, in combination with the ability of the nanofibers to form a dense network held together by strong inter-fibrillar bonds (high cohesive energy density), it has been suggested that nanocellulose might act as a barrier material. Although the number of reported oxygen permeability values are limited, reports attribute high oxygen barrier properties to nanocellulose films. One study reported an oxygen permeability of 0.0006 (cm³ μm)/(m² day kPa) for a ca. 5 μm thin nanocellulose film at 23° C. and 0% RH. In a related study, a more than 700-fold decrease in oxygen permeability of a polylactide (PLA) film when a nanocellulose layer was added to the PLA surface was reported. The influence of nanocellulose film density and porosity on film oxygen permeability has recently been explored. Some authors have reported significant porosity in nanocellulose films, which seems to be in contradiction with high oxygen barrier properties, whereas Aulin et al. measured a nanocellulose film density close to density of crystalline cellulose (cellulose Iβ crystal structure, 1.63 g/cm³) indicating a very dense film with a porosity close to zero. Changing the surface functionality of the cellulose nanoparticles can also affect the permeability of nanocellulose films. Films constituted of negatively charged cellulose nanowhiskers could effectively reduce permeation of negatively charged ions, while leaving neutral ions virtually unaffected. Positively charged ions were found to accumulate in the membrane.

Foams. Nanocellulose can optionally be used to make aerogels/foams, either homogeneously or in composite formulations. Nanocellulose-based foams are being studied for packaging applications in order to replace polystyrene-based foams. Svagan et al. showed that nanocellulose has the ability to reinforce starch foams by using a freeze-drying technique. The advantage of using nanocellulose instead of wood-based pulp fibers is that the nanofibrills can reinforce the thin cells in the starch foam. Moreover, it is possible to prepare pure nanocellulose aerogels applying various freeze-drying and super critical CO drying techniques. Aerogels and foams can optionally be used as porous templates. A wide range of mechanical properties including compression was obtained by controlling density and nanofibrill interaction in the foams. Cellulose nanowhiskers could also be made to gel in water under low power sonication giving rise to aerogels with the highest reported surface area (>600 m2/g) and lowest shrinkage during drying (6.5%) of cellulose aerogels. In another study by Aulin et al., the formation of structured porous aerogels of nanocellulose by freeze-drying was demonstrated. The density and surface texture of the aerogels was tuned by selecting the concentration of the nanocellulose dispersions before freeze-drying. Chemical vapor deposition of a fluorinated silane was used to uniformly coat the aerogel to tune their wetting properties towards non-polar liquids/oils. It is possible to switch the wettability behavior of the cellulose surfaces between super-wetting and super-repellent, using different scales of roughness and porosity created by the freeze-drying technique and change of concentration of the nanocellulose dispersion. Structured porous cellulose foams can however also optionally be obtained by utilizing the freeze-drying technique on cellulose generated by Gluconobacter strains of bacteria, which bio-synthesize open porous networks of cellulose fibers with relatively large amounts of nanofibrills dispersed inside. Olsson et al. demonstrated that these networks can be further impregnated with metalhydroxide/oxide precursors, which can readily be transformed into grafted magnetic nanoparticles along the cellulose nanofibers. The magnetic cellulose foam may allow for a number of novel applications of nanocellulose and the first remotely actuated magnetic super sponges absorbing 1 gram of water within a 60 mg cellulose aerogel foam were reported. Notably, these highly porous foams (>98% air) can be compressed into strong magnetic nanopapers, which may find use as functional membranes in various applications.

Plant Breeding is the art and science of changing the traits of plants in order to produce desired characteristics. Plant breeding can be accomplished through many different techniques ranging from simply selecting plants with desirable characteristics for propagation, to more complex molecular techniques such as cultigen and cultivar. Plant breeding has been practiced for thousands of years, since near the beginning of human civilization. It is now practiced worldwide by individuals such as gardeners and farmers, or by professional plant breeders employed by organizations such as government institutions, universities, crop-specific industry associations or research centers. International development agencies believe that breeding new crops is important for ensuring food security by developing new varieties that are higher-yielding, resistant to pests and diseases, drought-resistant or regionally adapted to different environments and growing conditions.

RNA Splicing. In molecular biology and genetics, splicing is a modification of the nascent pre-messenger RNA (pre-mRNA) transcript in which introns are removed and exons are joined. For nuclear encoded genes, splicing takes place within the nucleus after or concurrently with transcription. Splicing is needed for the typical eukaryotic messenger RNA (mRNA) before it can be used to produce a correct protein through translation. For many eukaryotic introns, splicing is done in a series of reactions which are catalyzed by the spliceosome, a complex of small nuclear ribonucleoproteins (snRNPs), but there are also self-splicing introns. Several methods of RNA splicing occur in nature; the type of splicing depends on the structure of the spliced intron and the catalysts required for splicing to occur. Spliceosomal. Introns. The word intron is derived from the term intragenic region, that is, a region inside a gene. The term intron refers to both the DNA sequence within a gene and the corresponding sequence in the unprocessed RNA transcript. As part of the RNA processing pathway, introns are removed by RNA splicing either shortly after or concurrent with transcription. Introns are found in the genes of most organisms and many viruses. They can be located in a wide range of genes, including those that generate proteins, ribosomal RNA (rRNA), and transfer RNA (tRNA). Spliceosomal introns often reside within the sequence of eukaryotic protein-coding genes. Within the intron, a donor site (5′ end of the intron), a branch site (near the 3′ end of the intron) and an acceptor site (3′ end of the intron) are required for splicing. The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), or polypyrimidine tract. Upstream from the polypyrimidine tract is the branchpoint, which includes an adenine nucleotide. The consensus sequence for an intron (in IUPAC nucleic acid notation) is: A-G-[cut]-G-U-R-A-G-U (donor site) . . . intron sequence . . . Y-U-R-A-C (branch sequence 20-50 nucleotides upstream of acceptor site) . . . Y-rich-N-C-A-G-[cut]-G (acceptor site). However, it is noted that the specific sequence of intronic splicing elements and the number of nucleotides between the branchpoint and the nearest 3′ acceptor site affect splice site selection. Also, point mutations in the underlying DNA or errors during transcription can activate a cryptic splice site in part of the transcript that usually is not spliced. This results in a mature messenger RNA with a missing section of an exon. In this way, a point mutation, which usually only affects a single amino acid, can manifest as a deletion in the final protein. Formation and activity. Splicing is catalyzed by the spliceosome, a large RNA-protein complex composed of five small nuclear ribonucleoproteins (snRNPs, pronounced ‘snurps’). Assembly and activity of the spliceosome occurs during transcription of the pre-mRNA. The RNA components of snRNPs interact with the intron and are involved in catalysis. Two types of spliceosomes have been identified (major and minor) which contain different snRNPs.

The major spliceosome splices introns containing GU at the 5′ splice site and AG at the 3′ splice site. It is composed of the U1, U2, U4, U5, and U6 snRNPs and is active in the nucleus. In addition, a number of proteins including U2 small nuclear RNA auxiliary factor 1 (U2AF35), U2AF2 (U2AF65) and SF1 are required for the assembly of the spliceosome. The spliceosome forms different complexes during the splicing process: Complex E, the U1 snRNP binds to the GU sequence at the 5′ splice site of an intron; Splicing factor 1 binds to the intron branchpoint sequence; U2AF1 binds at the 3′ splice site of the intron; U2AF2 binds to the polypyrimidine tract; Complex A (pre-spliceosome), the U2 snRNP displaces SF1 and binds to the branchpoint sequence and ATP is hydrolyzed; Complex B (pre-catalytic spliceosome), the U5/U4/U6 snRNP trimer binds, and the U5 snRNP binds exons at the 5′ site, with U6 binding to U2. Complex B, the U1 snRNP is released, U5 shifts from exon to intron, and the U6 binds at the 5′ splice site. Complex C (catalytic spliceosome). U4 is released, U6/U2 catalyzes transesterification, making the 5′-end of the intron ligate to the A on intron and form a lariat, U5 binds exon at 3′ splice site, and the 5′ site is cleaved, resulting in the formation of the lariat. Complex C* (post-spliceosomal complex). U2/U5/U6 remain bound to the lariat, and the 3′ site is cleaved and exons are ligated using ATP hydrolysis. The spliced RNA is released, the lariat is released and degraded, and the snRNPs are recycled. This type of splicing is termed canonical splicing or termed the lariat pathway, which accounts for more than 99% of splicing. By contrast, when the intronic flanking sequences do not follow the GU-AG rule, noncanonical splicing is said to occur (see “minor spliceosome” below). The minor spliceosome is very similar to the major spliceosome, but instead it splices out rare introns with different splice site sequences. While the minor and major spliceosomes contain the same U5 snRNP, the minor spliceosome has different but functionally analogous snRNPs for U1, U2, U4, and U6, which are respectively called U11, U12, U4atac, and U6atac. Unlike the major spliceosome, it is found outside the nucleus, but very close to the nuclear membrane. Trans-splicing is a form of splicing that joins two exons that are not within the same RNA transcript.

Self-Splicing. Self-splicing occurs for rare introns that form a ribozyme, performing the functions of the spliceosome by RNA alone. There are three kinds of self-splicing introns, Group I, Group II and Group III. Group I and II introns perform splicing similar to the spliceosome without requiring any protein. This similarity suggests that Group I and II introns may be evolutionarily related to the spliceosome. Self-splicing may also be very ancient, and may have existed in an RNA world present before protein. Two transesterifications characterize the mechanism in which group I introns are spliced: 3′OH of a free guanine nucleoside (or one located in the intron) or a nucleotide cofactor (GMP, GDP, GTP) attacks phosphate at the 5′ splice site, 3′OH of the 5′exon becomes a nucleophile and the second transesterification results in the joining of the two exons. The mechanism in which group II introns are spliced (two transesterification reaction like group I introns) is as follows: The 2′OH of a specific adenosine in the intron attacks the 5′ splice site, thereby forming the lariat, the 3′OH of the 5′ exon triggers the second transesterification at the 3′ splice site thereby joining the exons together.

tRNA Splicing. tRNA (also tRNA-like) splicing is another rare form of splicing that usually occurs in tRNA. The splicing reaction involves a different biochemistry than the spliceosomal and self-splicing pathways. In the yeast Saccharomyces cerevisiae, a yeast tRNA splicing endonuclease heterotetramer, composed of TSEN54, TSEN2, TSEN34, and TSEN15, cleaves pre-tRNA at two sites in the acceptor loop to form a 5′-half tRNA, terminating at a 2′,3′-cyclic phosphodiester group, and a 3′-half tRNA, terminating at a 5′-hydroxyl group, along with a discarded intron. Yeast tRNA kinase then phosphorylates the 5′-hydroxyl group using adenosine triphosphate. Yeast tRNA cyclic phosphodiesterase cleaves the cyclic phosphodiester group to form a 2′-phosphorylated 3′ end. Yeast tRNA ligase adds an adenosine monophosphate group to the 5′ end of the 3′-half and joins the two halves together. NAD-dependent 2′-phosphotransferase then removes the 2′-phosphate group.

Evolution. Splicing occurs in all the kingdoms or domains of life, however, the extent and types of splicing can be very different between the major divisions. Eukaryotes splice many protein-coding messenger RNAs and some non-coding RNAs. Prokaryotes, on the other hand, splice rarely and mostly non-coding RNAs. Another important difference between these two groups of organisms is that prokaryotes completely lack the spliceosomal pathway. Because spliceosomal introns are not conserved in all species, there is debate concerning when spliceosomal splicing evolved. Two models have been proposed: the intron late and intron early models.

Biochemical Mechanism. Spliceosomal splicing and self-splicing involves a two-step biochemical process. Both steps involve transesterification reactions that occur between RNA nucleotides. tRNA splicing, however, is an exception and does not occur by transesterification. Spliceosomal and self-splicing transesterification reactions occur via two sequential transesterification reactions. First, the 2′OH of a specific branchpoint nucleotide within the intron, defined during spliceosome assembly, performs a nucleophilic attack on the first nucleotide of the intron at the 5′ splice site forming the lariat intermediate. Second, the 3′OH of the released 5′ exon then performs a nucleophilic attack at the last nucleotide of the intron at the 3′ splice site, thus joining the exons and releasing the intron lariat. Alternative splicing. In many cases, the splicing process can create a range of unique proteins by varying the exon composition of the same mRNA. This phenomenon is then called alternative splicing. Alternative splicing can occur in many ways. Exons can be extended or skipped, or introns can be retained. It is estimated that 95% of transcripts from multiexon genes undergo alternative splicing, some instances of which occur in a tissue-specific manner and/or under specific cellular conditions. Development of high throughput mRNA sequencing technology can help quantify the expression levels of alternatively spliced isoforms. Differential expression levels across tissues and cell lineages allowed computational approaches to be developed to predict the functions of these isoforms. Given this complexity, alternative splicing of pre-mRNA transcripts is regulated by a system of trans-acting proteins (activators and repressors) that bind to cis-acting sites or “elements” (enhancers and silencers) on the pre-mRNA transcript itself. These proteins and their respective binding elements promote or reduce the usage of a particular splice site. However, adding to the complexity of alternative splicing, it is noted that the effects of regulatory factors are many times position-dependent. For example, a splicing factor that serves as a splicing activator when bound to an intronic enhancer element may serve as a repressor when bound to its splicing element in the context of an exon, and vice versa. In addition to the position-dependent effects of enhancer and silencer elements, the location of the branchpoint (i.e., distance upstream of the nearest 3′ acceptor site) also affects splicing. The secondary structure of the pre-mRNA transcript also plays a role in regulating splicing, such as by bringing together splicing elements or by masking a sequence that would otherwise serve as a binding element for a splicing factor. Experimental manipulation of splicing Splicing events can be experimentally altered by binding steric-blocking antisense oligos such as Morpholinos or Peptide nucleic acids to snRNP binding sites, to the branchpoint nucleotide that closes the lariat, or to splice-regulatory element binding sites.

Radio-Frequency Identification (RFID) is the wireless use of electromagnetic fields to transfer data, for the purposes of automatically identifying and tracking tags attached to objects. The tags contain electronically stored information. Some tags are powered by electromagnetic induction from magnetic fields produced near the reader. Some types collect energy from the interrogating radio waves and act as a passive transponder. Other types have a local power source such as a battery and may operate at hundreds of meters from the reader Unlike a barcode, the tag does not necessarily need to be within line of sight of the reader and may be embedded in the tracked object. RFID is one method for Automatic Identification and Data Capture (AIDC). RFID tags are used in many industries. For example, an RFID tag attached to an automobile during production can be used to track its progress through the assembly line; RFID-tagged pharmaceuticals can be tracked through warehouses; and implanting RFID microchips in livestock and pets allows positive identification of animals. Since RFID tags can be attached to cash, clothing, and possessions, or implanted in animals and people, the possibility of reading personally-linked information without consent has raised serious privacy concerns.

Solar Cell, or Photovoltaic Cell, is an electrical device that converts the energy of light directly into electricity by the photovoltaic effect, which is a physical and chemical phenomenon. It is a form of photoelectric cell, defined as a device whose electrical characteristics, such as current, voltage, or resistance, vary when exposed to light. Solar cells are the building blocks of photovoltaic modules, otherwise known as solar panels, solar cells. Solar cells are described as being photovoltaic irrespective of whether the source is sunlight or an artificial light. They are used as a photodetector (for example infrared detectors), detecting light or other electromagnetic radiation near the visible range, or measuring light intensity. The operation of a photovoltaic (PV) cell requires 3 basic attributes: The absorption of light, generating either electron-hole pairs or excitons. The separation of charge carriers of opposite types. The separate extraction of those carriers to an external circuit. In contrast, a solar thermal collector supplies heat by absorbing sunlight, for the purpose of either direct heating or indirect electrical power generation from heat. A “photoelectrolytic cell” (photoelectrochemical cell), on the other hand, refers either to a type of photovoltaic cell (like that developed by Edmond Becquerel and modern dye-sensitized solar cells), or to a device that splits water directly into hydrogen and oxygen using only solar illumination.

Surface Modification. The surface modification of nanocellulose is currently receiving a large amount of attention. Nanocellulose displays a high concentration of hydroxyl groups at the surface, which can be reacted. However, hydrogen bonding strongly affects the reactivity of the surface hydroxyl groups. In addition, impurities at the surface of nanocellulose such as glucosidic and lignin fragments need to be removed before surface modification to obtain acceptable reproducibility between different batches. Cellulose nano fiber can optionally be modified as cationic. The cationic cellulose increases the affinity for anions.

Surface Acoustic Wave (SAW) devices based on polycrystalline diamond have recently achieved success as microwave filters. This is due in part to the large acoustic wavelength of diamond at microwave frequencies, a consequence of its high surface wave velocity, and the resulting ability to use photolithography for transducer fabrication. Since nanocrystalline diamond has a smooth surface and is elastically isotropic, it may offer considerable advantages over thick films of polycrystalline diamond. Studies have been made of the propagation of surface waves on nanocrystalline diamond prepared by microwave plasma chemical vapor deposition (CVD) on silicon substrates. Films were synthesized on 75-mm Si wafers using input gas mixtures consisting of Ar with 1% CH₄ and 0-4% H. The deposition parameters studied included pressure, 2.45 GHz microwave power, and total gas flow rate. Film thicknesses up to 23 μm were produced. SAW transducers were fabricated by photolithography on as-grown nanocrystalline diamond surfaces covered with a 1-3 μm over layer of oriented polycrystalline piezoelectric ZnO prepared by reactive dc sputtering. The device response was analyzed with frequency and time domain methods. The resonant frequencies of the devices agree with the results of numerical solutions for sound propagation in layered media. Several surface acoustic modes exist at frequencies between 0.5 and 1 GHz that exhibit appreciable dispersion. The surface waves in nanocrystalline diamond over distances varying from 0.1 to 3 mm with low attenuation. For a film with mean grain size of approximately 30 nm, the SAW velocity is similar to test devices on thick polycrystalline diamond. Nanocrystalline diamond is a highly attractive substrate material for SAW devices, possessing the high sound velocity of diamond but requiring less materials processing.

Safety Aspects. Health, safety and environmental aspects of nanocellulose have been recently evaluated. Processing of nanocellulose does not cause significant exposure to fine particles during friction grinding or spray drying. No evidence of inflammatory effects or cytotoxicity on mouse or human macrophages can be observed after exposure to nanocellulose. The results of toxicity studies suggest that nanocellulose is not cytotoxic and does not cause any effects on inflammatory system in macrophages. In addition, nanocellulose is not acutely toxic to Vibrio fischeri in environmentally relevant concentrations.

Nanocellulose Applications. The properties of nanocellulose (e.g. mechanical properties, film-forming properties, viscosity etc.). makes it an interesting material for many applications and the potential for a multi-billion dollar industry.

Composite. As described above the properties of the nanocellulose makes an interesting material for reinforcing plastics. Nanocellulose has been reported to improve the mechanical properties of, for example, thermosetting resins, starch-based matrixes, soy protein, rubber latex, poly (lactide). The composite applications may be for use as coatings and films, paints, foams, packaging.

Food Additives. Nanocellulose can optionally be used as a low calorie replacement for today's carbohydrate additives used as thickeners, flavor carriers and suspension stabilizers in a wide variety of food additives, food ingredients and food products that is useful for producing fillings, crushes, chips, wafers, soups, gravies, puddings etc. The food packaging applications were early recognized as a highly interesting application field for nanocellulose due to the rheological behavior of the nanocellulose gel.

Hygiene and Absorbent Products. Different nanocrystalline (NC) applications in this field include: super water absorbent (e.g. for incontinence pads material, Nanocellulose used together with super absorbent polymers, use of nanocellulose in tissue, non-woven products or absorbent structures, use as antimicrobial films.

Emulsion and Dispersion. Apart from the numerous applications in the area of food additives, the general area of emulsion and dispersion applications in other fields has also received some attention. Oil in water applications were early recognized. The area of non-settling suspensions for pumping sand, coal as well as paints and drilling muds was also explored by the early investigators.

Oil Recovery. Hydrocarbon fracturing of oil-bearing formations is a potentially interesting and large-scale application. Nanocellulose has been suggested for use in oil recovery applications as a fracturing fluid. Drilling muds based on nanocellulose have also been suggested.

Viral Inhibitor. NCC crystals can optionally be designed to adsorb viruses and disable them through the use of antiviral ointments and surfaces providing protection against viruses, spread by mosquitoes, by applying ointment containing nanocrystalline cellulose onto the skin. Nanocrystalline cellulose applied, in a non-liquid form, on hospital door handles could kill viruses and prevent them from spreading.

Medical, Cosmetic, Health and Pharmaceutical Industries. The use of nanocellulose in medical, cosmetic additives, and pharmaceutical products include a wide range of high-end applications have been suggested: Freeze-dried nanocellulose aerogel, resins used in sanitary napkins, tampons, diapers or as wound dressing, use of nano cellulose as a composite nanocrystalline (NC) coating agent in cosmetic additives, sugar substitute, sweeteners, artificial sweeteners, amino acid regulators, acidity regulators, anticaking agents, applications as taste masking agents, disintegrating agents, binders in granulation process, fillers in solid dosage forms, thickening and stabilizing agents, gelling agents, compressibility enhancers, coating agents, drug delivery systems, pharmaceutical coatings process, medical coating applications, topical ophthalmic protectant and lubricant and the like, antifoaming agents, antibacterial agents, anti-aging products, antioxidants, absorption blocking agents, carcinogen blocking agents, cellulose geltain capsules for dietary supplements, medications, vitamins, marijuana oils, cannabis oils, hash oils, hemp oils and other types of oils for cancer treatment, pharmaceutical uses and other medical uses, encapsulation products, cholesterol blocking agents, fat blocking agents, caloric blocking agents, blocking sugar absorption, neuromuscular blocking agents, food coloring, color retention agents, emulsifiers, natural or artificial flavors, flavor enhancers, flour treatment agent, glazing agents, humectants, tracer gas, preservatives, stabilizers, thickeners, smart packaging and intelligent labeling technology for food, beverages, pharmaceutical and household products, sunscreens, coatings e.g., for hair, eyelashes, eyebrows or nails, a dry solid nanocellulose composition in the form of tablets for treating intestinal orders, nanocellulose films for screening of biological compounds and nucleic acids encoding a biological compound, filter medium partly based on nanocellulose for leukocyte free blood transfusion, a buccodental formulation, comprising nanocellulose and a polyhydroxylated organic compound, powdered nanocellulose has also been suggested as an excipient in pharmaceutical compositions, nanocellulose in compositions of a photoreactive noxious substance purging agent, elastic cryo-structured gels for potential biomedical and biotechnological application.

Different Cellulose Applications. Activate the dissolution of cellulose in different solvents, regenerated cellulose products, such as fibers films, cellulose derivatives, tobacco filter additive, organometallic modified nanocellulose in battery separators, reinforcement of conductive materials, loud-speaker membranes, high-flux membrane, flexible electronic displays, computer components, lightweight body armor and ballistic glass.

Wood Pulp. Nanocellulose/CNF or NCC can be prepared from any cellulose source material, but wood pulp is normally used. The nanocellulose fibrils may be isolated from the wood-based fibers using mechanical methods, which expose the pulp to high shear forces, ripping the larger wood-fibers apart into nanofibers. For this purpose high-pressure homogenizers, ultrasonic homogenizers, grinders or microfluidizer. The homogenizers are used to delaminate the cell walls of the fibers and liberate the nanosized fibrils. This process is responsible for the high-energy consumptions associated with the fiber delamination. Values over 30 MWh/tonne are not uncommon. Pre-treatments are sometimes used to address this problem. Examples of such pre-treatments are enzymatic/mechanical pre-treatment and introduction of charged groups e.g. through carboxymethylation or TEMPO-mediated oxidation. Cellulose nanowhiskers, a more crystalline form of nanocellulose, are formed by the acid hydrolysis of native cellulose fibers commonly using sulfuric or hydrochloric acid. The amorphous sections of native cellulose are hydrolysed and after careful timing, the crystalline sections can be retrieved from the acid solution by centrifugation and washing. Cellulose nanowhiskers are rod like highly crystalline particles (relative crystallinity index above 75%) with a rectangular cross section. Their dimensions depend on the native cellulose source material, and hydrolysis time and temperature.

Nanocrystalline Powder. Consolidation of nanocrystalline powders can optionally be achieved by electrodischarge compaction, plasma-activated sintering, shock (explosive) consolidation, hot-isostatic pressing (HIP), Ceracon processing (the Ceracon process (CERAmic CONsolidation) involves taking a heated preform and consolidating the material by pressure against a granular ceramic medium using a conventional forging press), hydrostatic extrusion, strained powder rolling, and sinter forging. By utilizing the combination of high temperature and pressure, HIP can achieve a particular density at lower pressure when compared to cold isostatic pressing or at lower temperature when compared to sintering. It should be noted that because of the increased diffusivity in nanocrystalline (NC) materials, nanocrystalline (NC) components, sintering (densification) takes place at temperatures much lower than in coarse-grained materials. This is likely to reduce the grain growth. Nanocrystalline (NC) materials have been shown to exhibit increased strength/hardness, enhanced diffusivity, reduced density, higher electrical resistivity, increased specific heat, higher coefficient of thermal expansion, lower thermal conductivity, insulation, and superior soft magnetic properties in comparison to their coarse-grained counterparts. But, subsequent careful investigations on fully dense nanocrystalline ma-C. Suryanarayana, C. C. Koch/Nanocrystalline (NC) materials, nanocrystalline (NC) components, e.g., those produced by electrodeposition methods, have indicated that at least some of the “significant” changes in these properties could be attributed to the presence of porosity, cracks, and other discontinuities in the processed materials. Thus, it becomes important to obtain fully dense materials while simultaneously retaining nanometer-sized grains to unambiguously demonstrate the improvement in properties due to nanostructure processing.

Nanotechnology or Nanorobotics is the technology field creating machines or robots whose components are at or close to the scale of ananometer (10⁻⁹ meters). More specifically, nanorobotics refers to the nanotechnology engineering discipline of designing and/or building nanorobots, with devices ranging in size from 0.1-10 micrometers and/or constructed of nanoscale or molecular components.

Nanobots, nanoids, nanites, nanomachines or nanomites have also been used to describe these devices currently under research and/or development. Another definition is a robot that allows precision communications with nanoscale objects, or can optionally manipulate with nanoscale resolution. Such devices are more related to microscopy or scanning probe microscopy, instead of the description of nanorobots as molecular machine. Following the microscopy definition even a large apparatus such as an atomic force microscope can optionally be considered a nanorobotic instrument when configured to perform nanomanipulation. For this perspective, macroscale robots or microrobots that can optionally move with nanoscale precision can optionally also be considered nanorobots. Nanotechnology can optionally be used for the detection of diseases and/or conditions.

Nanocrystalline Coating Applications. The deposition of ultra hard nanocrystalline (NC) coating applications based on titanium nitride by a vacuum arc method with plasma assistance, investigations of their structural features, physical and mechanical properties are presented. The materials of the evaporated cathode were the sintered Ti—Al and Ti—Cu system materials. It should be noted that one of the chosen additional elements (Al) forms nitride compounds, and another (Cu) doesn't form that at the conditions of coating synthesis. Obtaining of experimental data for explanation of formation model of ultra hard nanocrystalline (NC) coating applications based on titanium nitride at addition of different elements in their structure was the main purpose of work. For achievement of that the methods such as the scanning electron microscopy, transmission electron microscopy, Auger spectrometry, the X-ray fluorescent and X-ray diffraction analysis with the use of synchrotron radiation were used. In addition the experimental results on research of near-edge fine structure of X-ray absorption spectrum (X-ray Absorption Near-Edge Structure, XANES) and extended fine structure of X-ray absorption (Extended X-ray Absorption Fine Structure, EXAFS) in the field of K-edge of titanium absorption in the samples of titanium nitride coating applications at copper and aluminum addition are also presented. Transmission electron diffraction microscopy and X-ray diffraction analysis with the use of synchrotron radiation has revealed the fact that ultra hard (NC) Ti—Cu—N coating consist of randomly oriented nanocrystallites of the main phase δ-TiN. The average size of crystallites of the main phase is d=18 nm. The presence of crystal phases of copper in the coating applications wasn't observed. But by means of the X-ray fluorescent analysis the availability of copper in the coating with concentration of 12 at. % was revealed. That corresponds to concentration of additional element in the evaporated cathode. The carried-out measurements of XAFS spectrum confirm that atoms of copper are concentrated in the fine films limiting the sizes of TiN crystallites and don't possess the regular structure. For the first time it was succeeded to directly show the main role of an additional element in formation of nanocrystalline structure of coating applications based on titanium nitride as the result of the complex experiment researches. According to the model nanocrystallization in Ti—Cu—N coating is due to the added atoms (Cu), which form an amorphous sheath with thickness of 2-3 monolayers (0.74 nm) around TiN grains that restrict the grain growth. Experimental data about structure of coating applications based on TiN with aluminum addition in synthesized coating applications of Ti—Al—N by vacuum arc deposition with plasma assistance have been obtained by transmission electron microscopy and X-ray powder diffraction with a synchrotron radiation. These testified that the coating applications have multiphase structure, which depends on modes of coating deposition. Thus the processes of formation and breakdown of crystallographic phases in synthesized Ti—Al—N coating applications are non-equilibrium, thereof the main crystallographic phase of areas of coherent dispersion—TiN, and area of localization of crystallographic phases of aluminum nitride and the intermetallic phases of AlTi—boundaries of the main phase of a coating.

Nanocellulose is a term referring to nano-structured cellulose. This can optionally be either cellulose nanofibers (CNF) also called microfibrillated cellulose (MFC), Nanocrystalline cellulose (NCC), or bacterial nanocellulose, which refers to nano-structured cellulose produced by bacteria. CNF is a material composed of nanosized cellulose fibrils with a high aspect ratio (length to width ratio). Typical lateral dimensions are 5-20 nanometers and/or longitudinal dimension is in a wide-range, typically several micrometers. It is pseudo-plastic and/or exhibits the property of certain gels or fluids that are thick (viscous) under normal conditions, but flow (become thin, less viscous) over time when shaken, agitated, or otherwise stressed. This property is known as thixotropy. When the shearing forces are removed the gel regains much of its original state. The fibrils are isolated from any cellulose containing source including wood-based fibers (pulp fibers) through high-pressure, high temperature and/or high velocity impact homogenization, grinding or microfluidization. Nanocellulose can optionally also optionally be obtained from native fibers by an acid hydrolysis, giving rise to highly crystalline and/or rigid nanopproducts (often referred to as CNC or nanowhiskers), which are shorter (100 s to 1000 nanometers) than the nanofibrils obtained through the homogenization, microfluiodization or grinding routes. The resulting material is known as nanocrystalline cellulose (NCC). All plant materials contain a minimum of 25% cellulose. Wood from trees is a little higher, between 40% and/or 50%. In addition to being used as strengtheners in plastics, the nanocrystals can optionally be used in ceramics and/or in biomedical applications such as a viral inhibitor, antiviral ointments, artificial joints, antibacterial medical coating applications, disposable medical equipment, coatings for medical applications, medical implants, breast implant devices, microchip implants, biosensor implants or other types of implants, medical prostheses, coatings for medical applications, medical implants, breast implant devices, microchip implants or other types of implants, orthopedic implants, dental implants or other medical products, surgical devices, wound care products, disease-fighting and anti-aging products, nanocrystalline cellulose anti-aging products, antioxidants, drug carrier for treatment of cancer or other diseases. Using cellulosic nanocrystals to strengthen plastics has advantages over the glass that is often used: Glass is heavier, harder on processing machinery and/or therefore more expensive to work with, and/or it stays in the ground for centuries. The cellulose nanocrystals will break down quickly in a landfill. Production of nanocrystalline cellulose (NCC) starts with “purified” wood, which has had compounds such as lignin and/or hemicellulose removed. It is then milled into a pulp and/or hydrolysed in acid to remove impurities before being separated and/or concentrated as crystals into a thick paste that can optionally be applied to surfaces as a laminate or processed into strand/ors, forming nanofibrils. These are hard, dense and/or tough, and/or can optionally be forced into different shapes and/or sizes. When freeze-dried, the material is lightweight, absorbent and/or good at insulating.

Nanocrystalline (NC) Material is a polycrystalline material with a crystallite size of only a few nanometers. These materials fill the gap between amorphous materials without any long range order and/or conventional coarse-grained materials. Definitions vary, but nanocrystalline material is commonly defined as a crystallite (grain) size below 100 nm. Grain sizes from 100-500 nm are typically considered “ultrafine” grains. The grain size of a NC sample can optionally be estimated using x-ray diffraction. In materials with very small grain sizes, the diffraction peaks can optionally be broadened. This broadening can optionally be related to a crystallite size using the Scherrer equation (applicable up to ˜50 nm), a Williamson-Hall plot, or more sophisticated methods such as the Warren-Averbach method or computer modeling of the diffraction pattern. The crystallite size can optionally be measured directly using transmission electron microscopy. Other properties of nanocrystalline metals, apart from increased strength and/or hardness, including higher electrical resistance, increased specific heat capacity, thermal expansion, optical properties, mechanical properties, elastic properties, strength & hardness, ductility & toughness, electrical properties, magnetic properties, chemical properties, catalytic properties, barrier properties, nanocrystalline cores for large power transformers, lower thermal conductivity, insulation and/or improved thermal properties, optical properties, mechanical properties, elastic properties, strength & hardness, ductility & toughness, electrical properties, chemical properties, magnetic properties.

Nanocrystalline Cellulose (NCC) can optionally be used to improve the performance of polyvinyl acetate (PVA) as a wood adhesive. NCC can be added to PVA at different loadings (1%, 2% and 3%) and the blends were used as binder for wood. Block shear tests were done to evaluate bonding strength of PVA at different conditions; dry and wet conditions, at the elevated temperature (100° C.). The mechanical properties of PVA film and its composites with NCC were measured by nanoindentation technique. Thermal stability and structure of nanocomposites were studied by thermogravimetric analysis and atomic force microscopy (AFM). The block shear tests demonstrate that NCC can improve bonding strength of PVA in all conditions. Hardness, modulus of elasticity (MOE) and creep of PVA film were also changed positively by the addition of NCC. Thermal stability of PVA was significantly improved as NCC was added to PVA. Structural studies revealed that variations in shear strength and other properties can be related to the quality of NCC dispersion in the PVA matrix.

Nanocrystals can optionally include a material product having at least one dimension smaller than 100 nanometers (a nanoproduct) and/or composed of atoms in either a single- or poly-crystalline arrangement. The size of nanocrystals distinguishes them from larger crystals. For example, silicon nanocrystals can optionally provide efficient light emission while bulk silicon does not and/or can optionally be used for memory components. When embedded in solids nanocrystals can optionally exhibit much more complex melting behavior than conventional solids and/or can optionally form the basis of a special class of solids. They can optionally behave as single-domain systems (a volume within the system having the same atomic or molecular arrangement throughout) that can optionally help explain the behavior of macroscopic samples of a similar material without the complicating presence of grain boundaries and/or other defects. Semiconductor nanocrystals having dimensions smaller than 10 nm are also described as quantum dots. Nanocrystalline cellulose (NCC) exhibit remarkable thermal, optical, mechanical, elastic, strength, toughness, magnetic and chemical properties, which can be exploited in a wide variety of structural and/or nanostructural applications. Potential uses have been identified in the automotive, electronic, aerospace, flexible screens, flexible electronic displays, flat panel displays, bendable batteries, wearable batteries, ultra absorbent aerogels, clothing, transportation fuels, biofuels, liquid fuels, chemical, fuel, and/or lubrication industries with applications ranging from flat panel displays to disposable medical equipment, coatings for medical applications, medical implants, breast implant devices, microchip implants or other types of implants, artificial heart valves, artificial ligaments, hip joints, and the like, and other medical coating applications

Nanocrystalline (NC) Materials can optionally include, without limitation, e.g., phosphors, carbides, nickel, yttrium, ceramics, composite, grains, silicon, etc. Nanocrystalline material can optionally be classified into different categories depending on the number of dimensions in which the material has nanometer modulations. Thus, they can be classified into (a) layered or lamellar structures, (b) filamentary structures, and (c) equiaxed nanostructured materials. A layered or lamellar structure is a one-dimensional (1D) nanostructure in which the magnitudes of length and width are much greater than the thickness that is only a few nanometers in size. One can also visualize 8 C. Suryanarayana, C. C. Koch/Nanocrystalline (NC) materials Table 1 Classification of nanocrystalline (NC) materials. Dimensionality Designation Typical method(s) of synthesis One-dimensional (1D) Layered (lamellar) Vapor deposition Electrodeposition Two-dimensional (2D) Filamentary Chemical vapor deposition Three-dimensional (3D) Crystallites Gas condensation (equiaxed).

Nanocrystalline Cores have very high permeability over low frequency to high frequency up to 30 Mhz. They are very suitable for common mode choke to used as EMC filter to compress conducted common mode noise. Compared to traditional ferrite core, nanocrystalline core has a lot of advantages as high inductance, good filter effective, small size and volume, lower turns of copper wire, lower power consumption and high efficiency. Nanocrystalline cores have high curie temperature about 560° C., much higher than traditional ferrite core about 200° C. High curie temperature make nanocrystalline core excellent thermal stability, and can continuous working at up to 120° C. environment. Nanocrystalline cores are the best choice for application of common mode choke. Features, Material: Fe-based Nanocrystalline core, Saturation flux density induction: 1.25 T, Permeability @ 10 KHz: >50000, Permeability @ 100 KHz: >10500, Curie temperature (° C.): 560, Stacking factor: 0.78. Saturation magnetostriction (*10̂-6): <2, Resistivity (μΩ.cm): 115, Ribbon thickness: 25 μm. Core shapes: Troidal core. Applications for nano crystalline cores include: EMC Filter, Switched mode power supply, Computer power supply, Communication and network power supply, Laser and X-ray power supply, Welding equipment and Electrical plating power supply, Solar energy equipment and Wind power generator, Household electrical appliance, Uninterruptable power supply (UPS), Frequency converted, Inducted heating equipment, high-speed railway power supplies.

Nanocrystalline Copper. Nanocrystalline copper electrodes can optionally be used as the catalyzes for the electrochemical conversion of carbon monoxide to alcohols. The electrochemical conversion of CO2 and H2O into liquid fuel is ideal for high-density renewable energy storage and could provide an incentive for CO2 capture. However, efficient electrocatalysts for reducing CO2 and its derivatives into a desirable fuel are not available at present. Although many catalysts can reduce CO2 to carbon monoxide (CO), liquid fuel synthesis requires that CO is reduced further, using H2O as a H⁺ source. Copper (Cu) is the only known material with an appreciable CO electroreduction activity, but in bulk form its efficiency and selectivity for liquid fuel are far too low for practical use. In particular, H2O reduction to H2 outcompetes CO reduction on Cu electrodes unless extreme over potentials are applied, at which point gaseous hydrocarbons are the major CO reduction products. Nanocrystalline Cu prepared from Cu2O (‘oxide-derived Cu’) produces multi-carbon oxygenates (ethanol, acetate and n-propanol) with up to 57% Faraday efficiency at modest potentials (−0.25 volts to −0.5 volts versus the reversible hydrogen electrode) in CO-saturated alkaline H2O. By comparison, when prepared by traditional vapor condensation, Cu nanoparticles with an average crystallite size similar to that of oxide-derived copper produce nearly exclusive H2 (96% Faraday efficiency) under identical conditions. Our results demonstrate the ability to change the intrinsic catalytic properties of Cu for this notoriously difficult reaction by growing interconnected nanocrystallites from the constrained environment of an oxide lattice. The selectivity for oxygenates, with ethanol as the major product, demonstrates the feasibility of a two-step conversion of CO2 to liquid fuel that could be powered by renewable electricity.

Nanometer Dimensions are at the atomic dimension scale. Nanotechnology refers to the study, creation and/or application of molecular materials with a product size that is typically less than one nanometer (nm) is one billionth, or 10-9, of a meter. By comparison, typical carbon to carbon bond lengths, or the spacing between these atoms in a molecule, are in the range 0.12-0.15 nm. The significance of a polymer nano-coating is that is can optionally form a very tight bond with the surface of most materials; including glass, paint, plastic, rubber, aluminum, chrome, aluminum, stainless steel, kevlar, cast iron, fabrics, and/or leather will have surface imperfections i.e. peaks and/or valleys, micro-fissures when viewed under high magnification. These undulations allow a nano-coating polymer to form a tight molecular bond with the surface it's applied to. Surfaces sealed with nanotechnology sealants repel water, oil and/or dirt, have antistatic characteristics and/or protect against chemical and/or biological damage. Water, oil and/or dirt can optionally be removed easily, but if the car is very dirty it can optionally be cleaned with a high pressure hose and/or a microfiber cloth. Nanotechnology polymers form a very tight matrix chain-link structure, which forms both a very strong bond and/or one that is not easily breached by chemicals or detergents. This type of nanotechnology coating with its small particulate size are much smaller than those of water; making them hydrophobic (water resistant). Due to their size they fill any surface irregularities (micro fissures), which results in a flat surface, one that reflects light without hindrance.

Nanocrystalline Cellulose Acetate (NCCA). Highly flexible nanocomposite films of nanocrystalline cellulose acetate (NCCA) and graphene oxide (GO) were synthesized by combining NCCA and GO sheets in a well-controlled manner. By adjusting the GO content, various NCCA/GO nanocomposites with 0.3-1 wt % GO were obtained. Films of these nanocomposites were prepared using the solvent casting method. Microscopic and X-ray diffraction (XRD) measurements demonstrated that the GO nanosheets were uniformly dispersed in the NCCA matrix. Mechanical properties of the composite films were also studied. The best GO composition of the samples tested was 0.8 wt %, giving tensile strength of 157.49 MPa, which represents a 61.92% enhancement compared with NCCA. On the other hand, the composite films showed improved barrier properties against water vapor. This simple process for preparation of NCCA/GO films is attractive for potential development of high-performance films for electrical and electrochemical applications.

Nanocrystalline Hydroxyapatite (HAp) Powders can optionally be synthesized using a simple method with chitosan-polymer complex solution. To obtain HAp nanopowders, the prepared precursor was calcined in air at 400-800° C. for 2 h. The phase composition of the calcined samples was studied by X-ray diffraction (XRD) technique. The XRD results confirmed the formation of HAp phase with a small trace of monotite phase. With increasing calcination temperature, the crystallinity of the HAp increased, showing the hexagonal structure of HAp with the lattice parameter a in a range of 0.94030-0.94308 nm and c of 0.68817-0.68948 nm. The particles sizes of the powder were found to be 55.02-73.36 nm as evaluated by the XRD line broadening method. The chemical composition of the calcined powders was characterized by FTIR spectroscopy. The peaks of the phosphate carbonate and hydroxyl vibration modes were observed in the FTIR spectra for all the calcined powders. TEM investigation revealed that the prepared HAP samples consisted of rod-like nanoparticles having the particles size in the range of 100-300 nm. The corresponding selected-area electron diffraction (SAED) analysis further confirmed the formation of hexagonal structure of HAp.

Nanocrystalline TiO₂ can optionally be used as a photocatalysts to deal with environmental pollutions, such as water purification and making saltwater drinkable, wastewater treatment and air purification. Here a sonochemical method for directly preparing anatase nanocrystalline TiO₂ has been established. Nanocrystalline TiO₂ were synthesized by the hydrolysis of titanium tetrabutyl in the presence of water and ethanol under a high-intensity ultrasonic irradiation (20 kHz, 100 W/cm²) at 363 K for 3 h. The structure and particles sizes of the product were dependent upon the reaction temperature, the acidity of the medium and the reaction time. Characterization was accomplished by using various different techniques, such as powder X-ray diffraction (XRD), transmission electron microscopy (TEM), thermogravimetry differential thermal analysis (TG-DTA) and Fourier transform infrared (FTIR) spectroscopy. The TEM images showed that the particles of TiO₂ were columnar in shape and the average sizes were ca. 3 nm×7 nm. The formation mechanism of nanocrystalline TiO₂ under a high intensity ultrasonic irradiation was also investigated. The hydrolytic species of titanium tetrabutyl in water condensed to form a large number of tiny gel nuclei, which aggregated to form larger clusters. Ultrasound irradiation generated a lot of local hot spots within the gel and the crystal structural unit was formed near the hot spots with the decrease of the gel nuclei, which lead to form nanocrystal particles.

Nanocrystalline TiO₂ Coated-Fabric for UV Shielding and Antibacterial Functions. Due to excellent photocatalytic and optical properties of titanium dioxide (TiO2), it has been applied in several products such as food packaging plastics, materials for vehicles or for buildings and sunscreen-protecting cosmetics. In this present work, the synthesized as well as commercial TiO2 was coated onto a household curtain fabric for anti-microbial, and other health properties in sunscreens, cleansers, complexion treatments, creams and lotions, shampoos, and specialized makeup, and ultraviolet (UV) shielding functions. The coating was performed by inducing the deposition of TiO2 layer from the Ti precursor onto the fabric surface pre-treated with silane adhesive agent so as to improve the adhesion. Ag nanoparticles were also incorporated in some samples to further improve the antibacterial function. Antibacterial activities of the coated fabric were evaluated by standard qualitative test (the Kirby-Bauer test (AATCC 147)). Efficiency for UV shielding was evaluated by measuring a UV-Vis reflection of the coated fabrics both before and after subjecting to several washing cycles. The result showed that the TiO2-coated fabrics developed had potential as antibacterial and UV shielding for the garment and curtain industry.

Nanocrystalline Tungsten Carbide (WC) with a high surface area and containing minimal free carbon was synthesized via a polymer route. Its physical properties, including solubility in acid solution, electronic conductivity, and thermal stability, were thoroughly studied at two elevated temperatures: 95° C. and 200° C. Compared to commercially available WC, this in-house synthesized WC showed lower solubility in acidic media at 200° C., higher electronic conductivity (comparable to that of carbon black), as well as higher thermal stability. However, this material exhibited low electrochemical stability in acidic media when subjected to potential cycling at potentials larger than 0.7 V vs. RHE, due to the electrooxidation of WC. The major product of WC electrooxidation is WO₃, which was confirmed by X-ray photon spectroscopy measurements. Pt was uniformly deposited on the high surface area WC to form a 20 wt % of Pt supported catalyst for the oxygen reduction reaction (ORR). The ORR mass activity was then obtained using the rotating disk electrode technique.

Plasma-Enhanced Chemical Vapor Deposition (PECVD) is a process used to deposit thin films from a gas state (vapor) to a solid state on a substrate. Chemical reactions are involved in the process, which occur after creation of a plasma of the reacting gases. The plasma is generally created by RF (AC) frequency or DC discharge between two electrodes, the space between which is filled with the reacting gases.

Pesticides are substances meant for attracting, seducing, and then destroying, or mitigating any pest. They are a class of biocide. The most common use of pesticides is as plant protection products (also known as crop protection products), which in general protect plants from damaging influences such as weeds, plant diseases or insects. This use of pesticides is so common that the term pesticide is often treated as synonymous with plant protection product, although it is in fact a broader term, as pesticides are also used for non-agricultural purposes. The term pesticide includes all of the following: herbicide, insecticide, insect growth regulator, nematicide, termiticide, molluscicide, piscicide, avicide, rodenticide, predacide, bactericide, insect repellent, animal repellent, antimicrobial, fungicide, repellent disinfectant (antimicrobial), and sanitizer. In general, a pesticide is a chemical or biological agent (such as a virus, bacterium, antimicrobial, or disinfectant) that deters, incapacitates, kills, or otherwise discourages pests. Target pests can include insects, plant pathogens, weeds, mollusks, birds, mammals, fish, nematodes (roundworms), and microbes that destroy property, cause nuisance, or spread disease, or are disease vectors. Although pesticides have benefits, some also have drawbacks, such as potential toxicity to humans and other desired species. According to the Stockholm Convention on Persistent Organic Pollutants, 9 of the 12 most dangerous and persistent organic chemicals are organochlorine pesticides.

Petroleum-Based Consumer Products, Packaged Goods and Other End Use Products with Nanocrystalline (NC) Products. Below is a non-limiting partial list of petroleum-based consumer products, packaged goods and other end use products that can optionally be replaced with nanocrystalline (NC) products e.g., ammonia, anesthetics, antifreeze, antihistamines, antiseptics, artificial limbs, artificial turf, aspirin, awnings, balloons, ballpoint pens, bandages, basketballs, bearing grease, bicycle tires, boats, cameras, candles car battery cases, car enamel, cassettes, caulking, cd player, cd's, clothes, clothesline, cold cream, cosmetics, credit cards, combs, cortisone, crayons, curtains, dashboards, denture adhesive, dentures, deodorant, detergents, dice, diesel, dishes, dishwasher, dresses, drinking cups, dyes, electric blankets, electrician's tape, enamel, epoxy, eyeglasses, fan belts, faucet washers, fertilizers, fishing boots, fishing lures, fishing rods, floor wax, folding doors, food preservatives, football cleats, football helmets, footballs, glycerin, golf bags, □golf balls, guitar strings, hair coloring, hair curlers, hand lotion, heart valves, house paint, ice chests, ID cards, ice cube trays, ink, insect repellent, insecticides, life jackets, linings, linoleum, lipstick, luggage, model cars, mops, motor oil, motorcycle helmet, movie film, nail polish, nylon rope, oil filters, paint, paint brushes, paint rollers, panty hose, parachutes, percolators, perfumes, petroleum jelly, pillows, plastic bottles, plastic water bottles, plastic wood, purses, putty, refrigerant, refrigerators, roller skates, roofing, rubber cement, rubbing alcohol, safety glasses, shag rugs, shampoo, shaving cream, shoe polish, shoes, shower curtains, skis, slacks, soap, soft contact lenses, solvents, speakers, sports car bodies, sun glasses, surf boards, sweaters, synthetic rubber, natural rubber, fabric or wire, carbon black and other chemical compounds, telephones, tennis rackets, tents, tires, toilet seats, tool boxes, tool racks, toothbrushes, toothpaste, transparent tape, trash bags, tv cabinets, umbrellas, upholstery, vaporizers, vitamin capsules, watches, water pipes, wheels, yarn, etc.

Polymer is a large molecule, or macromolecule, composed of many repeated subunits. Because of their broad range of properties, both synthetic and/or natural polymers play an essential and/or ubiquitous role in everyday life, Polymers range from familiar synthetic plastics such as polystyrene to natural polymers such as DNA and/or proteins that are fundamental to biological structure and/or function. Polymers, both natural and/or synthetic, are created via polymerization of many small molecules, known as monomers. Their consequently large mass relative to small molecule compounds produces unique physical properties, including toughness, viscoelasticity, and/or a tendency to form glasses and/or semicrystalline structures rather than crystals. The term “polymer” derives from the ancient Greek word [symbol] (polus, meaning “many, much”) and/or [symbol] (meros, meaning “automotive products and/or parts, electronics”), and/or refers to a molecule whose structure is composed of multiple repeating units, from which originates a characteristic of high relative molecular mass and/or attendant properties. The units composing polymers derive, actually or conceptually, from molecules of low relative molecular mass. Polymers consist of repeating molecular units which usually are joined by covalent bonds. Here is a closer look at the chemistry of monomers and polymers. Monomers are small molecules which may be joined together in a repeating fashion to form more complex molecules called polymers. Polymers. A polymer may be a natural or synthetic macromolecule comprised of repeating, units of a smaller molecule (monomers). Polymers are produced by living organisms or polymeric biomolecules. Since they are polymers, polymers contain monomeric units that are covalently bonded to form larger structures. There are three main classes of polymers, classified according to the monomeric units used and the structure of the polymer formed: polynucleotides (RNA and DNA), which are long polymers composed of 13 or more nucleotide monomers; polypeptides, which are short polymers of amino acids; and polysaccharides, which are often linear bonded polymeric carbohydrate structures. Cellulose is the most common organic compound and polymer on Earth. About 33% of all plant matter is cellulose. The cellulose content of cotton is 90%, while wood's is 50%.

Polymer Characterization is the analytical branch of polymer science. The discipline is concerned with the characterization of polymeric materials on a variety of levels. The characterization typically has as a goal to improve the performance of the material. As such, many characterization techniques should ideally be linked to the desirable properties of the material such as strength, impermeability, thermal stability, and optical properties. Characterization techniques are typically used to determine molecular mass, molecular structure, morphology, thermal properties, and mechanical properties.

Pulp is a lignocellulosic fibrous material prepared by chemically or mechanically separating cellulose fibers from wood, fiber crops or waste paper. The wood fiber sources required for pulping can optionally 45% sawmill residue, 21% logs and/or chips, and/or 34% recycled paper. Pulp is one of the most abundant raw materials world wide.

RF Fibers, also known as chipless EMFID, is a kind of EMFID biomagnetic sensors tag that does not make use of any integrated circuit technology to store information data. Fibers or materials are used that reflect a portion of the reader's signal back; the unique return signal can optionally be used as an identifier. Thin threads, fine wires or even labels or laminates—RF fibers are available in many forms. At volume, they range in cost from ten cents to twenty-five cents per unit. RF fibers can optionally be used in more environments using EMFID biomagnetic sensors tags with electronic circuitry. They tend to work over a wider temperature range; these tags also are less sensitive to RF interference. RF fibers are sometimes used in anti-counterfeiting with documents. However, since the tags cannot transmit a unique serial number, they are less usable in the supply chains.

Silicon is a chemical element with symbol Si and atomic number 14. It is a tetravalent metalloid, more reactive than germanium, the metalloid directly below it in the table. Controversy about silicon's character dates to its discovery; it was first prepared and characterized in pure form in 1823. In 1808, it was given the name silicium (from Latin: silex, hard stone or flint), with an -ium word-ending to suggest a metal, a name, which the element retains in several non-English languages. Silicon is the eighth most common element in the universe by mass, but very rarely occurs as the pure free element in nature. It is most widely distributed in dusts, sands, planetoids, and planets as various forms of silicon dioxide (silica) or silicates. Over 90% of the Earth's crust is composed of silicate minerals, making silicon the second most abundant element in the Earth's crust (about 28% by mass) after oxygen. Most silicon is used commercially without being separated, and indeed often with little processing of compounds from nature. These include direct industrial building-use of clays, silica sand and stone. Silicate goes into Portland cement for mortar and stucco, and when combined with silica sand and gravel, to make concrete. Silicates are also in white ware ceramics such as porcelain, and in traditional quartz-based soda-lime glass and many other specialty glasses. More modern silicon compounds such as carbide form abrasives and high-strength ceramics. Silicon is the basis of the widely used synthetic polymers called silicones. Elemental silicon also has a large impact on the modern world economy. Although most free silicon is used in the steel refining, aluminum-casting, and fine chemical industries (often to make fumed silica), the relatively small portion of very highly purified silicon that is used in semiconductor electronics (<10%) is perhaps even more critical. Because of wide use of silicon in integrated circuits, the basis of most computers, a great deal of modern technology depends on it. Silicon is an essential element in biology, although only tiny traces of it appear to be required by animals. However, various sea sponges as well as microorganisms like diatoms and radiolaria secrete skeletal structures made of silica. Silica is often deposited in plant tissues, such as in the bark and wood of chrysobalanaceae and the silica cells and silicified trichomes of sativa, horsetails and many grasses.

Steels are alloys of iron and carbon, widely used in construction and different nanocrystalline (NC) applications because of their high tensile strengths and low costs. Carbon, other elements, and inclusions within iron act as hardening agents that prevent the movement of dislocations that otherwise occur in the crystal lattices of iron atoms. The carbon in typical steel alloys may contribute up to 2.1% of its weight. Varying the amount of alloying elements, their formation in the steel either as solute elements, or as precipitated phases, retards the movement of those dislocations that make iron so ductile and weak and thus controls qualities such as the hardness, ductility and tensile strength of the resulting steel. Steel's strength compared to pure iron is only possible at the expense of ductility, of which iron has an excess.

Spherical Cellulose Nanocrystal (SCNC) suspension can optionally be prepared by hydrolysis of microcrystalline cellulose with a mixture of sulfuric acid and hydrochloric acid under ultrasonic treatment. The mechanism of SCNC formation and the liquid, non-liquid crystalline properties of their suspensions were investigated. A suspension of spherical particles was usually inclined to form crystallization colloids rather than liquid, non-liquid crystals at high concentration. However, a SCNC suspension with high polydispersity (49%) was observed to form the liquid, non-liquid crystalline phase, and the liquid, non-liquid crystalline textures changed with increasing concentration. This observation offers an approach to the liquid, non-liquid crystal formation of highly polydisperse spherical nanoparticles.

Spirulina is a dietary supplement and cyanobacterium that can be consumed by humans and other animals. There are two species, Arthrospira platensis and Arthrospira maxima. Arthrospira is cultivated worldwide; used as a dietary supplement as well as a whole food; and is also available in tablet, flake and powder form. It is also used as a feed supplement in the aquaculture, aquarium and poultry industries.

Seedling is a young plant sporophyte developing out of a plant embryo from a seed. Seedling development starts with germination of the seed. A typical young seedling consists of three main parts: the radicle (embryonic root), the hypocotyl (embryonic shoot), and the cotyledons (seed leaves). The two classes of flowering plants (angiosperms) are distinguished by their numbers of seed leaves: monocotyledons (monocots) have one blade-shaped cotyledon, whereas dicotyledons (dicots) possess two round cotyledons. Gymnosperms are more varied. For example, pine seedlings have up to eight cotyledons. The seedlings of some flowering plants have no cotyledons at all. These are said to be acotyledons. Part of a seed embryo that develops into the shoot, bearing the first true leaves of a plant. In most seeds, for example the sunflower, the plumule is a small conical structure without any leaf structure. Growth of the plumule does not occur until the cotyledons have grown above ground. This is epigeal germination. However, in seeds such as the broad bean, a leaf structure is visible on the plumule in the seed. These seeds develop by the plumule growing up through the soil with the cotyledons remaining below the surface. This is known as hypogeal germination.

Sugar is the generalized name for sweet, short-chain, soluble carbohydrates, many of which are used in food. They are carbohydrates, composed of carbon, hydrogen, and oxygen. There are various types of sugar derived from different sources. Simple sugars are called monosaccharides and include glucose (also known as dextrose), fructose and galactose. The table or granulated sugar most customarily used as food is sucrose, a disaccharide. (In the body, sucrose hydrolyses into fructose and glucose.) Other disaccharides include maltose and lactose. Longer chains of sugars are called oligosaccharides. Chemically-different substances may also have a sweet taste, but are not classified as sugars. Some are used as lower-calorie food substitutes for sugar described as artificial sweeteners. Sugars are found in the tissues of most plants, but are present in sufficient concentrations for efficient extraction only in sugarcane and sugar beet. Sugarcane refers to any of several species of giant grass in the genus Saccharum that have been cultivated in tropical climates in South Asia and Southeast Asia since ancient times. A great expansion in its production took place in the 18th century with the establishment of sugar plantations in the West Indies and Americas. This was the first time that sugar became available to the common people, who had previously had to rely on honey to sweeten foods. Sugar beet, a cultivated variety of Beta vulgaris, is grown as a root crop in cooler climates and became a major source of sugar in the 19th century when methods for extracting the sugar became available. Sugar production and trade have changed the course of human history in many ways, influencing the formation of colonies, the perpetuation of slavery, the transition to indentured labor, the migration of peoples, wars between sugar-trade-controlling nations in the 19th century, and the ethnic composition and political structure of the new world. The world produced about 168 million tonnes of sugar in 2011. The average person consumes about 24 kilograms (53 lb.) of sugar each year (33.1 kg in industrialized countries), equivalent to over 260 food calories per person, per day.

Synthesis of Nanocrystalline Bulk and Powder materials can optionally include the properties of nanocrystalline substances change considerably when the size of crystallites decreases below a threshold value. Such changes arise when the average size of crystal grains does not exceed 100 nm and are most pronounced when grains are less than 10 nm in size. Ultrafine-grain substances should be studied considering not only their composition and structure, but also particles size distribution. Ultrafine-grain substances with grains 300 to 40 nm in size on the average are usually referred to as submicrocrystalline, while those with grains less than 40 nm in size on the average are called nanocrystalline. Nanosubstances and nanomaterials may be classified by geometrical shape and the dimensionality of their structural elements. The main types of nanomaterials with respect to the dimensionality include cluster materials, fibrous materials, films and multilayered materials, and also polycrystalline materials whose grains have dimensions comparable in all the three directions.

Titanium is a chemical element with symbol Ti and atomic number 22. It is a lustrous transition metal with a silver color, low density and high strength. It is highly resistant to corrosion in sea water, aqua regia and chlorine. Titanium was discovered in Cornwall, Great Britain, by William Gregor in 1791 and named by Martin Heinrich Klaproth for the Titans of Greek mythology. The element occurs within a number of mineral deposits, principally rutile and ilmenite, which are widely distributed in Earth's crust and lithosphere, and it is found in almost all living things, rocks, water bodies, and soils. The metal is extracted from its principal mineral ores via the Kroll process or the Hunter process. Its most common compound, titanium dioxide, is a popular photocatalyst and is used in the manufacture of white pigments. Other compounds include titanium tetrachloride (TiCl₄), a component of smoke screens and catalysts; and titanium trichloride (TiCl₃), which is used as a catalyst in the production of polypropylene. Titanium can be alloyed with iron, aluminium, vanadium, and molybdenum, among other elements, to produce strong, lightweight alloys for aerospace (jet engines, missiles, and spacecraft), military, industrial process (chemicals and petro-chemicals, desalination plants, pulp, and paper), automotive, agri-food, medical prostheses, coatings for medical applications, medical implants, breast implant devices, microchip implants or other types of implants, orthopedic implants, dental and endodontic instruments and files, dental implants, sporting goods, jewelry, mobile phones, and other applications. The two most useful properties of the metal are corrosion resistance and the highest strength-to-density ratio of any metallic element. In its unalloyed condition, titanium is as strong as some steels, but less dense. There are two allotropic forms and five naturally occurring isotopes of this element, ⁴⁶Ti through ⁵⁰Ti, with ⁴⁸Ti being the most abundant (73.8%). Although they have the same number of valence electrons and are in the same group in the periodic table, titanium and zirconium differ in many chemical and physical properties.

Titanium Dioxide is also known as titanium (IV) oxide or titania is the naturally occurring oxide of titanium, chemical formula TiO2. When used as a pigment, it is called titanium white, Pigment White 6 (PW6), or CI 77891. Generally it is sourced from ilmenite, rutile and anatase. It has a wide range of applications, from paint to sunscreen to food coloring. When used as a food coloring, it has E number E171.

Ultrananocrystalline Diamond (UNCD) as a structural material for complex micro-electro mechanical systems (MEMS) is enormous due to its excellent chemical, mechanical and barrier properties, but it has so far not been extensively explored, mostly due to intrinsic stress problems. The N-UNCD can utilize semiconducting at its thermal and barrier properties. Fifteen pairs of oriented slender beams (from 90 to 200 μm length) provide the driving force and are capable of generating a linear displacement on a central moving shuttle up to almost 2 μm. An ‘in-house’ built optical-based detection system was used to assess the motion of the actuator, with an accuracy of 0.4 nm. These results pave the way for development of diamond-based MEMS technology that could be applicable in many fields, including bio-medicine, optics, and sensors and actuators for space applications, where precision displacement is demanded along with robust materials, as well as general applications that require sliding surfaces.

Wood Pulp is a type of material that is created by processing wood collected from trees, and/or serves as the basis for the creation of a multiple paper-based products. Several different processes are utilized to reduce the wood into a form that is ideal for manufacturing different types of paper goods, including paper used in printing books, magazines, and/or newspapers. The resulting paper product can optionally also be used to create other paper products, including disposable paper plates, paper towels, and/or other common household items, microprocessor in athletic shoes, detergents for washing, fabric softener. The process of reducing wood into wood pulp will often include the use of some sort of grinding machinery to create fine chips that can optionally be refined using pressure and/or steam. This will often involve introducing the tiny chips to a steaming process that helps to soften the fibers, making the product more malleable. From there, pressure is exerted to create thin sheets that are ideal for use as newsprint for newspapers as well as paper that can optionally be eventually worked into everything from mailing envelopes to paper used in printing books and/or magazines.

Wet End means that portion of the nanocrystalline (NC) product making process prior to a press section where a liquid medium such as water typically comprises more than 45% of the mass of the substrate, additives added in a wet end typically penetrate and distribute within the slurry.

Dry End means that portion of the nanocrystalline (NC) product making process including and subsequent to a press section where a liquid medium such as water typically comprises less than 45% of the mass of the substrate, dry end includes but is not limited to the size press portion of a nanocrystalline (NC) product making process, additives added in a dry end typically remain in a distinct coating layer outside of the slurry.

Flocculant means a composition of matter which when added to a liquid carrier phase within which certain products are thermodynamically inclined to disperse, induces agglomerations of those products to form as a result of weak physical forces such as surface tension and adsorption, flocculation often involves the formation of discrete globules of products aggregated together with films of liquid carrier interposed between the aggregated globules, as used herein flocculation includes those descriptions recited in ASTME 20-85 as well as those recited in Kirk-Othmer Encyclopedia of Chemical Technology, 5th Edition, (2005), (Published by Wiley, John & Sons, Inc.).

Surface Strength means the tendency of a substrate, component, or additive to resist damage due to abrasive force.

Dry Strength means the tendency of a substrate, component, or additive to resist damage due to shear force(s), it includes but is not limited to surface strength.

Wet Strength means the tendency of a substrate, component, or additive to resist damage due to shear force(s) when rewet.

Wet Web Strength means the tendency of a substrate, component, or additive to resist shear force(s) while the substrate is still wet.

Substrate means a mass containing paper fibers going through or having gone through a nanocrystalline (NC) product making process, substrates include wet web, paper mat, slurry, paper sheet, and paper products.

Paper Product means the end product of a nanocrystalline (NC) product making process it includes but is not limited to writing paper, printer paper, tissue paper, cardboard, paperboard, and packaging paper.

NCC or NCC Core means nano-crystalline cellulose. NCC Core is a discrete mass of NCC crystal onto which polymers can optionally be grafted. an NCC or NCC core can optionally or can optionally not have been formed by acid hydrolysis of cellulose fibers and NCC or NCC core can optionally or can optionally not have been modified by this hydrolysis to have functional groups appended thereto including but not limited to sulfate esters.

Nanocrystalline Cellulose (NCC), Nanocrystalline (NC) Polymers, Nanocrystalline (NC) Plastics, or other nanocrystals of cellulose composites or structures means a composition of matter comprising at least an NCC or NC material core with at least one polymer chain or polymer or microcrystalline cellulose (MCC) or nanocrystalline composites, cores or other forms of nanocrystalline extending therefrom.

NCC Coupling means a composition of matter comprising at least two NCC cores, the coupling can optionally be a polymer linkage in which at least in part a polymer chain connects the two NCC cores, or it can optionally be an NCC twin in which two (or more) NCC cores are directly connected to each other by a sub polymer linkage (such as epoxide) and/or direct bonding of one or more of the NCC cores' atoms.

Slurry means a mixture comprising a liquid medium such as water within which solids such as fibers (such as cellulose fibers) and optionally fillers are dispersed or suspended such that between >99% to 45% by mass of the slurry is liquid medium.

Spirulina is a cyanobacterium that can be consumed by humans and other animals. There are two species, Arthrospira platensis and Arthrospira maxima. Arthrospira is cultivated worldwide; used as a dietary supplement as well as a whole food; and is also available in tablet, flake and powder form. It is also used as a feed supplement in the aquaculture, aquarium and poultry industries. Protein. Dried spirulina contains about 60% (51%-71%) protein. It is a complete protein containing all essential amino acids, though with reduced amounts of methionine, cysteine and lysine when compared to the proteins of meat, eggs and milk. It is, however, superior to typical plant protein, such as that from legumes. The U.S. National Library of Medicine said that spirulina was no better than milk or meat as a protein source, and was approximately 30 times more expensive per gram.

Other Nutrients. Spirulina's lipid content is about 7% by weight, and is rich in gamma-linolenic acid (GLA), and also provides alpha-linolenic acid (ALA), linoleic acid (LA), stearidonic acid (SDA), eicosapentaenoic acid (EPA), docosahexaenoic acid (DHA), and arachidonic acid (AA). Spirulina contains vitamins B₁ (thiamine), B₂ (riboflavin), B₃ (nicotinamide), B₆ (pyridoxine), B₉ (folic acid), vitamin C, vitamin A, and vitamin E. It is also a source of potassium, calcium, chromium, copper, iron, magnesium, manganese, phosphorus, selenium, sodium, and zinc. Spirulina contains many pigments which may be beneficial and bioavailable, including beta-carotene, zeaxanthin, 7-hydroxyretinoic acid, isomers, chlorophyll-a, xanthophyll, echinenone, myxoxanthophyll, canthaxanthin, diatoxanthin, 3′-hydroxyechinenone, beta-cryptoxanthin, and oscillaxanthin, plus the phycobiliproteins c phycocyaninand allophycocyanin. Vitamin B₁₂ controversy. Spirulina is not considered to be a reliable source of Vitamin B₁₂. Spirulina supplements contain predominantly pseudovitamin B₁₂, which is biologically inactive in humans. Companies which grow and market spirulina have claimed it to be a significant source of B₁₂ on the basis of alternative, unpublished assays, although their claims are not accepted by independent scientific organizations. The American Dietetic Association and Dietitians of Canada in their position paper on vegetarian diets state that spirulina cannot be counted on as a reliable source of active vitamin B₁₂. The medical literature similarly advises that spirulina is unsuitable as a source of B₁₂.

Sugar Substitute is a food additive that provides a sweet taste like that of sugar while containing significantly less food energy. Some sugar substitutes are natural and some are synthetic. Those that are not natural are, in general, called artificial sweeteners. An important class of sugar substitutes is known as high-intensity sweeteners. These are compounds with many times the sweetness of sucrose, common table sugar. As a result, much less sweetener is required and energy contribution is often negligible. The sensation of sweetness caused by these compounds (the “sweetness profile”) is sometimes notably different from sucrose, so they are often used in complex mixtures that achieve the most natural sweet sensation. If the sucrose (or other sugar) that is replaced has contributed to the texture of the product, then a bulking agent is often also needed. This may be seen in soft drinks or sweet tea that are labeled as “diet” or “light” that contain artificial sweeteners and often have notably different mouthfeel, or in table sugar replacements that mix maltodextrins with an intense sweetener to achieve satisfactory texture sensation. In the United States, seven intensely sweet sugar substitutes have been approved for use. They are stevia, aspartame, sucralose, neotame, acesulfame potassium (Ace-K), saccharin, and advantame. Cyclamates are used outside the U.S., but have been prohibited in the U.S. since 1969. There is some ongoing controversy over whether artificial sweetener usage poses health risks. The U.S. Food and Drug Administration regulates artificial sweeteners as food additives. Food additives must be approved by the FDA, which publishes a Generally Recognized as Safe (GRAS) list of additives. (Stevia is exempt under FDA's GRAS policy due to its being a natural substance in wide use well before 1958, and has been approved by FDA). The conclusions about safety are based on a detailed review of a large body of information, including hundreds of toxicological and clinical studies. The majority of sugar substitutes approved for food use are artificially synthesized compounds. However, some bulk natural sugar substitutes are known, including sorbitol and xylitol, which are found in berries, fruit, vegetables, and mushrooms. It is not commercially viable to extract these products from fruits and vegetables, so they are produced by catalytichydrogenation of the appropriate reducing sugar. For example, xylose is converted to xylitol, lactose to lactitol, and glucose to sorbitol. Other natural substitutes are known but are yet to gain official approval for food use. Some non-sugar sweeteners are polyols, also known as “sugar alcohols.” These are, in general, less sweet than sucrose but have similar bulk properties and can optionally be in a wide range of food ingredients and food products. Sometimes the sweetness profile is ‘fine-tuned’ by mixing with high-intensity sweeteners. As with all food ingredients or food products, the development of a formulation to replace sucrose is a complex proprietary process.

Surfactant is a broad term, which includes anionic, nonionic, cationic, and zwitterionic surfactants. Enabling descriptions of surfactants are stated in Kirk-Othmer, Encyclopedia of Chemical Technology, Third Edition, volume 8, pages 900-912, and in McCutcheon's Emulsifiers and Detergents, both of which are incorporated herein by reference.

Size Press means the part of the nanocrystalline (NC) product making process machine where the dry component is rewet by applying a water-based formulation containing surface additives such as starch, sizing agents and optical brightening agents, a more detailed descriptions of size press is described in the reference Handbook for Pulp and Paper Technologists, 3rd Edition, by Gary A. Smook, Angus Wilde Publications Inc., (2002).

Vapor-Liquid-Solid Method (VLS) is a mechanism for the growth of one-dimensional structures, such as nanowires, from chemical vapor deposition. The growth of a crystal through direct adsorption of a gas phase on to a solid surface is generally very slow. The VLS mechanism circumvents this by introducing a catalytic liquid alloy phase which can rapidly adsorb a vapor to supersaturation levels, and from which crystal growth can subsequently occur from nucleated seeds at the liquid-solid interface. The physical characteristics of nanowires grown in this manner depend, in a controllable way, upon the size and physical properties of the liquid alloy.

Yeasts are eukaryotic microorganisms classified in the kingdom Fungi, with 1,500 species currently described (estimated to be 1% of all described fungal species). Yeasts are unicellular, although some species with yeast forms may become multicellular through the formation of strings of connected budding cells known as pseudohyphae, or false hyphae, as seen in most molds. Natural yeast is a dietary supplement. Yeast size can vary greatly depending on the species, typically measuring 3-4 μm in diameter, although some yeasts can reach over 40 μm. Most yeasts reproduce asexually by mitosis, and many do so by an asymmetric division process called budding. By fermentation, the yeast species Saccharomyces cerevisiae converts carbohydrates to carbon dioxide and alcohols—for thousands of years the carbon dioxide has been used in baking and the alcohol in alcoholic beverages. It is also a centrally important model organism in modern cell biology research, and is one of the most thoroughly research edeukaryotic microorganisms. Researchers have used it to gather information about the biology of the eukaryotic cell and ultimately human biology. Other species of yeasts, such as Candida albicans, are opportunistic pathogens and can cause infections in humans. Yeasts have recently been used to generate electricity in microbial fuel cells, and produce ethanol for the biofuel industry. Yeasts do not form a single taxonomic or phylogenetic grouping. The term “yeast” is often taken as a synonym for Saccharomyces cerevisiae, but the phylogenetic diversity of yeasts is shown by their placement in two separate phyla: the Ascomycota and the Basidiomycota. The budding yeasts (“true yeasts”) are classified in the order Saccharomycetales.

Vitamin is a dietary supplement and an organic compound and a vital nutrient that an organism requires in limited amounts. An organic chemical compound (or related set of compounds) is called a vitamin when the organism cannot synthesize the compound in sufficient quantities, and must optionally be obtained through the diet; thus, the term “vitamin” is conditional upon the circumstances and the particular organism.

Nanomaterials (nanocrystalline (NC) materials) are materials possessing grain sizes on the order of a billionth of a meter. They manifest extremely fascinating and useful properties, which can be exploited for a variety of structural and non-structural applications.

### Nanomaterials Applications

Since nanomaterials possess unique, beneficial chemical, physical, and mechanical properties, they can optionally be used for a wide variety of applications. These applications include, but are not limited to, the following:

### Next-Generation Computer Chips

The microelectronics industry has been emphasizing miniaturization, whereby the circuits, such as transistors, resistors, and capacitors, are reduced in size. By achieving a significant reduction in their size, the microprocessors, which contain these components, can run much faster, thereby enabling computations at far greater speeds. However, there are several technological impediments to these advancements, including lack of the ultrafine precursors to manufacture these components; poor dissipation of tremendous amount of heat generated by these microprocessors due to faster speeds; short mean time to failures (poor reliability), etc. Nanomaterials can help the industry break these barriers down by providing the manufacturers with nanocrystalline starting materials, ultra-high purity materials, materials with better thermal conductivity, and longer-lasting, durable interconnections (connections between various components in the microprocessors).

### Improved Insulation Materials

Nanocrystalline (NC) materials synthesized by the sol-gel technique result in foam like structures called “aerogels.” These aerogels are porous and extremely lightweight; yet, they can loads equivalent to 100 times their weight. Aerogels are composed of three-dimensional, continuous networks of particles with air (or any other fluid, such as a gas) trapped at their interstices. Since they are porous and air is trapped at the interstices, aerogels are currently being used for insulation in offices, homes, etc. By using aerogels for insulation, heating and cooling bills are drastically reduced, thereby saving power and reducing the attendant environmental pollution. They are also being used as materials for “smart” windows, which darken when the sun is too bright (just as in changeable lenses in prescription spectacles and sunglasses) and they lighten themselves, when the sun is not shining too brightly.

### Low-Cost Flat-Panel Displays

Flat-panel displays represent a huge market in the laptop (portable) computers industry. However, Japan is leading this market, primarily because of its research and development efforts on the materials for such displays. By synthesizing nanocrystalline phosphors, the resolution of these display devices can be greatly enhanced, and the manufacturing costs can be significantly reduced. Also, the flat-panel displays constructed out of nanomaterials possess much higher brightness and contrast than the conventional ones owing to their enhanced electrical and magnetic properties.

### Tougher and Harder Cutting Tools

Cutting tools made of nanocrystalline (NC) materials, nanocrystalline (NC) components, such as tungsten carbide, tantalum carbide, and titanium carbide, are much harder, much more wear-resistant, erosion-resistant, and last longer than their conventional (large-grained) counterparts. They also enable the manufacturer to machine various materials much faster, thereby increasing productivity and significantly reducing manufacturing costs. Also, for the miniaturization of microelectronic circuits, the industry needs microdrills (drill bits with diameter less than the thickness of an average human hair or 100 μm) with enhanced edge retention and far better wear resistance. Since nanocrystalline carbides are much stronger, harder, and wear-resistant, they are currently being used in these microdrills.

### Elimination of Pollutants

Nanocrystalline (NC) materials possess extremely large grain boundaries relative to their grain size. Hence, nanomaterials are very active in terms of their of chemical, physical, and mechanical properties. Due to their enhanced chemical activity, nanomaterials can optionally be used as catalysts to react with such noxious and toxic gases as carbon monoxide and nitrogen oxide in automobile catalytic converters and power generation equipment to prevent environmental pollution arising from burning gasoline and coal.

### High Energy Density Batteries

Conventional and rechargeable batteries are used in almost all applications that require electric power. These applications include high energy density batteries, laptop computers, electric vehicles, home batteries, business batteries, solar batteries, flexible batteries, bendable batteries, wearable batteries, next-generation electric vehicles (NGEV) to reduce environmental pollution, personal stereos, cellular phones, cordless phones, toys, and watches. The energy density (storage capacity) of these batteries is quite low requiring frequent recharging. The life of conventional and rechargeable batteries is also low. Nanocrystalline (NC) materials synthesized by sol-gel techniques are candidates for separator plates in batteries because of their foam-like (aerogel) structure, which can hold considerably more energy than their conventional counterparts. Furthermore, nickel-metal hydride (Ni-MH) batteries made of nanocrystalline nickel and metal hydrides are envisioned to require far less frequent recharging and to last much longer because of their large grain boundary (surface) area and enhanced physical, chemical, and mechanical properties.

### High-Power Magnets

The strength of a magnet is measured in terms of coercivity and saturation magnetization values. These values increase with a decrease in the grain size and an increase in the specific surface area (surface area per unit volume of the grains) of the grains. It has been shown that magnets made of nanocrystalline yttrium-samarium-cobalt grains possess very unusual magnetic properties due to their extremely large surface area. Typical applications for these high-power rare-earth magnets include quieter submarines, automobile alternators, land-based power generators, motors for ships, ultra-sensitive analytical instruments, and magnetic resonance imaging (MRI) in medical diagnostics.

### High-Sensitivity Sensors

Sensors employ their sensitivity to the changes in various parameters they are designed to measure. The measured parameters include electrical resistivity, chemical activity, magnetic permeability, thermal conductivity, and capacitance. All of these parameters depend greatly on the microstructure (grain size) of the materials employed in the sensors. A change in the sensor's environment is manifested by the sensor material's chemical, physical, or mechanical characteristics, which is exploited for detection. For instance, a carbon monoxide sensor made of zirconium oxide (zirconia) uses its chemical stability to detect the presence of carbon monoxide. In the event of carbon monoxide's presence, the oxygen atoms in zirconium oxide react with the carbon in carbon monoxide to partially reduce zirconium oxide. This reaction triggers a change in the sensor's characteristics, such as conductivity (or resistivity) and capacitance. The rate and the extent of this reaction are greatly increased by a decrease in the grain size. Hence, sensors made nanocrystalline (NC) materials are extremely sensitive to the change in their environment. Typical applications for sensors made out of nanocrystalline (NC) materials are smoke detectors, ice detectors on aircraft wings, automobile engine performance sensor, etc.

Automobiles with Greater Fuel Efficiency

Currently, automobile engines waste considerable amounts of gasoline, thereby contributing to environmental pollution by not completely combusting the fuel. A conventional spark plug is not designed to burn the gasoline completely and efficiently. This problem is compounded by defective, or worn-out, spark plug electrodes. Since nanomaterials are stronger, harder, and much more wear-resistant and erosion-resistant, they are presently being envisioned to be used as spark plugs. These electrodes render the spark plugs longer-lasting and combust fuel far more efficiently and completely. A radically new spark plug design called the “railplug” is also in the prototype stages. This railplug uses the technology derived from the “railgun,” which is a spin-off of the popular Star Wars defense program. However, these railplugs generate much more powerful sparks (with an energy density of approximately 1 kJ/mm²). Hence, conventional materials erode and corrode too soon and quite frequently to be of any practical use in automobiles. Nevertheless, railplugs made of nanomaterials last much longer even the conventional spark plugs. Also, automobiles waste significant amounts of energy by losing the thermal energy generated by the engine. This is especially true in the case of diesel engines. Hence, the engine cylinders (liners) are currently being envisioned to be coated with nanocrystalline ceramics, such as zirconia and alumina, so that they retain heat much more efficiently and result in complete and efficient combustion of the fuel.

Aerospace Components with Enhanced Performance Characteristics

Due to the risks involved in flying, aircraft manufacturers strive to make the aerospace components with enhanced performance characteristics, stronger, tougher, and last longer. One of the key properties required of the aircraft components is the fatigue strength, which decreases with the component's age. By making the components out of stronger materials, the life of the aircraft is greatly increased. The fatigue strength increases with a reduction in the grain size of the material. Nanomaterials provide such a significant reduction in the grain size over conventional materials that the fatigue life is increased by an average of 200%-300%. Furthermore, components made of nanomaterials are stronger and can operate at higher temperatures, aircrafts can fly faster and more efficiently (for the same amount of aviation fuel). In space crafts, elevated-temperature strength of the material is crucial because the components (such as rocket engines, thrusters, and vectoring nozzles) operate at much higher temperatures than aircrafts and higher speeds. Nanomaterials are perfect candidates for spacecraft applications, as well.

### Better and Future Weapons Platforms

Conventional guns, such as cannons, 155 mm howitzers, and multiple-launch rocket system (MLRS), utilize the chemical energy derived by igniting a charge of chemicals (gun powder). The maximum velocity at which the penetrator can be propelled is approximately 1.5-2.0 km/sec. On the other hand, electromagnetic launchers (EML guns), or railguns, use the electrical energy, and the concomitant magnetic field (energy), to propel the penetrators/projectiles at velocities up to 10 km/sec. This increase in velocity results in greater kinetic energy for the same penetrator mass. The greater the energy, the greater is the damage inflicted on the target. For this and other reasons, the DoD (especially, the U. S. Army) has conducted extensive research into the rail guns. Since a railgun operates on electrical energy, the rails need to be very good conductors of electricity. Also, they need to be so strong and rigid that the railgun does not sag while firing and buckle under its own weight. The obvious choice for high electrical conductivity is copper. However, the railguns made out of copper wear out much too quickly due to the erosion of the rails by the hypervelocity projectiles and they lack high-temperature strength. The wear and erosion of copper rails necessitate inordinately frequent barrel replacements. In order to satisfy these requirements, a nanocrystalline (NC) coating applications, composite material made of tungsten, copper, and titanium diboride is being evaluated as a potential candidate. This nanocomposite possesses the requisite electrical conductivity, adequate thermal conductivity, excellent high strength, high rigidity, hardness, and wear/erosion resistance. This results in longer-lasting, wear-resistant, and erosion-resistant railguns, which can be fired more frequently and often than their conventional counterparts.

### Longer-Lasting Satellites

Satellites are being used for both defense and civilian applications. These satellites utilize thruster rockets to remain in or change their orbits due to a variety of factors including the influence of gravitational forces exerted by the earth. Hence, these satellites are repositioned using these thrusters. The life of these satellites, to a large extent, is determined by the amount of fuel they can carry on board. In fact, more than ⅓ of the fuel carried aboard by the satellites is wasted by these repositioning thrusters due to incomplete and inefficient combustion of the fuel, such as hydrazine. The reason for the incomplete and inefficient combustion is that the onboard igniters wear out quickly and cease to perform effectively. Nanomaterials, such as nanocrsytalline tungsten-titanium diboride-copper composite, are potential candidates for enhancing these igniters' life and performance characteristics.

### Longer-Lasting Medical Implants

Nanocrystalline cellulose (NCC) can optionally be used for coatings for medical applications, medical implants, breast implant devices, microchip implants or other types of implants such as coatings for medical applications, medical implants, breast implant devices, microchip implants or other types of implants, orthopedic implants and heart valves, are made of titanium and stainless steel alloys. These alloys are primarily used in humans because they are biocompatible, i.e., they do not adversely react with human tissue. In the case of coatings for medical applications, medical implants, breast implant devices, microchip implants or other types of implants, orthopedic implants (artificial bones for hip, etc.), these materials are relatively non-porous. For an implant to effectively mimic a natural human bone, the surrounding tissue must penetrate the implants, thereby affording the implant with the required strength. Since these materials are relatively impervious, human tissue does not penetrate the implants, thereby reducing their effectiveness. Furthermore, these metal alloys wear out quickly necessitating frequent, and often very expensive, surgeries. However, nanocrystalline zirconia (zirconium oxide) ceramic is hard, wear-resistant, corrosion-resistant (biological fluids are corrosive), and biocompatible. Nanoceramics can optionally be made porous into aerogels (aerogels can withstand up to 100 times their weight), if they are synthesized by sol-gel techniques. This results in far less frequent implant replacements, and hence, a significant reduction in surgical expenses. Nanocrystalline (NC) silicon carbide (SiC) is a candidate material for artificial heart valves primarily due to its low weight, high strength, extreme hardness, wear resistance, inertness (SiC does not react with biological fluids), and corrosion resistance.

### Ceramics

Ceramics, per se, are very hard, brittle, and hard to machine. These characteristics of ceramics have discouraged the potential users from exploiting their beneficial properties. However, with a reduction in grain size, these ceramics have increasingly been used. Zirconia, a hard, brittle ceramic, has even been rendered superplastic, i.e., it can be deformed to great lengths (up to 300% of its original length). However, these ceramics must possess nanocrystalline grains to be superplastic. In fact, nanocrystalline ceramics, such as silicon nitride (Si₃N₄) and silicon carbide (SiC), have been used in such automotive applications as high-strength springs, ball bearings, and valve lifters, because they possess good formability and machinability combined with excellent physical, chemical, and mechanical properties. They are also used as components in high-temperature furnaces. Nanocrystalline ceramics can be pressed and sintered into various shapes at significantly lower temperatures, whereas it would be very difficult, if not impossible, to press and sinter conventional ceramics even at high temperatures.

### Large Electrochromic Display Devices

An electrochromic device consists of materials in which an optical absorption band can be introduced, or an existing band can be altered by the passage of current through the materials, or by the application of an electric field. Nanocrystalline (NC) materials, nanocrystalline (NC) components, such as tungstic oxide (WO₃.xH₂O) gel, are used in very large electrochromic display devices. The reaction governing electrochromism (a reversible coloration process under the influence of an electric field) is the double-injection of ions (or protons, H⁺) and electrons, which combine with the nanocrystalline tungstic acid to form a tungsten bronze. These devices are primarily used in public billboards and ticker boards to convey information. Electrochromic devices are similar to liquid-crystal displays (LCD) commonly used in calculators and watches. However, electrochromic devices display information by changing color when a voltage is applied. When the polarity is reversed, the color is bleached. The resolution, brightness, and contrast of these devices greatly depend on the tungstic acid gel's grain size. Hence, nanomaterials are being explored for this purpose. Because nanocellulose is transparent, light and strong, it can optionally be in place of plastic or glass. Swap the—usually thick and stiff—separators inside batteries for something made of thin, flexible nanocellulose, and all of a sudden you end up with a mobile power source that bends a little. Combine it with a graphene shell, and you will have the flexible battery of the future.

### Fertilizers

Fertilizer is any organic or inorganic material of natural or synthetic origin (other than liming materials) that is added to a soil to supply one or more plant nutrients essential to the growth of plants. Conservative estimates report 30 to 50% of crop yields are attributed to natural or synthetic commercial fertilizer. European fertilizer market is expected to grow to [symbol] 15.3 billion by 2018. Mined inorganic fertilizers have been used for many centuries, whereas chemically synthesized inorganic fertilizers were only widely developed during the industrial revolution. Increased understanding and use of fertilizers were important parts of the pre-industrial British Agricultural Revolution and the industrial Green Revolution of the 20th century. Inorganic fertilizer use has also significantly supported global population growth—it has been estimated that almost half the people on the Earth are currently fed as a result of synthetic nitrogen fertilizer use. Mined inorganic fertilizers typically provide, in varying proportions: six macronutrients: nitrogen (N), phosphorus (P), potassium (K), calcium (Ca), magnesium (Mg), and sulfur (S); eight micronutrients: boron (B), chlorine (Cl), copper (Cu), iron (Fe), manganese (Mn), molybdenum (Mo), zinc (Zn) and nickel (Ni) (1987). The macronutrients are consumed in larger quantities and are present in plant tissue in quantities from 0.15% to 6.0% on a dry matter (0% moisture) basis (DM).

### Micronutrients

Micronutrients are consumed in smaller quantities and are present in plant tissue on the order of parts per million (ppm), ranging from 0.15 to 400 ppm DM, or less than 0.04% DM. Only three other macronutrients are required by all plants: carbon, hydrogen. New technologies for nanocrystalline cellulose (NCC) may optionally include nano fertilizers, pesticides and/or herbicides with at least one micronutrient, microingredient, growth hormone, crop additive, be able to be incorporated into and used by agricultural seeds and/or vegetable seeds of crops or genetically modified (GM) organisms or genetically modified (GM) crops or genetically engineered (GE) crops or genetically modified (GM) foods or ingredients or artificial ingredients in human and pet food or processed food and affect their germination, growth, crop yield, product quality and growth rates and support water uptake inside seeds, a process, which can affect seed germination, growth, crop yield, product quality and growth of seedlings through enhanced fertilizer uptake capacity, herbicide tolerance, insect tolerance, drought tolerance and increased food and/or vegetation production, agricultural products, industrial products, agricultural based products, compound feed, animal feed, agricultural commodities, fruits, food ingredients, food products, food packaging applications, food applications, food additives, organic food additives, organic products, soy bean, protein, soy products, milk production and/or dairy products and the like.

### Effects of Nanocrystalline Powders (Fe, Co and Cu) on the Germination, Growth, Crop Yield and Product Quality of Soybean Seeds

Super dispersive iron, cobalt and copper nanocrystalline powders were synthesized in a water-ethanol medium by the reduction method using sodium borohydride as a reducing agent and carboxymethyl cellulose as a stabilizer (for Fe and Co nanoparticles). Transmission electron microscopy micrographs and x-ray diffraction analyses of the freshly prepared nanocrystalline powders indicated that they were in a zerovalent state with particles sizes ranging from 20 to 60 nm. The soybean seeds may optionally be treated with an extra low nanocrystalline dose (not more than 300 mg of each metal per hectare) and then sowed on an experimental landfill plot consisting of a farming area of 180 m2. This pre-sowing treatment of soybean seeds, which does not exert any adverse effect on the soil environment, reliably changed the biological indices of the plant growth and development. In particular, in laboratory experiments, the germination rates of soybean seeds treated with zerovalent Cu, Co and Fe were 65%, 80% and 80%, respectively, whereas 55% germination was observed in the control sample; in the field experiment, for all of the nanoscale metals studied, the chlorophyll index increased by 7%-15% and the number of nodules by 20%-49% compared to the control sample, and the soybean crop yield increased up to 16% in comparison with the control sample.

### Orthopedics

Nanocrystalline apatite-based materials and stem cells are emerging research fields in orthopedic surgery and traumatology that have the potential of improving quality of life of the elderly and enhance health-related socio-economic challenges. Nanocrystalline apatite-based materials and especially calcium phosphate nano-materials exploit new physical, chemical and biological properties that have the possibility to increase surface area and improve tissue integration. Stem cells of adult origin decrease inflammation, increase vascularity and are able to replace degenerated tissue cells during the process of regeneration. The bone is the only human tissue that regenerates. Musculoskeletal disorders including osteoporotic fractures and osteoarthritis decrease quality of life in the elderly and cause severe burden on economics. Nanocrystalline calcium phosphate bioceramics have the ability to prevent or treat osteoporotic fractures when combined with stem cells. These materials may also be used for drug delivery purposes to treat bone infections when combined with stem cell as they can assist in treating osteoarthritis. Current research challenges are trying to overcome the toxicity and carcinogenesis with these cells and nanomaterials.

### Nanocrystalline Coating Applications

In recent years, near-nano (submicron) and nanostructured materials have attracted increasingly more attention from the materials community. Nanocrystalline materials are characterized by a microstructural length or grain size of up to about 100 nm. Materials having grain size of ˜0.1 to 0.3 μm are classified as submicron materials. Nanocrystalline materials exhibit various shapes or forms, and possess unique chemical, physical or mechanical properties. When the grain size is below a critical value (˜1.0-20 nm), more than 50 vol. % of atoms is associated with grain boundaries or interfacial boundaries. In this respect, dislocation pile-ups cannot form, and the Hall-Petch relationship for conventional coarse-grained materials is no longer valid. Therefore, grain boundaries play a major role in the deformation of nanocrystalline materials. Nanocrystalline materials exhibit creep and super plasticity at lower temperatures than conventional micro-grained counterparts. Similarly, plastic deformation of nanocrystalline coatings is considered to be associated with grain boundary sliding assisted by grain boundary diffusion or rotation. In this review paper, current developments in fabrication, microstructure, physical and mechanical properties of nanocrystalline materials and coatings will be addressed. Particular attention is paid to the properties of transition metal nitride nanocrystalline films formed by ion beam assisted deposition process.

### Nanocoatings in Medicine

Whilst nanotechnology particularly focuses upon the power of working at the nanoscale, often with reference to particles or atoms, nano-based materials or nanocoatings upon ordinary classed materials, are equally as potent. Nanocoatings are prevalent across a multitude of disciplines ranging from engineering through to medicine. A wide range of materials and techniques has been employed to produce nanocoatings for a given purpose. Nanocoatings are used to improve mechanical properties, offer novel functionality such as extreme water repellence (superhydrophobicity) or their implementation in the pharmaceutical, medical and dental industries (e.g. the coating of dental and medical implants).

### Active Packing, Intelligent Packaging and Smart Packaging for Food, Beverages, Pharmaceutical and Household Products

How Activated Packaging Systems Work. This consists of a matrix polymer, with an oxygen scavenging/absorbing component and a catalyst. The oxygen-scavenging component is a nylon polymermelt blended with the polymer at around the 5% level. The catalyst is a cobalt salt added at a low concentration (less than 200 ppm) that triggers the oxidation of the packaging. The oxygen-scavenging system remains active for periods of up to two years providing protection to oxygen sensitive products such as beer, wine, fruit juice and mayonnaise throughout their shelf-lives. Active food packaging systems using oxygen scavenging and anti-microbial technologies (e.g. sorbate-releasing LDPE film for cheese) have the potential to extend the shelf-life of perishable foods while at the same time improving their quality by reducing the need for additives and preservatives.

How Intelligent Packaging Works. In ‘intelligent’ packaging, the package function switches on and off in response to changing external/internal conditions, and can include a communication to the customer or end user as to the status of the product. A simple definition of intelligent packaging is ‘packaging which senses and informs’, and nowhere does this generate a more potent vision than within the smart home of the future.

Factors That Will Aid the Growth of Intelligent Packaging. Consumer and societal factors are likely to drive the adoption of smart packaging in the future. The growing need for information on packaging will mean there has to be a step change in providing this information. Consumers increasingly need to know what ingredients or components are in the product and how the product should be stored and used. Intelligent labelling and printing, for example, will be capable of communicating directly to the customer via thin film devices providing sound and visual information, either in response to touch, motion or some other means of scanning or activation. Voice-activated safety and disposal instructions contained on household and pharmaceutical products will be used to tell the consumer how they should be disposed of after consumption—information that can be directly used in the recycling industry to help sort packaging materials from waste streams. Drug delivery systems in smart packaging will be programmed to communicate patient information back to healthcare centres.

Quality Assurance Using Intelligent Labels. Another important need is for consumer security assurance, particularly for perishable food products. The question as to whether, for example, a chilled ready-meal is safe to use or consume is currently answered by ‘best by’ date stamping. However, this does not take into account whether the product has inadvertently been exposed to elevated temperatures during storage or transportation. In the future, microbial growth and temperature-time visual indicators based on physical, chemical or enzymatic activity in the food will give a clear, accurate and unambiguous indication of product quality, safety and shelf-life condition. Labels can be attached to the outside of packaging film, which monitors the freshness of seafood products. A barb on the backside of the tag penetrates the packaging film and allows the passage of volatile amines, generated by spoilage of the seafood. These are scanned passed a chemical sensor that turns label progressively bright pink as the seafood ages.

The invention can optionally provide the nanocrystalline (NC) products and/or other materials that can optionally be combined with other materials, e.g., plastic, aluminum, steel, kevlar, cast iron, fibers, alloys and/or composites that can optionally increase strength and/or hardness and/or multiple nanocrystalline (NC) applications.

The invention can also optionally include, but it not limited to, using or adding nanocrystalline cellulose (NCC), nanocrystalline (NC) plastics or nanocrystalline (NC) polymers or other nanocrystals of cellulose composites or structures with manganese phosphates are of considerable industrial interesting properties nowadays because of their wide applications in laser host, ceramic, dielectric, electric, magnetic, and catalytic processes, including but not limited to, manganese (III) phosphates such as Manganese dihydrogenphosphate dihydrate (Mn(H2PO4)2.2H2O), MnP3O9, MnPO4.H2O, MnPO4, MnHP2O7 and Mn3(PO4)3, which can be made according to known methods, as known in the art, e.g., Danvirutai et al., Journal of Alloys and Compounds 457 (2008) pp. 75-80, entirely incorporated by reference. The invention can also optionally include compositions and methods using the nanocrystalline cellulose ((NCC), nanocrystalline (NC) plastics or nanocrystalline (NC) polymers or other nanocrystals of cellulose composites or structures of the invention for use in fertilizers, pesticides and/or herbicides and/or with micronutrients added to fertilizers, such as insoluble micronutrients, smart macronutrients or smart micronutrients, optionally in applications including combining them with nitrogen-phosphorus-potassium (NPK) fertilizers and coating them on NPK fertilizers and seeds, and also in and used with controlled-release fertilizer of zinc encapsulated by a manganese hollow core shell (Soil Science and Plant Nutrition, v.61, (2), pp. 319-326 (2015)), e.g., macronutrients can include one or more of sources or compounds comprising one or more of calcium, carbon, hydrogen, magnesium, nitrogen, oxygen, phosphorus, potassium, or sulphur; and/or micronutrients can include one or more of sources or compounds comprising one or more of boron, chloride, cobalt, copper, iron, molybdenum, manganese, nickel, silicon, sodium, and/or zinc.

The invention can also include adding using or adding nanocrystalline cellulose (NCC), nanocrystalline (NC) plastics or nanocrystalline (NC) polymers or other nanocrystals of cellulose composites or structures to magnesium chloride, potassium chloride and/or sodium chloride; for use with hydroxyapatite, e.g., one or more of reconstruction of bone or teeth, chromotrography, gas sensors, filter to purify liquids, water purification and/or desalination (e.g., polyvinylidenefluoride-co-hexafluoropropylene (PVDF-HFP) membranes containing different amounts of nanocrystalline cellulose (NCC), as known in the art, e.g., Lalia et. al., Desalination v.332, pp. 134-141 (2014)), fertilizers, and drug carriers, based on properties including one or more of powder properties, e.g., particles size, surface area, and morphology, which improve the properties thereof, (e.g., as known in the art, e.g., Klinkaewnarong et al. Current Applied Physics 10 (2010) 521-525).

Nanocrystalline cellulose (NCC), nanocrystalline (NC) plastics or nanocrystalline (NC) polymers or other nanocrystals of cellulose composites or structures can optionally be used in batteries, e.g., NiMH, or Lithium (Li) batteries or rechargeable batteries or supercapacitors, as nanocrytalline metal hydrides, including, but not limited to, one or more of structure, electrochemical and electronic properties of nanocrystalline and polycrystalline TiFe-, LaNi5- and Mg2Ni-type phases, which can optionally be prepared by mechanical alloying (MA) followed by annealing or by induction melting method, respectively. The properties of hydrogen host materials can be modified substantially by alloying to obtain the desired storage characteristics, e.g., respective replacement of Fe in TiFe by Ni and/or by Mg, Cr, Mn, Co, Mo, Zr, or for Li batteries, e.g., LiMn2O4, γ-Fe2O3, fluorine-doped tin oxide and potassium manganese oxyiodide or nanocrystalline solid solutions AlySn1-yO2-y/2 (y=0.57, 0.4) as electrode materials for lithium-ion batteries (e.g., Becker et al. Journal of Power Sources, Volume 229, 1 May 2013, Pages 149-158, which can improve not only the discharge capacity but also the cycle life of these electrodes, e.g., nanocrystalline TiFe0.125Mg0.125Ni(0.75) powder, e.g., cobalt substituting nickel in LaNi4-xMn0.75Al0.25Cox alloy greatly improves the discharge capacity and cycle life of LaNi5 material, e.g., nanocrystalline LaNi3.75Mn0.75Al0.25Co0.25 powder.

Supercapacitors and batteries can optionally include nanocrystalline transition metal nitrides (TMN) based on vanadium nitride, that can optionally deliver a specific capacitance of 1,340 F/g when tested at low scan rates of 2 mV/s and 554 F/g when tested at high charging rates of 100 mV/s in the presence of a 1M KOH electrolyte; and/or using nanostructured vanadium nitride and controlled oxidation of the surface at the nanoscale can optionally be in supercapacitors used in e.g., cars, camcorders and lawn mowers to industrial backup power systems at hospitals and airports.

Nanocrystalline cellulose (NCC), nanocrystalline (NC) plastics or nanocrystalline (NC) polymers or other nanocrystals of cellulose composites or structures can optionally be used in inverter components and materials such as nanocrystalline soft magnetic materials, e.g., of Fe-based soft magnetic material.

Nanocrystalline cellulose (NCC), nanocrystalline (NC) plastics or nanocrystalline (NC) polymers or other nanocrystals of cellulose composites or structures of the invention can also optionally include nanocomposites fabricated by gelation and electrospinning, which can have advantages for improving mechanical properties of both nanocomposite hydrogels and electrospun nanocomposite fibers/mats, as used in the invention, which can optionally include, as known in the art, including multifunctional properties, nanocomposite hydrogels from CNCs and other stimuli responsive polymers, e.g., nanocomposite hydrogels reinforced with CNCs can include one or more of fast temperature, pH, and salt sensitivity, e.g., for controllable drug delivery systems, pharmaceutical coatings process, medical coating applications, topical ophthalmic protectant and lubricant and the like, and other applications, e.g., hydrophilicity, biodegradability, biocompatibility, low cost, and non-toxicity, e.g., tissue engineering. Electrospun nanocomposite fibers can optionally include improved fabrication, morphology, mechanical and/or thermal properties with designed and improved functional characteristics and properties, such as, but not limited to energy-related materials, sensor, barrier films, and tissue engineering scaffolds, as known in the art.

Nanocrystalline cellulose (NCC), nanocrystalline (NC) polymers, nanocrystalline (NC) plastics, or other nanocrystals of cellulose composites or structures of the invention can also optionally include using or adding at least one fertilizer, pesticide and/or herbicide with at least one micronutrient, microingredient, growth hormone, crop additive, that is incorporated into and used by agricultural seeds and/or vegetable seeds of crops or genetically modified (GM) organisms or genetically modified (GM) crops or genetically engineered (GE) crops or genetically modified (GM) foods or ingredients or artificial ingredients in human and pet food or processed food and affect their germination, growth, crop yield, product quality and growth rates and support water uptake inside seeds, a process, which can affect seed germination, growth, crop yield, product quality and growth of seedlings through enhanced fertilizer uptake capacity, herbicide tolerance, insect tolerance, drought tolerance and increased food and/or vegetation production, agricultural products, industrial products, agricultural based products, compound feed, animal feed, agricultural commodities, fruits, food ingredients, food products, food packaging applications, food applications, food additives, organic food additives, organic products, soy bean, protein, soy products, milk production and/or dairy products and the like.

Nanocrystalline cellulose (NCC), nanocrystalline (NC) polymers, nanocrystalline (NC) plastics, or other nanocrystals of cellulose composites or structures of the invention can also optionally include using or adding pesticides or herbicides for attracting, seducing, and then destroying, or mitigating any pest. They are a class of biocide. The most common use of pesticides is as plant protection products (also known as crop protection products), which in general protect plants from damaging influences such as weeds, plant diseases or insects. This use of pesticides is so common that the term pesticide is often treated as synonymous with plant protection product, although it is in fact a broader term, as pesticides are also used for non-agricultural purposes. The term pesticide includes all of the following: herbicide, insecticide, insect growth regulator, nematicide, termiticide, molluscicide, piscicide, avicide, rodenticide, predacide, bactericide, insect repellent, animal repellent, antimicrobial, fungicide, disinfectant (antimicrobial), and sanitizer. In general, a pesticide is a chemical or biological agent (such as a virus, bacterium, antimicrobial, or disinfectant) that deters, incapacitates, kills, or otherwise discourages pests. Target pests can include insects, plant pathogens, weeds, mollusks, birds, mammals, fish, nematodes (roundworms), and microbes that destroy property, cause nuisance, or spread disease, or are disease vectors. Although pesticides have benefits, some also have drawbacks, such as potential toxicity to humans and other desired species.

Nanocrystalline cellulose (NCC), nanocrystalline (NC) polymers, nanocrystalline (NC) plastics, or other nanocrystals of cellulose composites or structures of the invention can also optionally include using or adding entomopathogenic fungus or fungi that can act as a parasite of insects and kills or seriously disables them.

Nanocrystalline cellulose (NCC), nanocrystalline (NC) polymers, nanocrystalline (NC) plastics, or other nanocrystals of cellulose composites or structures of the invention can also optionally include using or adding superdispersive iron, cobalt and copper nanocrystalline powders were synthesized in a water-ethanol medium by the reduction method using sodium borohydride as a reducing agent and carboxymethyl cellulose as a stabilizer (for Fe and Co nanoparticles). Transmission electron microscopy micrographs and x-ray diffraction analyses of the freshly prepared nanocrystalline powders indicated that they were in a zerovalent state with particles sizes ranging from 20 to 60 nm. The soybean seeds were treated with an extra low nanocrystalline dose (not more than 300 mg of each metal per hectare) and then sowed on an experimental landfill plot consisting of a farming area of 180 m2. This pre-sowing treatment of soybean seeds, which does not exert any adverse effect on the soil environment, reliably changed the biological indices of the plant growth and development. In particular, in laboratory experiments, the germination rates of soybean seeds treated with zerovalent Cu, Co and Fe were 65%, 80% and 80%, respectively.

Nanocrystalline cellulose (NCC), nanocrystalline (NC) polymers, nanocrystalline (NC) plastics, or other nanocrystals of cellulose composites or structures of the invention can also optionally include using or adding nanocrystalline apatite-based materials and stem cells in orthopedic surgery and traumatology that have the potential of improving quality of life of the elderly and enhance health-related socio-economic challenges. Nanocrystalline apatite-based materials and especially calcium phosphate nano-materials exploit new physical, chemical and biological properties that have the possibility to increase surface area and improve tissue integration. Stem cells of adult origin decrease inflammation, increase vascularity and are able to replace degenerated tissue cells during the process of regeneration. The bone is the only human tissue that regenerates. Musculoskeletal disorders including osteoporotic fractures and osteoarthritis decrease quality of life in the elderly and cause severe burden on economics. Nanocrystalline calcium phosphate bioceramics have the ability to prevent or treat osteoporotic fractures when combined with stem cells. These materials may also be used for drug delivery purposes to treat bone infections when combined with stem cell as they can assist in treating osteoarthritis.

At least one embodiment of the invention is directed towards adding at least one nanocrystalline cellulose (NCC), nanocrystalline (NC) polymers, nanocrystalline (NC) plastics, or other nanocrystals of cellulose composites or structures to a substrate, component, or additive in a nanocrystalline (NC) product making process. The at least one nanocrystalline cellulose (NCC), nanocrystalline (NC) polymers, nanocrystalline (NC) plastics, or other nanocrystals of cellulose composites or structures can optionally be added in the wet end and/or in the dry end. The at least one nanocrystalline cellulose (NCC), nanocrystalline (NC) polymers, nanocrystalline (NC) plastics, or other nanocrystals of cellulose composites or structures can optionally be added as a coating outside of the substrate or can optionally be dispersed within the substrate. A coating can optionally partially or fully enclose the substrate. The at least one nanocrystalline cellulose (NCC), nanocrystalline (NC) polymers, nanocrystalline (NC) plastics, or other nanocrystals of cellulose composites or structures can optionally comprise linear, branched, cyclic, polymers extending from the NCC core and/or can optionally be an NCC Graft Polymer.

Optional NC components that can optionally be in the invention can include NC celluloses such as one or more of naturally occurring crystals such as those present in plant fibers. A typical cellulose bearing fiber comprises regions of amorphous cellulose and regions of crystalline cellulose. NCC can optionally be obtained by separating the crystalline cellulose regions from the amorphous cellulose regions of a plant fiber. Because their compact nature makes crystalline cellulose regions highly resistant to acid hydrolysis, NCC is often obtained by acid hydrolyzing plant fibers. NCC crystallites can optionally have 5-10 nm diameter and 100-500 nm length. NCC can optionally have a crystalline fraction of no less than 80% and often between 85% and 97%. See, e.g., U.S. 2011/0293932, 2011/0182990, 2011/0196094, and U.S. Pat. No. 8,398,901 (entirely incorporated herein by reference).

In at least one embodiment the composition added to a product substrate optionally comprises an NCC core with at least one polymer chain extending from the NCC core. NCC comprises a number of hydroxyl groups, which are possible anchor sites from which polymer chains can optionally extend. Without being limited by a particular theory or design of the invention or of the scope afforded in construing the claims, it is believed that because of one or more of Nanocrystalline cellulose (NCC) unique aspect ratio, density, anchor sites, rigidity and supporting strength, At least one nanocrystalline cellulose (NCC), nanocrystalline (NC) polymers, nanocrystalline (NC) plastics, or other nanocrystals of cellulose composites or structures are able to arrange polymer chains in unique arrangements that afford a number of unique properties that enhance product characteristics.

In at least one embodiment the at least one nanocrystalline cellulose (NCC), nanocrystalline (NC) polymers, nanocrystalline (NC) plastics, or other nanocrystals of cellulose composites or structures is optionally added in the wet end of a nanocrystalline (NC) product making process. In at least one embodiment the at least one nanocrystalline cellulose (NCC), nanocrystalline (NC) polymers, nanocrystalline (NC) plastics, or other nanocrystals of cellulose composites or structures is added as a coating in the size press of a nanocrystalline (NC) product making process. Detailed descriptions of the wet and dry ends of a nanocrystalline (NC) product making process and addition points for chemical additives therein are known in the art, e.g., similar to those described in the reference Handbook for Pulp and Paper Technologists, 3rd Edition, by Gary A. Smook, Angus Wilde Publications Inc., (2002). The at least one nanocrystalline cellulose (NCC), nanocrystalline (NC) polymers, nanocrystalline (NC) plastics, or other nanocrystals of cellulose composites or structures can optionally be added to the nanocrystalline (NC) product making process at any addition point(s) described therein for any other chemical additive and according to the methods and with any of the apparatuses also described therein.

In at least one embodiment the at least one nanocrystalline cellulose (NCC), nanocrystalline (NC) polymers, nanocrystalline (NC) plastics, or other nanocrystals of cellulose composites or structures is optionally formed by the derivatization of one or more hydroxyl groups on an NC crystal through condensation polymerization or grafting of vinyl monomers via radical polymerization to meet desired end user requirements.

In at least one embodiment the polymer attached to the NCC core is a polysaccharide. In at least one embodiment the polysaccharide at least one nanocrystalline cellulose (NCC), nanocrystalline (NC) polymers, nanocrystalline (NC) plastics, or other nanocrystals of cellulose composites or structures is used as viscosity modifier in enhanced oil recovery, as flocculants for wastewater treatment and filler strength agent in a nanocrystalline (NC) product making process.

In at least one embodiment the polymer attached to the NCC core is a vinyl polymer. In at least one embodiment it is a copolymer having structural units of at least two vinyl monomers including but not limited to acrylamide and acrylic acid. Polyacrylamide, polyacrylic acid, and 2-(methacryloyloxy)ethyl trimethylammonium chloride are efficient flocculants for water treatment and various applications. However, vinyl polymers show limited biodegradability and poor shear stability whereas NCC is shear stable but are less efficient as flocculants. Connecting non-ionic, anionic, and/or cationic vinyl monomers on an NCC core yields better performing polyelectrolyte flocculants, and filler materials.

In at least one embodiment the at least one nanocrystalline cellulose (NCC), nanocrystalline (NC) polymers, nanocrystalline (NC) plastics, or other nanocrystals of cellulose composites or structures is added to the nanocrystalline (NC) product making process alongside 2-(methacryloyloxy)-ethyl trimethylammonium chloride. In at least one embodiment the at least one nanocrystalline cellulose (NCC), nanocrystalline (NC) polymers, nanocrystalline (NC) plastics, or other nanocrystals of cellulose composites or structures added to a nanocrystalline (NC) product making process is exposed to shear in excess to what a non-at least one nanocrystalline cellulose (NCC), nanocrystalline (NC) polymers, nanocrystalline (NC) plastics, or other nanocrystals of cellulose composites or structures can optionally endure and still function, and continues to function.

In at least one embodiment the at least one nanocrystalline cellulose (NCC), nanocrystalline (NC) polymers, nanocrystalline (NC) plastics, or other nanocrystals of cellulose composites or structures is a branched polymer in which from a first chain of polymer structural units extending from the NCC core, one or more distinct other chains branch off from the first polymer chain and/or from other distinct chain branches. In at least one embodiment the first chain is comprised of a different variety of monomer units than one or more of the branch chains. Differences in chain compositions allows for versatile polymer arrangements as a means of imparting a variety of functional groups to a polymer. It also permits one to combine the best properties of two or more polymers in one physical unit. For example the first chain can optionally be selected for its capacity to support or position functionally active polymer branches according to a geometry, which has superior effects.

In at least one embodiment the polymer chain/branch is optionally formed or grown according to one or more of: a grow-to method, a grow-from method, and/or a grow-through method. In the grow-to approach an end group of a pre-formed polymer is coupled with a functional group on the NCC core. In the growing-from approach, the growth of the polymer chain occurs from initiation sites attached to the NCC core. In the growing-through approach a vinyl macro-monomer of cellulose is copolymerized from the NCC core with low molecular weight co-monomer.

Representative examples of vinyl monomers which can optionally be used for any of the three growth or synthesis approaches include, but are not limited to vinyl acetate, acrylic acid, sodium acrylate, ammonium acrylate, methyl acrylate, acrylamide, acrylonitrile, N,N-dimethyl acrylamide, 2-acrylamido-2-methylpropane-1-sulfonic acid, sodium 2-acrylamido-2-methylpropane-1-sulfonate, 3-acrylamidopropyl-trimethyl-ammonium chloride, diallyldimethylammonium chloride, 2-(dimethylamino)ethyl acrylate, 2-(acryloyloxy)-N,N,N-trimethylethanaminium chloride, N,N-dimethylaminoethyl acrylate benzyl chloride quaternary salt, 2-(acryloyloxy)-N,N,N-trimethylethanaminium methyl sulfate, 2-(dimethylamino)ethyl methacrylate, 2-(methacryloyloxy)-N,N,N-trimethylethanaminium chloride, 2-(methacryloyloxy)-N,N,N-trimethylethanaminium methyl sulfate, 3-(dimethylamino)propyl methacrylamide, methacrylic acid, methacrylic anhydride methyl methacrylate, methacryloyloxy ethyl trimethyl ammonium chloride, 3-methacrylamidopropyl-trimethyl-ammonium chloride, hexadecyl methacrylate, octadecyl methacrylate, docosyl acrylate, n-vinyl pyrrolidone, 2-vinyl pyridine, 4-vinyl pyridine, epichlorohydrin, n-vinyl formamide, n-vinyl acetamide, 2-hydroxyethyl acrylate glycidyl methacrylate, 3-(allyloxy)-2-hydroxypropane-1-sulfonate, 2-(allyloxy)ethanol, ethylene oxide, propylene oxide, 2,3-epoxypropyltrimethylammonium chloride, (3-glycidoxypropyl)trimethoxy silane, epichlorohydrin-dimethylamine, vinyl sulfonic acid sodium salt, Sodium 4-styrene sulfonate, caprolactam and any combination thereof.

In at least one embodiment addition of an at least one nanocrystalline cellulose (NCC), nanocrystalline (NC) polymers, nanocrystalline (NC) plastics, or other nanocrystals of cellulose composites or structures to a nanocrystalline (NC) product making process, furnish or slurry can improve at least drainage retention. As shown in the Examples, At least one nanocrystalline cellulose (NCC), nanocrystalline (NC) polymers, nanocrystalline (NC) plastics, or other nanocrystals of cellulose composites or structures used alongside starch, a cationic flocculant and an acrylic acid polymer have superior retention performance to such drainage programs lacking the at least one nanocrystalline cellulose (NCC), nanocrystalline (NC) polymers, nanocrystalline (NC) plastics, or other nanocrystals of cellulose composites or structures. Improved retention of fines, fillers, and other components of the furnish decreases the amount of such components lost to the whitewater and hence reduces the amount of material wastes, the cost of waste disposal and the adverse environmental effects. It is generally desirable to reduce the amount of material employed in a nanocrystalline (NC) product making process.

In at least one embodiment adding the at least one nanocrystalline cellulose (NCC), nanocrystalline (NC) polymers, nanocrystalline (NC) plastics, or other nanocrystals of cellulose composites or structures to a nanocrystalline (NC) product making process furnish or slurry improves wet strength. As known in the art, e.g., as described in U.S. Pat. No. 8,172,983 (entirely incorporated by reference), a high degree of wet strength in product is desired to allow for the addition of more filler (such as PCC or GCC) to the product. Increasing filler content results in superior optical properties and cost savings (filler is cheaper than fiber).

In at least one embodiment, the at least one nanocrystalline cellulose (NCC), nanocrystalline (NC) polymers, nanocrystalline (NC) plastics, or other nanocrystals of cellulose composites or structures is added as a coating or as part of a coating during size press of a nanocrystalline (NC) product making process. The at least one nanocrystalline cellulose (NCC), nanocrystalline (NC) polymers, nanocrystalline (NC) plastics, or other nanocrystals of cellulose composites or structures can optionally be added as a coating applied during a size press operation and can optionally be added alongside starch, sizing agents or any other additive added during the size press.

In at least one embodiment the at least one nanocrystalline cellulose (NCC), nanocrystalline (NC) polymers, nanocrystalline (NC) plastics, or other nanocrystals of cellulose composites or structures added to the nanocrystalline (NC) product making process is an NCC graft polymer. The graft polymer can optionally comprise two or more NCC cores. The NCC graft polymer can optionally include a single polymer chain bridging between the NCC cores. The NCC Graft can optionally also include two or more NCC cores with distinct polymer chains that are cross-linked to each other. As such an at least one nanocrystalline cellulose (NCC), nanocrystalline (NC) polymers, nanocrystalline (NC) plastics, or other nanocrystals of cellulose composites or structures can optionally be cross-linked to at least one other At least one nanocrystalline cellulose (NCC), nanocrystalline (NC) polymers, nanocrystalline (NC) plastics, or other nanocrystals of cellulose composites or structures where the cross-linkage is located at one of the structural units of the polymer and not at the NCC core. The cross linkage can optionally be achieved by one or more polymer cross-linking agents known in the art. The NCC graft polymer can optionally be in the form of a hydrogel, resin as described in US 2011/0182990 (entirely incorporated herein by reference).

In at least one embodiment at least one nanocrystalline cellulose (NCC), nanocrystalline (NC) polymers, nanocrystalline (NC) plastics, or other nanocrystals of cellulose composites or structures of the invention is added to a commercial process. The at least one nanocrystalline cellulose (NCC), nanocrystalline (NC) polymers, nanocrystalline (NC) plastics, or other nanocrystals of cellulose composites or structures, or nanocrystalline (NC) product, can optionally comprise a mixture comprising one or more of: a) at least one nanocrystalline cellulose (NCC), nanocrystalline (NC) polymers, nanocrystalline (NC) plastics, or other nanocrystals of cellulose composites or structures mixed with a polymer additive that is not an at least one nanocrystalline cellulose (NCC), nanocrystalline (NC) polymers, nanocrystalline (NC) plastics, or other nanocrystals of cellulose composites or structures, b) at least one nanocrystalline cellulose (NCC), nanocrystalline (NC) polymers, nanocrystalline (NC) plastics, or other nanocrystals of cellulose composites or structures mixed with a polymer additive that is an at least one nanocrystalline cellulose (NCC), nanocrystalline (NC) polymers, nanocrystalline (NC) plastics, or other nanocrystals of cellulose composites or structures, and/or c) a polymer additive which comprises at least one nanocrystalline cellulose (NCC), nanocrystalline (NC) polymers, nanocrystalline (NC) plastics, or other nanocrystals of cellulose composites or structures. In at least one embodiment the polymer additive is a polymer made up of one or more of at least one nanocrystalline cellulose (NCC), nanocrystalline (NC) polymers, nanocrystalline (NC) plastics, or other nanocrystals of cellulose composites or structures, non-ionic, water-soluble monomers, anionic monomers, cationic monomers, and any combination thereof. The polymer additives can optionally be manufactured according any process known in the art, e.g., but not limited to, as described in one or more of: Emulsion Polymerization and Emulsion Polymers, by Peter A. Lovell et al, John Wiley and Sons, (1997), Principles of polymerization, Fourth Edition, by George Odian, John Wiley and Sons, (2004), Handbook of RAFT Polymerization, by Christopher Barner-Kowollik, Wiley-VCH, (2008), Handbook of Radical Polymerization, by Krzysztof Matyjaszewski et al, John Wiley and Sons, (2002), Controlled/Living Radical Polymerization: Progress in ATRP, NMP, and RAFT: by K. Matyjaszewski, Oxford University Press (2000), and Progress in Controlled Radical Polymerization: Mechanisms and Techniques, by Krzysztof Matyjaszewski et al, ACS Symposium Series 1023 (2009). The polymer additives can optionally be manufactured according any process including but not limited to Solution, emulsion, inverse-emulsion, dispersion, atom transfer radical polymerization (ATRP), Reversible addition-fragmentation-chain transfer polymerization (RAFT), and ring opening polymerization.

The at least one nanocrystalline cellulose (NCC), nanocrystalline (NC) polymers, nanocrystalline (NC) plastics, or other nanocrystals of cellulose composites or structures, or product, of the invention can optionally used in any of commercial product or process, such as, but not limited to, one or more of any known product, e.g., but not limited to, as described herein or as known in the art.

Optional examples of components used in making or using a nanocrystalline (NC) product of the invention can optionally include non-ionic, water-soluble monomers suitable for use in a polymer additive can include one or more of: acrylamide, methacrylamide, N,N-dimethylacrylamide, N,N-diethylacrylamide, N-isopropylacrylamide, N-vinylformamide, N-vinylmethylacetamide, N-vinyl pyrrolidone, 2-vinyl pyridine, 4-vinyl pyridine, epichlorohydrin, acrylonitrile, hydroxyethyl methacrylate, hydroxyethyl acrylate, hydroxypropyl acrylate, hydroxypropyl methacrylate, hexadecyl methacrylate, octadecyl methacrylate, glycidyl methacrylate, 3-(glycidoxypropyl)trimethoxy silane, 2-allyloxy ethanol, docosyl acrylate, N-t-butylacrylamide, N-methylolacrylamide, epichlorohydrin-dimethylamine, caprolactam, and the like.

Optional examples of components used in making or using a nanocrystalline (NC) product of the invention can optionally include anionic monomers which can optionally include one or more of: acrylic acid, and its salts, including, but not limited to sodium acrylate, and ammonium acrylate, methacrylic acid, and its salts, including, but not limited to sodium methacrylate, and ammonium methacrylate, 2-acrylamido-2-methylpropanesulfonic acid (AMPS), the sodium salt of AMPS, sodium vinyl sulfonate, styrene sulfonate, maleic anhydride, maleic acid, and it's salts, including, but not limited to the sodium salt, and ammonium salt, sulfonate itaconate, sulfopropyl acrylate or methacrylate, or other water-soluble forms of these or other polymerisable carboxylic or sulphonic acids and crotonic acid and salts thereof. Sulfomethylated acrylamide, allylsulfonate, sodium vinyl sulfonate, itaconic acid, acrylamidomethyl butanoic acid, fumaric acid, vinylphosphonic acid, vinylsulfonic acid, vinylsulfonic acid sodium salt, allylphosphonic acid, 3-(allyloxy)-2-hydroxypropane sulfonate, sulfomethyalted acryamide, phosphono-methylated acrylamide, ethylene oxide, propylene oxide and the like.

Optional examples of components used in making or using a nanocrystalline (NC) product of the invention can optionally include cationic monomers which can optionally include one or more of: dialkylaminoalkyl acrylates and methacrylates and their quaternary or acid salts, including, but not limited to, dimethylaminoethyl acrylate methyl chloride quaternary salt, dimethylaminoethyl acrylate methyl sulfate quaternary salt, dimethylaminoethyl acrylate benzyl chloride quaternary salt, dimethylaminoethyl acrylate sulfuric acid salt, dimethylaminoethyl acrylate hydrochloric acid salt, dimethylaminoethyl methacrylate methyl chloride quaternary salt, dimethylaminoethyl methacrylate methyl sulfate quaternary salt, dimethylaminoethyl methacrylate benzyl chloride quaternary salt, dimethylaminoethyl methacrylate sulfuric acid salt, dimethylaminoethyl methacrylate hydrochloric acid salt, dialkylaminoalkylacrylamides or methacrylamides and their quaternary or acid salts such as acrylamidopropyltrimethylammonium chloride, dimethylaminopropyl acrylamide methyl sulfate quaternary salt, dimethylaminopropylacrylamide sulfuric acid salt, dimethylaminopropyl acrylamide hydrochloric acid salt, methacrylamide propyltrimethylammonium chloride, dimethylaminopropyl methacrylamide methyl sulfate quaternary salt, dimethylaminopropyl methacrylamide sulfuric acid salt, dimethylaminopropyl methacrylamide hydrochloric acid salt, diethylaminoethylacrylate, diethylaminoethylmethacrylate, diallyldiethylammonium chloride, diallyldimethyl ammonium chloride and 2,3-epoxypropyltrimethylainmonium chloride. Alkyl groups are generally C1-4 alkyl.

The present invention optionally provides a method of making a composite, comprising two or more of: (a) providing an aqueous mixture comprising partially hydrolyzed cellulose in a dissolution media; (b) providing a solution comprising a aliphatic polyester in a polar organic solvent; (c) combining the mixture with the solution to form a precipitate; and then (d) washing the precipitate with water to remove solvent and dissolution media and produce a wet composite; and (e) drying the wet composite to produce a dry composite.

The washing step can optionally be carried out continuously or as a batch process by any suitable technique, such as by mixing and separating (e.g., by settling, filtration, or centrifugation), washing of a “cake,” dialysis, and combinations thereof. Washing can optionally be carried out with distilled water, or the water may contain additional ingredients such as salts, buffers, etc. Specific washing steps can optionally be repeated and/or continued until the desired degree of washing is achieved. In one or more optional embodiments, the washing step is carried out until the wet composite has a neutral pH (e.g., a pH between 6 and 7).

The drying step can optionally be carried out by any suitable means. In one or more optional embodiments, the drying step is carried out at room temperature, with heating (e.g., baking), or during cooling (e.g., chilling or freezing). The drying step can be carried out at any suitable pressure, including atmospheric pressure or at a reduced pressure (e.g., as in freeze drying).

The dry composite so produced is preferably rigid. In one or more optional embodiments, the composite so produced has (i) a storage modulus represented by an integer between 1 or 5 gigapascals, up to 20, 25, or 35 gigapascals, at a temperature of 20 degrees C., and/or (ii) a storage modulus represented by an integer between 0.1 or 1 gigapascals, up to 10 or 20 gigapascals, at a temperature of 100 degrees Centigrade.

In one or more optional embodiments, the dry composite so produced is porous. In one or more optional embodiments, the dry composite so produced has a density of 0.01, 0.05 or 0.1 grams per cubic centimeter, up to 0.5, 1, 5 or 10 grams per cubic centimeter. In one or more optional embodiments, the composite has a residual weight of about 1%, 2% or 5% to 10%, 15%, or 20% at a temperature of 400 degrees C.

If desired, the combining step, and the optional washing and/or dialyzing step, can be carried out in a form or mold. In this case the method can further comprises the step of: (e) releasing the composite from the form or mold to produce a composite product (optionally having a shape corresponding to the shape of the form or mold), optionally followed by the steps of: (f) cutting or grinding the product to further define the features thereof, and/or (g) grinding the product to form a particulate composite.

Thus the method of the invention can optionally be used for the purpose of producing different Nanocrystalline (NC) products, which can include, but are not limited to, an insulating product, as can optionally be used for architectural or building purposes, or configured for refrigeration, chilling and/or freezing apparatus. In addition, products of the invention can optionally be configured for use as a tissue engineering scaffold, as can optionally be used for bone or soft tissue regeneration in vitro or in vivo. Particulate composites produced by the methods of the present invention are useful as, among other things, a pharmaceutical tablet filler or excipient.

### Description of Non-Limiting Exemplary Embodiments Potential Aspects or Elements of the Claimed Invention that can Optionally be Excluded or Negatively Claimed

The present invention can optionally also in particular claimed embodiments exclude or negatively claim one or more aspects, e.g., to more particularly recite or exclude embodiments or elements that might occur in cited or other published art, as presented herein. Accordingly, the present invention can optionally exclude, not include, or not provide, one of more, or any combination of any element, feature, component or step disclosed herein.

A number of implementations have been described. Nevertheless, it can optionally be understood that various modifications can optionally be made. For example, elements of one or more implementations can optionally be combined, deleted, modified, or supplemented to form further implementations. As yet another example, the logic flows depicted in the figures do not require the particular order shown, or sequential order, to achieve desirable results. In addition, other steps can optionally be provided, or steps can optionally be eliminated, from the described flows, and/or other components can optionally be added to, or removed from, the described systems. Accordingly, other implementations are within the scope of the following claims.

While this invention can optionally be embodied in many different forms, there are described in detail herein specific preferred embodiments of the invention. The present disclosure is an exemplification of the principles of the invention and is not intended to limit the invention to the particular embodiments illustrated. All patents, patent applications, scientific papers, and any other referenced materials mentioned herein are incorporated by reference in their entirety. Furthermore, the invention encompasses any possible combination of some or all of the various embodiments mentioned herein, described herein and/or incorporated herein. In addition the invention encompasses any possible combination that also specifically excludes any one or some of the various embodiments mentioned herein, described herein and/or incorporated herein.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to.” Those familiar with the art can optionally recognize other equivalents to the specific embodiments described herein which equivalents are also intended to be encompassed by the claims.

All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10; that is, all subranges beginning with a minimum value of 1 or more, (e.g. 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range. All percent, ratios and proportions herein are by weight unless otherwise specified.

### Examples

The foregoing can optionally be better understood by reference to the following examples, which are presented for purposes of illustration and are not intended to limit the scope of the invention. In particular the examples demonstrate representative examples of principles innate to the invention and these principles are not strictly limited to the specific condition recited in these examples. As a result it should be understood that the invention encompasses various changes and modifications to the examples described herein and such changes and modifications can optionally be made without departing from the spirit and scope of the invention and without diminishing its intended advantages. It is therefore intended that such changes and modifications be covered by the appended claims.

### Example #1

A number of at least one nanocrystalline cellulose (NCC), nanocrystalline (NC) polymers, nanocrystalline (NC) plastics, or other nanocrystals of cellulose composites or structures are made according to a growing-from approach. A 4-neck, 1.5 L reactor is fitted with a) an overhead mechanical stirrer connected to a metal shaft and a conical stirrer, b) a nitrogen inlet and sparge tube, c) a claisen adapter fitted with a reflux condenser d) a temperature probe (RTD) inserted through Teflon connector and temperature is controlled by Athena. To the reactor is added a 562.5 mL of pH adjusted NCC (1.14×10−6 mol, 2.81 g, pH=2) dispersion and purged with N2 for 30 min and then ceric ammonium nitrate (CAN, 1.12×10−3 mol, 6.17 g) is allowed to react with NCC backbone for 15 min under N2 at R.T. The reactor is set to 70° C. and then 52.41 g of acrylamide (7.38×10-1 mol), 17.08 g of acrylic acid (3.16×10−1 mol) and water (84.67 g) are added to reactor at 42° C. Reaction mixture is heated to 70° C. and is maintained at 70° C. for 6 h. At 45 min 160 ppm of sodium hypo phosphite is added. Reaction is monitored by HNMR analysis of reaction aliquots (quenched with 500-1000 ppm of hydroquinone) and reached 92% conversion in 6 h (Table 2). Post modification is carried out using potassium persulfate (KPS, 500 μmol) and sodium metabisulfite (SBS, 3500 μmol) to burn out residual monomers. The nitrogen sparge is maintained throughout the reaction. The final pH of polymer is adjusted to 3.5 with NaOH and submitted to application testing. All samples are submitted for residual acrylamide and acrylic acid analyses. Results are that at least one nanocrystalline cellulose (NCC), nanocrystalline (NC) polymers, nanocrystalline (NC) plastics, or other nanocrystals of cellulose composites or structures are then added to a paper furnish. The alkaline furnish had a pH of 8.1 and is composed of 80% by weight cellulosic fibers and 20% precipitate calcium carbonate diluted to a consistency of 0.5% by weight. The fiber consists of ⅔ bleached hardwood kraft and ⅓ bleached softwood kraft. The retention performance of NCC and polymer-grafted NCC is evaluated using the Britt Jar test method.

500 ml of furnish is charged in Britt jar and mixed at 1250 rpm. Starch Solvitose N is then charged at 10 lb./ton dry weight at 5 seconds. Cationic flocculant 61067 is change at 20 seconds. Then at 55 seconds, NCC or At least one nanocrystalline cellulose (NCC), nanocrystalline (NC) polymers, nanocrystalline (NC) plastics, or other nanocrystals of cellulose composites or structures is charged. Drain started at 60 seconds and ended at 90 seconds. The drain (filtrate) is collected for turbidity measurement. The turbidity of the filtrate is inversely proportional to the furnish retention performance. The turbidity reduction % is proportional to the retention performance of the retention program. The higher the turbidity reduction %, the higher the retention of fines/or fillers. Two commercially available products, Nalco 8677 Plus (a polyacrylic acid polymer) and Nalco 8699 (a silica product), are tested for retention performance as references.

At the tested dosage range of 0.5 lb./ton to 2.0 lb./ton, NCC is expected to provide and an additional 25% to 40% turbidity reduction in comparison to a blank example, which are expected to be more well-performed than the two references 8677 Plus and 8699. At least one nanocrystalline cellulose (NCC), nanocrystalline (NC) polymers, nanocrystalline (NC) plastics, or other nanocrystals of cellulose composites or structures with acrylic acid (NCC/AA) and acrylamide/acrylic acid (NCC/AM/AA) is expected to show at least more 25% more turbidity reduction and at least 15% more turbidity reduction respectively than the blank. The results are expected to reveal that both NC cellulose and at least one NC polymers, or plastic significantly improve turbidity reduction of tested furnish, which are expected to provide better retention efficiency and cost reduction in Nanocrystalline (NC) product production.

### Example #2

The experiments are to contrast the ability of NCC and at least one polymer, or plastic to increase sheet dry strength in comparison to a conventional polyacrylamide based dry strength agent N-1044. At least one nanocrystalline cellulose (NCC), nanocrystalline (NC) polymers, nanocrystalline (NC) plastics, or other nanocrystals of cellulose composites or structures used in this example is 6653-145. The product contains 60% hardwood and 20% softwood and 20% precipitated calcium carbonate (PCC) as filler. 18 lb/ton cationic starch Stalok 310 is added as conventional dry strength agent, and various doses of NCC, polymer, or plastic and N-1044 are added after cationic starch. 1 lb/ton N-61067 is added as retention aid. The treated furnish is used to make hand sheet using Noble & Wood hand sheet mold. The composition is pressed using a static press and dried by passing it once through a drum dryer at about 105° C. The resulting hand sheets are allowed to equilibrate at 23° C. and 50% relative humidity for at least 12 hours before testing. Five duplicate hand sheets are made for each condition and the mean values are reported.

Addition of dry strength agents N-1044 and the nanocrystalline (NC) product of the invention are expected to provide improved filler retention and filler content into the sheet. Sheet product properties are compared at fixed ash content 20% based on the relationship of strength and filler content assuming sheet strength (ZDT and tensile index) decreases linearly with ash content. NCC alone did not increase sheet strength significantly. On the other side, at least one nanocrystalline cellulose (NCC), nanocrystalline (NC) polymers, nanocrystalline (NC) plastics, or other nanocrystals of cellulose composites or structures are expected to increase ZDT and tensile strength by at least 20% as compared to NCC alone. At least one nanocrystalline cellulose (NCC), nanocrystalline (NC) polymers, nanocrystalline (NC) plastics, or other nanocrystals of cellulose composites or structures is expected to be more effective than N-1044 especially at low dose 2 lb/ton.

### Example #3

Laboratory experiments are conducted to measure the ability of the NC C alone and at least one nanocrystalline cellulose (NCC), nanocrystalline (NC) polymers, nanocrystalline (NC) plastics, or other nanocrystals of cellulose composites or structures to increase the surface strength of paper as a nanocrystalline (NC) product of the invention. Base paper containing 16% ash and that has not been passed through a size press is coated using the drawdown method with solutions containing the desired chemistry. The mass of the paper before and after coating is used to determine specific chemical dose. The paper is dried by passing it once through a drum dryer at about 95° C. and allowed to equilibrate at 23° C. and 50% relative humidity for at least 12 hours before testing.

Surface strength is measured using TAPPI (Technical Association of Pulp and Paper Industries) method T476 om-01. In this measurement, the surface strength is inversely proportional to the amount of mass lost from the surface of the paper after having been systematically “rubbed” on a turn table by two abrasion wheels. The results are reported in mg of lost material per 1000 revolutions (mg/1000 revs): the lower the number the stronger the surface.

A first study compares the performance of the NCC with a copolymer of AA/AM known to increase paper surface strength. As part of the study, two blends of the NCC with the copolymer are tested.

The first three conditions span a range of starch dose within which the conditions containing the NCC, the copolymer and the blends are dosed. After accounting for the strengthening effect of starch, the abrasion loss results are expected to demonstrate that the NCC and the AA/AM copolymer have a similar level of performance. The effect is expected to be further enhanced when the additives are blended in a 50:50 and a 33:67 NCC:AA/AM ratio.

Next, a study is designed to determine whether growing an AA/AM copolymer on to the surface of the NCC improves the paper surface strength and compare its performance with that of the NCC. As part of this study, three at least one nanocrystalline cellulose (NCC), nanocrystalline (NC) polymers, nanocrystalline (NC) plastics, or other nanocrystals of cellulose composites or structures varying in the AA/AM monomer ratio are tested. The first three conditions span a range of starch dose within which the conditions containing the NCC and at least one nanocrystalline cellulose (NCC), nanocrystalline (NC) polymers, nanocrystalline (NC) plastics, or other nanocrystals of cellulose composites or structures are dosed. After accounting for the starch dose in each of the conditions, the abrasion loss results are expected to demonstrate that the grafting of the AA/AM copolymer on to the surface of the NCC is an improvement over the NCC. The surface strength performance is not expected to be affected, however, by the AA/AM monomer ratio in the 30/70 to 70/30 range.

Next, a study is designed to simultaneously compare surface strength performance as a function of all of the conditions (i.e., unmodified, modified with an anionic polymer of different mole ratios, and blends of the unmodified NCC with the AA/AM copolymer).

The first two conditions only contain starch, while the others contain about 1 or 3 lb/t of the additive. On conditions 15-18, the unmodified NCC:AAAM blends are prepared in a 10:90 mass ratio. The contributions of the multiple variables in this study are better elucidated with a regression analysis of the results. The model for the analysis resulted in a correlation coefficient of 0.80 with all variables (starch, the AA/AM copolymer, NCC, At least one nanocrystalline cellulose (NCC), nanocrystalline (NC) polymers, nanocrystalline (NC) plastics, or other nanocrystals of cellulose composites or structures, and the blends of AA/AM copolymer and the NCC) statistically contributing to the model. From highest to lowest, the magnitude of their contribution to strengthening the paper surface is expected to be the following: 1. Blends of AA/AM copolymer and NCC; and then 2. AA/AM copo, showing that Nanocrystalline (NC) products of the invention are expected to have enhanced strength, performance, and durability as compared to similar known materials, such as NCC alone.

### Example #4

Acid hydrolysis of cellulose is a popular method for isolating nanocrystalline cellulose (NCC) from cellulose fibers. Since the first publication related to the extraction (Mukherjee & Woods, 1953; Revol, Godbout, Dong, Gray, Chanzy & Maret, 1994; Revol, Bradford, Giasson, Marchessault & Gray, 1992) and use of NCC as reinforcing fillers based nanocomposites (Favier, Chanzy & Cavaille, 1995), they have attracted a great deal of interest in the nanocomposites field (Noishiki, Nishiyama, Wada, Kuga & Magoshi, 2002; Qi, Cai, Zhang & Kuga, 2009; Roman & Winter William, 2006) due to their appealing intrinsic properties such as nanoscale dimensions, high surface area, unique morphology, low density, and mechanical strength. Cellulose nanocomposites have been prepared using solution casting (Favier, Chanzy & Cavaille, 1995). In situ polymerization (Wu Q, 2002) and melt intercalation (Chazeau, Cavaillé, Canova, Dendievel & Boutherin, 1999).

Materials. PLA under the commercial name PLA 4060D (poly-D/L-lactide or PDLLA) is provided in the form of pellets. PLA 4060D has about 11 to 13% D-lactide content and has a density of 1.24 g/c.c. It has an amorphous morphology and melting temperature in the range of 150-180 C. Microcrystalline cellulose (MCC) is provided by FMC Bio Polymer (Avicel-PH101). Sulfuric acid, 95%-97%, Reagent Grade, is purchased from Scharlau. Tetrahydrofuran (THF) solvent is purchased from Sigma-Aldrich.

Processing. PLA is dissolved in a solvent such as THF. At the same time, microcrystalline cellulose (MCC) is hydrolyzed in a different container via hydrolyzing with 64%, 65% or 66% H2SO4 at ambient for 30, 60, 120 or 180 minutes. A ratio of 1 g:10 ml is adopted for the hydrolysis reactions (MCC: H2SO4). The two mixtures are then mixed with constant stirring. Upon mixing and washing, a white material is precipitated. The product is collected and washed with DI water through centrifugation and dialysis. The samples are dried and stored. Using this procedure 4 samples are produced at loading levels of 1%, 5%, 10%, 15%, 30% and 50% (w/w) of MCC (the weight percent are taken with reference to the starting material MCC).

Characterization. DMA: The dried nanocomposite samples are ground in a variable speed mill, using a 1 mm Sieve. The fine powder is used for the DMA experiment. The powder is contained in metal pockets (Perkin Elmer part no: N533-0322) and the DMA is run in the single cantilever mode from 25° C. to 240° C. at a ramp rate of 2° C./min at a constant frequency of 1 Hz. This is a comparative test; different tests will give different numbers of the storage modulus of the same material.

TGA and DSC: Thermogravimetric analyzer (TGA): Thermogravimetric analyses of the various samples (about 10-15 mg) are done with Perkin Elmer (TGA 4000) with a heating rate of 10° C./min up to 800° C. in nitrogen environment. Differential scanning calorimeter (DSC): The sample, 6-10 mg, is analyzed 87 by increasing the temperature at a rate of 2° C./min in nitrogen environment.

SEM: The morphology of the nanocomposite is characterized using a FEI SEM under high vacuum mode and low acceleration voltage. The samples are sputter coated with Au or Carbon.

XRD: X-ray diffractograms of the neat polymer and the nanocoating applications or composite material are obtained on an X-ray diffractometer (PANalytical, X'PertPro). The scan is conducted for duration of 30 minutes for the scan range of 7−70° 2θ.

Results and Discussion: The coating applications or composite material are expected to form immediately upon mixing. The resulting material is expected to be white, hard and different from MCC and PLA in physical appearance. The conditions used to prepare the acid/cellulose mixture, are chosen to open the cellulose structure and free nanocrystalline cellulose (NCC) whiskers and at the same time minimize hydrolysis of amorphous cellulose. Sulfuric acid concentration is 64%, which is the concentration reported (Revol, Godbout, Dong, Gray, Chanzy & Maret, 1994; Revol, Bradford, Giasson, Marchessault & Gray, 1992) to open the cellulose structure and at which NCC is extracted. After 30 minutes in 64% sulfuric acid, it is expected that cellulose amorphous part is dissolved and separated from NCC. PLA is expected to be soluble in THF and amorphous cellulose is expected to be soluble in sulfuric acid with the NCC dispersed therein. At the same time, upon mixing, the THF is expected to act as anti-solvent for dissolved cellulose. Dissolved cellulose which exists together with the partially hydrolyzed cellulose can be precipitated (regenerated) with the addition of an excess of a polar solvent (anti-solvent) like THF (for more information on dissolved cellulose precipitation. Acid mediated networked cellulose:

Preparation and characterization. Carbohydrate Polymers. PLA precipitates as well in the process. The co-precipitating cellulose is expected to enhance bonding between the NCC and the PLA matrix.

DMA, TGA and DSC: Dynamic Mechanical Analysis (DMA) data of the PLA nanocoating applications or composite material are generated with various loading levels of MCC, compared with neat PLA. It is expected to be seen that the storage modulus (E′) of all the blends are significantly improved over a wide range of temperature compared to that of the neat PLA. The storage modulus improvement is expected to be a function of cellulose content. The improvement for all the nanocomposites is expected to be most obvious below the glass transition temperature of PLA (50° C. to 60° C.). The modulus curve is expected to show a drop for all the samples around the glass transition temperature and is expected to flatten out at a much lower temperature for the neat PLA (at 80° C.), whereas for the nanocomposites they flatten out are expected to be at around 130 to 140° C.). The expected steady increase in the storage modulus of the composite, with MCC content is expected to be indicative of the fact that efficient dispersion and blending of cellulose in the PLA matrix is possible even at high loading levels.

Tan δ, also called damping, is a dimensionless property and is the ratio of loss to storage modulus. Tan δ curves for the various samples expected to show that the Tan δ peaks of the nanocomposites will increase in 130 magnitude (highest for NCC50) and shift towards a lower temperature as compared to the neat PLA. Mathew et al (Mathew Aji, Chakraborty, Oksman & Sain, 2006) also noticed this behavior of increase in magnitude of Tan δ peaks in their work with PLA nanocomposites through extrusion method.

The DSC thermograms of various samples are expected to show that the Tg of the nanocomposites are slightly shifted towards a lower temperature as compared to the neat PLA. This is in agreement with the Tan δ peaks shifting towards a slightly lower temperature as compared to the neat PLA. It is also expected to be evident from the thermograms that the introduction of the crystallinity into the otherwise almost completely amorphous PLA, which is indicated by the exothermic activity in the DSC traces for the nanocomposite.

TGA data is expected to reveals that all the nanocomposites have the onset of thermal degradation at a much lower temperature than neat PLA. However the nanocomposites are expected to be seen to be more resilient and have a residual weight of about 5% at 400° C. at which point the PLA is expected to lose all of its weight. The nanocomposites are expected to eventually completely lose their weight at around 750° C.

Nanocrystalline cellulose particles have a greater number of free end chains due to their smaller particles size, introduced as a result of the hydrolysis treatment. The end chains start decomposing at lower temperature (Staggs, 2006), consequently, causing an increase of the char yield of these hydrolyzed samples (Piskorz, Radlein, Scott & Czernik, 1989). Also sulfate groups, introduced during hydrolysis with sulfuric acid could possibly be acting as a flame retardant (Roman & Winter, 2004). It is expected to be observed from the d-TGA curves (derivative weight loss curves), that there is a shift towards the positive direction in terms of the temperature at which maximum weight loss occurs.

SEM images of the nanocomposites are expected to be observed that there is micro/nanoporosity introduced in the polymer matrix, which could be made possible by the solvent escaping/leaching through the matrix during the drying process. Micro/nanoporosity is an important attribute for a potential bio medical application in tissue engineering and scaffolds (Lee et al., 2005; Paul & Robeson, 2008; Traversa et al., 2008). The presence of micro and nanopores could serve as potential active site for cell growth, blood vessel invasion, nutrient and metabolic waste transport. It is worth mentioning here that NCC50 exhibited more pores and variations than NCC30.

Diffraction patterns of Neat PLA and the nanocoating applications, composite material (NCC50) expected to show that the predominantly amorphous PLA is characterized by a broad peak. The nanocomposite is expected to have sharp and intense peaks that are characteristic of crystalline PLA. Thus, the dissolved PLA in THF upon precipitation is expected to be more ordered. This can indicate that PLA precipitates in a slower rate than cellulose and it is possible that cellulose provide the backbone for PLA solidification.

### CONCLUSION

Nanocrystalline (NC) composition and products of the invention, exemplified by polymer blends of poly (lactic acid) (PLA) and cellulose are prepared using a novel solvent mixing method, expecting to yield significant improvement in the mechanical and thermal stability of the generated material as nanocrystalline (NC) compositions and products of the invention. The co-precipitating cellulose during the composite processing method is expected to have enhanced the bonding between the nanocrystalline cellulose (NCC) and PLA matrix. The storage modulus of the nanocomposites is expected to be increased as a function of the cellulose content, indicating good dispersion of cellulose during processing. The nanocomposites are expected to have porous morphology and enhanced crystallinity. The tunable nature of the nanocomposite, prepared using this method, makes it a suitable for various Nanocrystalline (NC) products, as further described herein.

### REFERENCES

Bastioli, C. (2001). Global Status of the Production of Biobased Packaging Materials. Starch-Stärke, 189 53(8), 351-355.
Chazeau, L., Cavaille, J. Y., Canova, G., Dendievel, R., & Boutherin, B. (1999). Viscoelastic properties of plasticized PVC reinforced with cellulose whiskers. Journal of Applied Polymer Science, 71(11), 1797-1808.
Favier, V., Chanzy, H., & Cavaille, J. Y. (1995). Macromolecules, 28, 6365.
Hashaikeh R and Abushammala H. Acid mediated networked cellulose: 194 Preparation and characterization. Carbohydrate Polymers (2010), d. j. c.
Lee, Y. H., Lee, J. H., An, I.-G., Kim, C., Lee, D. S., Lee, Y. K., & Nam, J.-D. (2005). Electrospun dual-porosity structure and biodegradation morphology of Montmorillonite reinforced PLLA nanocomposite scaffolds. Biomatenials, 26(16), 3165-3172.
Lunt, J. (1998). Large-scale production, properties and commercial applications of polylactic acid polymers. Polymer Degradation and Stability, 59 (1-3), 145-152.
Mathew Aji, P., Chakraborty, A., Oksman, K., & Sain, M. (2006). The Structure and Mechanical Properties of Cellulose Nanocomposites Prepared by Twin Screw Extrusion. Cellulose Nanocomposites (Vol. 938, pp., 114-131): American Chemical Society.
Mukherjee, S. M., & Woods, H. J. (1953). X-ray and electron microscope studies of the degradation of cellulose by sulphuric acid. Biochimica et Biophysica Acta, 10, 499-511.
Noishiki, Y., Nishiyama, Y., Wada, M., Kuga, S., & Magoshi, J. (2002). Mechanical properties of silk fibroin-microcrystalline cellulose composite films. Journal of Applied Polymer Science, 86(13), 3425-3429.
Paul, D. R., & Robeson, L. M. (2008). Polymer nanotechnology: Nanocomposites. Polymer, 49(15), 187-3204.
Piskorz, J., Radlein, D. S. A. G., Scott, D. S., & Czernik, S. (1989). Pretreatment of wood and cellulose for production of sugars by fast pyrolysis. Journal of Analytical and Applied Pyrolysis, 16(2), 127-142.
Qi, H., Cai, J., Zhang, L., & Kuga, S. (2009). Properties of Films Composed of Cellulose Nanowhiskers and a Cellulose Matrix Regenerated from Alkali/Urea Solution. Biomacromolecules, 10(6), 1597-1602.
Revol, J.-F. o., Godbout, L., Dong, X.-M., Gray, D, G., Chanzy, H., & Maret, G. (1994). Chiral nematic suspensions of cellulose crystallites; phase separation and magnetic field orientation. Liquid Crystals, 16(1), 127-134.1
Revol, J. F., Bradford, H., Giasson, J., Marchessault, R. H., & Gray, D. G. (218 1992). Helicoidal self-ordering of cellulose microfibrils in aqueous suspension. International Journal of Biological Macromolecules, 14(3), 170-172.
Roman, M., & Winter William, T. (2006). Cellulose Nanocrystals for Thermoplastic Reinforcement: Effect of Filler Surface Chemistry on Composite Properties. Cellulose Nanocomposites (Vol. 938, pp. 99-113): American Chemical Society.
Roman, M., & Winter, W. T. (2004). Effect of Sulfate Groups from Sulfuric Acid Hydrolysis on the Thermal Degradation Behavior of Bacterial Cellulose. Biomacromolecules, 5(5), 1671-1677.
Sinha Ray, S., Yamada, K., Okamoto, M., Fujimoto, Y., Ogami, A., & Ueda, K. (2003). New polylactide/layered silicate nanocomposites. 5. Designing of materials with desired properties. Polymer, 44(21), 6633-6646.
Staggs, J. E. J. (2006). Discrete bond-weighted random scission of linear polymers. Polymer, 47(3), 897-906.
Tharanathan, R. N. (2003). Biodegradable films and composite nanocrystalline (NC) coating applications: past, present and future. Trends in Food Science & Technology, 14(3), 71-78.
Traversa, E., Mecheri, B., Mandoli, C., Soliman, S., Rinaldi, A., Licoccia, S., Forte, G., Pagliari, F., Pagliari, S., Carotenuto, F., Minieri, M., & Di Nardo, P. (2008). Tuning hierarchical architecture of 3D polymeric scaffolds for cardiac tissue engineering. Journal of Experimental Nanoscience, 3(2), 97-110.
Wu Q, L. X., Berglund L A. (2002). In: 23rd Risø international symposium on materials science, sustainable natural and polymeric composites-science and technology.

### Example #5

The recent spread of high-frequency electronic and communication devices has led to a rise in the amount of electromagnetic (EM) waves, causing harmful effects on human body and other nearby devices to malfunction. As concern about the effect of EM wave grows, the devices are required to have electromagnetic compatibility (EMC). Fe-based nanocrystalline magnetic materials such as Finemet alloys have excellent soft magnetic properties including large saturation magnetization and high relative permeability in the high frequency range. One application of the Finemet type alloy is an EM wave absorber, which absorbs the generated EM waves to transform into heat. FeSiBNbCu alloys exhibit excellent soft magnetic properties when nanocrystalline bcc-Fe(Si) phases that is formed by the crystallization annealing are embedded uniformly in the amorphous matrix. Numerous studies have been made on the effect of grain size of crystalline bcc-Fe(Si) phase on the magnetic properties of FeSiBNbCu alloy, in which the optimum magnetic properties can be acquired when the grain size is controlled to the range 10˜15 nm.

The objective of this study is to investigate the effect of the crystallization annealing conditions on the EM wave absorption behavior of a FeSiBNbCu alloy. The relative volume fractions of nanocrystals produced at various crystallization conditions are quantified using a differential scanning calorimeter (DSC) method 2. Experimental procedure Amorphous ribbons with a nominal composition of Fe73Si16B7Nb3Cu1 (at %) alloy are annealed at temperatures ranging from 500° C. to 650° C. for 1 hour under nitrogen atmosphere to investigate the crystallization behavior.

The DSC analysis is carried out using as-fabricated ribbons of 21 mg at temperatures ranging from 300° C. to 720° C. and heating rates ranging from 5 to 2° C./min. After annealing, the ribbons are pulverized and sieved to several classes of particles size. The powder with sizes of <45, 45˜53, and 53˜75 μm are mixed uniformly with the volume ratio of 1:2:7, respectively. Subsequently, the mixture is formed to 2.79 mm thick inductor cores of 6.35 mm outer diameter and 2.79 mm inner diameter under a pressure of 18 ton/cm2 without binder. The permeability is measured under the frequency range of 10˜1000 kHz. In order to identify the interrelations between the crystallization behavior and electromagnetic wave absorption, the ribbons are pulverized prior to crystallization annealing. Crystallization annealing is carried out at 500˜650° C. for 1 hour, followed by a tape-casting to produce a thin sheet of 0.5 mm thick after mixing with a binder. The EM wave absorption properties are measured by a two-port coaxial method using a network analyzer (Agilent Technologies, model N5260A). Results and discussion Crystallization behavior shows the variation of initial permeability of annealed FeSiBNbCu alloy on annealing temperature. The initial permeability increased to 540° C. and decreased thereafter. It has been reported that the magnetic properties of FeSiBNbCu alloy are significantly dependent on the grain size of bccFe(Si) phase. Optimized magnetic properties can be acquired when the grain size is controlled to the range of 10-15 nm using annealing temperatures in the range of 500˜600° C.

### Example #6

Fe-based nanocrystalline powder sheets with dielectric TiO₂ powder additives are provided to improve the characteristics of electromagnetic (EM) wave absorption. The amorphous ribbons of Fe₇₃Si₁₆B₇Nb₃Cu₁ (at. %) alloys are prepared by a planar flow casting (PFC) process, and the ribbons are pulverized using an attrition mill. Fe-based flake powder crystallized at 550° C. for 1 h is mixed with a nano-sized and a micro-sized TiO₂ powder. The powder mixtures are then tape-cast with binders to become EM wave-absorbing sheets. The absorbing properties of the fabricated sheet sample, such as complex permittivity and permeability, are measured by a network analyzer. The properties of EM wave absorption improved with the increase of TiO₂ powder as micro- or nano-sized powder in the mixture. The mixture with micro-sized TiO₂ powder is slightly more effective in causing power loss of EM waves than the mixture with nano-sized TiO₂ powder.

### Example #7

Nanocrystalline soft magnetic Fe₇₃Si₁₆B₇Nb₃Cu₁ (at. %) powders are mixed with fine multi-walled carbon nanotube (MWNT) powders and polyurethane based binders. The mixtures are tape-cast, dried and then cold-rolled to form EM wave absorption sheets. The MWNT powders are added to the Fe-based powders up to 1 wt. % to improve the EM wave absorption properties. The processed sheets with 0.5 mm in thickness are cut into toroidal shape to measure the S-parameter, permeability, permittivity, and power loss at the high frequency of 10 MHz to 10 GHz. As a result, improved absorption properties are obtained from the sheets incorporating MWNT. The results are caused by the increase of dielectric loss of the absorption sheets, which is due to the addition of MWNT powder inducing a notable increase in complex permittivity.

### Example #8

The amorphous (at %) alloy strip is pulverized using a jet mill and an attrition mill to get flake-shaped powder. The flake powder is mixed with dielectric powder and its dispersant to increase the permittivity. The powders covered with dielectric powders and its dispersant are mixed with a binder and a solvent and then tape-cast to form sheets. The absorbing properties of the sheets are measured to investigate the roles of the dielectric powder and its dispersant. The results showed that the addition of powders and its dispersant improved the absorbing properties of the sheets noticeably. The powder sheet mixed with 5 wt % of powder and 1 wt % of dispersant showed the best electromagnetic wave absorption rate because of the increase of the permittivity and the electrical resistance.

The foregoing is illustrative of the present invention, and is not to be construed as limiting thereof. The invention is defined by the following claims, with equivalents of the claims to be included therein.

1. A method for producing nanocrystalline (NC) products comprising a nanocrystalline (NC) composition, comprising:
(a) combining nanocrystalline cellulose (NCC) and at least one of nanocrystalline (NC) polymers, nanocrystalline (NC) plastics, with at least one substrate and optionally further comprising at least one component or additive, the at least one NCC in at least a partial dry form, to provide a nanocrystalline (NC) composition, wherein at least the NCC, is substantially distributed on the surface of the substrate, and wherein the at least one NCC comprises at least one branched polymer having at least a first polymer chain extending from at least one nanocrystalline (NC) cellulose core and at least one branch diverting away from the first polymer chain;
(b) processing the nanocrytalline (NC) composition using at least one of vapor processing, solid state processing, or liquid processing; and
(c) processing the nanocrystalline (NC) composition with at least one metal alloy and at least one coating agent, structural bulk material, ceramic, to increase at least three of strength hardness, compressibility, strength, corrosion resistance, higher electrical resistance, increased specific heat capacity, ductility, and lower thermal conductivity.

2. A method according to claim 1, wherein the nanocrystalline (NC) product comprises a combination of two or more of nanocrystalline cellulose (NCC), nanocrystalline (NC) plastics, nanocrystalline (NC) polymers or nanocrystals of cellulose composites or structures.

3. (canceled)

4. (canceled)

5. A method according to claim 1, further comprising (c) tracking the reflection or absorption of the wavelengths from one or more sensors to provide diagnostic or therapeutic information, wherein the information comprises one or more of changes in a reflection spectrum generated from reflecting the wavelengths off of said skin, joint or tissue.

6. (canceled)

7. (canceled)

8. A method according to claim 1, wherein the processing in step (c) provides at least a 10% increase in at least one of the tensile strength or hardness of at least a portion of the resulting nanocrystalline (NC) product.

9. A method according to claim 1, wherein the at least one nanocrystalline cellulose (NCC), nanocrystalline (NC) polymers, or nanocrystalline (NC) plastics, comprises at least one first branch of the at least one first polymer chain bonded to an nanocrystalline (NC) cellulose core and the first polymer chain is made up of one or more monomers selected from:
vinyl acetate, acrylic acid, sodium acrylate, ammonium acrylate, methyl acrylate, acrylamide, acrylonitrile, N,N-dimethyl acrylamide, 2-acrylamido-2-methylpropane-1-sulfonic acid, sodium 2-acrylamido-2-methylpropane-1-sulfonate, 3-acrylamidopropyl-trimethyl-ammonium chloride, diallyldimethylammonium chloride, 2-(dimethylamino)ethyl acrylate, 2-(acryloyloxy)-N,N,N-trimethylethanaminium chloride, N,N-dimethylaminoethyl acrylate benzyl chloride quaternary salt, 2-(acryloyloxy)-N,N,N-trimethylethanaminium methyl sulfate, 2-(dimethylamino)ethyl methacrylate, 2-(methacryloyloxy)-N,N,N-trimethylethanaminium chloride, 3-(dimethylamino)propyl methacrylamide, 2-(methacryloyloxy)-N,N,N-trimethylethanaminium methyl sulfate, methacrylic acid, methacrylic anhydride, methyl methacrylate, methacryloyloxy ethyl trimethyl ammonium chloride, 3-methacrylamidopropyl-trimethyl-ammonium chloride, hexadecyl methacrylate, octadecyl methacrylate, docosyl acrylate, n-vinyl pyrrolidone, 2-vinyl pyridine, 4-vinyl pyridine, epichlorohydrin, n-vinyl formamide, n-vinyl acetamide, 2-hydroxyethyl acrylate glycidyl methacrylate, 3-(allyloxy)-2-hydroxypropane-1-sulfonate, 2-(allyloxy)ethanol, ethylene oxide, propylene oxide, 2,3-epoxypropyltrimethylammonium chloride, (3-glycidoxypropyl)trimethoxy silane, epichlorohydrin-dimethylamine, vinyl sulfonic acid sodium salt, sodium 4-styrene sulfonate, caprolactam and any combination thereof;
non-ionic, water-soluble monomers selected from one or more of: acrylamide, methacrylamide, N,N-dimethylacrylamide, N,N-diethylacrylamide, N-isopropylacrylamide, N-vinylformamide, N-vinylmethylacetamide, N-vinyl pyrrolidone, 2-vinyl pyridine, 4-vinyl pyridine, epichlorohydrin, acrylonitrile, hydroxyethyl methacrylate, hydroxyethyl acrylate, hydroxypropyl acrylate, hydroxypropyl methacrylate, hexadecyl methacrylate, octadecyl methacrylate, glycidyl methacrylate, 3-(glycidoxypropyl)trimethoxy silane, 2-allyloxy ethanol, docosyl acrylate, N-t-butylacrylamide, N-methylolacrylamide, epichlorohydrin-dimethylamine, caprolactam, and any combination thereof;
anionic monomers selected from one or more of acrylic acid; methacrylic acid, 2-acrylamido-2-methylpropanesulfonic acid (AMPS), sodium vinyl sulfonate, styrene sulfonate, maleic anhydride, maleic acid, sulfonate itaconate, sulfopropyl acrylate, polymerisable carboxylic or sulphonic acids, crotonic acid, sulfomethylated acrylamide, allylsulfonate, sodium vinyl sulfonate, itaconic acid, acrylamidomethyl butanoic acid, fumaric acid, vinylphosphonic acid, vinylsulfonic acid, vinylsulfonic acid sodium salt, allylphosphonic acid, 3-(allyloxy)-2-hydroxypropane sulfonate, sulfomethyalted acryamide, phosphono-methylated acrylamide, ethylene oxide, propylene oxide, and any salts or combinations thereof; and
cationic monomers selected from one or more of dialkylaminoalkyl acrylates, methacrylates and their quaternary or acid salts.

10. A method according to claim 9, wherein at least one second branch of the first polymer chain comprises a different selection of monomers than the at least one first branch of the at least one first polymer chain, the different selection being different in at least one selected from monomer type, or monomer ratio.

11. (canceled)

12. (canceled)

13. A method according to claim 1, wherein the combining step (a) comprises blending the nanocrystalline cellulose (NCC), nanocrystalline (NC) polymers, or nanocrystalline (NC) plastics, with a polymer to provide a blend, and adding the blend to the substrate, component, or additive, wherein the blend is substantially distributed on the surface of the substrate, component, or additive, and wherein the at least one nanocrystalline cellulose (NCC), nanocrystalline (NC) polymers, or nanocrystalline (NC) plastics comprises a nanocrystalline (NC) cellulose-core which consists essentially the nanocrystalline (NC) crystallites having a diameter of 5-10 nm.

14. (canceled)

15. (canceled)

16. (canceled)

17. A method according to claim 1, wherein the nanocrystalline (NC) cellulose is selected from one or more of naturally occurring crystals obtained by separating the crystalline cellulose regions from the amorphous cellulose regions of a plant fiber.

18. A method according to claim 17, wherein the nanocrystalline (NC) crystallites are 100-500 nm length and comprise between 85% and 97% of the nanocrystalline (NC) cellulose.

19. A method according to claim 1, wherein the combining step (a) further comprises:
providing an aqueous mixture comprising partially hydrolyzed forms of the nanocrystalline cellulose (NCC), nanocrystalline (NC) polymers, or nanocrystalline (NC) plastics;
providing a solution comprising the substrate, component or additive in a polar organic solvent;
combining the mixture with the solution to form a precipitate; and
washing the precipitate with water to remove solvent and dissolution media and produce a wet composite of the nanocrystalline (NC) composition; and
drying the wet composite to produce a dry composite as the nanocrystalline (NC) composition.

20. (canceled)

21. A method according to claim 19, wherein the washing step is carried out until the wet composite has a pH between 6 and 7.

22. (canceled)

23. A method according to claim 19, wherein the dry composite produced is rigid and has (i) a storage modulus of between 1-5 and 20-35 gigapascals, at a temperature of 20 degrees C., or (ii) a storage modulus between 0.1-1 gigapascals and 10-20 gigapascals, at a temperature of 100 degrees Centigrade.

24. A method according to claim 19, wherein dry composite is porous and has a density of 0.01 to 10 grams per cubic centimeter and a residual weight of about 1-20% at a temperature of 400 degrees C.

25. A method according to claim 1, wherein the metal alloy is selected from iron or titanium based nanocrystalline magnetic materials that absorb or reflect electromagnetic energy in the range of 10 to 100 kHz that are provided with crystal diameters in the range of 10-15 nm.

26. A method according to claim 25, wherein the iron or titanium based nanocrystalline magnetic material is selected from a FeSiBNbCu alloy, dialectric TiO2 powder, or BaTiO3 powder.

27. (canceled)

28. (canceled)

29. (canceled)

30. (canceled)

31. A product or device comprising at least one nanocrystalline (NC) product made according to a method of claim 1.

32. A product comprising at least one nanocrystalline (NC) product made according to a method of claim 1, further comprising at least one fertilizer, pesticide and/or herbicide, micronutrient, microingredient, growth hormone, crop additive, organic or genetically modified, engineered plant seeds, plants, cellulose in foods, or food ingredient.

33. A method for plant, seed, or food production, comprising:
(a) providing at least one nanocrystalline (NC) product made according to a method of claim 32;
(b) using the at least one nanocrystalline (NC) product for affecting one or more of plant or plant seed germination, growth, crop yield, plant product quality, growth rate, water uptake, fertilizer uptake, herbicide tolerance, insect tolerance, drought tolerance, and vegetation production; for use in one or more of agricultural products, industrial products, agricultural based products, compound feed, animal feed, agricultural commodities, fruits, food ingredients, food products, food packaging applications, food applications, food additives, organic food additives, organic products, soy bean, protein, soy products, milk production and/or dairy products.

34. A method for plant, seed, or food production, comprising:
(a) adding at least one providing at least one nanocrystalline (NC) product made according to a method of claim 32 to the soil or nutrients used to generate, mutate or cross-breed or generate seeds or plants configured for growing plants in food production.

35. A method according to claim 34, wherein the at least one nanocrystalline (NC) product is used in one or more fertilizer, pesticide, herbicide, micronutrient, microingredient, growth hormone, crop additive, organic or genetically modified, engineered plant seed, plant, food, or food ingredient.

36. (canceled)

37. (canceled)
